SEQUENCE LISTING (293 sequences)

SEQ ID NO: 1 - DNA, 20 bp, Homo sapiens
ctggctcttcctgccctaat

SEQ ID NO: 2 - DNA, 20 bp, Homo sapiens
tttctggtgggcatgtatgt

SEQ ID NO: 3 - DNA, 197 bp, Homo sapiens
ctggctcttcctgccctaataccggctgcccgtacgggactgctcacctcctgcagggagccggacgtctgtggcgatctccctcccgccatgacaccccctacctgtcctccatcatatgggacacacacacacacacacacacacccctacgcacacccacaccccacatgcacatcatacatgcccaccagaaa

SEQ ID NO: 4 - DNA, 22 bp, Homo sapiens
tggaaggccctctttaacagta

SEQ ID NO: 5 - DNA, 20 bp, Homo sapiens
gccaccctaaccctaccaag

SEQ ID NO: 6 - DNA, 159 bp, Homo sapiens
tggaaggccctctttaacagtaggtatttgaagtgttataaaaaaaaaaaaaaggtgaatttttcttttatttctcagtttgaaagaacagctttattcttggttattcctaatgtccacctagtcctcttttacttttcttggtagggttagggtggc

SEQ ID NO: 7 - DNA, 26 bp, Homo sapiens
cacatatttgtaggaactctcaaagc

SEQ ID NO: 8 - DNA, 23 bp, Homo sapiens
gcattacacaacctctttaccag

SEQ ID NO: 9 - DNA, 189 bp, Homo sapiens
cacatatttgtaggaactctcaaagcgttttccaataagaattaaattgcaaatgacaattaagtttttaaaccagtccccaaaatcttaatttgattgtagttacaaaagaactagttcaagttcgtgtgtgtgtgtgtgtgtgtgtgtgtgtgtgtgtgtgtgtctggtaaagaggttgtgtaatgc

SEQ ID NO: 10 - DNA, 26 bp, Homo sapiens
aaaccatttaacacaggataaactca

SEQ ID NO: 11 - DNA, 21 bp, Homo sapiens
gggtacacttccatctgacca

SEQ ID NO: 12 - DNA, 185 bp, Homo sapiens
aaaccatttaacacaggataaactcatagttacattaaaagataggaaaatacacacacacacacacacacacacacacacataccacacaaacacacatacatgcacacacacacacatttcggttactagttggtttcagtcaaggataaaaattcttaaattggtcagatggaagtgtaccc

SEQ ID NO: 13 - DNA, 21 bp, Homo sapiens
gacggatttcagagtcaccaa

SEQ ID NO: 14 - DNA, 20 bp, Homo sapiens
tgcagaagtcctctgtttgc

SEQ ID NO: 15 - DNA, 381 bp, Homo sapiens
gacggatttcagagtcaccaaggatggccaatgatgtggtggttaagagcatgaacactggtgcttcacggcctgggttcgggtcctgactcaatgcttactggctgtgtgttttggaaaaggcccttaatctctctctgtttcagcttcccatctataaaatgtggataatgacaatacatacctcatgcagttattagaaagattcaatgagttattatttataaactgctcaaaacagcaccatgtacatagaaagtgctcgttaaatggatggatggatggatggatggatggatggatggatggatggatgggtgcatggatggatggatgaatagatcaatggatggataaacaggcaaacagaggacttctgca

SEQ ID NO: 16 - DNA, 20 bp, Homo sapiens
ccgatgggtatttgttccac

SEQ ID NO: 17 - DNA, 20 bp, Homo sapiens
gaggaaaggacacagggaca

SEQ ID NO: 18 - DNA, 170 bp, Homo sapiens
ccgatgggtatttgttccacgttttctattttagtcagttctacctttagagttctttacacacacacacacacacacacacacacacagcatctcacttaattttattcatccttcaaagttcatcttaggtcatttcttcccctcctttgtccctgtgtcctttcctc

SEQ ID NO: 19 - DNA, 25 bp, Homo sapiens
tttctgaaactccataaactcatca

SEQ ID NO: 20 - DNA, 25 bp, Homo sapiens
gaactctaccaagtttgtcttctgg

SEQ ID NO: 21 - DNA, 178 bp, Homo sapiens
tttctgaaactccataaactcatcagattatttttactttaaatgctataaacctgaagtatttctttacttgacacacacacacacacacacacacacacacactcatacacatttcatacttttgcatcaaagctggtcataaaattggtaccagaagacaaacttggtagagttc

SEQ ID NO: 22 - DNA, 20 bp, Homo sapiens
acatcctcttccagcagaca

SEQ ID NO: 23 - DNA, 21 bp, Homo sapiens
tggaagctgctaaggagaaca

SEQ ID NO: 24 - DNA, 373 bp, Homo sapiens
acatcctcttccagcagacacccacaaagtactattcagtttgcactgtaacaaatgttatttctgggcctcagtgagataatggtaagtgaatgtaattcactctcattaatatattaaaatgagtatgaattttaaattagaaggaacaagtccatggtcgaagaattgaaattggatttatgtgatttgacttcgtagtcatttatctacaatactcattgatactaattgcacagtttcctcttcacattcccactgggcagcacgtgtgtgtgtgtgtgtgtgtgtgtgtgtgtgtgcatgtgttatgtatttgaattaaaagacactgagaagtagcgcctaaaaatgttctccttagcagcttcca

SEQ ID NO: 25 - DNA, 20 bp, Homo sapiens
tcttccgccctgtgtctatc

SEQ ID NO: 26 - DNA, 20 bp, Homo sapiens
tcaagcggaagatttgtcct

SEQ ID NO: 27 - DNA, 257 bp, Homo sapiens
tcttccgccctgtgtctatctaggtcaggcttctcaaacctcaccatggcagatgcatcatttggagaccttgtgaaaatgtagactctgattccctaggtcaagggctgagattctgcatttctttcaaaatcccaggtgatgctgctgctgctgctgctgctgctgctgctgctgctgctggtctagaccacattttcagaagtaaggatttaaacaatcagcacccagggagctaggacaaatcttccgcttga

SEQ ID NO: 28 - DNA, 20 bp, Homo sapiens
tcttccgccctgtgtctatc

SEQ ID NO: 29 - DNA, 20 bp, Homo sapiens
tcaagcggaagatttgtcct

SEQ ID NO: 30 - DNA, 257 bp, Homo sapiens; identical to SEQ ID NO: 27

SEQ ID NO: 31 - DNA, 20 bp, Homo sapiens
gaaagaagctgcaaacagca

SEQ ID NO: 32 - DNA, 20 bp, Homo sapiens
gttgatccagaggtcggtgt

SEQ ID NO: 33 - DNA, 366 bp, Homo sapiens
gaaagaagctgcaaacagcaacctggtctttgactgcacaataatcctctaaggttcagatcgtctcaaccagagttaaattctaacagagagagagagagagagagagaacgagagagagagagagagattgatctggattcaggcttcctagatgcagtctatccaactcaggcagcagtgaacgaggaatacaggctctttcccacatgtttggaatcctggccctgagccctgagctgtgcattccatttatcctctttgtgggctgaacagatgaaattgctttagctaaaggaagtggcacgaatttacttatttattagatgtgcaggatacatccatcacaccgacctctggatcaac

SEQ ID NO: 34 - DNA, 20 bp, Homo sapiens
ccacttccaatgcagacctt

SEQ ID NO: 35 - DNA, 27 bp, Homo sapiens
tgcatgtatataatgagtagggagaga

SEQ ID NO: 36 - DNA, 412 bp, Homo sapiens
ccacttccaatgcagaccttgttctataaagaatatctagcactttcacatgtttctgaaggaagtgtattatttgtagcccctttttggagaaaaattattctgcttcaaggtatttattctacggatatactaacatgtgtcaaagaatacaatctcgagtctttagtgttgtttctggagtaaaatattgaaaataatcaaaatgctcatcaatagaaggctggctaaataaagtcggcttatataatggaatatcacgtggccagtaaaaaagaatcaaacagctctctatatatcaatattttgcagtgtatatattaaacttttaaaaagcatacaaaacactgtttctattctactaccattttggggtgggagactttctctccctactcattatatacatgca

SEQ ID NO: 37 - DNA, 20 bp, Homo sapiens
tgccggtataggtgtgactg

SEQ ID NO: 38 - DNA, 22 bp, Homo sapiens
tgtttcttgctgatttcttcca

SEQ ID NO: 39 - DNA, 293 bp, Homo sapiens
tgccggtataggtgtgactgaacaatacatccattggtagactactatgctatatttgtaggatatactataacattctacacacacacacacacacacacacacacacacacacacacacataataatcttctataacagggttctaactgttcatatggaggcatctcaaaaatatattttgaagtgatcaaatgcgaggtgcagaacaaggagtacagcatgatctcattcctgttaaaatatatgcaaatacatgctttattttccctggaagaaatcagcaagaaaca

SEQ ID NO: 40 - DNA, 20 bp, Homo sapiens
tcacctcttcacggacaaag

SEQ ID NO: 41 - DNA, 23 bp, Homo sapiens
tcttaagtccatctctgcacaag

SEQ ID NO: 42 - DNA, 309 bp, Homo sapiens
tcacctcttcacggacaaaggggaataacctcagagtatgacataaaatatccactaaataaaaaatactggttgggtatggtggctcacgcctctaatcccaacattttgggaggctgagtggggaggaccatttgaggccaggagatcaagaccagcttgggcaacataaaaaggccctatctctatttcacaaacacacacacacacacacacacacacacacacacacacacaaaaagaaaaaaaaaattaaagaaaaaatactttaggaaattctaaactacttgtgcagagatggacttaaga

SEQ ID NO: 43 - DNA, 20 bp, Homo sapiens
ttcagatggctcagggtagc

SEQ ID NO: 44 - DNA, 20 bp, Homo sapiens
agaagctgcaggatggagaa

SEQ ID NO: 45 - DNA, 265 bp, Homo sapiens
ttcagatggctcagggtagccccacccacactccctcccagagacagtcaattttacaacaaatattctgagttatctaggctgaccctttttttcccccacagaggaggaaatgggctcaaagtaagtgacttctcaatcagccatcaaagtagagtagaggcaggactgctaactccccgtgtggaatgtattcccctgtgatcatcacctgtactcacactgttcttgagccagaccccaaattctccatcctgcagcttct

SEQ ID NO: 46 - DNA, 20 bp, Homo sapiens
agccagaaattgaggaagtg

SEQ ID NO: 47 - DNA, 20 bp, Homo sapiens
ctgcaagctctttcagttga

SEQ ID NO: 48 - DNA, 109 bp, Homo sapiens
agccagaaattgaggaagtgctcaaacacacacacacacacacacacacacacacacacacaaaggagtatgtcataggtacagagaagtcaactgaaagagcttgcag

SEQ ID NO: 49 - DNA, 21 bp, Homo sapiens
gacggatttcagagtcaccaa

SEQ ID NO: 50 - DNA, 20 bp, Homo sapiens
tgcagaagtcctctgtttgc

SEQ ID NO: 51 - DNA, 381 bp, Homo sapiens; identical to SEQ ID NO: 15

SEQ ID NO: 52 - DNA, 1001 bp, Homo sapiens
ctcaaaaaccaaaggtgtgatgaaggtgctacagtttgaactctttaaaggaaggcatcggccatatagagtgagccacaggggaggacttctcccgtttccctgtagaatgggttaccaagttaaaggagtcaattatcccgtcctatctggagaaagcattcctcagatgaataaactggaaacggaaaactggagaaggtgtttttatttcttttcgtaattaggacatcatttacaagacttatatttcttggatgttccccaaatttttcacatagagctggcattactagaaacttaaatacttgttgcttttaattatattgaattccaccgtgggagcttaaaggctaggcattttgtgatgggtgtgcattctactcccaaatgtaataactagaatagaaattccagaaaaggaaaagtatttatcaaacactgaagctgctttgagaaatggctttgtcaagttaactggttatcattagatttattacrgtggttaggaaaaactgacctcgtagatgtctgtctataacaatgcaatcatctgcttagaataatgccccgcgttagacagctgtaaacacaagaactttcccttgcgagttcaataatcttagcaacagttctctttccaaacaggccaagaaagatatgttgctttgggaaactggaaatcaacagaccaaaacagccagaagaaatgggtggagagaagatagagcccgttcactctgcagtctccgcaggggtacagagtgatggcagccatgggtgcccttgtaagtctctgtcccagctcccaaccctgccacctggggccaccaccatgattccctgcccggccctgcacacatgggctgcaaaaatgctgaggaaaaaggagatttcaaactaattcatccccaagttacaaacgtggttcatggagctttagtaaaaattatttttaaatttttactttgatccacagacatgcgacttgaaccagattc

SEQ ID NO: 53 - DNA, 1001 bp, Homo sapiens
tgcattccagcctgggtgacagagcaagaccctgtcacacacgtacacacacgcaaaaatgacagagaggcagaattctcctaagtggaaatgaaatacagaataccatgatttagttttcctgtagttctttccctaacgtttgacaatagctttccttttgggtgatcagtgtcctttggttttacctcatagccctgtgaggttgccgtgttgagtcttgttttcataccacattgacggtcctttctagtggcctgaaggtttttgttattattttgaaaagctttattgatatataattcacataccatacagttcactcatttgaagtggacatttcaatatttggaagcctattcacagcatatgcgcaaccattaccacagccaattttaggataattttttctttctgttttttactgtggggttttgcagtgaaaaccagaaaacctgctagacaaattccaaaagagctgtaacacgcgatttcagaacrtttaatcacctcaagaagaaacctgaaggatccttccgtcgccgcctctatctctgtcccctccagccctcagaaacaactaatctatgttctttctttaaaaaaaaaaaatctttgaagccttcataaatcagccctttgatttaaatctccatctcactccgccactatttttgatcaattcttcaccagagcttcatcttgacatgtgctctgccacagtgctaaggaacagagtgaccccccaccccactcccgacagaagcagccccagagagagaagcagagggtcagggtcagggtcagcaccgagtgtgctcgggtgaactgcaagtcttgacttagtcttgaggacctcctcagtcttgcaccccttccttcagcaacacctgccgggatgcgtctttcggcctcctctgaaatacaaaaacattttgtggtctagctgctcactgtattttcactctgtggttttctttaatttacacccctcttctact

SEQ ID NO: 54 - DNA, 1001 bp, Homo sapiens
tacacatgaaagttgacttggctgaatataaaatgcttttagatgcttctccattgttttctgactgtagtagtacaaagaggtcagaagtcagtctggtatttgttcttccatcaacaacttgtttgggattgggggtggtatttcctgtgtggataacttgcagcacttcctcttcttcttttttttttttggtctttgtaactaaaaaatgtggtcaatatgtgtctaggtgtgggtgttttaaaattgattttacctggaatttgtgagcccagtcaatctatatactccagtctttttccagcctgaaaatgttttcttcaataaagtcattatcacttatttctgttgttctggtttcttgattagtaatactgttaagtcttaaactgaattcccattgtttatatttatcagaatctatcacttttcttagttaactatttattttcacttatcatgtctaactctatgctcttttcctgtaaaagacctctyaaggttcacctccaaatcaacgtttccattttctacactgtcaattttgcttctttccacctccatgagggattttaattcttggattgcatttttttttgacatccattcttatcgcatctctctttgtatcttgtcttcctaacttttcatcttatctctgtgtgtggttttctgtaattcatagaccatgtcttcctgcaatccaagatgtttttaaaattttcttttgtttcctgtagtaaaactatttcacggggaaatttggcaaactggtgatgcccttggaatagtcaccatacacttgatagtttacaaatgtgtcagcatgtaaatttgtgtttcattttcatataccccaacatcttataatggagggaaaggcaagtctttgttttccaaggtcttggctcttttagccgcaaagtggtgctaacagctccttcatgttccaggagcctctggagaaactgcttccataaagtgtttgggaattctgg

SEQ ID NO: 55 - DNA, 20 bp, Homo sapiens
gctttagaaggcggaggtag

SEQ ID NO: 56 - DNA, 20 bp, Homo sapiens
gagggggttaaaggtgtcat

SEQ ID NO: 57 - DNA, 221 bp, Homo sapiens
  misc_feature 113, 128: n = A,T,C or G
gctttagaaggcggaggtagtaggtaggtagataggtagatgatagatagatagatagatagatagatagatagatagatagatacagatatacagatagagttgtatacatnaaatatatattatgnaaatatatacataagaaggatgacattaacaggcattttctagtaaattaagagttagccaggaaatgtaaccatgacacctttaaccccctc

SEQ ID NO: 58 - DNA, 1001 bp, Homo sapiens
gaagaacagaggcgactcacagtttccgtgataatgataagctgcagacgactatttagagcatcccaacatttatttcaaagtaaagacagtagaaaacaactggactgcaagatgggagtcttggtcactcactgtgtgatattaacagagtcactcgacctccttggactcagtttcttcttgtctaaaatggggctgttgtcctcactcagctctaaaggctcctcttaaagcaaaagtgatggttcttggaatttcttttatttctccagtgagaatcacttcaatcttcaggcaagatacctgcctgtctcctgcccctctctcccattctgtcccggatattgtgaagctacttcttcagtttcatgaacctggattttggccaaacccttgatcattcatcttagaagctagatttccttttcgaagccacaactctgggaaaggtcttcacagccagttcctgatgttgctgagctgatcttgtccattctsagtcaaggtaggatgacagctccccgtgagaaaaaaaaataggtgttgcataagagaacatcttggctatttatgaaagattttctatgcttctgttttaagtttgtttttcaattacaaaagggactcattcttttgtataaaatttggaaagctaagttaagtttagagaagagggtaaaatcattcttaatcccataattctaccatggagaaattttgttagtattttggtgtattctcaatttcctctgcagttttttacattgttgaaatcatgctatttatactatttcatcctttcttcccactgaaaattgtatgataagcatttcctcatgtcactgaagtcactgataagtaatattttaatagcaccataatattttattttgtgggttttgtcctaaggttgaacagataggttgtttctagttttatttttttaaaaatattattagcaatgctgagatgaacatttgtgtgtatatatctctgga

SEQ ID NO: 59 - DNA, 22 bp, Homo sapiens
gaccatgattaagcaaaacaaa

SEQ ID NO: 60 - DNA, 19 bp, Homo sapiens
tcgctcagaaacaaaccaa

SEQ ID NO: 61 - DNA, 222 bp, Homo sapiens
  misc_feature 32, 113, 116: n = A,T,C or G
gaccatgattaagcaaaacaaataacacaaancaaaaatcttcctatttcccagagtcctgggtttatcacaaatgctattaaggttacgagttttgtcctttgataaaaganganccacgtttggaaattgtcattaccctttatttttcaacacacacacacacacacacacacacacacacacacacacacacacactcctacattggtttgtttctga

SEQ ID NO: 62 - DNA, 1001 bp, Homo sapiens
caaggaattgctacagcacatgctgttggggtgcctggtgtggggctcctagagggctcctttaagcctgcctctccctctctggtagttgtaactagaaagggtattcaggaaaaaacacaaatttctctctaggtcttctcagcctccttaccaggcagcaagagctgagagaacttggagtagaatattctaaaccttgctcctgtatctgctttcttgccttaagagaaaaatcttttcccccagattctgctgtctttacactcattctcatcttaccgatctctttaaaatttcagtcattctcggagaccatagggcagaacgcaaagaacataacataggagtcaaatggagccgaacacttcagtcactcacgtgatggctgtgtgtccttgggtaagttctgtagcttctctgagccccaacttccttataacatcattgaagtcctaacagctgtgagaatgacacatgatgcctgcaaatttcataaawcagtgcttggtggttagtagttggttttgaaaaggttatgctaaaattccagggtgatacttttctaggtagtccctttttgcaggtagctttcagaggtaaaacctcagaccccaacacggtccacctctgcatttttttttttttttttttgacatggagtctcgctctgtgcccaggctggagtgcagtggcgtgatgtcggctcactgcaagctccgcatcccgggttcacgccattctcctgcctcagcctcccgagtagctgggactagaggctcaggacaccacgctcggctaattttttgtattttttagtagagaccgggtttcaccgtgttagccaggatggtctcgatcttctaacctcgtgatccgtccgcctcggcctccctaagtgctgggattacaggcgtgagccaccgcgcccggcctttttgtttgcttgttttttgagatggtttcttggtctgttgcccagactctagtgcagtggcacg

SEQ ID NO: 63 - DNA, 20 bp, Homo sapiens
gtcctctgggtgtttgcagt

SEQ ID NO: 64 - DNA, 20 bp, Homo sapiens
caggctctgctctccttagc

SEQ ID NO: 65 - DNA, 259 bp, Homo sapiens
gtcctctgggtgtttgcagtgctgagtgcattggggtttgtgtgtgtgtgtgtgtgtgtgtgtgtgtgtgtgagagagagagagacagagagagggagagaggagcacagtagcttgtgcaaagacctcctttgctatagaagcctgattccaaacctgtcttctttcccagaagtaattacaatacacattgctgcttctcttcaatgtgcctgtgttctggaagctgtgtgtctccagctaaggagagcagagcctg

SEQ ID NO: 66 - DNA, 25 bp, Homo sapiens
caaatcaatataccacttcaggact

SEQ ID NO: 67 - DNA, 20 bp, Homo sapiens
gcagtaggcacatggcaaat

SEQ ID NO: 68 - DNA, 168 bp, Homo sapiens
caaatcaatataccacttcaggactgggtgtgtgtgtgtgtgtgtgtgtgtgtgtgtgtttcttctcttccctcccctcctccccttcctcctcctccttctttagacaagtactatgttttaagatttaggtatataattctacttaatttgccatgtgcctactgc

SEQ ID NO: 69 - DNA, 24 bp, Homo sapiens
gagaatgcttgaccccaaaaaatc

SEQ ID NO: 70 - DNA, 24 bp, Homo sapiens
cctaagagagtgctatgtgctccc

SEQ ID NO: 71 - DNA, 162 bp, Homo sapiens
gagaatgcttgaccccaaaaaatcaagatcaaagatcagcctgggcaacaaagtgagaccctgtctacacacacacacacacacacacacacacacacacacagacacacacaaagtatacccaagtactacaaaaatgggagcacatagcactctcttagg

SEQ ID NO: 72 - DNA, 22 bp, Homo sapiens
cccagataagatcttggttcag

SEQ ID NO: 73 - DNA, 20 bp, Homo sapiens
accacggtgaccctcaatta

SEQ ID NO: 74 - DNA, 253 bp, Homo sapiens
cccagataagatcttggttcagaaaaaaatgttaaaacagccagtattatagaatttatatttaaattataatatagtctatataatttatatctaaaacgtgtgtgtgtgtgtgtgtgtgtgtgtgtatgaagttaggtggtaaataatccaattgacttgttaagttttgggctaataatatgcagagttatcagcaatagggaagactgaagactttgctcctcttagagtaattgagggtcaccgtggt

SEQ ID NO: 75 - DNA, 23 bp, Homo sapiens
cttcagattggaaagtcaggaga

SEQ ID NO: 76 - DNA, 22 bp, Homo sapiens
aaagctctcagcaaggacttta

SEQ ID NO: 77 - DNA, 240 bp, Homo sapiens
cttcagattggaaagtcaggagagattttcaatcttcgtttcttcccactaaatgtactaaaatagaaactgttgttgtttttaactaaaatcagagcagactggaattacggaaaagaatattatgaatggttctatatatatatatatatatatatatatatatatatatatatatatgtagacagaacttaacatttatgtttttttgttatttttaaagtccttgctgagagcttt

SEQ ID NO: 78 - DNA, 20 bp, Homo sapiens
gatcttggctggcagaagaa

SEQ ID NO: 79 - DNA, 21 bp, Homo sapiens
gctccgagaagaacatatgga

SEQ ID NO: 80 - DNA, 289 bp, Homo sapiens
  misc_feature 241: n = A,T,C or G
gatcttggctggcagaagaatagaatcaagaaaattttctcaaaggaagaagagaattgcactgaagctttgggaataaaaagaagttagccacgcaaagatagagtcttccaggtgaaggaaaggcatatacaaaggaatggcagtaagaaagaacaaatcatgttcaagaagctggaaggagttggccgtggctgagcgttgggtgagatgacagtggagaggtgaagaggccgacagngggggcagggccagaagcagagagggttccatatgttcttctcggagc

SEQ ID NO: 81 - DNA, 20 bp, Homo sapiens
tgcatatgtctggcctgtct

SEQ ID NO: 82 - DNA, 20 bp, Homo sapiens
tttcttcctggctttccttg

SEQ ID NO: 83 - DNA, 350 bp, Homo sapiens
tgcatatgtctggcctgtctcctggcacctctgctttctcttcatgaagcacccaggtaacccattatccagagctcttactaattctgttcagtgtttgtttcttgctgctggggcaggaggtggagaacaaagggaatgagggaacattgagaaatttctcttcattgtgaccagctagggcaaattgtccttggtcttctaacccagcagcaagtattcattgcgaaaacacacacacacacacacacacacacacacacacacacacacacacgcatgccatttatgcaaaacacattagtgagggtatttttcctctttaagcaccaaggaaagccaggaagaaa

SEQ ID NO: 84 - DNA, 21 bp, Homo sapiens
gcactcacagctttgcaagta

SEQ ID NO: 85 - DNA, 20 bp, Homo sapiens
tccctgagtggagaatctgg

SEQ ID NO: 86 - DNA, 138 bp, Homo sapiens
gcactcacagctttgcaagtattgctgctcagtgaaaatgtaagtgccatacatgtgtaccatcacacacacacacacacacacacacacacacacacacacacacccccttctagacccagattctccactcaggga

SEQ ID NO: 87 - DNA, 20 bp, Homo sapiens
aggatcagcatggaatttgg

SEQ ID NO: 88 - DNA, 18 bp, Homo sapiens
cccatccgtaaatgttgc

SEQ ID NO: 89 - DNA, 383 bp, Homo sapiens
  misc_feature 303: n = A,T,C or G
aggatcagcatggaatttggccaaaacagatataagtcagatttaggtctcaagcattgaggcctgatgcagcatttatttatttatttagagacagggtctctgtcgcaagactggagtgcactgctgcaacctcagttcactgcaatctcagccttccgggctcaagctattctcccacctcagcctcctgaatagcaggggctacaggtatgcaccaccacacccggctaattttttgtagttttagtagaggcagagttttgccacattgcccaggctggtcttgaactcctgagctcncacttgcctcagcctcccaaagtgctgggattacaggtatgagccactgtacctggcctgatgcaacatttacggatggg

SEQ ID NO: 90 - DNA, 21 bp, Homo sapiens
tcctgagtccaggctatttca

SEQ ID NO: 91 - DNA, 21 bp, Homo sapiens
gcctccagagtacatggacag

SEQ ID NO: 92 - DNA, 303 bp, Homo sapiens
tcctgagtccaggctatttcataagtgaattatgaaactattatttttttctgaattgaaaaataaatgattataaaagaaaaaattaagaaaaaagtgaaagttatctatatttctaccatcagagacaactgctgttaacagcctggatatattctttcaggctttttctattctcttttacacacacacacacacacacacacgtgtgtgcatgcacacttaataagacctaaaataactgcattttgttaaagttacatgttgaaggaaaaaagtctactgtccatgtactctggaggc

SEQ ID NO: 93 - DNA, 1001 bp, Homo sapiens
ctagataacttaaaaaatgttttttttcttcaggcttatgctcatactaacaagctctgtcgaattatttcaatgtgcggaataaaaggcaagaattattttctggtgcagtttagaccttggatgagtagggttatgcagctgtttgctgcagtagttttggggagacacacacctgacttaagctatgtgaatttggatatgaagttccaagtgtaagatatgaaccaaaggatttctcttaacgtaacgatggaactcaagcctgaactatttttgttcattaacaacctggcagttattttttcagaataaggagatttatgaaagagctgaagtctgggcttcattttgcgtgtacatttgcttccgctgttgccggatggttggtaaaggaaattgatagagtttttaaagtgaggactgtattgtttactttatgtgttgttttaaagtaggaaggaacacagtcgccctgctatcagcctctggtttcttgtsccagtggcgctaagagtcaactcttctgcctgacagtgcctgctcctaccgtgcctgtgctgagatagctcctcctggcttcagggcctttatggctgaaacttcaattatatatataaaatatataaaataattattaatataacttaatataataatatataataacttttttgagacagagtcttgctctgtcggccaggctggagtgcagtggcatgatctcggctcactgcaccctccgtctcccggattcaagcgattctccatacctcagcctcttgagtagctgggattacaggcgcccagcagggtttccccatgttggccaggctggtcttgaactcctgacctcaggagatccacctgccttggcctcccagagtgctgggattacaggcgtgagcccctctgcccggccaactttgtatttttgctcaaagtttgatctgtacattttgaatcatttttatcctttttccaatttcccaactaaccaa

SEQ ID NO: 94 - DNA, 1001 bp, Homo sapiens
gttacatgatgaccattagttaaatgaactaaagaatgattgagcttatattctgtagtatcgtatttggaagttgtgtgttcaataaaactcttttagtataattcaggccaataggtattaatattaatgaatgtcagtaaatggaagctatgtttttaccttctagcacaaacatctttagaaattttattacgactgtgtatgtgtgtccagtggctgactttccaagcagttattagaggagatctgagtttttagcttctgcattatgattcatgttgaatatttatggaagagaagtgtttctacaaatatgtaaaaatattggtgagtgaaagaaatggctcccagtatgacagaagaaaatatcctaaagagatccacagttatctgcagtttccccaaggttgtgtttacataaaaaagacattgttttatgttctagcatcaagagatgattttacgatataacaagttccacaaagaactctcgtaagrtggttctcagtcccggcataactgctacggagatcacagagcaatattattctctggatttattgggtttgctgcattctgttagcatcattcatatttttctcccatgggtaccactttcctctcttttcctaataccaagatatggagactcatttatgccgtggagtgtgatgctgggaaatgaatgcttgcttattacctctctccacaggacctttcatgaccatacgtcgatgtctgccgcctcagtataaataggcacattcagaaatgtgttctctagtgaagggcatgttggcttggtggaaagcacagggacttcacgtctggactgcgagtcagagctgtgcgtcatgtgcttactggctgtgtgaccttggataaatttgcctcagttttctcatttgtaaaacagacagtcgctatttctgggaatagatgagataataaggaaagaacctagaatggtacctggctcctgccagttgcacagaatg

SEQ ID NO: 95 - DNA, 22 bp, Homo sapiens
tggcggttgttattaatacgtg

SEQ ID NO: 96 - DNA, 22 bp, Homo sapiens
tccattctcattctcattctca

SEQ ID NO: 97 - DNA, 299 bp, Homo sapiens
tggcggttgttattaatacgtgatttcacttttcatttatttcatttttatgtccattgtggcttctaacctcatatttcacacatagcaggtactcagtaaatacttaataaatcaatgaatgcaagtaatgactatgtatatactagtggagaaggaaggaggggagggaaaggagaggagaggcgaagagaggtgggccaggcagaggagagaagagagggagggagagggagagagagagggagagggagagggagagggagagagaggagaatgagaatgagaatgagaatgga

SEQ ID NO: 98 - DNA, 1001 bp, Homo sapiens
ttgtaggacttttagaaaacatggggttgtgcctttggccacacgcatgcttgtggatctacaagaacagcggtcctgtaactcttcagggaaggggcaccacatatctgtcctgtcaccatggcaaagctggaagggtctgcagagctacccagcatgctgctggtgttgttgtaaccaagcagagggcaagattctcgccatgagaattgatgtacatgtctagcatgtgaagcatcctaagggctgaggtgggttcctgaaacctgtggaggaaaatgctcagtgcaagaagccaaagaaaaaggcaccaggctcagcgggagcacccgcctggagaagcatactttgtgaggatcagcagaaaggagctgagtgtggaagctgtccccaagtcatggcacaaaagtattcaaaagaaaggatttctggattgttttttaaaaaacaaaactgtgatgtaaatgatgaattgtgctctgtggtctgattaggaatgtragtggatccagagtacagtggggctgaggcagtggaagtatttttttgtgtttttttttttaacttttaggtcagggatacgtgtgcatgtttgtttaatgggtaaacttgtgtcacgggggttcgttgtacagattattttgtcacccggataccaagcctagcaccccaatagttattttttctgctcttgtccttcctcctgccctctacactcaaggaggccccagtgtcttttgttcccatctttgtgtccatgtgttcacatcatttagctcccacttctaagtaaaaacatgaggtatttggtttcctgttcctgtgttagtttgctaaggataatatccgccagctccatccatgttgctgcgaaagacatgatgtcgttcttttttatggtggcatagtactccatggtgtatatgtaccacattttctttttacattctgtcattgggcattaggttgattctacatctttgctattgtgaatagtgctg

SEQ ID NO: 99 - DNA, 21 bp, Homo sapiens
tcaaagggaagtgtcttggtg

SEQ ID NO: 100 - DNA, 21 bp, Homo sapiens
ccctccagagttcacagaatg

SEQ ID NO: 101 - DNA, 137 bp, Homo sapiens
  misc_feature 102: n = A,T,C or G
tcaaagggaagtgtcttggtgtctcactggcacatatccagcatgatgttggtaaataaccgagtcccggtgtggcgtatttctccctgaatcttgactganaaactactgaagcccattctgtgaactctggaggg

SEQ ID NO: 102 - DNA, 1001 bp, Homo sapiens
gtgatactgatgacagtggtctgaaaactggcctttggaagtcatagacacaatgaatttacctgtcaccaccaccacctcccctaggaacttctgaaggacatctacattccgtagaaataaagttttaaattgaaggaaaaaaatattcaaacttacatcatgacttaagcacctaagagacttaaagaacatatcaaaattacaactgtgtcactgaatcaaatttacatttttgacacaatcattacaaaatcattacttggtaagaattttccaatagtcctactggattgtttttatttagaattaccttaagattcctgcatttctactcacaattttaatctgtcattactcatgaatatctgtgtctatgagattttttattatgagattttagtttcccttaagatttgggttctcatatgaaatcttcaggaagaaccttaaagaaagttcaaattttcataaagcccttttccaaacacattgacactscaaattttgacctgactggtaaagatctgtgattgtgattgttcaaatgtgattctctaaaaatacctaagaggccgaccactacatcttccgcactcatgaaaggcagttttccagatctgacatgtcctatgggttcactacataaattggctagggcaagttctactaactagtacactccattctcttgctaactagcacactcctgttaactagaatgccccactctccacctctgcctactaagggtaccactgaataacaaaccctccaacaacagatggggtaggaagagcagtctgtcttgtcagagtggaaaccaacagggaggctgggctcccattagaacatgtgcagttaccgcatgttccttcagtgtcttatccaaatgctccctctcttccagctctttcccctgcttttagacttcactcagaacacagccacgtacacaacaatttccagggcagcctccacccctgggatcctagaaagtt

SEQ ID NO: 103 - DNA, 1001 bp, Homo sapiens
tgtttgcctaataacagtgcattgaaatatatgtttgttttgtgtggtttttttgcatcagttttgttttataacaaaaggctaaaaataagtatttaaagaaaatagtgcatactatattttatttgctgatattcataatgatcaccagattattgaaatttatgagtaattttgctataaataagcctgttttctttgtttaaacacacacacacacattttcacactcacaccttcaaagccacataatagaatgtttagcttaaacctgcagccgctagttgaaatgttgcttcatggagttttatcctcctaacaacctgtgtcctaagtcacattcctctccagaaatgtggacattgaccatattccagtccctgagacgctgtttcagccacacgtggcaccccagacccttgcccacctgcatcctggtcattcatcctcctcctcatggggtcatttcttgatccctattaagcattaaaaggggattamatatctctctacttgcagctaatgttttgcttggtttggccaagaacattttaagttttaaaaacctggggctattggagtgggaccatgggcaaaggtcaggacaggctagctactaaaatggcctgccacggaccttgtacgtgaaggttgaaggattctggtgctctctggtgccatcgctgttagtcgttgtgcagcacagaaatattttattcaacaaactctgcagactcctgaactttaggggtgggctgccttctgcctggtgctctgcacagatcctggagctctcgtggtcatttatgtgcagtgaagctgctccactcacctacagcttgtccttttccagagaatccctatcatcctcccctcatcccaaggaatgcaacaaaggaaaattaatagtgaatgcttttgccggagacctgtggatacttaatttttatagatactcaataaatatttatttatattcactagcagcaagcaattcactt

SEQ ID NO: 104 - DNA, 20 bp, Homo sapiens
gactttcctaaaagcccagc

SEQ ID NO: 105 - DNA, 20 bp, Homo sapiens
gcatcttgcatggtgtattg

SEQ ID NO: 106 - DNA, 170 bp, Homo sapiens
gactttcctaaaagcccagccagttcagatgataggtgcagacacatcatattgcatatattcacattacacacacacacacacacacacacacacacactctcacccttctctttgctggggaaaggtttgttgcagaagttaccattccaatacaccatgcaagatgc

SEQ ID NO: 107 - DNA, 20 bp, Homo sapiens
  misc_feature 10: n = A,T,C or G
aatcacctanactactgcca

SEQ ID NO: 108 - DNA, 23 bp, Homo sapiens
atctgatggggagttatgtattc

SEQ ID NO: 109 - DNA, 241 bp, Homo sapiens
aatcacctatactactgccacataagcactatcaataaattttatcaatctcttcctgggtgcctaccagatgtgtgcatgcacgcgtgcacacacacacacacacacacacacaaatttcttccactgcattcattacagcatgcttttctctcttaccactatattgggaatacttccccatgtcactaaaacttttagaaaacaccatttataatgaatacataactccccatcagat

SEQ ID NO: 110 - DNA, 20 bp, Homo sapiens
gccattcgtgtggtctgata

SEQ ID NO: 111 - DNA, 20 bp, Homo sapiens
aaatgtttctgctgccatcc

SEQ ID NO: 112 - DNA, 268 bp, Homo sapiens
gccattcgtgtggtctgataacagcagcagcattaagttcccgtccattggctgcaagcagggaggaaaaaaggccccagcgcctactgcctgctttcctgcctgcgttaatatcatctcttatcttaccaactaacatataggggtgtgtgtgtgtgtgtgtatttatgtgtgtgtgtgtgtgtgtgtgtgtgtctgggtatatatacacacacatttatattcgttaatttccgtggaaaagaaagggatggcagcagaaacattt

SEQ ID NO: 113 - DNA, 21 bp, Homo sapiens
ccatggcctatgacctattca

SEQ ID NO: 114 - DNA, 20 bp, Homo sapiens
tctcctcccagcagtcacat

SEQ ID NO: 115 - DNA, 147 bp, Homo sapiens
ccatggcctatgacctattcaggctctgtgtgtgtgtgtgtgtgtgtgtagtgtgtagtgtgtagggaaagatacacggtggatgaatgagagctggggctggggatatcaagcctattgactccccatgtgactgctgggaggaga

SEQ ID NO: 116 - DNA, 1001 bp, Homo sapiens
cctgggcctgcaggtggctgcgaagggaggaggaggaggggaggtgggcagtggcgctggcctccctgcgtggacccacttcctcccacgctgtgctcagagaatcttctggagaccgcagctgtgcctgggaggccatccttgtgcctaggaggacagggaagagggtggatctcagacacaggcaggctgggaggtctgcacaggtgtggccatagaacatggacgcctccagtacgcaggcacaggcagctcagggccgggagcgaggcccgtctcagcaggcggtgtcagccgcggagtgggtaggtcctctgaggacgatcacacctgtgggcaagagcacacccgggctctgggccaagtaagcctgtgaatcccactggcgttgtgaacccggagcccttgggatccgattttttatttgctatttggatacagctgtaagagatgacagattattttacatccctcagttctccgaacttgccttggaccagraatgtcaggccctcaccgtgcctttttctcttctccaaactctctggtgctgcctggagcagatggcaccccccacagacgtcgtccttattgttgtcaccagaatattccatttccacagccacctggcatcccaaagccttccttcagtgggcagcctcttcacaggcaaatgctagcgatggttcaagtcacacggccagcacatactccatttccaaggaggtcattgctaactctaaatctacccctgttagttagccaaccccacgtgctcattcttagagaggttctgttccctgaaaacagtctggagccaaatgctgtgtgagctggggcccggtcatggaaacagaaaacttccattccgtcaagctggatggattctacagaaggaattcggtgtttacagaatcgttagcagggctgttcgcgtgaaggtcagggaaaagcaccccaagatttcaggataccaagaagttactgaaattgccaaaagt

SEQ ID NO: 117 - DNA, 20 bp, Homo sapiens
gtgctttgctgacatctgga

SEQ ID NO: 118 - DNA, 20 bp, Homo sapiens
ggacagggtggactcacaaa

SEQ ID NO: 119 - DNA, 412 bp, Homo sapiens
gtgctttgctgacatctggaaattccacagaggctggtggagcgatcagctggagtgaagtgagacagacctgagggaaaatgctagctctgcctcttatagattgagtgaccctgcagaagtcacatgatcattctgaggctcagtttctttgtgtgtaaaacagcgataatcatacccatgttgcaggacttggggaagattaaatactatgcatacacacacatatatatgtgtgtgtgtgtgtgtgtatatgtatgtatgtatgtatatactttgtacagagcctgagatacagtaagtgttctctacatggtagatattattattgtcttcttgtaaaggagagaaggggattatttgctgagaactttaaaaaaatctcattcgcttttgtgagtccaccctgtcc

SEQ ID NO: 120 - DNA, 20 bp, Homo sapiens
ttccagtgcctgtttcacaa

SEQ ID NO: 121 - DNA, 20 bp, Homo sapiens
ctgggaggtcctttcttggt

SEQ ID NO: 122 - DNA, 141 bp, Homo sapiens
ttccagtgcctgtttcacaaagtatctgaatgaatgaatgaatgaatgagcagctgaatgtctttcttttttatggggccacatatgattgtctcctttgtagctatgccaggtagacataaccaagaaaggacctcccag

SEQ ID NO: 123 - DNA, 20 bp, Homo sapiens
ttgtgggctgtgtagagtgc

SEQ ID NO: 124 - DNA, 20 bp, Homo sapiens
gctgtgcccagaaacctaaa

SEQ ID NO: 125 - DNA, 250 bp, Homo sapiens
ttgtgggctgtgtagagtgctctaaacccagctcggcctttgctgtattagacagaagcacctcattcatatccctggggcccctgatggtgcagtggtctggctgtggtctgcacaccagctattctgttttgttttgttttgttttgttttttcctacctttttccaatcctcacaccttctgatcaacagccccagtagggtttaaaggtcctagagctacatgggatttaggtttctgggcacagc

SEQ ID NO: 126 - DNA, 20 bp, Homo sapiens
ttgcatggagatgaacaacc

SEQ ID NO: 127 - DNA, 21 bp, Homo sapiens
tccactcagagaaagcaagga

SEQ ID NO: 128 - DNA, 396 bp, Homo sapiens
ttgcatggagatgaacaaccaggtttgtggccacatcttgccgtgtgtgtgtgtgtgtgtgtgtgtgtgtgtgtgtgtgtgtgtattgagacagggtcttgctcttttgctcaggctggagtacaggcgggtgatcatagctcacttgcagcctcaaactcctgggctcaagcaatcctcccacctcagcctcctgagtagctgggtctacaggtgcagagcaccgcgcgtacctaattcttttaactttattttttgtagagacaggttctccccatgttgcccaggctggtctcaaactcctgggcacaagtgatccgcctgcctcagcctctcaaagtgctgggatttcaggcaagagccaccgggcctggttccttgctttctctgagtgga

SEQ ID NO: 129 - DNA, 20 bp, Homo sapiens
tgctgaatgtcagggtttga

SEQ ID NO: 130 - DNA, 20 bp, Homo sapiens
ccaccctagcaggtctctgt

SEQ ID NO: 131 - DNA, 361 bp, Homo sapiens
tgctgaatgtcagggtttgactgtttccataacaggaagctgctcactgtctcactgtattaaggaactctggtctacacaatagagttccaacaaaaccctaaacactccatttgctgggggaacctcattgaatccagctctcattgtttcttttataggctgaatcctgtatttacagtgagaggggtgtgtgtggctgtgtgtgcacgtgtgtgtgtgtgtgtgtgtgtgtgttcgcgcatgcacatgtgggtttaacaagatatgaagcctggcttgtcaccttccaagttctccacttgaacttgagcatagatcagggtgccatgattccccagacagagacctgctagggtgg

SEQ ID NO: 132 - DNA, 19 bp, Homo sapiens
ctgaagagcaaatggccct

SEQ ID NO: 133 - DNA, 19 bp, Homo sapiens
taagatcacatggccccct

SEQ ID NO: 134 - DNA, 335 bp, Homo sapiens
  misc_feature 171: n = A,T,C or G
agctgaagagcaaatggccctgggaagtattcctttagggttacacacacaccacacacacacacacacacacacacacacacacacacacacgaaaatctctaaagagcaatgagcatagcagcctggatggtgctcatccaaggataagtctccagacaaatagcacancagggggccatgtgatcttagttcacgaagacattcaataaagacccaacaaaacccacgcaacagtctatgtctctggccccctgcagggaccttgctctagcacacggagcagggtggggcatggccacagtggcccctactgccctgcacttcccacagct

SEQ ID NO: 135 - DNA, 1001 bp, Homo sapiens
ttccatgcattccacttctttctggatctctggtttcacaggcaagatgggacaggcagagagaacctgggcatgtgccctctgtggagaaagtgacttcagaaaccgctgaggctctattagcctgggattctaaactcggggggacatgaaaaactcaagagacgagtcatcaggctctatattcataagactcttctctgtgtgtgtgtgtgtctcttttcaaacaaatagcactgcgcagcatccttagagactacagccaaatgtccttcatgtattttctctacatttcaagaatctcgggaccatgcttcctatctaatgtgtgaccttgagagttaaaatcaaggggaaaaggtcaccgaattgggggcaagtttgagttcccgtcaccagccacaatctctatatcaaatggaggacaacacaccacctgggcctcagccaggtttgcctgaagcagggccaggcagcctcaaggcctccatggtaggctgrggacatggggacgtggggaaagggggtgcagggaaactgggaactaggaggggagcgtgagaaagagggaataaatgcgtacgcggatgaagaggaacagcaggaggagatgaaggcggcgcacagggcagaacggcagacacagggctgggaaggtggcagggccggactccagaacctcagctgagcgttttcttctcctgtgtcccagggatggtgtgaagtgtctacaggcatccgagtgaacccaaagggagagtttggctggcacacggggagacgggccaaggcgcggcgggcgagggcggcacaagcatggcgctgcgacaccactgctgggagcagggctgaaaggtgtcttttgctgtaaggactttcataaggcagtcccaatccaaagactggctttaatttcacggccttagcctctcagtttcttaagccttctgaggacctcctgatcatgacaattaagtcactatttacagccatgtgacaga

SEQ ID NO: 136 - DNA, 1001 bp, Homo sapiens
atgtggatgatctaccactataggtgtaatctttaacatcatcttattccttcttaaagtaagttatccgcttgtaaactgcttatttctttggggcattgtccccataaactttttataaagcatcagtgatttcaccattccacccaagcttcaccataaatttggtgtttgttcttgcttcaattttagcagaattcatgttgttctgaaagggggctctttcaaattgatgtcttagtgcctcaaactagatcatgttctaacatgttataacaagttattacaagtgtattttggtgcaaaaaaattgaaatccatgcataatatgacctttccatgaagttttggaagacctctcctatgcttatgcatacactccccaaacgtatcaatccagttgctattgcccaaggaacagaaggctcatcactccatggagggtttttcctgcagcccctacctaagaccttctcactttctctgacagtcctatcatcrtgtcgtaaaaggcctgcccacttagtccaacacactggaaatggatgattgacaacatgtttatttacccatcccctgggggaaagtctcagattttgtgaggttgttgcccctgcaatgtgctttaaactcagctttctgttgcttgtgtctctgggtcagaagaatttgtcagtgataatgtttttgttaaagtcctatgcccagttaatgccaactcagcgctctcatcccctagggctcctgtaatcatttttcttgccttctcttacagtttctgtatgttatagaagttcaaagaagacaaactctagccaagagcagtgtgaagaaaagaagacgctatattaatcacagtccagggatgccttctggcttcctggcagcaattccggcctgagattccttctctgtgcatacttcctgtcaacattgtgtgatgtcaagctgtggccgtcacaaaagtactgtgaacacctgtaaatcccaactatcaaaaa

SEQ ID NO: 137 - DNA, 1001 bp, Homo sapiens
ttgttttgatcctaagaaaaatgggtgtcattttatccaggaatctaagaattataataataaattaataaagtgaatgtgataatcaaactgtgaggatacgaacaacataagatttaatgatcgttgtcaaaaccagtccgtagggctgtggaactttatcgtacaattcgactttgatatgtgtttaaatatattttctaagttatccacaacccaaaacaggaccccttagaggtaatctagaggaatccctcacgttacagacagagccactggttaagggtctagagtcacacagggagttactgcagaatcactactggaaccctgtgctctttctgcagggattcggatattttggttggatttgcattcttacgtcaatgtatgttctccaactctgctcttacatattgaaaggcaggcagctatttttaaacaccctgcctattagccttcggaacataataataatggcaagcaccctttattgcttcrccgagctgcagacacccttctagggtgtgaacagagctcagtaaagatagcagcctcaggtctgtgtgttgctttgagccacgagctggtctgcaggcagcagccatgggccgtgcctgtgttggtatgtttaagaacattggcgaatacaggaattacatggactaggtttagaaaacaaacagtaacgtacaaaaaggaaggtttgatatggactgcaaggacataaagcaggtgcacatgcgtgcactaccagaatagctacacggtgggaaggaattccagaaccacgtgagaaagagttgttaggacaatgcagtcgtgaaataccatgtttccaaccctatcactctattttaaaatagataataattataatttttattaatatcaaacaaattagctttgggacctatggccctaacttaggggtcacggctgcagtcccctttcttgcagacctggcaggctgcgcagataactgcccccagcgttggcca

SEQ ID NO: 138 - DNA, 21 bp, Homo sapiens
ccagacatttcacacactgga

SEQ ID NO: 139 - DNA, 20 bp, Homo sapiens
tttgccagaactagcggtgt

SEQ ID NO: 140 - DNA, 140 bp, Homo sapiens
ccagacatttcacacactggaacatatatacagtacacacacacacacacacacacacacacacacacacatgctagcatgaaacatctgaagtacacagccatcctttgaaaggaccccacaccgctagttctggcaaa

SEQ ID NO: 141 - DNA, 20 bp, Homo sapiens
aaatcgcagctacacacagc

SEQ ID NO: 142 - DNA, 20 bp, Homo sapiens
tttctgcaggtgttgcaagt

SEQ ID NO: 143 - DNA, 259 bp, Homo sapiens
aaatcgcagctacacacagcaaagactaacagtatttacttaaaaatattgtgtgtgtttatatatatatatatatatatatatacttattatatatcttttttgtgattttttttcttttcctttttttttgtgcccaagtagagatacgatgcgattgaaacgatgccctagaacagaaatattctttaaaggaacaatactttgaaaaataaaaaaaaatttaaatcgttgaacatacttgcaacacctgcagaaa

SEQ ID NO: 144 - DNA, 20 bp, Homo sapiens
ggtgaaagacagaagcacca

SEQ ID NO: 145 - DNA, 20 bp, Homo sapiens
tggtgggaagccttaaattg

SEQ ID NO: 146 - DNA, 185 bp, Homo sapiens
ggtgaaagacagaagcaccaaacagtctttgaaatgggtcagttattacaattttgactttttatatatatgtatatatatatatatatatattctagttttcctctttgtgttatttttttttttaaaaaagcacaaatgaaaaatgaagaattctttccagatcaatttaaggcttcccacca

SEQ ID NO: 147 - DNA, 23 bp, Homo sapiens
ataaagagggtgtgtatgtgtgc

SEQ ID NO: 148 - DNA, 27 bp, Homo sapiens
ctcatcttctctctacagatgtactcg

SEQ ID NO: 149 - DNA, 210 bp, Homo sapiens
ataaagagggtgtgtatgtgtgcatatatatagagagagaggcgagtatatatacatatatatatatagagagagaaagagatagggtgtgtgtatagatagagagaaagagggtgtgtgtgtttatatataaagagagggcgagtatatctatatgtagagagtgtatatatctatagagggcgagtacatctgtagagagaagatgag

SEQ ID NO: 150 - DNA, 20 bp, Homo sapiens
gcaggacaggacctgagaac

SEQ ID NO: 151 - DNA, 20 bp, Homo sapiens
ccacatcgctattggaggat

SEQ ID NO: 152 - DNA, 399 bp, Homo sapiens
gcaggacaggacctgagaaccagatacgcctgcaggtgcctgtccctctgcgccccccgggtggtgttagggctccctgtgcacggaggcctgcaatcatttggacaacacatggttaccaggtgtctgctatgtgccaaacgatggtcacaggagggtgagaaagacagtctccacgttcaagagtacaaagtccgtgatccaggaagacaatgaggcagccactgtgtctcatttctggatgaatggatgtcacaaagccatggaagtggttcagtggcttccatatcactaggctacctcgcctgtctctctctctctctctctctctctgtctctctctctctctcagagcaggctacctaggattttacttgcaatcctccaatagcgatgtgg

SEQ ID NO: 153 - DNA, 20 bp, Homo sapiens
tctaagattcgccagcttcc

SEQ ID NO: 154 - DNA, 20 bp, Homo sapiens
attctagggcttgcaggtca

SEQ ID NO: 155 - DNA, 278 bp, Homo sapiens
tctaagattcgccagcttcccccgccagagagcgtccagcactcacttctaagatcaccccttctcccactgagacagctagccttgcacaaggcattcccaagcaagctccccaacaatataaggagaagaaagagaaggagtggctacacacacacacacacatacacacacacacacacctcttagttgtcattttgaacctaattgttttaacaccagctgtcacatctgcagaattctcttctctggtactagtgacctgcaagccctagaat

SEQ ID NO: 156 - DNA, 20 bp, Homo sapiens
cccaaagtcatgaaatgaga

SEQ ID NO: 157 - DNA, 22 bp, Homo sapiens
acaacatacctgttaggaggtg

SEQ ID NO: 158 - DNA, 386 bp, Homo sapiens
  misc_feature 142, 149: n = A,T,C or G
agctccatttcactaataaggagacagatgtggaggttggggagttggtcccaggtcacccaactggggagggcagaggttggggagggacaggagtcaataacccaaagtcatgaaatgagaaaggaagtaaacacttggntggagantcacacacacacacacacacacacacacacacacacacctcctaacaggtatgttgtctgcaacaaggcaaaaataattcattaatatctcatttaaacttgagggcgagggaattcctgaaccacctctctggagcaaataatggaaattggaaattgattgtcatttacctttgaggaagacttcgggatgtgccatgtctttggtatagggctgcgtggtgttgtgacgcatgt

SEQ ID NO: 159 - DNA, 1001 bp, Homo sapiens
gattggcttttactctatgggcaacagagagccatggcaggctttccaggaagggagtgacatgcaccttagacaggtcagcctgacagcagcttaaaactagatggaatgggagacaactttgtccctaagctcagtcccctaaagataccagcacatgactgtcaggcccctgctgggacagctgcccctccctaggcctgtccattctcttacctccctcctgcctctgatggggaaggggtgatgggttggaagtgggtgtgtgcaacatttaccatggccaggtctgctctgtgctctgtccccacccagcacacccatctccatccataccggccagccttgcctgttccctcacagtgatgcataagctgggcttctcctgcggtgtgatactaatgtactagccaaaccctgagaggccacatatggtgggtgagggatgtgggactgccagactgccagccagtgccctgaagactctgcatttcatatgcrtacacatttagtagtagtgtgaccctgggccagttactgattctttctgagcttcggtttcctcatctgtaaaatggggatgatgatacttaccttaaagggctgccatgaggtcgaaagacaaactatgacaaacagccagtctcgtgcccagcccagcgtgggtgcaagctatctggtggctgctcggatgatgatgatgacgatgacaacgatgacgtagcaccccatttccagctcacaccatcgggatcaccgccagcatcagcagcatcatcaagccatcttcctgcgttgtggcagcttgggcccccactggccatgcaggagccaggagatcaaatcatgaatggggctctttgcacttcaggcaaagtgcaactccaggaaagagagaagattaaggccaaatctctgcacccaaacaggatccaagaagtggggtaatctgggactcatcacatctacataaagggaggaggaagccccagggtggcctg

SEQ ID NO: 160 - DNA, 1001 bp, Homo sapiens
tcacacatcaaaatgtggacatttaattcattttaatcgagaaattaaatgcatctgccttgcttcctctcctggggctcttccatctcaggaaattcccacaccagcaggtctggacaagtcctcggcagtaacttcactcagcctgaattcttcttcctttccccacggctctgactccaagttctgatcatcaagttgaaagggaaacttacaaccaaaggagatgtaaacaagaatagtctctgtcagttcagtggagagagagagagaagctttaatgggcactagtcagtcagaggcttattctgcaagtgttcattaggaatcagtggaatttccactgtttccctggtgtcacttgggctgctgcctcttggcctgtgtcaaagacaacaaaggaaaatggtccttgcccctcgaggtgggactggatgccaaccagcccgacaggcagtgggtggttcacggttctgttcccactggaggatgctcttgtckgcctaccctctcgcctgagacctggaaggaagtgcatgcccaagggtgccagttggaggggagctagcagtcagaccaggctggtgtaggctttgcagacagagactcacctccttccactgccagaagatgctgccgtcgggtgaggagctgtgacctgggcagaggaaattcaaggagccaatttctgctctgtacatagaaaaggtggtcctctcctgtttgttcggggggcatctctgaagcccagctccactctttaccatcttgctaagaaccaggagtctggaacatctcccaaagtctacgtggggctcaatatcatgtgcaatcactttgcaccccgttacgaatgtgggagcaagagttggtcaattttggaagggcttggttaagacagctggtaaacctcagctgagataatatctctattctcctctccaaagaggttggcagcttcaccgggcaaacagtgcccagagaggcctgcataagccaca

SEQ ID NO: 161 - DNA, 1001 bp, Homo sapiens
cacggatagaaggccaccactgagcaactgtaagtgtgcaagtccaatcagaccacttccagaaggtgctttcccctacaactaagacagcattcacacttaacccttgtagcaacttcctacactgagaaacacaacagaattttgctgtatgattctcatcttctcagaaaaatgtgttgtctctttgatctgcctaattaggctaattgaactaggaatcaaagcagtttctggggaggaaggtaggaagttctgtttttagtttggctatgatttgtcccaatcattttatgctacaaaagcttttgttggcgttggcctccgagtcagtgctttgaaaggtggccgcaaatgtgatttatgggaaggtgctgccgggggcatgcactttatgggcaggtggtgccggaggaagtggttaggagacagtttcctcacccatctcctggagagacctccatctcccttacccaccctgcagtggtaccacgcacatckgacgaaagaggctgtcgctaaaacgctttgaaaagcatacacacgtgcacacacacaatgctcacgggtagtatttgcagtacagaattctagtactgtgcacctcagctacagacatcccaatttttgaaagtgtccataatttatagcaagagatatttgggtaagtgcagaaattatacacgagagtcattgaaactgagtttataagagtcaaaaattggaaagaacctgaataacaagaattgtaaactgctggacttccagcaagagggagctggttatattcatgcagagcggccttgaaaaagatgccgtgattggataacgtacactgtacacggctgagaacaaaggaatctgaaatgacaatgaatggagtattagcagcagtgacctagtgaattttgttctgttcatttttgtgcactctctaaaattatttacaaattatgtcattttttatgataaaaagttgtctgaattttggaaaaacaagg

SEQ ID NO: 162 - DNA, 1001 bp, Homo sapiens
tttattgtgaacttgcagaaaatggaaaggattatgctttaaagacagttggcttggctggatagaaaagatccctctgtcctgtttccctgtcctccttcccacatcgatttaaaaaattagatgcaaatgcaaaatccttaaattatagatttatgataaatttaaattctggtagaatcaaggttttataacatttaaagtgtctgacactaagtgtatataatcttttaagaaacgtcttcttaacagcgcatggtattctgtgactgttcgtgtaccatgaatattcttattgggttctagagttagttactgactcttgaagatgggcatctaatggtcctcctgtggaagtggagagcagctctccactgtttgataacatttaaagccaagggtgaaccactcaagaaacatttggtggttataatatttttttgttgttgttaagtaccatcaataaaactgaaaaatctcttaagtacctgactcctgcartgatacaactgcagtgataaaacttttagctttttacatcaggggtattaggtattttctcacagaaatagccttttgaggtgaaattcacataacatacaattaaccattgtaaaatgaacaattcagtggcgtgtaagagtatgtttacaatgttgagcaaccatcacctctgtctagttgcaaaatgttttcatcactccaaaagaaactcctttattcatcatagcccaaagttggaagtattttcttgattgggctcttgattacatggatgcatctgagtcattgaattgaagcctaagatgtgcttaatttcactgtgtgtaagtttcacctcagttaacaagagagaacagaacaaaccaaaaatcttaattcttttgaaaaaaagactttctggctgctttattaaagaagccaggggaacaaggttaaaaggaaatcagttagcagtgaccaaggcaagagatgatggtggcttggctgaagatggtgac

SEQ ID NO: 163 - DNA, 1001 bp, Homo sapiens
ggatggcatctgaatcctggatttcccagacctcagaaccagaaggaatacatttccattgtttaagccacccaggcaatgatatttctgttataaaagcccaaactaagatacccacacagagaacacctacacacagtgtggttacaggttgcatcatttctttttctttttcaatatttgcatattctctaaattttctacaatgacccaccacatgaattcttttaaaagaaaaaaatggtaaatatgaaatagaatagtagtgttgacccttaagaggaaaaagatggtagaagacactatgttgcttacagtagactacaaatgtgcgtgaaatttgtaaataaaagatgaatacttataaatgtcaccacctccctctctgatgtttctgaaaccagagcatatgtggttaaccttgctctagctccagtccatccatccatcatcatgctaaaacatacagctgtaggcagtggagaagagctgtatgtggtsaggaaagcgggagacaggaattccagaaatgtctactaaagcagtgctttaagttttaatttattcaagaaaccaatacatatcagagcataagtgagaaaaagaaaacaattataaaaaatacaaaggagtccaggataatagaaatctttcttcattcacatattctagctagaatagtgagaagaaattctccctcaaacgtggacagtcccttacatcttcagccgacacggaagtcttatctgagaatagaatctctgctacactaacctaggagacggccaggcaactgctgcggtatacccatcaccccagtgttctggaagaaaaagacagcagggagaagttctctttagaaccagctcttctacaccaaatgaactcaggagacaatgaatggaaacaccatgccatggtgtgagcaatgcaatgtggagcacaagcagcggagagtctgctgaagaagctactcccctgaaataggaaagaagaaaacc

SEQ ID NO: 164 - DNA, 20 bp, Homo sapiens
gccagccagactggattaag

SEQ ID NO: 165 - DNA, 20 bp, Homo sapiens
agccgagaagacctgtgaag

SEQ ID NO: 166 - DNA, 257 bp, Homo sapiens
gccagccagactggattaagaccccccgtcaatgacctcgtttaaccctagttacctctttcaaggtccaaacatagtcatactgggggtcagggcttcacatatgaatttgctgagggggcttgagggatgcacaattcagtccataaacgctgtatatatttatttgatgtagttttgttttaaataaaaagtgtgtgtgtgtgtgtgtgtgtgtgtgtgtgtatctaaagtaggcttcacaggtcttctcggct

SEQ ID NO: 167 - DNA, 1001 bp, Homo sapiens
gccaagaaatgacatgttgatcctcaactagcttgtggacagagtgtttcttttctggtcattcctttcagccactgatataaacaaatataattatccaatcaaaattctgaatgatgagaagtttcctatgcagtcctaagcatactggttttactttccatagttcagcaaaaatattactggattactggggctttaaaatggcccaagctgtagcccacagatctgcactagctcacagaatgccacggtttggtttgtttctgactatgatcacagagtaatactaacaaaatcttgctatttgaaggaattattaatttttgaattacaattagaatacaattagattattccacattacccagtgaattattattataggtgccaacattcacagtttaatccaatgaagaaactgagcctatataaaaataaccaccaccaaagcagaagaaaagctacgtgaagaactgaactcaatcttaatggttcctkcagataactactcccaattgacccaaataaaccaatttactgggtcaagagagagcatgaaggaactaaggactctgttagaagtgaggaaatatggaattactcgtgcatgtagcatgtataacatacagaacaagcatttctgaaaatgtgagcagtatcaataggttggataactttagccccaaaaactctactactactgctttttggaaataattaaaaatatctcaatacagtttataaactttgataaagtcaatataaaagtaataacatcatataaaccggtcttttgctcatttgaactcctgacatggggattataagccataacagatttcttttttcaaatatctgaaatacaaggaataattttctttaaatgagttgcaatataccaaccagtattgggctggtttctgtgatttcctcttaattggtggtagcagcagtaatcctctaattcttaggatggacaactgacttttgaatatctc

SEQ ID NO: 168 - DNA, 1001 bp, Homo sapiens
gatcctcaactagcttgtggacagagtgtttcttttctggtcattcctttcagccactgatataaacaaatataattatccaatcaaaattctgaatgatgagaagtttcctatgcagtcctaagcatactggttttactttccatagttcagcaaaaatattactggattactggggctttaaaatggcccaagctgtagcccacagatctgcactagctcacagaatgccacggtttggtttgtttctgactatgatcacagagtaatactaacaaaatcttgctatttgaaggaattattaatttttgaattacaattagaatacaattagattattccacattacccagtgaattattattataggtgccaacattcacagtttaatccaatgaagaaactgagcctatataaaaataaccaccaccaaagcagaagaaaagctacgtgaagaactgaactcaatcttaatggttccttcagataactactcccaaytgacccaaataaaccaatttactgggtcaagagagagcatgaaggaactaaggactctgttagaagtgaggaaatatggaattactcgtgcatgtagcatgtataacatacagaacaagcatttctgaaaatgtgagcagtatcaataggttggataactttagccccaaaaactctactactactgctttttggaaataattaaaaatatctcaatacagtttataaactttgataaagtcaatataaaagtaataacatcatataaaccggtcttttgctcatttgaactcctgacatggggattataagccataacagatttcttttttcaaatatctgaaatacaaggaataattttctttaaatgagttgcaatataccaaccagtattgggctggtttctgtgatttcctcttaattggtggtagcagcagtaatcctctaattcttaggatggacaactgacttttgaatatctcagtaatgagatctccatt

SEQ ID NO: 169 - DNA, 23 bp, Homo sapiens
ggaagctgatgaggtgtatatgg

SEQ ID NO: 170 - DNA, 20 bp, Homo sapiens
gagtctgaggtgggagcatc

SEQ ID NO: 171 - DNA, 242 bp, Homo sapiens
ggaagctgatgaggtgtatatggatactctgtgctatctttaagcttttctgtaaacataaaaaacctaaaattattttaaaataaaaggtatgtatgtatgtatgtatgtatgtatgtatgtatgtatgatttttagagatgcagtctctctctgttgcccaggctggtgtgcagtggcgtgatcatagctcactgcagcctcgaattcctggacccaagggatgctcccacctcagactc

SEQ ID NO: 172 - DNA, 1001 bp, Homo sapiens
catactgcatacaagccaagaacataaaatgaacctctcagtcttacccttcctgcaactgaggacccgcttgccggcactcagtaggacacgtgattaaaagtgtggcttgtgaggccaaactgcatggttctgaaacctggttctaccatttacaagctgtatgacattaggcaaattacttaccttctttaagccacagtttcctccttgagacaggtggacattaacagtactagctcatgaatttagttggccgtttcaatgagttaatacacatcagctgttactaacatccaccatatattcccagaggggtacccaattctttggggtctcaatgacccttgtccttcaccctctagaaagcatgtcatcagagaataacaaacattatcttcaacttacttgatccactgctgcatataatttaagtaagtcattctcaaaacttactttactaataacatagtctatacaacccccaagtaatgaccacartgcagtctgttacgacagctatggcaaatactgacctagatcgcgagagaaaagaacagctgctgtcctcacagctgccccgcctcactttctgctaacagacgctgcttctgtatggccatcagcttgccatgtgctttcaggcaggctggacccatccccattccctacatcagcagcatcagcttcaatcaggaacttgtgaaaaacacaaattgtcagtccccaatccaaactagagcagaaactcttcaggtggggcctggcaatctgtgttttgataagtcctccaagtcattctgatgcagaccagtctgaaaactactgaccaagaaccactgaactaataatggcaactgcgtatctctaagtttagaaatggggtatacaacaattctagccaaggaggggcaacttctagaaattttgcttactcttaaaaatgaacacaaagaaggtaccttatctcttctggcctttagaatgttgttgattagaga

SEQ ID NO: 173 - DNA, 1001 bp, Homo sapiens
cttcagcttcaattcaggtagagcagtgaggtttgaaagtgcctcaagcagagcccacagttctctgatcctttacaatatcacactctgtaattgtgtggcatagcagccatgctaggaacgaggtcaattacttaggtactcgctagactttttccttttctccacccctggggtccaggctcttttcccagcacttactcagggctgtcattagccctttctcctcagtttcatcgcccctgcatttacgttattctaagtcttctcccctatgggttcctgtggggaaaataaaagatccgaaagggaaaaaagcagaaaagaatgaaataaagtgaaaattcaagaggttcttgttttaagtccctatcttaaaagatatatggctttgtcactttcaaaagcattacattataaggtatgtggccaaaacacaatcaataaacaaacacacgcagacagatacaactaaatacacacaaacatacatgccacaayagagagggtctttgattcttaggatcccccttttcttttccatccattaattcctaactacactgttcttctctaaccatgtaactatttctcaatatccatttgtcacatgtaaaatattctcaagaccactcctagccttgtatacctgagacctgtctcccataccaacaccatcacttaattaagaaacaatggcactaaagctttgcttacaaatctgtgaaacaaaggtcatcccacctgcctaccttcccacttcaccttactaataggaggtttaaaggagatatgtgcttaagtacaccaaagaaccagaggtaccaacagggttaagatacgccttgaatccaagaaaatcccctgaagcagcatgtcaatactgagtaacacaaccattccctaggctatcacctttttttttttttttttttttttttttgagacagagtgtcgctttgtcacccaggctggagtgcaatggcacgatcttggctcac

SEQ ID NO: 174 - DNA, 21 bp, Homo sapiens
agccacacaggtcacagattt

SEQ ID NO: 175 - DNA, 23 bp, Homo sapiens
ttctgacattcttaatgggcttt

SEQ ID NO: 176 - DNA, 248 bp, Homo sapiens
agccacacaggtcacagattttggctttttaagaagaaacaagagccctcatgcagacccctggtacagtctcaactggtggagatactatgtaaaggagcttttaaattattaaatagcctctaaataaatacatattttatatatatatatacacacatacacacacacacacacacacacacacacacacttatattacatttattagtaacctaatttttaaaagcccattaagaatgtcagaa

SEQ ID NO: 177 - DNA, 1001 bp, Homo sapiens
cctgtgatgggatggcaccctgtccaggactggtgcccgccgtgtgccctgagctcctaggataggctctgaccacctgtgaccaggtggaataagtgggtaagaattatctcacgttgcattaatctttcttaaatataggtatggctcacatttatttcaacgtttaatgctagaagtgttttggtctttacttaaaagtttggtggtgtttttgtgaacagaaatatgccacagaaacttaatcttgtttgtatcaattagcctatgggaaaactggtttccatatacgtagtttcacttcaaattgcagtttctaagaactcactgatgacagtgaagatttactgtatggggttttagagtaaatttctaaatgtacgtacaatttttcacattttttaaatatctatttggtgatctatatattcaacagatgagaatcagtagtcacttttagggatagtttcctgggagatggcacccaataaagtctccaaygatgggacatgattttgaaagagtacattagctgtgctcacaaaccaagatccaatctttcctcaaccagatgaacttttccttaagacctgaaacactgatgagtcttgggcacatggctacaatacttttcattgagtccctgaaggccatttttacctcaatgaaatatcatctaaagaaaaattatttaaaactccagttgtataatttcaagatagtttagtgtatttagtatgactcactcttcattaaacttcacaactatttttaaaagctaatttaaatagttacctgtttgagctgatcgatggaaacagggcttgggctatttctgtaccaccctcagactaagaatgctttttatatttttcgaggggactgtgcatcagaggccttctgtggctacacatcttaaaatacttctttacagaaaaagcttgccaagtcccgaatcaaaacagaaatcaaagttttaaagggaaatcgtctcttgtact

SEQ ID NO: 178 - DNA, 1001 bp, Homo sapiens
ggtaagaattatctcacgttgcattaatctttcttaaatataggtatggctcacatttatttcaacgtttaatgctagaagtgttttggtctttacttaaaagtttggtggtgtttttgtgaacagaaatatgccacagaaacttaatcttgtttgtatcaattagcctatgggaaaactggtttccatatacgtagtttcacttcaaattgcagtttctaagaactcactgatgacagtgaagatttactgtatggggttttagagtaaatttctaaatgtacgtacaatttttcacattttttaaatatctatttggtgatctatatattcaacagatgagaatcagtagtcacttttagggatagtttcctgggagatggcacccaataaagtctccaatgatgggacatgattttgaaagagtacattagctgtgctcacaaaccaagatccaatctttcctcaaccagatgaacttttccttaagacctgaaacaytgatgagtcttgggcacatggctacaatacttttcattgagtccctgaaggccatttttacctcaatgaaatatcatctaaagaaaaattatttaaaactccagttgtataatttcaagatagtttagtgtatttagtatgactcactcttcattaaacttcacaactatttttaaaagctaatttaaatagttacctgtttgagctgatcgatggaaacagggcttgggctatttctgtaccaccctcagactaagaatgctttttatatttttcgaggggactgtgcatcagaggccttctgtggctacacatcttaaaatacttctttacagaaaaagcttgccaagtcccgaatcaaaacagaaatcaaagttttaaagggaaatcgtctcttgtactctgcaatcaatagcatttttttttatacatacacacacatagacacattcatgcccccccatccccatcccactttaatctggaaggtacctgatcta

SEQ ID NO: 179 - DNA, 20 bp, Homo sapiens
tgcagacagcacgttgtaaa

SEQ ID NO: 180 - DNA, 19 bp, Homo sapiens
aggctggtgctcctgaaat

SEQ ID NO: 181 - DNA, 116 bp, Homo sapiens
  misc_feature 48: n = A,T,C or G
aatctttccatcccacagaatctttccaacattacagaatctatccanttgcataagcctgactaggcaattgaccttatgaataagtctatagtatcaaatgatgttgaagacag

SEQ ID NO: 182 - DNA, 19 bp, Homo sapiens
cagcccagcaacattcact

SEQ ID NO: 183 - DNA, 20 bp, Homo sapiens
gtggtagagggttgccttca

SEQ ID NO: 184 - DNA, 174 bp, Homo sapiens
cagcccagcaacattcactgcagattttgtagagagctgcatatccaaattccaccagtctcaaatcagaaaacaacgctaaaacagagctgtagaccgctcaactggatggtgccattataaaatgcaaaatgccttttcctttttactctcctgaaggcaaccctctaccac

SEQ ID NO: 185 - DNA, 20 bp, Homo sapiens
gcaaacaacatggctagcag

SEQ ID NO: 186 - DNA, 20 bp, Homo sapiens
tgtttcttggcaaagtggaa

SEQ ID NO: 187 - DNA, 403 bp, Homo sapiens
gcaaacaacatggctagcaggtattaaaacagcagaccatgttcctgcagtatttcaagcaaaaccatctaactgggaaaaaaaatttttttaataaaatccttcctcagtaaatactgcttttgaagtatagctatgttagaagaaataacttactaaaattagcatgtcttttaataagttaactttaggaaatatttagagatatattctaatcttgaaaaaagatgtaaaaaaaaaactagacagtaaagtcacaggcactttatatcaatgcagaggaaagttaagatcagaaaaaaaaaaaatactaccctacatacaactacaaaagctaaattgacattttaaatgtacttttcagtttgccctaaaatctggacttccactttgccaagaaaca

SEQ ID NO: 188 - DNA, 1001 bp, Homo sapiens
tttgaagccagatagccaaaatagggcaagctacatggttacagttgttcctgatcagatgaaatgaacattttacagttaaaaaaaagaatgagggggaaaaaaatccctgaattttctcattgacttccctagatttttgaactcatttttgtgattctgtctacttctccattcactaaagtcttctaataatgccaataactgtctttagaatgttaagagtacaaattaggtaatatttatatggctggaggttctatggcagaaaggtgcgtttgacaacttcaatagttactttgatactattgaatactatggcacctatgagttttgggagtggcagggtagatggggatactacattttaggacacagcttttcatgagtatatatgccagtgtgaaatctctgaagactttagaaaaattactaatagtgaatttttactcccatacattgggaagaggggagtgattccaaaatcaacttttagaaacmagccatataactgtatccatgtatttcatgctatgatttaagcctcatactccctatggtatgtaaaactcatactcatatgtaagcctcatactccctatggtagtaaaacttaaggccagcaggtaaagattatttctgcatatagatgggattctgtttctttgctgaatttgaatgaataacaccttacatggcataaatatagagtaggattgcccaggtatgaaccccaatttcactaaaatagtaacatgaataatgtgagcaagattacctcttcaaatctcagttttcaccttgatataatagaaataacaacagtgacttttctgaaaagttgctgggcagagtaaaggtggtaatcctttcaaggatctcaatatgatacctgataggcagctaagcactagagagtaactgctattattattactgttgttattattatgtttgcataatactgacatgtttctacttaaattctatcgctgagtgta

SEQ ID NO: 189 - DNA, 24 bp, Homo sapiens
aaagttgcatagcttcctcagttt

SEQ ID NO: 190 - DNA, 20 bp, Homo sapiens
ttaaaccactggctttcctg

SEQ ID NO: 191 - DNA, 176 bp, Homo sapiens
aaagttgcatagcttcctcagttttaatgtttgaaatgtctttttcttaatggcaggaatactgggcttagaagttgtattagttagggctcttccgagaaacagaatgagagagagagagagagagagagagagagagagagagacctatcactgcaggaaagccagtggtttaa

SEQ ID NO: 192 - DNA, 1001 bp, Homo sapiens
tatttgagaaagggtgttgttggatcagtcggacttcctgtcctgattgcagtagtgggtggggtgaatttccttctagcagcgtggaaaaggggcatgggaatcaatgcaggtggaacagtggttcctgatgtgacgtaggcaaccattggacattgggcttttttacatcctcagattcaagagcctcttgaaaatgtctcattttgatcatatcgagttctgtctgtgaaagcgatggcaagtctgggttaactagtgaactagtctagtcgagttagcttaagactctttcttataatgcatggacatgtaaaaatcaggaatttcttggtgaaaaaatttgtttccttagaaccagaacaacccataatgcaaacgcataaaaaagatttgcaaattgatgtcctcagtctctctagatacatttcaggtgttcaagatccacgtatagctagtggtgaccatattgacatcatggaaatacctactgggccgtgmtggtttacaccatactctctgaaacaccgcttaggcatttaccccatgattctgtgtatgactgcttttagtagctgctgctgctatttgctaccacgaaggccgcctcctcctcccgtggtcggtaggtaagtttaggttcttgatctcaccacacaaaagaatttgagagtgactccaaaggaagagtagccaaagaagcttattgtaaagcgaaagtaccctctgagaggctgagtgggctgcttaaagggagagacagcaactagtgccttcagaggaattccttttgcgggaattgttcgtatatattcataaaatactggtgaggtcaagtacgtaaagacagacctgcggttgacacatgcgctcagcatctgcatgctgtaacatgcaatgcatgtatcattagcatataaaatctccgcctaggggtgtgtttttttactattaaaatgaagaaaaggttactatgagctaaaccttgagcctagctgcac

SEQ ID NO: 193 - DNA, 20 bp, Homo sapiens
ctggaatggaggaatgcttg

SEQ ID NO: 194 - DNA, 19 bp, Homo sapiens
tccacaaagccattggaaa

SEQ ID NO: 195 - DNA, 304 bp, Homo sapiens
ctggaatggaggaatgcttgaatatagccagttccattgaggtaagtattttggaagcaaaatctaatgaaacataattttatattatgactcagtgtagctcttccatttcttcattagataatttagtcatgttctctgactcaaatactgaagactgataggaaaagcctcaccctggttcatcgtcatatgagtgtaatggaactttcttgacttccagcagtgtctggtgttactcacgttatatgagtagctcaattccatgagttgcttggaattccatttccaatggctttgtgga

SEQ ID NO: 196 - DNA, 1001 bp, Homo sapiens
tgccagacactgttctaagccttttacacacattatctctcttaatgcttcaacaacactatgaagtaggtatgttatttcccccattttacagttgaggaaactgaggcatagagtggttacatgactttcctactgcactgctaggatttggaatttcagtccggcattctcattcccatctgactgtagacctctaggctgtatcatccttttttacagttactaacccaccctgatttcaaataatttacataagtttatttaggtaataactggattttgagccaagaccttactgactagccaaaaactgatccccagaaatacttagacctttttattaagctttattaattgatgtcgaggtgttatttcatttttcttctagaattggtatgcacatttttttgttctttttttcatcccgtcaacacttttgagtgtgtgttatgtggcagatgcctttgttagatactagaagcaaagaaatcagcttcmgtaagactaaaattgtatctggtgataaacacaatgttagagaaacattctgggtgcccatcattattagaccatgtttgcttaatactaatttgtcagttagaatatgtttccagttgtggatgtttcttttttgtctttcttttctttttgcccccaggcattgtctactctggactccatcactctgatgtaccctttcttttaccgtcctatgtttgaagtgatagaagatggctggcattccttccttcctgagcaagaatttgaactctattcttcagctgtgagttaacttttgagaactgtggattatgagaagtaacccaataccttatttgacttgtgaaaatgatcacttcttttgaagagtaataaggtgaagttgacttatccattcctaatcttaatatatttaaaaggattgaagccatgcagagtatgatctctgatcacaaaggaattagattaataatcagtaatactaagatatctaggaa

SEQ ID NO: 197 - DNA, 1001 bp, Homo sapiens
aattagaaagtggttatcaaacaatgtaaataatgaagaccctgggggtctttccagacattcatatttgtaagctatcctggttgtttctgcacaacaagccctttcttaaagaaactagaaaaataaataggacataaatgtcaaaaagtgtataatttttatgtttatattataggcttctcagaaacaaaaaggttagaaagtttttttatgcttagctatttttaattaaaatagaatcccaaatataacaaaggacttttgtgtacagtaatgttctctgggttaaggtttaacaccaaacctgatgtgaccagattctgtttttatcctcctgccagcttcttggaagcctgtaaaatactctttgttttgttgttgttgagagttctaatgccgattgagctttttgacaaatctattgatttttcaacactttgtttctctaccaaaagtcttgtattctatcttctttcatactgagaagaaattgtcctmgtaagaggagcactcaataatggttgttataaattaattactttaatggcagtgttctttcttgatcagatgtaagttgaagctacagcagaagacgatgtctttgtggtcctgggttaatcagcccagtgagctgagtaaattcaccaatcccctctttgaagccaacaaccttgtcatctggccttcagttgctccgcagagtcttccactgtgggaaggtaaaccacgcatcctttgcaaacttcttaacggtcaggtgtgcatgcggctgcctgtgagtgtgtgctgttggtgatgtatgaagatggtgagctggacgtggccctcagacctgtgtgaattgtcattctcagtgtgggcatgtttttctctttcaaatcagttatctagccacactttttttttttttcagttaccattgagaaattaacagtgtttctttacattgctgtttatgttggatatttttctagataagaaagtaccttactctttgc

SEQ ID NO: 198 - DNA, 20 bp, Homo sapiens
ggaccagaaatgggcaatag

SEQ ID NO: 199 - DNA, 21 bp, Homo sapiens
ctcttcagttctgagggttgc

SEQ ID NO: 200 - DNA, 153 bp, Homo sapiens
ggaccagaaatgggcaatagttacaatagttgatcctctgttctggaagctttgaaatttatcagagaatgaagtcattcagtacatctgataaagttttgttgttgttgttgttgttgttgttttaattgggcaaccctcagaactgaagag

SEQ ID NO: 201 - DNA, 20 bp, Homo sapiens
aacggagaaagagggtgtcc

SEQ ID NO: 202 - DNA, 20 bp, Homo sapiens
cccttccagttgcaggagta

SEQ ID NO: 203 - DNA, 382 bp, Homo sapiens
  misc_feature 155: n = A,T,C or G
aacggagaaagagggtgtccatagcctacagaactttctctcagaacttctaggtcagtgctgttctttgggaatctaatatgagccacatatataatttaaaaatttctattaatcacacaagagtaaaaaaaacaggtgaaatgaattgtaantgttttatttaacttaccttactaaaaatattttccatttaacatacaatatgaaattcattaacggatagtcacatttttaaacgccatatcttcaaaatctggtgtttgacagcacatttcagttcaaactagctacgttgcaaggatttaatagccctatgtggctagtgactattgtatggaacattatcgttctagaccctctactcctgcaactggaaggg

SEQ ID NO: 204 - DNA, 1001 bp, Homo sapiens
tctagctttcagatcatccccacgtaaagttcagactttaccagcccagagagtttaaaaaaaaaaaaagagagagagagaaagcgaatgtggattgagcctttacactgaccgcgcagtttgcacagtgcttttcatagattgactgcttttattaaacgctctcaacagtctattaggatggcatggtgattgccccctttctgaggacgcggaaacttgagatttggcgaggcaagaagccaggcgcacacagctaggcgggccgcgggccgcgaccccctggctggtccgtgctctccccctggggaggggtgcaggctgccaggaaaggtgccccctgcgtggccctgggggtgtttcttcctctttgtctcttcttaggcatctgatctcatctcttaagtgggaagagtcggggtggtggaagtagagggtatgggacacggtggacctacctcacttggtagttagtaactgcctcaccttgggcgggtcagyggattctgaacaatggggaaaaggtcccagcttcagggttgctgtgagggtttaagaagagttcaggaaagcagatgcttcaccaacgctccgtagttaccaggcgcctgatttttccttggatcattactattaagaggatgcattggtgatgatgatgatgtaatgagtcagaggttttaaagcccagactgccttgaaaatgcgtctggtaaaccttcttgctccttaaagcagaataagattggagtgggggaacgcagtgaaaatgaaggtgggcatggacatataagtattaagttagaagtggggagggggcagggggcattggcgccaggaagttgtaaactgggcaattatcacccagtccagagcagggaaggcccgttgtgaggggctaggcatgaaggtaccagcagcgtacatgctcctgcagacccctgaggctggaaggaaggagcgggcagtgggagagtaataggtttaagcacgtttgcaag

SEQ ID NO: 205 - DNA, 1001 bp, Homo sapiens
tttacactgaccgcgcagtttgcacagtgcttttcatagattgactgcttttattaaacgctctcaacagtctattaggatggcatggtgattgccccctttctgaggacgcggaaacttgagatttggcgaggcaagaagccaggcgcacacagctaggcgggccgcgggccgcgaccccctggctggtccgtgctctccccctggggaggggtgcaggctgccaggaaaggtgccccctgcgtggccctgggggtgtttcttcctctttgtctcttcttaggcatctgatctcatctcttaagtgggaagagtcggggtggtggaagtagagggtatgggacacggtggacctacctcacttggtagttagtaactgcctcaccttgggcgggtcagtggattctgaacaatggggaaaaggtcccagcttcagggttgctgtgagggtttaagaagagttcaggaaagcagatgcttcaccaacgctccgtagttacsaggcgcctgatttttccttggatcattactattaagaggatgcattggtgatgatgatgatgtaatgagtcagaggttttaaagcccagactgccttgaaaatgcgtctggtaaaccttcttgctccttaaagcagaataagattggagtgggggaacgcagtgaaaatgaaggtgggcatggacatataagtattaagttagaagtggggagggggcagggggcattggcgccaggaagttgtaaactgggcaattatcacccagtccagagcagggaaggcccgttgtgaggggctaggcatgaaggtaccagcagcgtacatgctcctgcagacccctgaggctggaaggaaggagcgggcagtgggagagtaataggtttaagcacgtttgcaagtggaggcggagagaggacaagggctgggggggttggagtttgctgggtctctgggggcaatattgatctatgttaggcgagttttctcactcttcagatac

SEQ ID NO: 206 - DNA, 1001 bp, Homo sapiens
tggtttctccctgcctcttttccctttcatatcccagtccacttctaatggaggatgggattctgcctcatgtcaccagaggtggatatgaatctgttcatactggttttgaatgattttgtcacccatagcagataagcttcaaagttcatgaaaataatgaaggccaagattgagttcctgccccaagaaattccagacctgtgtctggctttcatgagatttttctcttctaatgcccttgcttctcctctttctcggaaccactccatgctggtaagtgttgtctctgaaacgaatgttacctgtattggtctctgtcctagcatgggggagatcattgcatttctaagcgctgcaccacgttcctgggaagattggaagtaagcagcagttatatcagtgcaacctaggacttacgtagttagctaagactgaaaactagtctcactcagttattacattctgggaataattgaactgtttagatttgcattaaascttcacttttttttcttcttcatctaggggctcttggccagctgggagtggggcttgctaatcttttgaggtaagagccctaaaaacttgaaatttaaaatctgagttgttaagtatatggagctcattgggatgccttttaaacttcttttctctctcctcttgctccttaccattgttaagatatatctaaataactgctatatatagctatagatatagatatatagagatatagatatagatacagattttttttttttgagttggagcctcagtctgtcacccaggctgtagtgcagtggtgcaatcccggctcactataacctccacctcctgggttcaagtgattctcctgcctcagcctcccgagtagctgggactacaggcacataccaccacgcctggctaattttttttatttttggtagagatgggctctcgctatgttgcccaggctggtcttctaactcctggcctcaagtgatctgcccgcctca

SEQ ID NO: 207 - DNA, 1001 bp, Homo sapiens
aaaggtccatttagttcacaacccttttcacgttcgtggtttcaatttatgttccttgcaggtccattcatttattctgatatcttggattacaagaatcttcgggagatcgtggtaaacaaccgcatcacctggctgtttcattacagcgctttgctcagcgcctttggagaagcaaatgtttccctggcgagagcagtgaatataactggtaagcatctggctctggctggatgtgatttatttgccagtttttctagttctttaagaagagatgttttcagattctgatagtgtctgttcatttcaggcctgcataacatcctggatgtcgctgcggaacacaatctgcaattgtttgtgcctagcacgattggggcttttggacccacctctccccggaacccaacccccgatctctgtattcagagacccaggaccatctatggggtgtccaaggtccacgcggagctcatgggagaagtaagcatcactcagctrgattgctgaatgtgccctggctgtcacgatttgctgtttgctttctcattcgttttgcctccaaggcctggtgattcatccctggaggaactttacctcttcttggatcccagccccagagtcgcttacttaactcactgggtttgccatgtagcaggtgtctccagctcctgaaacctcctcagccatatgggaacactcagcacttcctgggtgccccgtgcccagccccgatctcttcatttgctgcttgtcttgtactccaccattctttctggctcctagtattggtagccattggtagtaactctaaaacctcaaacatcttgggtttgttttgtttgtttgtttgttttatgagacagaatcttgctctgtcacccaggctggagtgtggtggcgtgatctcagctcatagcagcctccgcctcctgggttcaagggatcctcatgcctcagcctccgaagtagctgggattataggcacgtgccaccacacc

SEQ ID NO: 208 - DNA, 20 bp, Homo sapiens
acctctttccagataagccc

SEQ ID NO: 209 - DNA, 20 bp, Homo sapiens
ccaatggtttcggttactgt

SEQ ID NO: 210 - DNA, 213 bp, Homo sapiens
acctctttccagataagcccttgaggtctcgggctgacctacacacacacacacacacacacacacccccccccacacacacacacgacagagaacatgccataaacatccttgaacccatgcaggaaagcccatcccatattctgaaaaaatgccaaattaggtttttctttctttttggaaatcagtcattacagtaaccgaaaccattgg

SEQ ID NO: 211 - DNA, 19 bp, Homo sapiens
aacccagcatcctacaaag

SEQ ID NO: 212 - DNA, 17 bp, Homo sapiens
catctggaacccatgag

SEQ ID NO: 213 - DNA, 273 bp, Homo sapiens
aacccagcatcctacaaagaaaatacatggtctgtctacccaaggttagagtgggaggggatgtgagagtttgcagggaggtgtgctggcccttatgtgatctgtgataagacatcacctttatgcccaccccaacagacagaggttggaaaataacaataccagacacacacacacacacacacacacacacacacacacacacacacacacgattccagcagccactcagaaagaaaacaaggaaatgactttgctcatgggttccagatg

SEQ ID NO: 214 - DNA, 1001 bp, Homo sapiens
agtatcatccttcacaaagttctttctattctttctactgtacaaagttttctgttgtcaaatagcaagagatctctgttttctacttggaatgggcctggagaagggagacagcacccgctccctccaccccttgtccctgagcacagcatggtgacctgccaagccagagggtgacctggacactcataactcaatgcagggccaactgtagcctctggccggtgtccctgagtgagggcaaagttgtaataacacttgttctctcctttctccaatttgctcccaagctccattgctttcgttcaggccctcccccttctagactgggcagttccgcatccttggagctcatttctctgtcttcagaatctgatgctccaattcatcccatgtgtggctgccaaggtctttctaaaactcaaatgtggccctatcaccgcacagggtaaagccaccataaactcctctgtgtttgagaacaagggccaagtctcccaytgaggcctccagggagtggacagtctgggtctcctttcttctccaagcacgctgggcccatctgtcctgtccctgaggactccctggcacacatgacacttcagagcttttgccaactccactccctgcctgaaatgcccatctccttcagagagcttctatgtatccttggaggtccagtcctaatgtccctgcctccgataagacctctccccatcttcctctcgccctgctcctgtccccgccaggcatgacaaatctcttcccacagtgggcccaacagggaggcagatggtagaacaggttttgggccaggtgccaggtgcacgtggctcttcatcctggttccccaccgcacacctggagagctgagtgcttttcctgaggtcacgcagaaggttaccagcctggctctggagctgtctctttgccacatcgtggggtgtctttaaggtgaccttgaatgtgcttgaagctgttttatgtcctatttgcagacc

SEQ ID NO: 215 - DNA, 20 bp, Homo sapiens
ctgggaatccgagattgaaa

SEQ ID NO: 216 - DNA, 20 bp, Homo sapiens
ggccataatcaaggcagaat

SEQ ID NO: 217 - DNA, 288 bp, Homo sapiens
ctgggaatccgagattgaaatgaaagaaatcgaaagatctttgcctacatacagaggtccagtaatgggatagggaatatattatccccgggatagcgccactgtactccagccaggatgacagagactccatctcaaataaataaataaataaataaataaataaataaataaataaatacataaataaagtgcctctttgttaaggcagttgcttctatttctacttttttaaccaaagctaattgctaatgtgttaaagtacgagattctgccttgattatggcc

SEQ ID NO: 218 - DNA, 1001 bp, Homo sapiens
aagatatgaggaaagagaaagggcatgagcaaaggacatttttgcagcatgtttatgatcttgagaaaatggaaacagctggggtgtgcggcagaagaagtggggaaaatgacaacggttcattaaacctcacgatcagatgctgacagcccctcacaggttgctgcagacaaaacagggaacgacaggaaaaagatgaccgtgatacgctctgctaaaagcaggtcgcaaaacaggatgtagataatgatcccattttgcttttttacaaaaaaaaaaaaaggccatggaaaattacatatcacgaatgttcagagtggctgtctctggatgatggcattggagttaattttatctttcactctattttctgaatttcctatatcaaaagcaaattgatggtgtgaaggggaaagcatatttaatgtgattcctaaaaggctcagccctccctgcatggattgagcactgaaagaagagggttctgtcacctctttcgtsctgacccttgccttttctaatgttgctcagaggcacacagacgtatttgctttaagtaattgcttgtctgtttttaatatcacattttgaaaaggtatttagacaacatgagtttattactttctgtttaacccaaatccttcagaggtacttaaagcaaaatgtaaagtcctcttatccctttgtgaatttcagtccccagaagtctcactgttagtagtttgatttttaccaaaaatgtccaggtattttcttttcatctgcaaatgtgttaatagactcctttttttaaatttcacacaagcaggattatatcatacaaaacattctgcaatttactcttttcatgtaacaataatgtatcctgggtatttttctttgccagttcagatctcttttatccttttactaatttatttacctatctattcatttgcttaacttgattttattattatacaagttatccatgaatattgttttcaaaaatttaaacagtc

SEQ ID NO: 219 - DNA, 1001 bp, Homo sapiens
atacacatgcaaacacatacacatgtccacgcatgcacatatacacacacacgcacacatatacatgtgcacatatgcacagatgcaatgaacacgtgtgcaacacatgtacacaccttacacgtacatatgcacacacacacacaactccaaagcaagacccctctgcttctccgagccacagcagtgaatgcaagacagggatggaagcaggggagtgagttctacccttcgtggcctccggggtgtccttgagcctctcaagcctcagtttactggtgtctatgtgaggatagactagtttcacagctcaaaggcaggcggtccttcagtgctgagaaatcttcatctcagagccaggccctgcctgcccagggcagtccagacataccacagaggcaggggatccaggttttgtgaaactgaagctgataggatctgaggtcgtctttacaaaggacaccaaattgtcagaagccatcagggacggggcctcagagmagccaggcaagtgaggggtctaaagcaccagcttgggaagcgtcactgcgtggagagcgggctcctgggctcatcgcccgaggcacccgacacaagtgcagcctacaaaatggagagaaaagcccttgatgaatgaactccctaaggccaggctcgggttccttagagactgggggcacagctgcacccgggcagggtcggggagacagtttgcagcctctgggctgaggctggggtgggggtgtggaggggctgtggcaacagcatggcgtacgcctctgggtgtccttttgcaagtaggtgatgagagaggcacattggctgagggaaactggaggatggaagggggttgaggcaggggaactgacaggagaggaaagagccttaagtcaaacaggaccgcggaaaaccaagcgtccacaacgagaacgaggggtccgtgcctgacccctggcggggaggcgtggtactgctcgaggtaggcgcggactcggggaacc

SEQ ID NO: 220 - DNA, 20 bp, Homo sapiens
gcagcctctaaccacatgct

SEQ ID NO: 221 - DNA, 20 bp, Homo sapiens
ctttgcatggcttcctatgg

SEQ ID NO: 222 - DNA, 380 bp, Homo sapiens
gcagcctctaaccacatgctgaccatgccaatggctctctaagcacacatgtacacacacacactctcacacacataaaaacacagactcacacacacacggacaaacacaaacacatacacagactcacacagacacgcaaactcacacacagacagacacacacacagacacacagactcacacacacaaactcacacagacacacaaatacacagactcagactcaaacacaaactcacacaaacacatttacacaaactcacaaactcacacacacaaacacacacacaaacacgcaaacttacacacacatgagcagacacacacccggcccttctgggctcttcttttcttactccataggaagccatgcaaag

SEQ ID NO: 223 - DNA, 20 bp, Homo sapiens
gaatgggcacatccataggt

SEQ ID NO: 224 - DNA, 19 bp, Homo sapiens
cgcccttccttatccctct

SEQ ID NO: 225 - DNA, 257 bp, Homo sapiens
gaatgggcacatccataggttctgattttgacacatggccaagactatcaagtgaggggaaagggtgcagaaaaacacatacatgcagcatgatgtacacacacacacacacacacacaattttatgttcatcacacacatgcatatttgtgtaaacatgcagcaaagggatcccagtgataccaaccaaagagagccccgtgacctccgaggagggagcggctggggctgtcagcgcagagggataaggaagggcg

SEQ ID NO: 226 - DNA, 25 bp, Homo sapiens
gagactgacaatctcctcgtcttat

SEQ ID NO: 227 - DNA, 25 bp, Homo sapiens
ctattgcctagcttagcacatttga

SEQ ID NO: 228 - DNA, 125 bp, Homo sapiens
gagactgacaatctcctcgtcttatccacgttctcactccaaattcattaagttaaatacacacacacacacacacacacacacacacactaagacagtttcaaatgtgctaagctaggcaatag

SEQ ID NO: 229 - DNA, 21 bp, Homo sapiens
cctaagcatttcttggcttcc

SEQ ID NO: 230 - DNA, 22 bp, Homo sapiens
cagtgagagcaccctactttga

SEQ ID NO: 231 - DNA, 153 bp, Homo sapiens
cctaagcatttcttggcttcccccaggtgccctgtttttgaattaacctgagattatggcagaccacaagggctgcatcacaccaagttctccccaagatttgccatatttcctctaccaccaggtggggttcaaagtagggtgctctcactg

SEQ ID NO: 232 - DNA, 20 bp, Homo sapiens
tccacagcagggttcaataa

SEQ ID NO: 233 - DNA, 20 bp, Homo sapiens
cccactcatccatctatcca

SEQ ID NO: 234 - DNA, 275 bp, Homo sapiens
tccacagcagggttcaataagtgattgctgctcattacctagctatacaggtagatatggatggatggatggatggatggaaggatggatgatggatggatggaaggatagatagatggttggataggtggattgatagatgatggatggatggatggatggatggataaatggataaatggatggatggatggatggatatctggatggatggataaatggatggatggatggatggatgaatagattattagatggatagatggatgagtggg

SEQ ID NO: 235 - DNA, 20 bp, Homo sapiens
ggctcgctccagctttatct

SEQ ID NO: 236 - DNA, 19 bp, Homo sapiens
gggtgatgcatagcagacg

SEQ ID NO: 237 - DNA, 268 bp, Homo sapiens
ggctcgctccagctttatctgcctcttaggtgtgaccaaattgtcgtgtgtgcgtgtgtgtgtgtgtgtgtgtgtgtgtgtgtgtgtgtgttggctccaaaggtttattcacgaatagatcccaaagaaatgtcacagagaaatagtgacttgaagtccaaagaggaaaaaaagggaggccgcaggcacatgatggatctgtgcaatagtcatacgtaagccgccgtgatgtccacaccacggagaccccgtctgctatgcatcaccc

SEQ ID NO: 238 - DNA, 1001 bp, Homo sapiens
aaaaactcctggcagacccttccgggatcacgcgtggctcaactcgggggccgtagctacgatccccgcgcagacgccggaatccggggcccggtccccgcgcggggtgcggcgctcgcggggggggggggggggatggggtcggtccctctcgggaacggctgctgttgtttctttagatactgaatataatttctccctcctccaccccactcgctgttcttaacaattttatttattggtttactattgtcttgtgaacgtttcttgtctcctccttgccttttttcatcccctttctctcttcatttctctctttttccttaattctgttgcaaagtttccttttcttgcttaatcaaaattctccccgcttactttgttctttgcccacagcattcgttcttcttttctccttgcctgcctgtcttctttcccgctgttcttggccgtgggcagacccggctgatgtaaggactgcagcttttccctggcatactmtgcgccttcagatgtggtctgcgtctgcctgggtctcttcccacctcaatctgagatccttgcccctcacaataaattcgtttttattcattctgatgtttgtctacagaagttactcgataaagatgttttgtttcatgaatcaaaaggcttcttgtctgtgaattattttaatttctggatattaaactgcacagtagctattttatttgcctttaataaatttcttaggtttttacctctaactaatggcacattttaaataattttccaagcactaggtggtgtctgacaagattgattcactcaaaaacgatgcagaatttcttaaatgtagaatcttttaaaacggtgtcggatggcttctcctgctacatcgtttatttgtagcttccactaactctaaagattgaacaggaaactgatatggtagaaatagataactttgccttgttcactagctaagattttatttgctttctgttagatcacagtagtgc

SEQ ID NO: 239 - DNA, 24 bp, Homo sapiens
aattcctggatattcctaccactt

SEQ ID NO: 240 - DNA, 20 bp, Homo sapiens
gatccttactccagcccaca

SEQ ID NO: 241 - DNA, 359 bp, Homo sapiens
aattcctggatattcctaccacttactatttgttgtcgttgtttctattgtttttgagagaaggtcttgctccattgcccaggctggagtgcagtggcgtgatcatggctcactgcagtctttacctccagggttcaaggaatcctcacacctcagcctcctgagtagctggaattactaccatgcccagctaacgtctatattttttggaggtagggttttgccatgttgcccaggctggtcttgaactcatgagctcaagtgatactcctgcctcagcctcccaatgtgctgggattacaggcataagccatcgtgcctggcctcagtgagtggttttgtgggctggagtaaggatc

SEQ ID NO: 242 - DNA, 19 bp, Homo sapiens
agatcacgctccagggatt

SEQ ID NO: 243 - DNA, 25 bp, Homo sapiens
tcccacactacactgatgtaaagaa

SEQ ID NO: 244 - DNA, 390 bp, Homo sapiens
agatcacgctccagggattcctgcgtcctttaataagattctggggtgggcacagttctggggtggacatggtggctcacgcccataatcccagaactttggaaggctgaggtgggaggatcgcttgagcttaggagttcaagaccagtctgtacaacacagtgagagcttgtctctcccaaaaaaaaaaaaaaaaaaaaaaaaattagcaaggcatggcagcatgcacctgtagtcccagatacttgggaggctgaggtgggaggattgcttgagcctaggaggttgaggctgcagtgagccgagatcgcagcactgtactccagcctgggggacagagtgagaccctgtctcacaaaaagtttttctttacatcagtgtagtgtggga

SEQ ID NO: 245 - DNA, 1001 bp, Homo sapiens
gggaggcagaggttgcagtgagctgagatcgcaccattgcactctagcctgggcaacaagagtgaaactccgtctcaaaaagagaaaagaagtctcacaaagggctgggcacagtggctcatgcatgtagtctcagcactttgggaggctgaggctggagtatcgcttgagcccaggggttcaaggctggactgagttatgactgcaccactgtactccagcctgggtgacagagtgaccctgtctctaataaaaagaataaaataaatacagtcttacaaaggatacaatagaaccaaatgctcaaaacattagtgacaatctggattttctttatatattttggcactaattttcctaaggtaaatatttattatatctttatgcaaaaggaaaagtaatcttactaactttgaaagggaaaaagagagagcaaggtttgcgtggacctcagtgtgaggtgagaggcctagggctggaggctctgaatgtgatacctgsactgaaatccaggtgtcccgcctcccagcccaggacgtgggtgatcactgcaactttttcctcttctcgtgctcaggggaactctcagtgtctgggattagggagcaggggctgaagtcagagtgaggaagagcaagagcagcccgaggtggtcttctctttccaaggaaagggcattgtttctgtgcgctctagattctcagatgtgagagctgggcataaacaaagaattaatcctctgtgtcttttcttgtctgttccccccaactcagtagatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctggtaagcacgtgcctcgcagcctcctctgggcacctggctgcggagctctcgccttggt

SEQ ID NO: 246 - DNA, 20 bp, Homo sapiens
ttctggccttaggaaagtgc

SEQ ID NO: 247 - DNA, 21 bp, Homo sapiens
ccagaccacagaagctactcc

SEQ ID NO: 248 - DNA, 424 bp, Homo sapiens
ttctggccttaggaaagtgctagctgagctgaaatctcatgaatgttaggtcgtttgtgtacttcttatcaatgtaatgaagcttttgcacagaaagtctgtttgtttttgtgacatgtgttgccagtattgtttcaagtctgtcctctgtcctttgattgtgcttatgatgtctcttggcatttgggattttaaatttttatatcatcaacggtgggtatttttcttggttgcttgtaggtttccccttttgctaaaaaaaggccccttctgcccccagagaaagtcacatgccttctattttctgaagttttataacttgtaaaaatgtttagaagtgtagtctttatttgtgtggcctgacgtaggtaccataggatgctatgggctgtaaaaataactcggagtagcttctgtggtctgg

SEQ ID NO: 249 - DNA, 24 bp, Homo sapiens
gcatgtgaaattggacttgtactc

SEQ ID NO: 250 - DNA, 21 bp, Homo sapiens
cactgcaagcctagagaagga

SEQ ID NO: 251 - DNA, 292 bp, Homo sapiens
gcatgtgaaattggacttgtactccagagatatccatgtttgtattcatgtaaaaataatgtccttcttaattatctgggggtggtggtgtgtgcctttagtgccagctacttggaaggctgaggcaggagaatcacttggaccaaggaggcagaggttgcagtgagctgagatcgcgccattgcactccagcctgggtgacagagagagactctgtcccaaaaaataaaataaaataaaaataaatacataaaataaaataaaataaaagtccttctctaggcttgcagtg

SEQ ID NO: 252 - DNA, 20 bp, Homo sapiens
gaagatttggctctgttgga

SEQ ID NO: 253 - DNA, 25 bp, Homo sapiens
tgtcttactgctatagctttcataa

SEQ ID NO: 254 - DNA, 142 bp, Homo sapiens
gaagatttggctctgttggagacagactcatagatagatagatagatagatagatagatagatagatagatagatagatagatgatagatagatcttatttaaaagtttattaacttattatgaagctatagcagtaagaca

SEQ ID NO: 255 - DNA, 20 bp, Homo sapiens
tgggagatttcagcctttca

SEQ ID NO: 256 - DNA, 20 bp, Homo sapiens
tcaaagaccagtgccagaga

SEQ ID NO: 257 - DNA, 352 bp, Homo sapiens
tgggagatttcagcctttcaaaaaaatataatgtcttgtactatggattttcctggagtgaaagagaagaaaatctcttttggctcatctctttttactcctacacacacacacacacacacacacacacacacacacacactctatatgatagattataacagatgtatctttcaaaagtagaactgaaatttagacctaaaagataatatactttaattgttagagaggatatttttcctgttgaagggaacaatattcctatgtgtttaatacacaaatatatctgtgccagtacttgttaccccctgagacttcacacactacttatatctctggcactggtctttga

SEQ ID NO: 258 - DNA, 20 bp, Homo sapiens
tgggagatttcagcctttca

SEQ ID NO: 259 - DNA, 20 bp, Homo sapiens
tcaaagaccagtgccagaga

SEQ ID NO: 260 - DNA, 352 bp, Homo sapiens; identical to SEQ ID NO: 257

SEQ ID NO: 261 - DNA, 20 bp, Homo sapiens
tccatcccaactcaagatcc

SEQ ID NO: 262 - DNA, 21 bp, Homo sapiens
agcctggtctctaccataagc

SEQ ID NO: 263 - DNA, 405 bp, Homo sapiens
tccatcccaactcaagatcccaggtaacaataatacctgcttcttgatataaggattcaacaattttttaaagcgctgagaccatgcctgttacatagtaggcacttaacacacgctgattatttacatctaaatcttcacaaccaccctaagaagtacatgttattattcccatcttacaatagagaaaataagctcagattaattaattttcttgggtcttacagcaagtaagtgatggtactggtatctgtacttatattgaatggtttgactgtaaaattcttcttttctctatatcaaatagtcccacgaggaatgtgtgtgtgtgtgtgtgtgtgtgtgtgtgtgtgtgtgtgtattttaaatgagaaccaagcaaaagcttatggtagagaccaggct

SEQ ID NO: 264 - DNA, 23 bp, Homo sapiens
tccttgcaaatgtctctttcttc

SEQ ID NO: 265 - DNA, 20 bp, Homo sapiens
atgggaaggaatttgggact

SEQ ID NO: 266 - DNA, 171 bp, Homo sapiens
tccttgcaaatgtctctttcttccccctggtaccatacccctgtatctcttaagacaacacacacacacacacacacacacacacacacattctctccctctctcactccctacttttttccttcccactgagagattcaaaccttcaaaaagtcccaaattccttcccat

SEQ ID NO: 267 - DNA, 20 bp, Homo sapiens
caccattctgtcggctgtaa

SEQ ID NO: 268 - DNA, 20 bp, Homo sapiens
aaagggcttggtaactcctc

SEQ ID NO: 269 - DNA, 180 bp, Homo sapiens
caccattctgtcggctgtaaaagcacggcaccagcatctgctcggcttcttgtgaggcctcaggaagcttttactcatggttgaaggtgaatgcagagcaggtatatcacatggtgagagggggagtgagagagagagagagagagagagagagagagaggaggagttaccaagcccttt

SEQ ID NO: 270 - DNA, 20 bp, Homo sapiens
cacgaccacaccagcctaat

SEQ ID NO: 271 - DNA, 18 bp, Homo sapiens
aaaggcaggcaggcacag

SEQ ID NO: 272 - DNA, 195 bp, Homo sapiens
cacgaccacaccagcctaattttgtgtgtacgtgtgtgtgtgtgtgtgtgtgtgtgtgtgtgtgttttggtagaggcagagtttcactatgttgcccaggctggtcttgaactcctgggctcaagtgatctgccccacctcggcctcccgaagtgctgggattacaggtgtgagcctctgtgcctgcctgccttt

SEQ ID NO: 273 - DNA, 20 bp, Homo sapiens
gaatggaagcaaggatgagc

SEQ ID NO: 274 - DNA, 21 bp, Homo sapiens
gacgctggtctatttcaggtg

SEQ ID NO: 275 - DNA, 304 bp, Homo sapiens
gaatggaagcaaggatgagctgctgcatttctgtagctggcattcagctcaagaatacgtaaaaccagactcgtggttttttctttctttctttctttctttctttttgaatgtgaggcctttacagaaaaagaaaatgtcagtctgattatccagggcatgaggataaagagaagcccaaacaaaggtttcccccactccaccccacccaatatactgtggcactagaaaacgattccagaatcagaaactatatgctgacgtccattagccctcttagtagcacctgaaatagaccagcgtc

SEQ ID NO: 276 - DNA, 20 bp, Homo sapiens
caatcaagcctgtgtcgagt

SEQ ID NO: 277 - DNA, 20 bp, Homo sapiens
aggaaggcatttgaatgagc

SEQ ID NO: 278 - DNA, 169 bp, Homo sapiens
caatcaagcctgtgtcgagttaagaattaaatgggaggttgcagtgagccaatatcatgccactgcactccaggctgggcgacaggataagactccatctcaaaataaaaaaaataaaaaaataaaggtttgtatttcttttttcttaagctcattcaaatgccttcct

SEQ ID NO: 279 - DNA, 20 bp, Homo sapiens
ggatggcctttggtaactga

SEQ ID NO: 280 - DNA, 24 bp, Homo sapiens
ggaaatgaacatgataacatctgg

SEQ ID NO: 281 - DNA, 175 bp, Homo sapiens
ggatggcctttggtaactgatctcatgaccaatattaagctgtgagctctcttttccgaatttttacattatcctcttacaaccacctccctcaacacacacacacacacacacacacacacacacacacactctctctcacactccccacccagatgttatcatgttcatttcc

SEQ ID NO: 282 - DNA, 20 bp, Homo sapiens
ccatttacgctttggtctgc

SEQ ID NO: 283 - DNA, 20 bp, Homo sapiens
ccctttgtcaagtgctttca

SEQ ID NO: 284 - DNA, 102 bp, Homo sapiens
ccatttacgctttggtctgcagagactattaattatttggttgtttttgttttcatgtttgaataagcacagattctggcattgaaagcacttgacaaaggg

SEQ ID NO: 285 - DNA, 20 bp, Homo sapiens
ttccgaggtaagcctttgtg

SEQ ID NO: 286 - DNA, 20 bp, Homo sapiens
accctctttcagagccaggt

SEQ ID NO: 287 - DNA, 307 bp, Homo sapiens
ttccgaggtaagcctttgtggcccctgaccctaatacagaagagacactaatttattttcctgctctgtggtcccagagttatgtgaatttccttttgaaattcatcatgcatatttatttatttatttatttatttatttatttaagcatatttctctatcagagtatacctgtcaccatggcagggatttgtctgcctctttctctttcactgaagtacccacagtacccggcatagtgctggcgctgttcagggtgcccggtaaacttgtgtgaatgaatttttacctggctctgaaagagggt

SEQ ID NO: 288 - DNA, 20 bp, Homo sapiens
aatcgctgctacagggacac

SEQ ID NO: 289 - DNA, 24 bp, Homo sapiens
aactgcataaatatttgacgtgga

SEQ ID NO: 290 - DNA, 113 bp, Homo sapiens
aatcgctgctacagggacacacatatctctctatccatacacacacacacacacacacacacacacacacgtgtacgtatttctagtattccacgtcaaatatttatgcagtt

SEQ ID NO: 291 - DNA, 20 bp, Homo sapiens
gtccaggctcacctgaagtc

SEQ ID NO: 292 - DNA, 19 bp, Homo sapiens
cggagggagctaggaacag

SEQ ID NO: 293 - DNA, 138 bp, Homo sapiens
  misc_feature 106: n = A,T,C or G
gtccaggctcacctgaagtctgagattttgggagctttggagaattctggataaaatcccttactggacttagcaggaatctccgatctgtggagaagtctcctcnagagactgagcatctgttcctagctccctccg

(19) United States
(12) Patent Application Publication
(10) Pub. No.: US 2007/0111209 A1
(43) Pub. Date: May 17, 2007

(54) Inversion on chromosome 8p23 is a risk factor for anxiety disorders, depression and bipolar disorders

(76) Inventors: Soley Bjornsdottir, Grafarvogur (IS); Augustine Kong, Chicago, IL (US); Thorgeir E. Thorgeirsson, Reykjavik (IS)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/571,865

(22) PCT Filed: Sep. 17, 2004
(86) PCT No.: PCT/US04/30699

§ 371 Date: Mar. 14, 2006

Related U.S. Application Data
(60) Provisional application No. 60/504,307, filed on Sep. 19, 2003.

(51) Int. Cl. C12Q 1/68

(52) U.S. Cl. 435/6

(57) ABSTRACT

An association between psychiatric disorders and disorders comorbid with psychiatric disorders, and genetic markers in the 8p23 genomic region is described. Markers are also provided to diagnose or detect a susceptibility to disorders comorbid with panic disorder and independently of comorbidity with panic disorder. Methods and surrogate markers for detecting the orientation of the Inv8p23 inversion fragment, thereby diagnosing psychiatric disorders or comorbid disorders or a susceptibility to psychiatric disorders or comorbid disorders, are also disclosed. The methods described herein are also useful for determining responsiveness of drugs useful for treating psychiatric disorders.

### RELATED APPLICATIONS

This application is the U.S. National Stage of International Application No. PCT/US2004/030699, filed 17 Sep. 2004, published in English, and claims the benefit under 35 U.S.C. §119 or 365 of U.S. Provisional Application No. 60/504,307, filed Sep. 19, 2003, the entire teachings of which are incorporated herein by reference.

### BACKGROUND OF THE INVENTION

In general terms, panic disorder is a manifestation of anxiety in which feelings of extreme fear and dread strike unexpectedly and repeatedly for no apparent reason, accompanied by intense physical symptoms. Panic disorder is characterized by unexpected and repeated episodes of intense fear accompanied by physical symptoms that can include chest pain, heart palpitations, shortness of breath, dizziness or abdominal distress. About 1.7% of the adult U.S. population ages 18 to 54—approximately 2.4 million Americans—has panic disorder in a given year. Panic disorder affects about 1 out of 75 people worldwide. Women are twice as likely as men to develop panic disorder. Panic disorder typically strikes in young adulthood. Roughly half of all people who have panic disorder develop the condition before age 24.

Many people with panic disorder develop intense anxiety between episodes. It is not unusual for a person with panic disorder to develop phobias about places or situations where panic attacks have occurred, such as in supermarkets or other everyday situations. As the frequency of panic attacks increases, the person often begins to avoid situations where they fear another attack may occur or where help would not be immediately available. This avoidance can develop into agoraphobia, an inability to go beyond known and safe surroundings because of intense fear and anxiety.

Panic disorder can coexist with other comorbid disorders, e.g., depression, bipolar disorder (also known as manic-depressive illness; a brain disorder that causes unusual shifts in a person's mood, energy, and ability to function), obsessive-compulsive disorder (characterized by intrusive, unwanted, repetitive thoughts and rituals performed out of a feeling of urgent need), histrionic personality disorder, family denial and dysfunction, hypercholesterolemia and substance abuse. About 30% of people with panic disorder abuse alcohol and 17% abuse drugs, such as cocaine and marijuana, in unsuccessful attempts to alleviate the anguish and distress caused by their condition. Appropriate diagnosis and treatment of other disorders such as, for example, depression, bipolar disorder and substance abuse, are important to successfully treat panic disorder.

Heredity, other biological factors, stressful life events, and thinking in a way that exaggerates relatively normal bodily reactions are all believed to play a role in the onset of panic disorder. The exact cause or causes of panic disorder are unknown and are the subject of intense scientific investigation.

Studies in animals and humans have focused on pinpointing the specific brain areas and circuits involved in anxiety and fear, which underlie anxiety disorders such as panic disorder. Fear, an emotion that evolved to deal with danger, causes an automatic, rapid protective response that occurs without the need for conscious thought. It has been found that the body's fear response is coordinated by a small structure deep inside the brain, called the amygdala. The amygdala, although relatively small, is a very complicated structure, and recent research suggests that anxiety disorders are associated with abnormal activity in the amygdala.

Treatment for panic disorder can consist of taking a medication to adjust the chemicals in the body, or treatment might involve working with a psychotherapist to gain more control over your anxieties. Both types of treatment can be very effective. For many patients, the combination of medication and psychotherapy appears to be more effective than either treatment alone. Early treatment can help keep panic disorder from progressing. Therefore, early diagnosis of panic disorder is essential for providing effective treatment.

The symptoms associated with panic disorder (e.g., chest pain, heart palpitations, shortness of breath, dizziness or abdominal distress) often mimic symptoms of a heart attack or other life-threatening medical conditions. As a result, the diagnosis of panic disorder is frequently not made until extensive and costly medical procedures fail to provide a correct diagnosis or relief.

### SUMMARY OF THE INVENTION

A number of genetic disorders, both Mendelian and complex, are associated with genomic rearrangements. Such arrangements can cause the disorder directly, or it simply may be linked to the disorder without being a causative contributor.

Described herein is the association of a known inversion region on chromosome 8p with a psychiatric disorder, e.g., an anxiety disorder such as, for example, panic disorder (PD), and the identification of markers useful in detecting a particular allelic variant of the inversion fragment, including, for example, highly correlated genetic markers, microsatellite repeats, single nucleotide polymorphisms (SNPs) and small insertion/deletions (INDELs). These correlated markers, both individually and in combination, reliably serve as a diagnostic surrogate to FISH in detecting the chromosome 8p inversion status of an individual. Thus, the chromosome 8p inversion fragment, either in the inverted or reference (“common”) orientation, and any of its correlated genetic markers or marker haplotypes, serve as a diagnostic test for complex psychiatric disorders. Additionally, other inversion related markers or marker haplotypes associated with the identified markers and marker haplotypes can also be used as a diagnostic test for anxiety disorders such as, for example, panic disorder and bipolar disease. These inversion related markers can be used to determine either orientation of the inversion fragment (Inv8p23 genomic region). For the purposes of the methods described herein, either or both orientations of the inverted fragment can provide information related to a psychiatric disorder.

These inversion-related markers and marker haplotypes can also be used to discover new associations of the inversion to other disorders, or as a diagnostic for other disorders that are subsequently shown to be associated with this chromosome 8p inversion, e.g., comorbid disorders.

In one embodiment, the invention is directed to a method of diagnosing a psychiatric disorder or a comorbid disorder in an individual comprising detecting the orientation of the Inv8p23 genomic region, wherein the orientation of the Inv8p23 genomic region is indicative of a psychiatric disorder. In one embodiment, the psychiatric disorder is an anxiety disorder, e.g., panic disorder or bipolar disorder. In one embodiment, the inverted orientation of the Inv8p23 genomic region is indicative of panic disorder. In one embodiment, the orientation of Inv8p23 is determined by detecting one or more markers at one or more polymorphic sites, wherein the one or more polymorphic sites are in linkage disequilibrium with Inv8p23, and wherein a particular allele at the one or more polymorphic sites is indicative of a particular orientation of Inv8p23. For example, the one or more markers can be selected from the group consisting of: SG08S5, SG08S95, DG8S269, DG8S163, DG8S197, AF131215-2, DG8S127, SG08S120, DG8S179, SG08S27, DG8S261, SG08S71, SG08S32, SG08S517, SG08S70, SG08S102, SG08S73, SG08S76, SG08S26, DG8S242, SG08S15, DG8S257, SG08S138, DG8S161, SG08S520, DG00AAHBG, SG08S508, DG8S156, D8S1695 and DG8S170. In a particular embodiment, the one or more markers comprise the A allele for SG08S71 and the G allele for DG00AAHBG. In another embodiment, the inverted allele of Inv8p23 is detected by detecting a haplotype comprising one or more genetic markers. In one embodiment, one or more genetic markers of the haplotype are selected from the group consisting of: SG08S5, SG08S95, DG8S269, DG8S163, DG8S197, AF131215-2, DG8S127, SG08S120, DG8S179, SG08S27, DG8S261, SG08S71, SG08S32, SG08S517, SG08S70, SG08S102, SG08S73, SG08S76, SG08S26, DG8S242, SG08S15, DG8S257, SG08S138, DG8S161, SG08S520, DG00AAHBG, SG08S508, DG8S156, D8S1695 and DG8S170. In a particular embodiment, the haplotype comprises the A allele for SG08S71 and the G allele for DG00AAHBG.

For the embodiments of the present invention, surrogate markers can be used to identify the markers identified herein. Surrogate markers can be, for example in linkage disequilibrium with one or more markers selected from the group consisting of: SG08S71, DG8S197, SG08S73, DG8S332, AF131215-4, SG08S5, SG08S520, SG08S95, SG08S508, SG08S102, DG00AAHBG, SG08S70, DG8S161, DG8S298, SG08S506, SG08S15, DG8S249, DG8S148, DG8S269, DG8S127, SG08S93, D8S1695, SG08S517, AF131215-2, AF131215-1, DG8S242, DG8S136, D8S516, DG8S148, SG08S39, D8S1130, DG8S127, DG8S232, DG8S137, DG8S269, D8S550, SG08S507, SG08S507, DG8S245, DG8S197, D8S1825, SG08S27, SG08S27, DG8S257, D8S503, DG8S297, DG8S297, SG08S120, SG08S120, D8S351, DG8S159, D8S1695, D8S1759, SG08S26,SG08S26, D8S1130, DG8S221, D8S1130, D8S1759, DG8S307, DG8S153, DG8S277, DG8S192, D8S1695, DG8S265, DG8S257, DG8S127, DG8S163, DG8S163, DG8S156, DG8S261, DG8S179, SG08S138, SG08S32, SG08S76 and DG8S170. In particular embodiments, the surrogate marker is DG8S132.

For the embodiments of the present invention, the comorbid disorder is selected from the group consisting of: depression, bipolar disorder, obsessive-compulsive disorder, histrionic personality disorder, family denial and dysfunction, hypercholesterolemia and substance abuse. In particular embodiments, the comorbid disorder is selected from the group consisting of: depression, bipolar disorder and hypercholesterolemia.

In another embodiment, the invention is directed to a kit for diagnosing a psychiatric disorder or a comorbid disorder comprising at least one agent useful for detecting the orientation of the Inv8p23 genomic region, wherein the orientation of the Inv8p23 genomic region is indicative of the psychiatric disorder. In a particular embodiment, the psychiatric disorder is an anxiety disorder, e.g., panic disorder or bipolar disorder. In a particular embodiment, the inverted orientation of the Inv8p23 genomic region is indicative of panic disorder. In a particular embodiment, the orientation of Inv8p23 is determined by detecting one or more markers at one or more polymorphic sites, wherein the one or more polymorphic sites are in linkage disequilibrium with Inv8p23, and wherein a particular allele at the one or more polymorphic sites is indicative of a particular orientation if Inv8p23. In one embodiment, the one or more markers are selected from the group consisting of: SG08S5, SG08S95, DG8S269, DG8S163, DG8S197, AF131215-2, DG8S127, SG08S120, DG8S179, SG08S27, DG8S261, SG08S71, SG08S32, SG08S517, SG08S70, SG08S102, SG08S73, SG08S76, SG08S26, DG8S242, SG08S15, DG8S257, SG08S138, DG8S161, SG08S520, DG00AAHBG, SG08S508, DG8S156, D8S1695 and DG8S170. In a particular embodiment, the one or more markers comprise the A allele for SG08S71 and the G allele for DG00AAHBG. In another embodiment, the inverted allele of Inv8p23 is detected by detecting a haplotype comprising one or more genetic markers. In a particular embodiment, one or more genetic markers of the haplotype are selected from the group consisting of: SG08S5, SG08S95, DG8S269, DG8S163, DG8S197, AF131215-2, DG8S127, SG08S120, DG8S179, SG08S27, DG8S261, SG08S71, SG08S32, SG08S517, SG08S70, SG08S102, SG08S73, SG08S76, SG08S26, DG8S242, SG08S15, DG8S257, SG08S138, DG8S161, SG08S520, DG00AAHBG, SG08S508, DG8S156, D8S1695 and DG8S170. In a particular embodiment, the haplotype comprises the A allele for SG08S71 and the G allele for DG00AAHBG. In another embodiment, the kit detects a surrogate marker as described above. In a particular embodiment, bipolar disorder is comorbid with panic disorder, and one or more markers are selected from the group consisting of the markers listed in FIGS. 6A-6K. In another embodiment, bipolar disorder occurs without PD, and one or more markers are selected from the group consisting of the markers listed in FIGS. 7A-7K.

In another embodiment, the invention is directed to a method of diagnosing panic disorder or a comorbid disorder in an individual comprising determining the orientation of the Inv8p23 genomic region, wherein the orientation of the Inv8p23 genomic region is indicative of panic disorder. In a particular embodiment, the orientation of the Inv8p23 genomic region is determined by detecting one or more markers at one or more polymorphic sites, wherein the one or more markers are selected from the group consisting of: SG08S71, DG8S197, SG08S73, DG8S332, AF131215-4, SG08S5, SG08S520, SG08S95, SG08S508, SG08S102, DG00AAHBG, SG08S70, DG8S161, DG8S298, SG08S506, SG08S15, DG8S249, DG8S148, DG8S269, DG8S127, SG08S93, D8S1695, SG08S517, AF131215-2, AF131215-1, DG8S242, DG8S136, D8S516, DG8S148, SG08S39, D8S1130, DG8S127, DG8S232, DG8S137, DG8S269, D8S550, SG08S507, SG08S507, DG8S245, DG8S197, D8S1825, SG08S27, SG08S27, DG8S257, D8S503, DG8S297, DG8S297, SG08S120, SG08S120, D8S351, DG8S159, D8S1695, D8S1759, SG08S26, SG08S26, D8S1130, DG8S221, D8S1130, D8S1759, DG8S307, DG8S153, DG8S277, DG8S192, D8S1695, DG8S265, DG8S257, DG8S127, DG8S163, DG8S163, DG8S156, DG8S261, DG8S179, SG08S138, SG08S32, SG08S76 and DG8S170. In a particular embodiment, the inverted orientation of the Inv8p23 genomic region is indicative of panic disorder.

In another embodiment, the invention is directed to a method of diagnosing bipolar disorder associated with panic disorder in an individual comprising determining the orientation of the Inv8p23 genomic region, wherein the orientation of the Inv8p23 genomic region is indicative of bipolar disorder associated with panic disorder. In one embodiment, the orientation of the Inv8p23 genomic region is determined by detecting one or more markers at one or more polymorphic sites wherein the one or more polymorphic sites are in linkage disequilibrium with the Inv8p23 genomic region and wherein the one or more markers are indicative of the orientation of the Inv8p23 genomic region. In a particular embodiment, the one or more markers are selected from the group consisting of the markers listed in FIGS. 6A-6K.

In another embodiment, the invention is directed to a method of diagnosing bipolar disorder without associated panic disorder in an individual comprising determining the orientation of the Inv8p23 genomic region, wherein the orientation of the Inv8p23 genomic region is indicative of bipolar disorder without associated panic disorder. In one embodiment, the orientation of the Inv8p23 genomic region is determined by detecting one or more markers at one or more polymorphic sites wherein the one or more polymorphic sites are in linkage disequilibrium with the Inv8p23 genomic region and wherein the one or more markers are indicative of the orientation of the Inv8p23 genomic region. In a particular embodiment, the marker is selected from the group consisting of the markers listed in FIGS. 7A-7K.

In another embodiment, the invention is directed to a method for determining the orientation of the Inv8p23 inversion fragment comprising detecting one or more surrogate markers. In one embodiment, one or more surrogate markers are selected from the group consisting of: SG08S5, SG08S95, DG8S269, DG8S163, DG8S197, AF131215-2, DG8S127, SG08S120, DG8S179, SG08S27, DG8S261, SG08S71, SG08S32, SG08S517, SG08S70, SG08S102, SG08S73, SG08S76, SG08S26, DG8S242, SG08S15, DG8S257, SG08S138, DG8S161, SG08S520, DG00AAHBG, SG08S508, DG8S156, D8S1695 and DG8S170.

In another embodiment, the invention is directed to a method for predicting the efficacy of a drug for treating a psychiatric disorder or a comorbid disorder in a human patient, comprising determining the orientation of the Inv8p23 genomic region, wherein the orientation of the Inv8p23 genomic region is indicative of responsiveness or non-responsiveness to the drug in the human patient. In a particular embodiment, the drug is selected from the group consisting of: amine reuptake inhibitors, selective serotonin reuptake inhibitors, selective norepinephrine reuptake inhibitors, combined serotonin-norepinephrine reuptake inhibitors, combined dopamine-norepinephrine reuptake inhibitors, monoamine oxidase inhibitors, reversible/selective inhibitors of monoamine oxidase-A; 5-HT 2A receptor antagonists, combined 5-HT 2A antagonists with serotonin reuptake inhibition, tricyclic drugs, and combined 5-HT 2A, 5-HT 2C and alpha-2 antagonism. In a particular embodiment, the drug is s selective serotonin reuptake inhibitor. In one embodiment, the drug is selected from the group consisting of: venlafaxine, sertraline, paroxat, fluoxetine, escitalopram and citalopram. In another embodiment, the psychiatric disorder is anxiety disorder. In a particular embodiment, the anxiety disorder is panic disorder. In another embodiment, the psychiatric disorder is depression. In another embodiment, the psychiatric disorder is bipolar disorder. In one embodiment, the orientation of Inv8p23 is determined by detecting one or more markers at one or more polymorphic sites wherein the one or more polymorphic sites are in linkage disequilibrium with the Inv8p23 genomic region and wherein the one or more markers are indicative of the orientation of the Inv8p23 genomic region. In a particular embodiment, the one or more markers are selected from the group consisting of: DG8S269, SG08S95, SG08S5, SG08S71 and SG08S73. In one embodiment, the drug is venlafaxine. In a particular embodiment, the drug is fluoxetine. In another embodiment, the drug is Citalopram.

### BRIEF DESCRIPTION OF THE DRAWINGS

FIGS. 1A through 1C show the region of Inv8p23 with positions according to NCBI Build 33 of the human genome. FIG. 1A depicts the assembly, or the inverted variant, and FIG. 1B depicts the alternate assembly, which in fact is the common form of the polymorphism. FIG. 1C depicts the positions of sequenced BACs (bacterial artificial chromosomes) against the sequence of NCBI Build 33, and deCODE's genetic marker map.

FIGS. 2A and 2B show the results of FISH measurements for an individual heterozygous for the inversion polymorphism (FIG. 2A), and a map of the region on which the locations of the probes used to determine orientations is indicated (FIG. 2B).

FIG. 3 is a table showing the results of the determination of the orientation of chromosomes for both individuals with panic disorder and controls.

FIG. 4 is a table showing the results of the linkage disequilibrium analysis, and lists all markers that serve as surrogates for determining the orientation without using FISH measurements. Markers are provided as described in Example 4, and allele numbers are as follows: For SNPs each nucleotide (A, C, G, T) has a numeric code such that: A=0, C=1, G=2, T=3; for microsatellites and INDELs, the allele number is reported as the offset from the smaller of the two alleles of CEPH sample 1347-02 (CEPH genomic repository); thus allele 0 serves as a (CEPH) reference allele.

FIGS. 5A through 5D show a table that lists allelic association to panic disorder with marker names and alleles indicated. Markers are provided as described in Example 4, and allele numbers are as follows: For SNPs each nucleotide (A, C, G, T) has a numeric code such that: A=0, C=1, G=2, T=3; for microsatellites and INDELs, the allele number is reported as the offset from the smaller of the two alleles of CEPH sample 1347-02 (CEPH genomic repository); thus allele 0 serves as a (CEPH) reference allele.

FIGS. 6A through 6K show a table that lists allelic associations to bipolar disorder, with marker names and alleles indicated. Markers are provided as described in Example 4, and allele numbers are as follows: For SNPs each nucleotide (A, C, G, T) has a numeric code such that: A=0, C=1, G=2, T=3; for microsatellites and INDELs, the allele number is reported as the offset from the smaller of the two alleles of CEPH sample 1347-02 (CEPH genomic repository); thus allele 0 serves as a (CEPH) reference allele.

FIGS. 7A through 7K show a table that lists allelic associations to bipolar disorder in the absence of panic disorder, with marker names and alleles indicated. Markers are provided as described in Example 4, and allele numbers are as follows: For SNPs each nucleotide (A, C, G, T) has a numeric code such that: A=0, C=1, G=2, T=3; for microsatellites and INDELs, the allele number is reported as the offset from the smaller of the two alleles of CEPH sample 1347-02 (CEPH genomic repository); thus allele 0 serves as a (CEPH) reference allele.

FIGS. 8A through 8C show a table that lists all markers named in the application along with the position as it is in the most recent build of the human genome (NCBI Build 33).

FIGS. 9A1-9A3, 9B1-9B3 and 9C1-9C4 are tables that lists known genes in the inverted region.

FIG. 10 is a graph showing gene names and relative position according to NCBI Build 33.

FIGS. 11A1-11A3, 11B1-11B12, 11C1-11C8, 11D1-11D8 and 11E1-11E8 are tables listing raw data used for FIGS. 4-7, for the orientation, panic disorder, bipolar disorder, and bipolar disorder without panic disorder. FIGS. 11A1-11A3 show the correlation of 120 markers to the orientation of the Inv8p23 inversion fragment. FIGS. 11B1-11B12 show the allelic frequencies (joint with orientation) of 120 markers on the inverted and common alleles of the Inv8p23 inversion fragment. FIGS. 11C1-11C8 show the association of 120 markers to panic disorder. FIGS. 11D1-11D8 show the association of 120 markers to bipolar disorder. FIGS. 11E1-11E8 show the association of 120 markers to bipolar disorder without panic disorder.

FIGS. 12A and 12B show a table that lists allele frequencies for markers strongly correlated to the orientation (e.g., the markers of FIGS. 5A-5D).

FIGS. 13A and 13B show association of particular markers (positions for NCBI Build 34) with responsiveness to drugs for psychiatric disorders. FIG. 13A (top panel) shows the association of markers for responsiveness to the combination of all patients taking Effexor, Fluoxetine and Citalopram/Escitalopram. FIG. 13A (bottom panel) shows the association of markers for responsiveness to the drug, Effexor. FIG. 13B (top panel) shows the association of markers for responsiveness to the drug, Fluoxetine. FIG. 13B (bottom panel) shows the association of markers for responsiveness to the drug, Citalopram/Escitalopram.

### DETAILED DESCRIPTION OF THE INVENTION

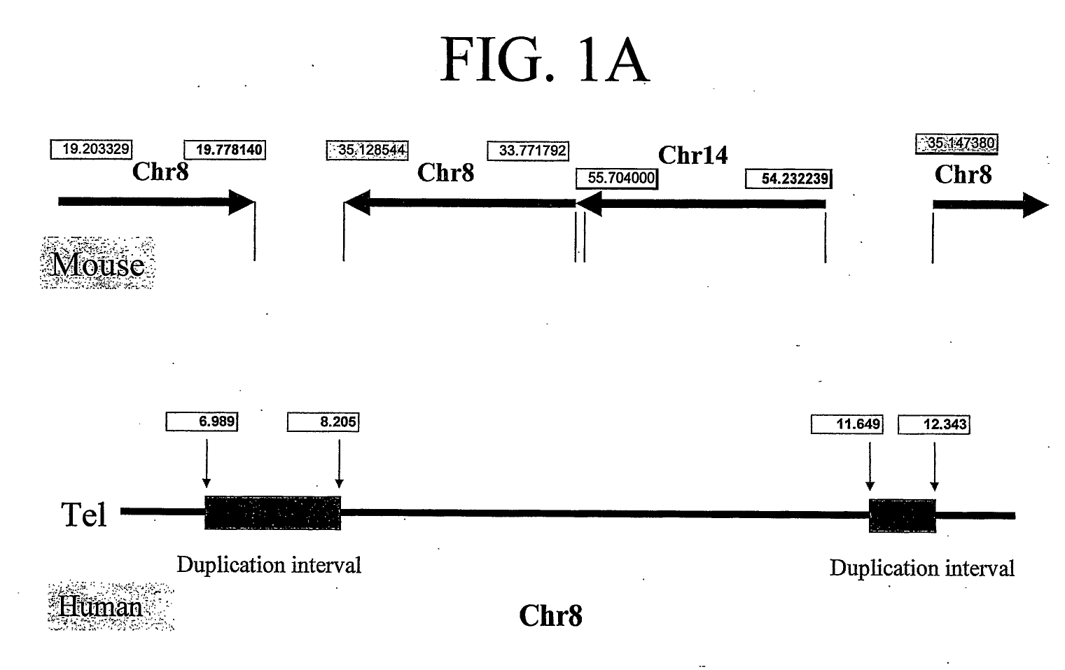

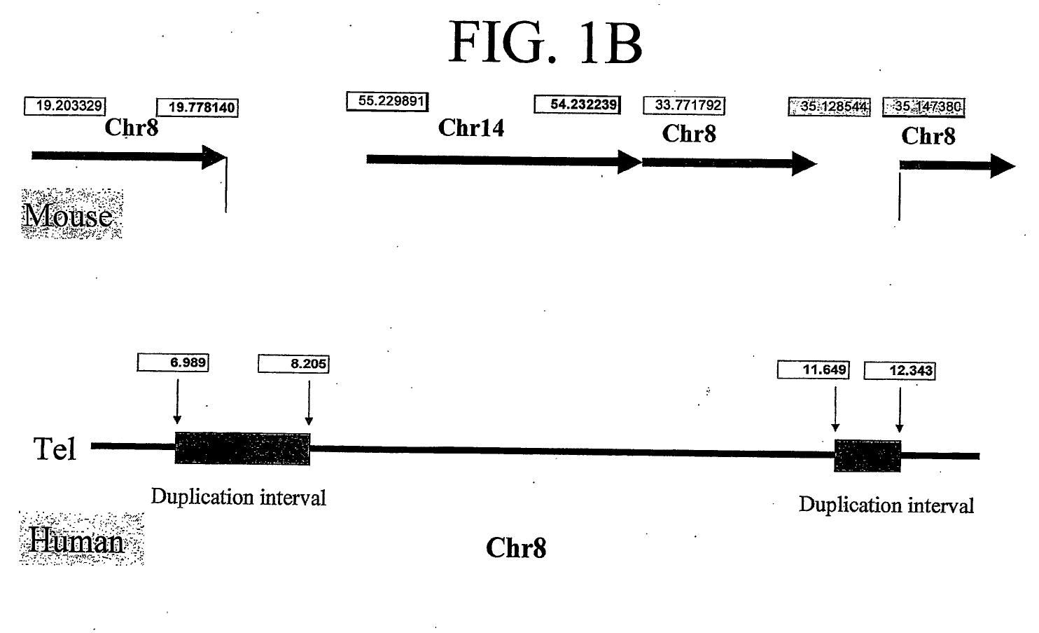

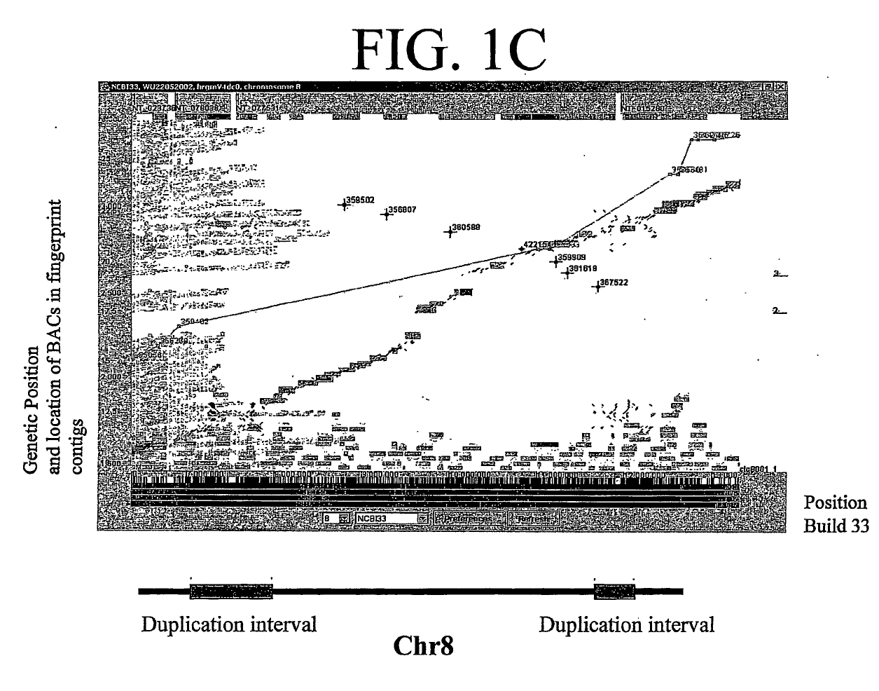

The invention builds on analysis of phenotype data, genotype data, and results from Fluorescence In-situ Hybridization (FISH) experiments. The analysis shows that carriers of the inverted form of an inversion polymorphism involving an unusual 6 MB region on the 8p23 of chromosome 8 (FIGS. 1A-1C), have an increased risk of developing psychiatric disorders. A psychiatric disorder results in a disruption of a person's thinking, feeling, moods and ability to relate to others. Reported herein is the discovery of the association between the less frequent form of the inversion polymorphism on chromosome 8p23 (Inv8p23) and Panic Disorder (PD). Chromosomes were initially studied by FISH, and subsequently identified surrogates for the inversion were identified by analyzing allelic association of microsatellite markers and single nucleotide polymorphisms (SNPs) in the region in a group of individuals with known status for Inv8p23, alleviating the need for further FISH. As used herein, the “region” or “genomic region” of Inv8p23 is the 3-5 MB region on the p-arm of chromosome 8 described above. The “Inv8p23 inversion fragment” is that sequence that is found in different orientations in a population.

The region of Inv8p23 exhibits extensive linkage disequilibrium (recombination is supressed in heterozygotes, but not in homozygotes of either orientation for PD). Analysis of FISH data found the less frequent form of Inv8p23 in strong association with PD with a risk ratio of near 1.5 for carriers of one copy compared to non-carriers. These results were confirmed in a larger sample using the surrogate markers (used herein to refer to markers that can be used to determine the orientation of the Inv8p23 inversion fragment). Elevated risk ratios were also detected for bipolar disorder (BPD) and depression severe enough to require medication. The observation brings psychiatric disorders into the realm of genomic disorders, and opens the possibility that other complex phenotypes are similiarily influenced by the orientation of DNA segments. The location and structure of Inv8p23 is shown in FIGS. 1A-C.

Linkage Disequilibrium (LD) refers to a non-random assortment of two genetic elements. For example, if a particular genetic element (e.g., “alleles” at a polymorphic site; see below) occurs in a population at a frequency of 0.25 and another occurs at a frequency of 0.25, then the predicted occurrance of a person's having both elements is 0.125, assuming a random distribution of the elements. However, if it is discovered that the two elements occur together at a frequency higher than 0.125, then the elements are said to be in linkage disequilibrium since they tend to be inherited together at a higher rate than what their independent allele frequencies would predict. Roughly speaking, LD is generally correlated with the frequency of recombination events between the two elements. Allele frequencies can be determined in a population by genotyping individuals in a population and determining the occurence of each allele in the population. For populations of diploids, e.g., human populations, individuals will typically have two alleles for each genetic element (e.g., a marker or gene).

Disclosed herein, for example, are data describing a particular genomic marker, comprising the Inv8p23 genomic region. This marker has two alleles, the inverted allele and the reference allele. The allele frequency of the inverted allele is significantly lower than the allele frequency of the reference allele, therefore individuals that are homozygous for the inverted allele are rare (“Hz rare” individuals), and individuals who are homozygous for the reference allele are common (“Hz common” individuals) in the population.

Many different measures have been proposed for assessing the strength of linkage disequilibrium (LD). Most capture the strength of association between pairs of biallelic sites. Two important pairwise measures of LD are r² (sometimes denoted Δ²) and |D′|. Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. So, a value of |D′| that is <1 indicates that historical recombination has occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present. It is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value can be 0.3, 0.4, 0.5, 0.6, 0.7, 0.8, 0.9 or 1.0.

Additional marker that are in LD with the Inv8p23 marker are referred to herein as “surrogate” markers. Such a surrogate is a marker for another marker or another surrogate marker. Surrogate markers are themselves markers and are indicative of the presence of another marker, which is in turn indicative of either another marker or an associated phenotype.

Genetic markers are particular “alleles” at “polymorphic sites”. Genetic markers can include “polymorphisms”, which are particular alleles at polymorphic sites. A nucleotide position at which more than one sequence is possible in a population (either a natural population or a synthetic population, e.g., a library of synthetic molecules) is referred to herein as a “polymorphic site”. Where a polymorphic site is a single nucleotide in length, the site is referred to as a single nucleotide polymorphism (“SNP”). For example, if at a particular chromosomal location, one member of a population has an adenine and another member of the population has a thymine at the same position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele. “Markers” are genetic elements, e.g., SNPs, genes, polymorphisms, drug resistance, restriction sites, etc., or combinations of genetic elements, e.g., haplotypes, that can be used to indicate a particular characteristic. For example, if a particular SNP is demonstrated to be “associated” (see below) with a particular phenotype, then the detection of the particular SNP is indicative of the particular phenotype. In this example, the SNP is used as a marker.

Populations of individuals exhibiting genetic diversity do not have identical genomes; in other words, there are many polymorphic sites in a population. In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a disease or abnormal phenotype). Alleles that differ from the reference are referred to as “variant” alleles.

An individual at risk for or to be diagnosed with a psychiatric disorder, e.g., an anxiety disorder, PD or a comorbid disorder is an individual who has the inverted allele (Inv8p23) of the inversion polymorphism on chromosome 8, described above. This allele can be identified directly by methods known in the art, or by identification and orientation of any of the markers identified herein. Additionally, the markers described herein can themselves serve as predictors of susceptibility to or as an indicator of a psychiatric disorder, anxiety disorder, PD or a comorbid disorder. As used herein, a “comorbid disorder” refers to a disorder existing simultaneously with and usually independently of another medical condition, e.g., PD. Examples of disorders comorbid with PD include, but are not limited to, depression, bipolar disorder (BPD; also known as manic-depressive illness), obsessive-compulsive disorder (OCD), histrionic personality disorder, family denial and dysfunction, hypercholesterolemia and substance abuse.

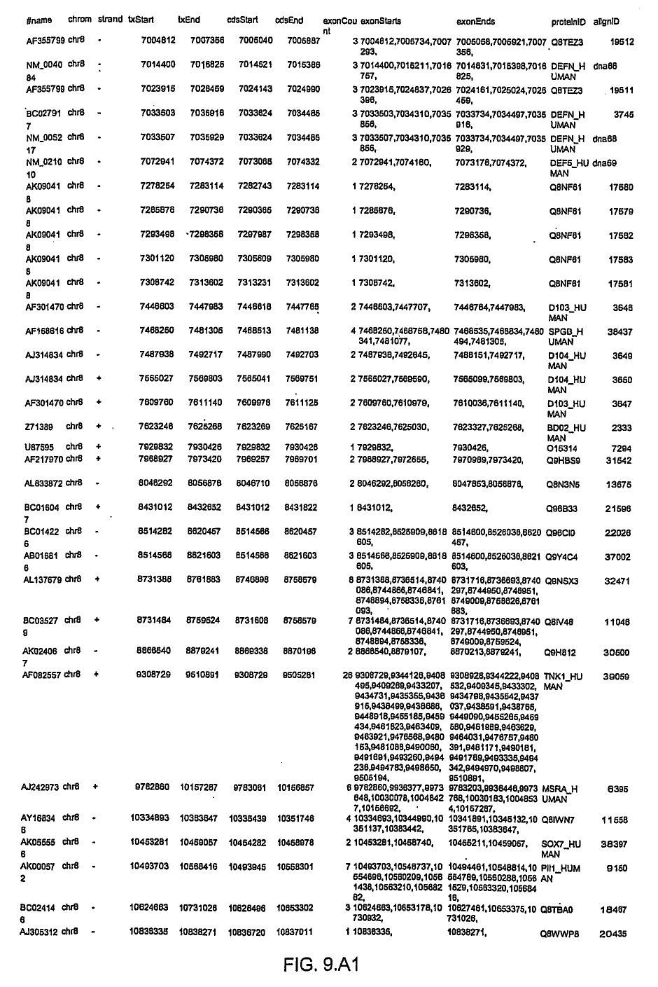

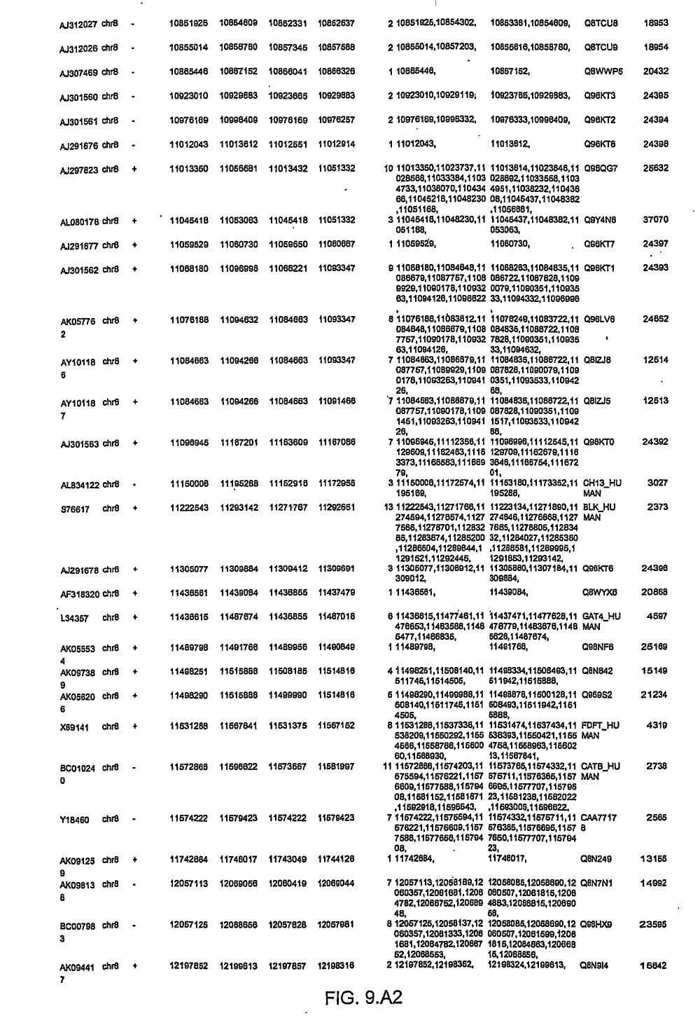

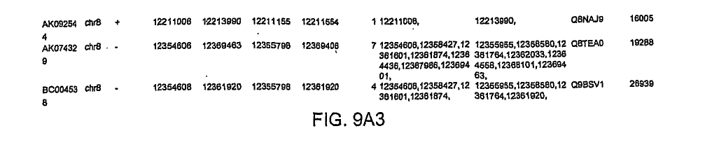

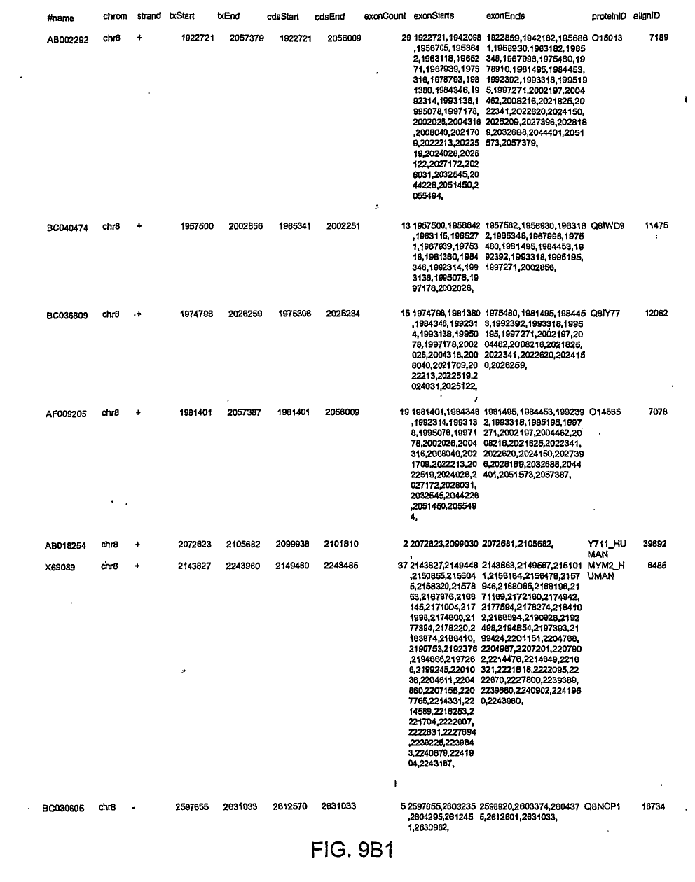

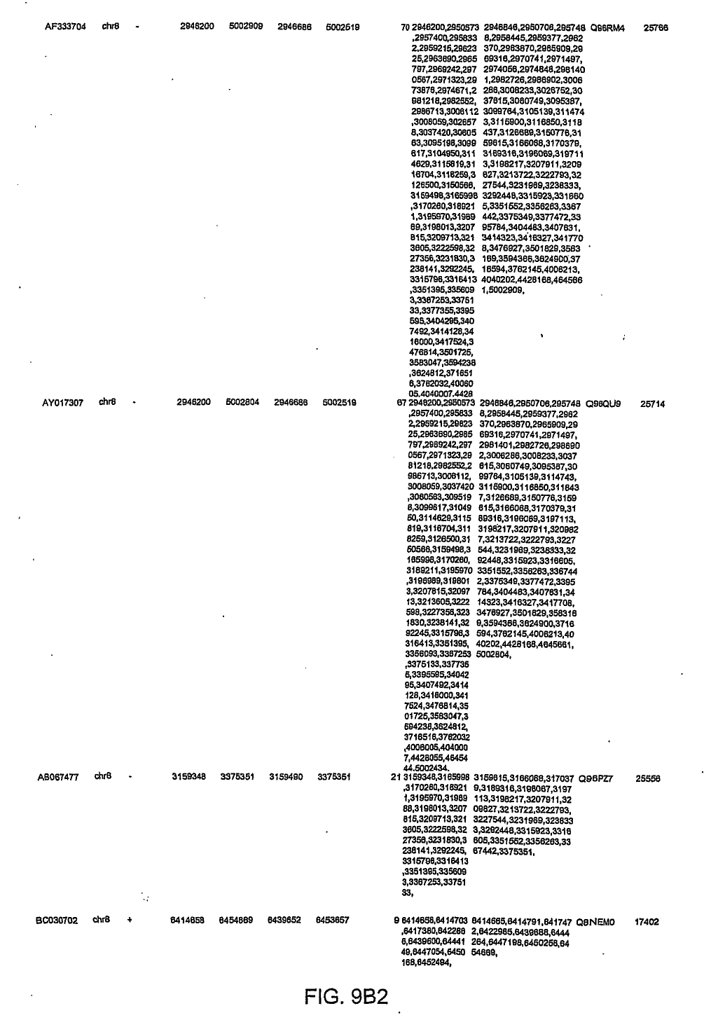

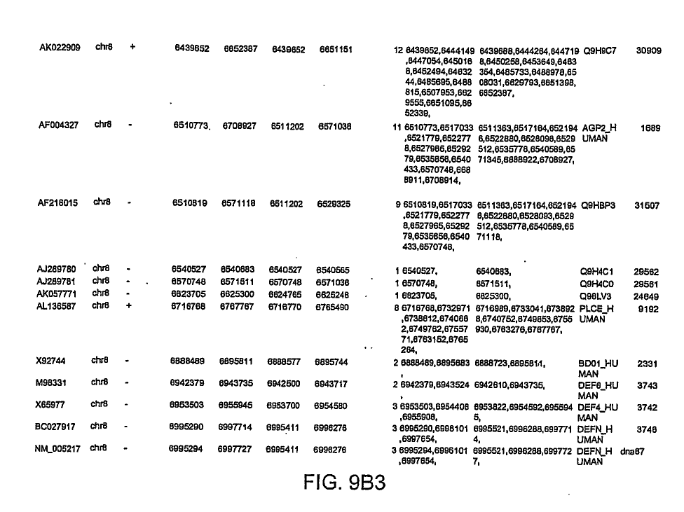

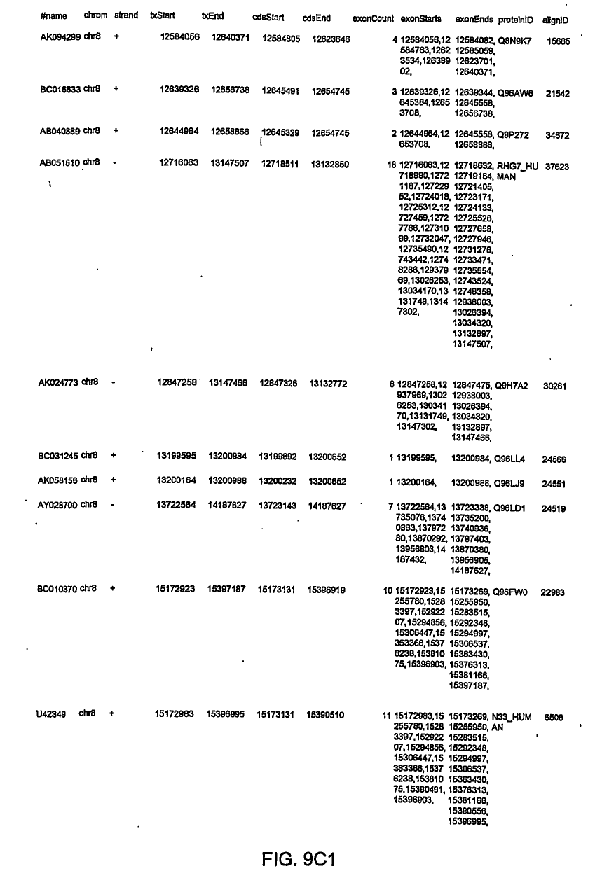

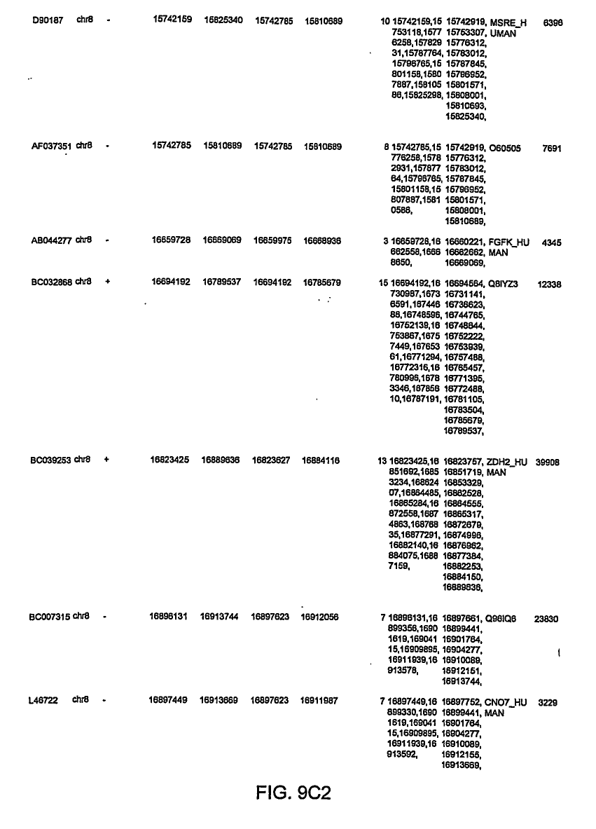

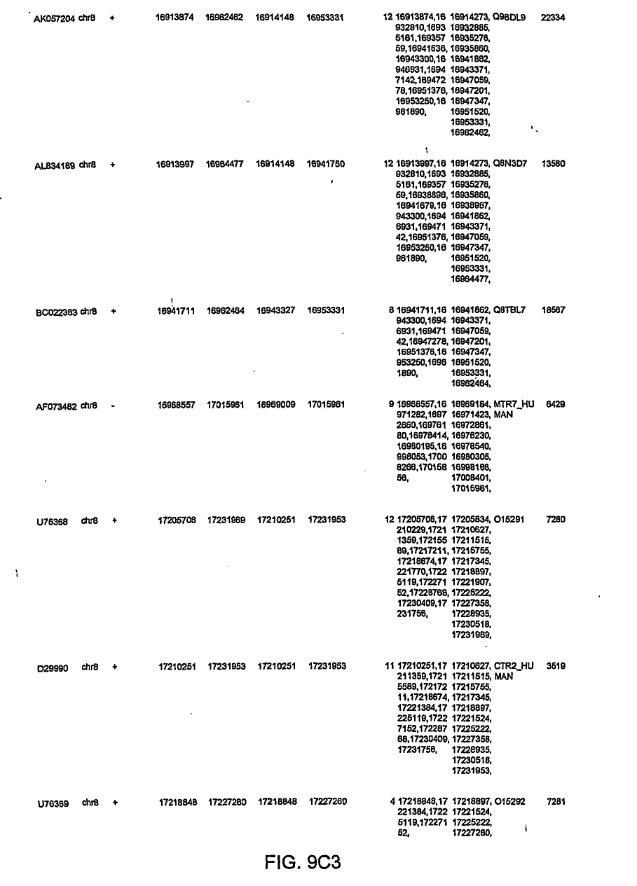

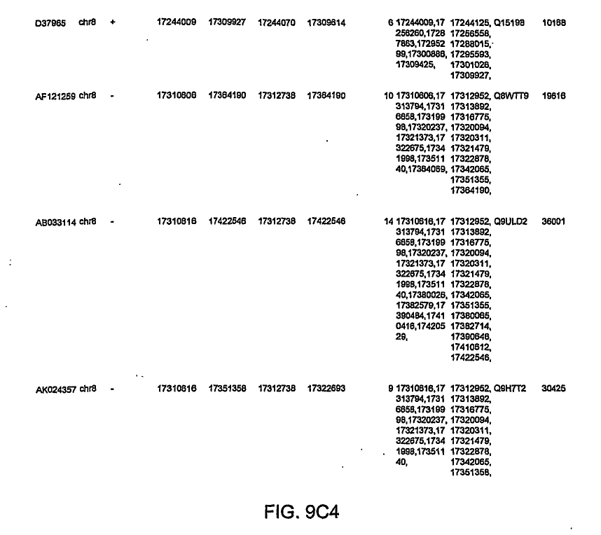

Inv8p23 is herein demonstrated to be associated with PD and comorbid disorders, and the Inv8p23 genomic region contains several genes (FIGS. 9A1-9A3, 9B1-9B3 and 9C1-9C4). The term “gene,” as used herein, refers to not only the sequence of nucleic acids encoding a polypeptide, but also the promoter regions, transcription enhancement elements, splice donor/acceptor sites, splice enhancer and silencer sequences and other regulators of splicing, and other non-transcribed nucleic acid elements. The likely result of the inversion polymorphism is the misexpression, e.g., no expression, increased expression, or reduced expression, of one or more of the genes affected by the inversion. Therefore, these genes will serve as potential targets for treating PD and comorbid disorders.

Additional variants can include changes that affect a polypeptide, e.g., the polypeptides that result from expression of one or more genes affected by Inv8p23. These sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by a nucleic acid in the Inv8p23 region. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with PD and/or one or more comorbid disorders or a susceptibility to PD and/or one or more comorbid disorders can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of the polypeptide. The polypeptide encoded by the reference nucleotide sequence is the “reference” polypeptide with a particular reference amino acid sequence, and polypeptides encoded by variant alleles are referred to as “variant” polypeptides with variant amino acid sequences.

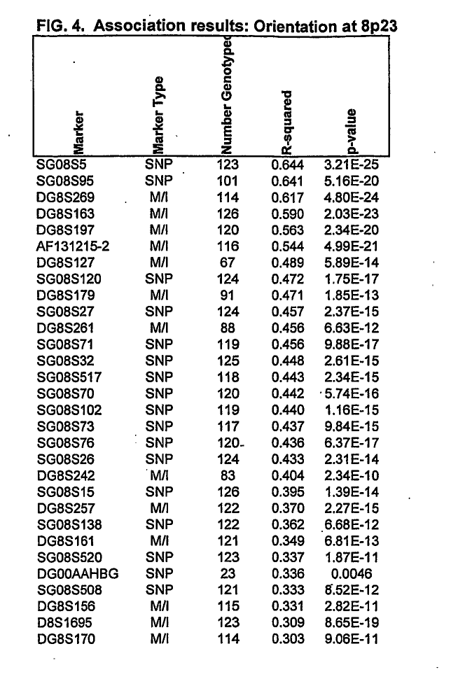

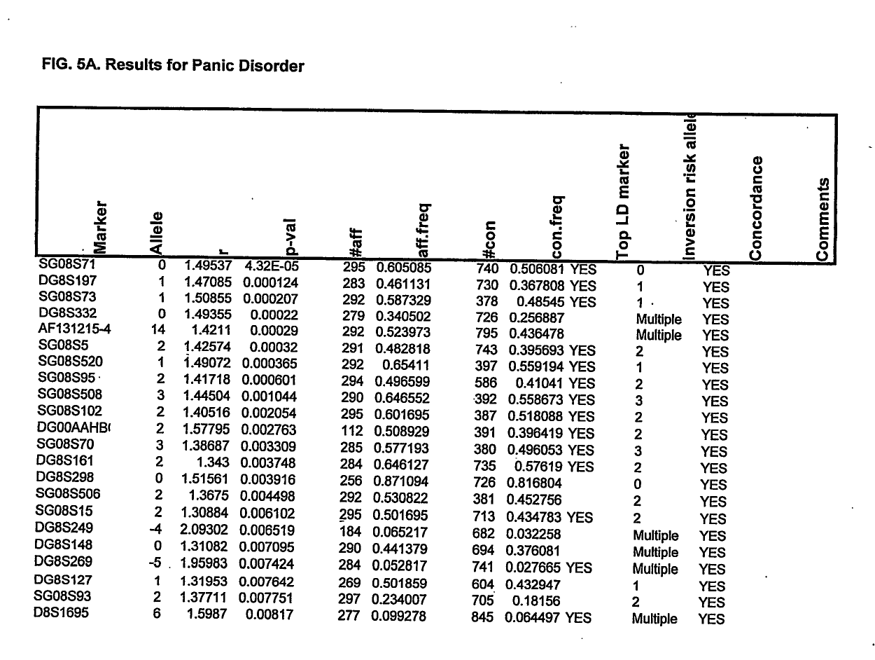

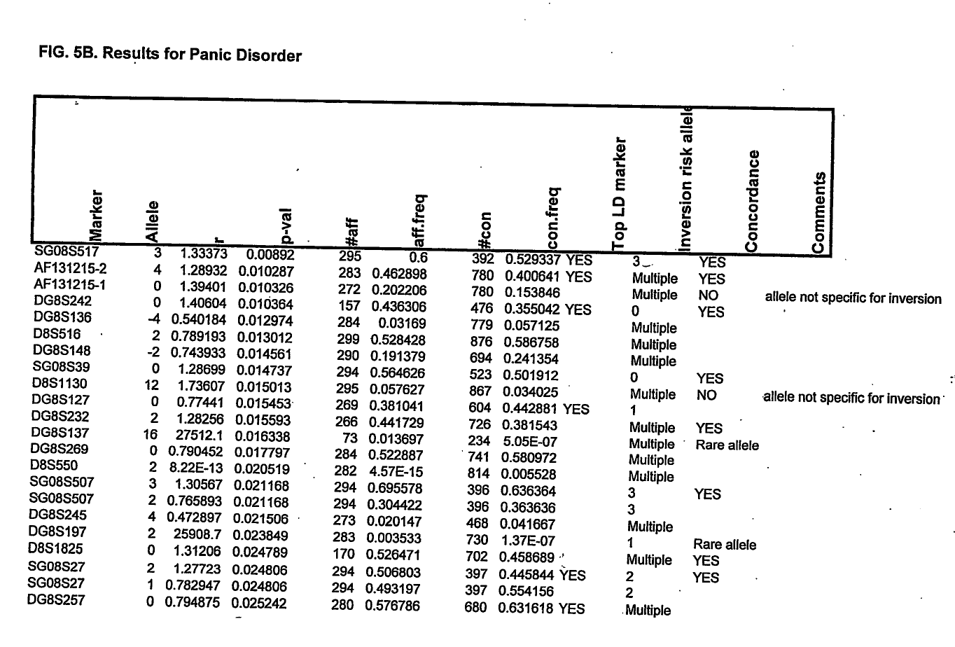

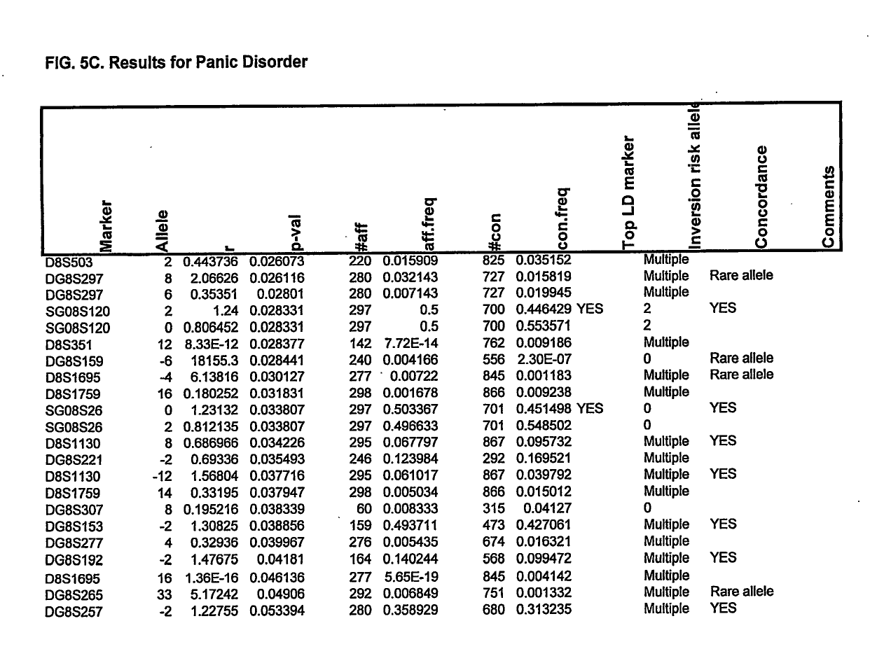

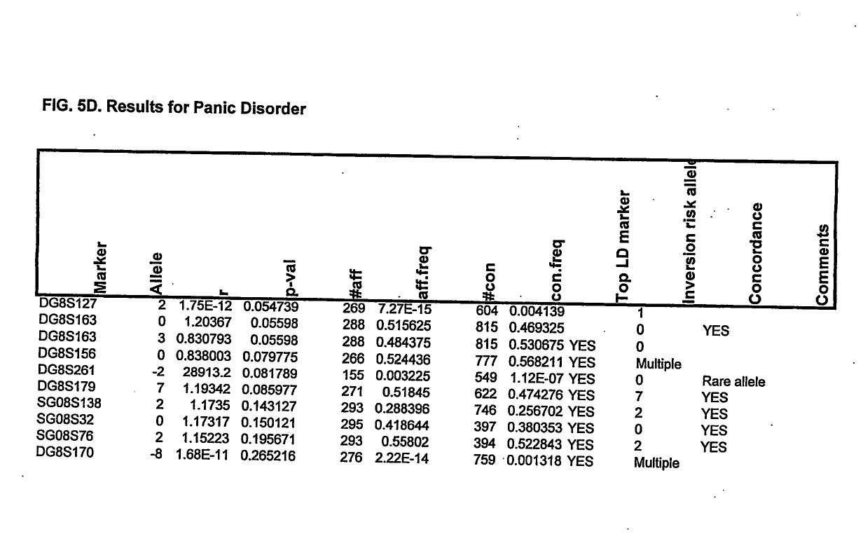

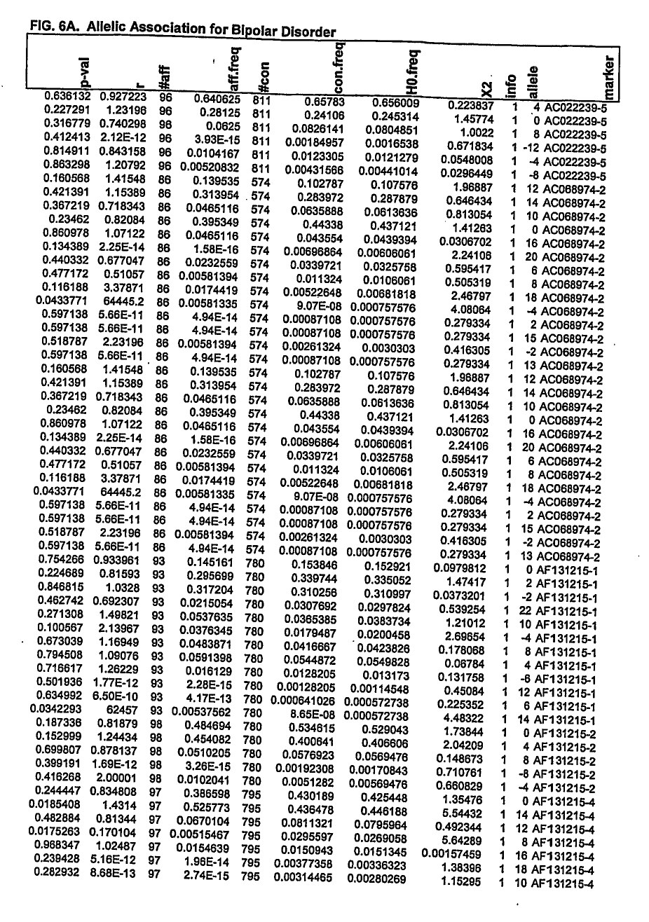

In certain methods described herein, an individual can be diagnosed with or identified as being susceptible to a psychiatric disorder, e.g., anxiety disorder such as, for example, PD or a comorbid disorder in an individual who has the Inv8p23 allele. As identified herein, this is the “at-risk” genotype, and it can also be used to diagnose individuals affected by PD or a comorbid disorder. As used herein, “genotype” refers to an accounting of one or more genetic elements (e.g., an allele at a particular polymorphic site on one or both copies of the chromosome) of a particular individual. The significance associated with an at-risk genotype can be measured by an odds ratio. In a further embodiment, the significance is measured by a percentage. In one embodiment, significance is demonstrated with an odds ratio of at least about 1.0, including but not limited to: 1.0, 1.1, 1.2, 1.3, 1.4, 1.5, 1.6, 1.7, 1.8 and 1.9 (or higher for some alleles in FIGS. 9A-C; see association data provided in FIGS. 4, 5A-D, 6A-K, 7A-K, 9A-C, 11A-E, and 13A-B). In one embodiment, an odds ratio of at least 1.0 is significant. In another embodiment, an odds ratio of at least about 1.5 is significant. In another embodiment, a significant increase in risk is at least about 1.7 is significant. In another embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In one embodiment, a significant increase in risk is at least about 50%. It is understood that identifying whether a risk is medically significant can also depend on a variety of factors, including the specific disease, the haplotype, and often, environmental factors.

An at-risk genotype (combination of one or more markers) is one where the genotype is more frequently present in an individual at risk for a psychiatric disorder, anxiety disorder, PD or a comorbid disorder, compared to the frequency of its presence in a healthy individual (control), and wherein the presence of the haplotype is indicative of PD and/or one or more comorbid disorders or susceptibility to PD and/or one or more comorbid disorders. A protective genotype is one where the genotype is more frequently present in an individual where the genotype is protective against being affected by PD or a comorbid disorder compared to the frequency of its presence in an individual with PD or a comorbid disorder. The presence of the protective genotype is indicative of a protection from PD and/or one or more comorbid disorders or protection from susceptibility to PD and/or one or more comorbid disorders as described above.

Standard techniques for genotyping for the presence of SNPs and/or microsatellite markers can be used, such as fluorescent-based techniques (Chen, et al., Genome Res. 9, 492 (1999)), PCR, LCR, Nested PCR and other techniques for nucleic acid amplification. In one embodiment, the method comprises assessing in an individual the presence or frequency of SNPs and/or microsatellites in determining the presence or absence of the Inv8p23 allele.

The invention includes nucleic acid molecules useful in detecting the presence or absence of the Inv8p23 allele. For example, probes, primers or labeled nucleic acids can be used to detect either the inversion allele itself, or to detect markers that are indicative of the presence or absence of the allele. In another embodiment, a nucleic acid of the invention; a nucleic acid complementary to a nucleic acid of the invention; or a portion of such a nucleic acid (e.g., an oligonucleotide as described below); or a nucleic acid encoding one or more polypeptides or nucleic acids that result from the expression of one or more genes contained in the Inv8p23 region, can be used in “antisense” therapy, in which a nucleic acid (e.g., an oligonucleotide) which specifically hybridizes to the mRNA and/or genomic DNA of a nucleic acid is administered or generated in situ, RNAi therapy, in which double-stranded RNA corresponding to a particular gene inactivates expression of the gene, or any other therapeutic regimen involving precise nucleic acid sequences contained in the Inv8p23 region.

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention is substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by PAGE or column chromatography such as HPLC. Preferably, an isolated nucleic acid molecule comprises at least about 50, 80 or 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 5 kb, 4 kb, 3 kb, 2 kb, 1 kb, 0.5 kb or 0.1 kb of nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Therefore, recombinant DNA contained in a vector is included in the definition of “isolated” as used herein. Also, isolated nucleotide sequences include recombinant DNA molecules in heterologous organisms, as well as partially or substantially purified DNA molecules in solution. In vivo and in vitro RNA transcripts of the DNA molecules of the present invention are also encompassed by “isolated” nucleotide sequences. Such isolated nucleotide sequences are useful in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis.

The present invention also pertains to variant nucleic acid molecules that are not necessarily found in nature but encode a polypeptide that results from the expression of one or more genes in the Inv8p23 region, a splicing variant of such a polypeptide or polymorphic variant thereof. Thus, for example, DNA molecules that comprise a sequence that is different from the naturally-occurring nucleotide sequence but, due to the degeneracy of the genetic code, encode a polypeptide expressed by a gene in the Inv8p23 region also the subject of this invention. The invention also encompasses nucleotide sequences encoding portions (fragments), or encoding variant polypeptides. Such variants can be naturally-occurring, such as in the case of allelic variation or single nucleotide polymorphisms, or non-naturally-occurring, such as those induced by various mutagens and mutagenic processes. Variations include, but are not limited to, addition, deletion and substitution of one or more nucleotides that can result in conservative or non-conservative amino acid changes, including additions and deletions.

Other alterations of the nucleic acid molecules of the invention can include, for example, labeling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequence via hydrogen bonding and other chemical interactions. Such molecules include, for example, those in which peptide linkages substitute for phosphate linkages in the backbone of the molecule.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence encoding polypeptides described herein, and, optionally, have an activity of the polypeptide).

Such nucleic acid molecules can be detected and/or isolated by specific hybridization (e.g., under high stringency conditions). “Specific hybridization,” as used herein, refers to the ability of a first nucleic acid to hybridize to a second nucleic acid in a manner such that the first nucleic acid does not hybridize to any nucleic acid other than to the second nucleic acid (e.g., when the first nucleic acid has a higher similarity to the second nucleic acid than to any other nucleic acid in a sample wherein the hybridization is to be performed). “Stringency conditions” for hybridization refers to the incubation and wash conditions, e.g., conditions of temperature and buffer concentration, that permit hybridization of a particular nucleic acid to a second nucleic acid; the first nucleic acid can be perfectly (i.e., 100%) complementary to the second, or the first and second can share some degree of complementarity that is less than perfect (e.g., 70%, 75%, 85%, 95%). For example, certain high stringency conditions can be used to distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (1998), the entire teachings of which are incorporated by reference herein). The exact conditions that determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions that will allow a given sequence to hybridize (e.g., selectively) with the most similar sequences in the sample can be determined.

Exemplary conditions are described in Krause, M. and S. Aaronson, 1991, Meth. Enzymol., 200:546-556. Also, in, Ausubel, et al., “Current Protocols in Molecular Biology”, John Wiley & Sons, (1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tₘ of 17° C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 minutes at room temperature; a moderate stringency wash can comprise washing in a prewarmed solution (42° C.) solution containing 0.2×SSC/0.1% SDS for 15 minutes at 42° C.; and a high stringency wash can comprise washing in prewarmed (68° C.) solution containing 0.1×SSC/0.1% SDS for 15 minutes at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

The percent homology or identity of two nucleotide or amino acid sequences can be determined by aligning the sequences for optimal comparison purposes (e.g., gaps can be introduced in the sequence of a first sequence for optimal alignment). The nucleotides or amino acids at corresponding positions are then compared, and the percent identity between the two sequences is a function of the number of identical positions shared by the sequences (i.e., % identity=# of identical positions/total # of positions×100). Where a position in one sequence is occupied by the same nucleotide or amino acid residue as the corresponding position in the other sequence, then the molecules are homologous at that position. As used herein, nucleic acid or amino acid “homology” is equivalent to nucleic acid or amino acid “identity”. In certain embodiments, the length of a sequence aligned for comparison purposes is at least 30%, for example, at least 40%, in certain embodiments at least 60%, and in other embodiments at least 70%, 80%, 90% or 95% of the length of the reference sequence. The actual comparison of the two sequences can be accomplished by well-known methods, for example, using a mathematical algorithm. One, non-limiting example of such a mathematical algorithm is described in Karlin, S. and Altschul, S., 1993, Proc. Natl. Acad. Sci. USA, 90:5873-5877. Such an algorithm is incorporated into the NBLAST and XBLAST programs (version 2.0) as described in Altschul, S et al., 1997, Nucleic Acids Res., 25:3389-3402. When utilizing BLAST and Gapped BLAST programs, the default parameters of the respective programs (e.g., NBLAST) can be used. In one embodiment, parameters for sequence comparison can be set at score=100, wordlength=12, or can be varied (e.g., W=5 or W=20).

Another preferred non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torelli, A and Robotti, C., 1994, Comput. Appl. Biosci., 10:3-5; and FASTA described in Pearson, W. and Lipman, D., 1988, Proc. Natl. Acad. Sci. USA, 85:2444-8.

The present invention also provides isolated nucleic acid molecules that contain a fragment or portion that hybridizes under highly stringent conditions to a nucleotide sequence comprising a nucleotide sequence or fragment of the Inv8p23 genomic region or a region in LD with the Inv8p23 genomic region. The nucleic acid fragments of the invention are at least about 15, preferably at least about 18, 20, 23 or 25 nucleotides, and can be 30, 40, 50, 100, 200 or more nucleotides in length. Longer fragments, for example, 30 or more nucleotides in length, which encode antigenic polypeptides described herein are particularly useful, such as for the generation of antibodies as described below. In one embodiment, the nucleotide sequences are fragments that comprise one or more polymorphic microsatellite markers. In another embodiment, the nucleotide sequences are fragments that comprise one or more single nucleotide polymorphisms in the Inv8p23 region.

In a related aspect, the nucleic acid fragments of the invention are used as probes or primers in assays such as those described herein. “Probes” or “primers” are oligonucleotides that hybridize in a base-specific manner to a complementary strand of nucleic acid molecules. By “base specific manner” is meant that the two sequences must have a degree of nucleotide complementarity sufficient for the primer or probe to hybridize. Accordingly, the primer or probe sequence is not required to be perfectly complementary to the sequence of the template. Non-complementary bases or modified bases can be interspersed into the primer or probe, provided that base substitutions do not inhibit hybridization. The nucleic acid template can also include “non-specific priming sequences” or “nonspecific sequences” to which the primer or probe has varying degrees of complementarities. Such probes and primers include polypeptide nucleic acids, as described in Nielsen, P. et al., 1991, Science, 254:1497-1500.

A probe or primer comprises a region of nucleic acid that hybridizes to at least about 15, for example about 20-25, and in certain embodiments about 40, 50 or 75, consecutive nucleotides of a nucleic acid of the invention, such as a nucleic acid comprising a contiguous nucleic acid sequence the Inv8p23 region, fragment thereof, or the complement. In certain embodiments, a probe or primer comprises 100 or fewer nucleotides, in certain embodiments, from 6 to 50 nucleotides, for example, from 12 to 30 nucleotides. In other embodiments, the probe or primer is at least 70% identical to the contiguous nucleic acid sequence or to the complement of the contiguous nucleotide sequence, for example, at least 80% identical, in certain embodiments at least 90% identical, and in other embodiments at least 95% identical, or even capable of selectively hybridizing to the contiguous nucleic acid sequence or to the complement of the contiguous nucleotide sequence. Often, the probe or primer further comprises a label, e.g., radioisotope, fluorescent compound, enzyme, or enzyme co-factor.

The nucleic acid molecules of the invention such as those described above can be identified and isolated using standard molecular biology techniques and the sequence information provided herein. For example, nucleic acid molecules can be amplified and isolated by the polymerase chain reaction using synthetic oligonucleotide primers designed based on one or more of the sequences contained in the Inv8p23 region, preferably those sequences that establish the orientation of the Inv8p23 inverted fragment (see generally PCR Technology: Principles and Applications for DNA Amplification (ed. H. A. Erlich, Freeman Press, NY, N.Y., 1992); PCR Protocols: A Guide to Methods and Applications (Eds. Innis, et al., Academic Press, San Diego, Calif., 1990); Mattila, P. et al., 1991, Nucleic Acids Res., 19:4967-4973; Eckert, K. and Kunkel, T., 1991, PCR Methods Appl., 1:17-24; PCR (eds. McPherson et al., IRL Press, Oxford); and U.S. Pat. No. 4,683,202). The nucleic acid molecules can be amplified using cDNA, mRNA or genomic DNA as a template, cloned into an appropriate vector and characterized by DNA sequence analysis.

Other suitable amplification methods include the ligase chain reaction (LCR) (see Wu, D. and Wallace, R., 1989, Genomics, 4:560-569; Landegren, U. et al., 1988, Science, 241:1077-1080), transcription amplification (Kwoh, D. et al., 1989, Proc. Natl. Acad. Sci. USA, 86:1173-1177), and self-sustained sequence replication (Guatelli et al., 1990, Proc. Nat. Acad. Sci. USA, 87:1874-1878) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be labeled (e.g., with radiolabel or other reporter molecule) and used as a probe for screening a cDNA library derived from human cells, mRNA in zap express, ZIPLOX or other suitable vector. Corresponding clones can be isolated, DNA can obtained following in vivo excision, and the cloned insert can be sequenced in either or both orientations by art recognized methods to identify the correct reading frame encoding a polypeptide of the appropriate molecular weight. For example, the direct analysis of the nucleotide sequence of nucleic acid molecules of the present invention can be accomplished using well-known methods that are commercially available (see, for example, Sambrook et al., Molecular Cloning, A Laboratory Manual (2nd Ed., CSHP, New York 1989); Zyskind et al., Recombinant DNA Laboratory Manual, (Acad. Press, 1988)). Using these or similar methods, the polypeptide and the DNA encoding the polypeptide can be isolated, sequenced and further characterized.

The nucleic acid sequences can be used to compare with endogenous DNA sequences in patients to identify genetic disorders (e.g., a predisposition for or susceptibility to PD or a comorbid disorder), and as probes, such as to hybridize and discover related DNA sequences or to subtract out known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. The nucleic acid sequences can additionally be used as reagents in the screening and/or diagnostic assays described herein, and can also be included as components of kits (e.g., reagent kits) for use in the screening and/or diagnostic assays described herein.

The nucleic acids, probes, primers, polypeptides and antibodies described herein can be used in methods of diagnosis of PD and/or one or more comorbid disorders or of a susceptibility to PD and/or one or more comorbid disorders, as well as in kits useful for diagnosis of PD and/or one or more comorbid disorders or a susceptibility to PD and/or one or more comorbid disorders. In one embodiment, the kit comprises primers as described herein, wherein the primers detect one or more of the markers identified herein.

In one embodiment of the invention, diagnosis of PD and/or one or more comorbid disorders or susceptibility to PD and/or one or more comorbid disorders is made by detecting the inversion Inv8p23 allele as described herein. The occurrence of this allele can result in altered expression of one or more genes contained in the Inv8p23 genomic region. For example, if the breakpoints of the inversion result in a frameshift alteration of a coding sequence of a gene, the frame shift can result in a change in the encoded amino acids, and/or can result in the generation of a premature stop codon, causing generation of a truncated polypeptide. For diagnostic applications, there could exist polymorphisms informative for prediction of disease risk that are in linkage disequilibrium with the functional polymorphism. Such a polymorphism can alter splicing sites, affect the stability or transport of mRNA, or otherwise affect the transcription or translation of the nucleic acid.

In a first method of diagnosing PD and/or one or more comorbid disorders or a susceptibility to PD and/or one or more comorbid disorders, hybridization methods, such as Southern analysis, Northern analysis, or in situ hybridizations, can be used (see Current Protocols in Molecular Biology, Ausubel, F. et al., eds., John Wiley & Sons, including all supplements through 1999). For example, a biological sample from a test subject (a “test sample”) of genomic DNA, RNA, or cDNA, is obtained from an individual suspected of having, being susceptible to or predisposed for PD and/or one or more comorbid disorders (the “test individual”). The individual can be an adult, child, or fetus. The test sample can be from any source that contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine the presence or absence of the Inv8p23 allele. The presence of the allele or splicing variant can be indicated by hybridization of the nucleic acid in the genomic DNA, RNA, or cDNA to a nucleic acid probe.

To diagnose a susceptibility to PD and/or one or more comorbid disorders, a hybridization sample is contacted by at least one nucleic acid probe. A preferred probe for detecting mRNA or genomic DNA is a labeled nucleic acid probe capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA or genomic DNA. The hybridization sample is maintained under conditions that are sufficient to allow specific hybridization to one or more markers in the Inv8p23 region. Specific hybridization, if present, is then detected using methods known in the art and described above. In one embodiment, specific hybridization of at least one of the nucleic acid probes is indicative of the presence of the Inv8p23 allele, and is therefore diagnostic for a susceptibility to PD and/or one or more comorbid disorders.

Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P. et al., 1994, Bioconjug. Chem., 5:3-7. The PNA probe can be designed to specifically hybridize to a gene having a polymorphism associated with a susceptibility to PD and/or one or more comorbid disorders.

In another method of the invention, analysis by restriction digestion can be used to detect a specific allele at a polymorphic site, if the polymorphism results in the creation or elimination of a restriction site, or alters the order of restriction sites in a sequence. If a restriction site is not naturally created, one can be created by PCR that depends on the polymorphism and allows genotyping. A test sample containing genomic DNA is obtained from the individual. Nucleic acid amplification methods, including but not limited to Polymerase Chain Reaction (PCR), Transcription Mediated Amplifications (TMA), and Ligase Mediate Amplification (LMA), can be used to amplify genomic regions. The digestion pattern of the relevant DNA fragment indicates the presence or absence of one or more markers or of the orientation of the Inv8p23 inversion fragment itself, and therefore indicates the presence or absence of this susceptibility to PD and/or one or more comorbid disorders. RFLP analysis can be conducted as described in the art (see Current Protocols in Molecular Biology, supra). Amplification techniques based upon detection of sequence of interest using reverse dot blot technology (linear array or strips) can be used and are described, for example, in U.S. Pat. No. 5,468,613.

Sequence analysis can also be used to detect one or more markers described herein or the Inv8p23 allele. A test sample of DNA or RNA is obtained from the test individual. PCR or other appropriate methods can be used to amplify the region, and/or its flanking sequences, if desired. The sequence can be determined using standard methods. The sequence of the region is compared with the known nucleic acid sequence, as appropriate. In one embodiment, the presence of at least one of the markers of the invention indicates that the individual has a susceptibility to PD and/or one or more comorbid disorders.

Allele-specific oligonucleotides can also be used to detect the presence of the Inv8p23 allele, through the use of dot-blot hybridization of amplified oligonucleotides with allele-specific oligonucleotide (ASO) probes (see, for example, Saiki, R. et al., 1986, Nature, 324:163-166). An “allele-specific oligonucleotide” (also referred to herein as an “allele-specific oligonucleotide probe”) is an oligonucleotide of approximately 10-50 base pairs, preferably approximately 15-30 base pairs, that specifically hybridizes to a DNA sequence contained in the Inv8p23 region, and that contains a sequence suitable for determining the orientation of the Inv8p23 inversion fragment. An allele-specific oligonucleotide probe can be prepared, using standard methods (see Current Protocols in Molecular Biology, supra). A test sample of DNA is obtained from an individual. PCR can be used to amplify the Inv8p23 region and its flanking sequences. The amplified DNA is dot-blotted, using standard methods (see Current Protocols in Molecular Biology, supra), and the blot is contacted with an oligonucleotide probe. The presence of specific hybridization of the probe to the amplified DNA is then detected. Specific hybridization of an allele-specific oligonucleotide probe to DNA from the individual is indicative of the presence or absence of the Inv8p23 inversion, and is therefore indicative of a susceptibility to PD and/or one or more comorbid disorders.

The invention further provides allele-specific oligonucleotides that hybridize to the reference or variant allele of a nucleic acid comprising a single nucleotide polymorphism or to the complement thereof. These oligonucleotides can be probes or primers.

An allele-specific primer hybridizes to a site on target DNA overlapping a polymorphism and only primes amplification of an allelic form to which the primer exhibits perfect complementarity (Gibbs, R. et al., 1989, Nucleic Acids Res., 17:2437-2448). This primer is used in conjunction with a second primer that hybridizes at a distal site. Amplification proceeds from the two primers, resulting in a detectable product that indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures of 64° C. and 74° C. where in complex with complementary DNA or RNA, respectively, as opposed to 28° C. for both DNA and RNA for the corresponding DNA nonamer. Substantial increases in Tₘ are also obtained when LNA monomers are used in combination with standard DNA or RNA monomers. For primers and probes, depending on where the LNA monomers are included (e.g., the 3′ end, the 5′ end, or in the middle), the Tₘ could be increased considerably.

In another embodiment, arrays of oligonucleotide probes that are complementary to target nucleic acid sequence segments from an individual, can be used to identify one or more markers or polymorphic alleles in the Inv8p23 region. For example, in one embodiment, an oligonucleotide linear array can be used. Oligonucleotide arrays typically comprise a plurality of different oligonucleotide probes that are coupled to a surface of a substrate in different known locations. These oligonucleotide arrays, also described as “Genechips”™, have been generally described in the art, for example, U.S. Pat. No. 5,143,854 and PCT patent publication Nos. WO 90/15070 and 92/10092. These arrays can generally be produced using mechanical synthesis methods or light directed synthesis methods that incorporate a combination of photolithographic methods and solid phase oligonucleotide synthesis methods (Fodor, S. et al., 1991, Science, 251:767-777; Pirrung et al., U.S. Pat. No. 5,143,854 (see also PCT Application No. WO 90/15070) and Fodor et al., PCT Publication No. WO 92/10092 and U.S. Pat. No. 5,424,186) the entire teachings of each of which are incorporated by reference herein). Techniques for the synthesis of these arrays using mechanical synthesis methods are described in, e.g., U.S. Pat. No. 5,384,261, the entire teachings of which are incorporated by reference herein. In another embodiment, linear arrays or microarrays can be utilized.

Once an oligonucleotide array is prepared, a nucleic acid of interest is hybridized with the array and scanned for polymorphisms. Hybridization and scanning are generally carried out by methods described herein and also in, e.g., Published PCT Application Nos. WO 92/10092 and WO 95/11995, and U.S. Pat. No. 5,424,186, the entire teachings of which are incorporated by reference herein. In brief, a target nucleic acid sequence that includes one or more previously identified polymorphic markers is amplified by well-known amplification techniques, e.g., PCR. Typically, this involves the use of primer sequences that are complementary to the two strands of the target sequence both upstream and downstream from the polymorphism. Asymmetric PCR techniques can also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single polymorphism, arrays can include multiple detection blocks, and thus be capable of analyzing multiple, specific polymorphisms. In alternate arrangements, it will generally be understood that detection blocks can be grouped within a single array or in multiple, separate arrays so that varying, optimal conditions can be used during the hybridization of the target to the array. For example, it will often be desirable to provide for the detection of those polymorphisms that fall within G-C rich stretches of a genomic sequence, separately from those falling in A-T rich segments. This allows for the separate optimization of hybridization conditions for each situation.

Additional description of use of oligonucleotide arrays for detection of polymorphisms can be found, for example, in U.S. Pat. Nos. 5,858,659 and 5,837,832, the entire teachings of which are incorporated by reference herein.

Other methods of nucleic acid analysis can be used to detect one or more markers described herein or the Inv8p23 inversion allele. Representative methods include direct manual sequencing (Church, G. and Gilbert, W., 1988, Proc. Natl. Acad. Sci. USA, 81:1991-1995; Sanger, F. et al., 1977, Proc. Natl. Acad. Sci. USA, 74:5463-5467; Beavis et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V. et al., 1989, Proc. Natl. Acad. Sci. USA, 86:232-236), mobility shift analysis (Orita, M. et al., 1989, Proc. Natl. Acad. Sci. USA, 86:2766-2770), restriction enzyme analysis (Flavell, R. et al., 1978, Cell, 15:25-41; Geever, R. et al., 1981, Proc. Natl. Acad. Sci. USA, 78:5081-5085); heteroduplex analysis; chemical mismatch cleavage (CMC) (Cotton, R. et al., 1985, Proc. Natl. Acad. Sci. USA, 85:4397-4401); RNase protection assays (Myers, R. et al., 1985, Science, 230:1242-1246); use of polypeptides that recognize nucleotide mismatches, such as E. coli mutS protein, for example.

In one embodiment of the invention, diagnosis or detection of susceptibility to PD and or one or more comorbid disorders can be made by expression analysis by quantitative PCR (kinetic thermal cycling). This technique utilizing TaqMan® or Lightcycler® can be used to allow the identification of polymorphisms and whether a patient is homozygous or heterozygous.

Expression of one or more genes in the Inv8p23 region can be determined by a variety of methods, including enzyme linked immunosorbent assays (ELISAs), Western blots, immunoprecipitations and immunofluorescence. An alteration in expression can be, for example, an alteration in the quantitative polypeptide expression (i.e., the amount of polypeptide produced). Various means of examining expression or composition can be used, including spectroscopy, colorimetry, electrophoresis, isoelectric focusing, and immunoassays (e.g., David et al., U.S. Pat. No. 4,376,110) such as immunoblotting (see also Current Protocols in Molecular Biology, particularly chapter 10).

Kits (e.g., reagent kits) useful in the methods of diagnosis comprise components useful in any of the methods described herein, including for example, hybridization probes or primers as described herein (e.g., labeled probes or primers), reagents for detection of labeled molecules, restriction enzymes (e.g., for RFLP analysis), allele-specific oligonucleotides, antibodies, means for amplification of nucleic acid sequences in the Inv8p23 genomic region, or means for analyzing the orientation if the Inv8p23 inversion fragment, etc. In one embodiment, a kit for diagnosing susceptibility to PD and/or one or more comorbid disorders can comprise primers for nucleic acid amplification of the Inv8p23 region.

The invention provides methods (also referred to herein as “screening assays”) for identifying the presence of a nucleotide that hybridizes to a nucleic acid of the invention, as well as for identifying the presence of a polypeptide encoded by a nucleic acid of the invention. In one embodiment, the presence (or absence) of a nucleic acid molecule of interest (e.g., a nucleic acid that has significant homology with a nucleic acid of the invention) in a sample can be assessed by contacting the sample with a nucleic acid comprising a nucleic acid of the invention under stringent conditions as described above, and then assessing the sample for the presence (or absence) of hybridization. In another embodiment, high stringency conditions are conditions appropriate for selective hybridization. In another embodiment, a sample containing the nucleic acid molecule of interest is contacted with a nucleic acid containing a contiguous nucleotide sequence (e.g., a primer or a probe as described above) that is at least partially complementary to a part of the nucleic acid molecule of interest, and the contacted sample is assessed for the presence or absence of hybridization. In another embodiment, the nucleic acid containing a contiguous nucleotide sequence is completely complementary to a part of the nucleic acid molecule of interest. In any of these embodiments, all or a portion of the nucleic acid of interest can be subjected to amplification prior to performing the hybridization.

In another embodiment, the presence (or absence) of a polypeptide of interest, such as a polypeptide of the invention or a fragment or variant thereof, in a sample can be assessed by contacting the sample with an antibody that specifically binds to the polypeptide of interest (e.g., an antibody such as those described above), and then assessing the sample for the presence (or absence) of binding of the antibody to the polypeptide of interest.

In another embodiment, the invention provides methods for identifying agents (e.g., fusion proteins, polypeptides, peptidomimetics, prodrugs, receptors, binding agents, antibodies, small molecules or other drugs, or ribozymes) that alter (e.g., increase or decrease) the activity of the polypeptides described herein, or that otherwise interact with the polypeptides herein. For example, such agents can be agents that bind to polypeptides described herein; that have a stimulatory or inhibitory effect on, for example, activity of polypeptides of the invention; or that change (e.g., enhance or inhibit) the ability of the polypeptides of the invention to interact with other agents (e.g., receptors or other binding agents); or that alter posttranslational processing of the polypeptide (e.g., agents that alter proteolytic processing to direct the polypeptide from where it is normally synthesized to another location in the cell, such as the cell surface; agents that alter proteolytic processing such that more polypeptide is released from the cell, etc).

In one embodiment, the invention provides assays for screening candidate or test agents that bind to or modulate the activity of polypeptides described herein (or biologically active portion(s) thereof), as well as agents identifiable by the assays. Test agents can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds.

In other embodiments of the invention, assays can be used to assess the impact of a test agent on the activity of a polypeptide of the invention (i.e., one that results from the expression of one or more genes in the Inv8p23 inversion fragment or is disrupted as a result of the Inv8p23 inversion). The ability of the test agent to bind to a polypeptide of the invention can be determined, for example, by coupling the test agent to a radioisotope or enzymatic label such that binding of the test agent to the polypeptide can be determined by detecting the label, either directly or indirectly. Alternatively, test agents can be enzymatically labeled with, for example, horseradish peroxidase, alkaline phosphatase, or luciferase, and the enzymatic label detected by determination of conversion of an appropriate substrate to product. It is also within the scope of this invention to determine the ability of a test agent to interact with the polypeptide without the labeling of any of the interactants. For example, a microphysiometer can be used to detect the interaction of a test agent with a polypeptide of the invention without the labeling of either the test agent or polypeptide (McConnell, H. et al., 1992, Science, 257:1906-1912). As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and polypeptide.

This invention further pertains to novel agents identified by the above-described screening assays. Accordingly, it is within the scope of this invention to further use an agent identified as described herein in an appropriate animal model. For example, an agent identified as described herein (e.g., a test agent that is a modulating agent, an antisense nucleic acid molecule, a specific antibody, or a polypeptide-binding agent) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

The present invention also pertains to pharmaceutical compositions comprising agents described herein, particularly nucleotides encoding the polypeptides described herein; comprising polypeptides described herein and/or an agent that alters (e.g., enhances or inhibits) expression of one or more genes in the Inv8p23 region as described herein. For instance, a polypeptide, protein, an agent that alters expression, or a binding agent or binding partner, fragment, fusion protein or prodrug thereof, or a nucleotide or nucleic acid construct (vector) comprising a nucleotide of the present invention, or an agent that alters polypeptide activity, can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrolidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients can be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent can be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents that will be therapeutically effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the formulation will also depend on the route of administration, and the seriousness of the symptoms of PD, and should be decided according to the judgment of a practitioner and each patient's circumstances. Effective doses can be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit can also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

The present invention encompasses methods of treatment (prophylactic and/or therapeutic) for PD and/or one or more comorbid disorders using an agent identified herein. A “therapeutic agent” is an agent that effectively treats PD and/or one or more comorbid disorders. Representative therapeutic agents include the following: nucleic acids or fragments or derivatives thereof described herein, particularly nucleotides encoding the polypeptides described herein and vectors comprising such nucleic acids (e.g., a gene, cDNA, and/or mRNA, double-stranded interfering RNA, a nucleic acid encoding a polypeptide of the invention or active fragment or derivative thereof, or an oligonucleotide that can optionally comprise at least one polymorphism, antisense nucleic acids or small double-stranded interfering RNA, and other agents that alter (e.g., inhibit or antagonize) gene expression or polypeptide activity. More than one therapeutic agent can be used concurrently, if desired.

The term, “treatment” as used herein, refers not only to ameliorating symptoms associated with the disease, but also preventing or delaying the onset of the disease, and also lessening the severity or frequency of symptoms of the disease, preventing or delaying the occurrence of a second episode of the disease or condition; and/or also lessening the severity or frequency of symptoms of the disease or condition.

The therapeutic agent(s) are administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount that will be therapeutically effective in the treatment of a particular individual's disorder or condition will depend on the symptoms and severity of the disease, and can be determined by standard clinical techniques. In addition, in vitro or in vivo assays can optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses can be extrapolated from dose-response curves derived from in vitro or animal model test systems.

The present invention is also directed to methods for predicting efficacy of drug treatment of psychiatric disorders, anxiety disorders, PD and comorbid disorders. Current methods of treating such disorders with drugs have significant risks of substantial side effects. Thus, determining whether a patient will be effectively treated with a particular drug treatment will be useful. Drugs useful in treating psychiatric disorders include, for example, Amine Reuptake Inhibitors, e.g., Selective Serotonin Reuptake Inhibitors (e.g., fluoxetine, sertraline, paroxetine, fluvoxamine), Selective Norepinephrine Reuptake Inhibitors (e.g., desipramine, maprotiline), Combined Serotonin-Norepinephrine Reuptake Inhibitors (e.g., Selective (e.g., venlafaxine) or Non-selective (e.g., tertiary amine tricyclics, nortriptyline), Combined Dopamine-Norepinephrine Reuptake Inhibitors (e.g., Selective (e.g., bupropion)); Inhibitors of Enzymatic Metabolism, e.g., Irreversible/nonselective Monoamine Oxidase Inhibitors (e.g., phenelzine, tranylcypromine, isocarbozazid), Reversible/selective Inhibitor of Monoamine Oxidase-A (e.g., moclobimide); Receptor Antagonists, e.g., 5-HT 2A receptor antagonist (e.g., Nonselective (e.g., trazodone)), Combined 5-HT 2A antagonist with Serotonin Reuptake Inhibition (e.g., Nonselective (e.g., nefazodone), tricyclics, and Combined 5-HT 2A, 5-HT 2C and alpha-2 antagonists (e.g., Nonselective (e.g., mirtazipine)). Although psychiatric disorders, e.g., depression, have been treated by these drugs for several years, a significant fraction of patients are non-responsive or show little effect of the treatment. As there are risks associated with methods for treating psychiatric disorders, identification of patients that will be responsive to treatment is important. Methods described herein are used to identify markers that are associated with drug responsiveness.

The determination of drug responsiveness is accomplished by detecting one or more markers shown herein (see Example 5) to be associated with drug responsiveness. The present invention is directed, for example, in determining drug responsiveness of a human patient for a drug used to treat psychiatric disorder. A “responder” population was identified, and markers were identified in this population that indicated an association with drug responsiveness. These marker, markers in LD with these markers, and other markers associated with drug responsiveness are therefore useful for predicting drug responsiveness in a human patient. Identification of such a marker in the patient is indicative of drug responsiveness.

The invention will be further described by the following non-limiting examples. The teachings of all publications cited herein are incorporated herein by reference in their entirety.

### EXAMPLES

### Example 1

FISH experiments were initially conducted on material from the cell lines from individuals with PD to look for DUP25, a large duplication that has been reported to be associated with joint laxity and anxiety disorders in a Spanish population (Gratacos, M. et al., 2001. Cell, 106:367-379). The region of chromosome 8 became interesting as a recombination map of the human genome was constructed, and discrepancies in the recombination pattern in this region were noted. The average genetic order of the markers was opposite to that from the reported human genome sequence (Kong, A. et al., 2002. Nat. Genet., 31:241-247). The inversion polymorphism was first reported by Giglio, S. et al. (2001, Am. J. Hum. Genet., 68:874-883), who detected it from CEPH genetic data. Although efforts aimed at cloning the breakpoints have made significant progress (Giglio, S. et al., 2002. Am. J Hum. Genet., 71:276-285), the regions have not been narrowed to the extent necessary to design a simple PCR assay to determine the orientation. Until now, Inv8p23 had not been associated with any phenotype.

The evolutionary history of Inv8p23 has not been studied, and it is not known whether the inversion has occurred only once or multiple times. If the inversion has occurred only once, it is more likely that the common form is the ancestral one. This is supported by the analysis of mouse-human synteny in the region, which reveals reorganization of the human sequence in NCBI Build 33 (of the human genome) relative to the mouse sequence that is consistent with an inversion. The average genetic order is inconsistent with the physical order in NCBI Build 33, which thus represents the less frequent, or inverted, variant. However, more detailed studies of SNPs and haplotypes in the region are required before ancestral status can be assigned with certainty.

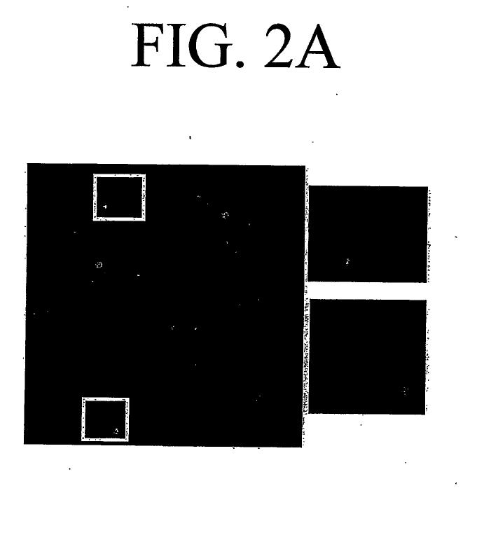

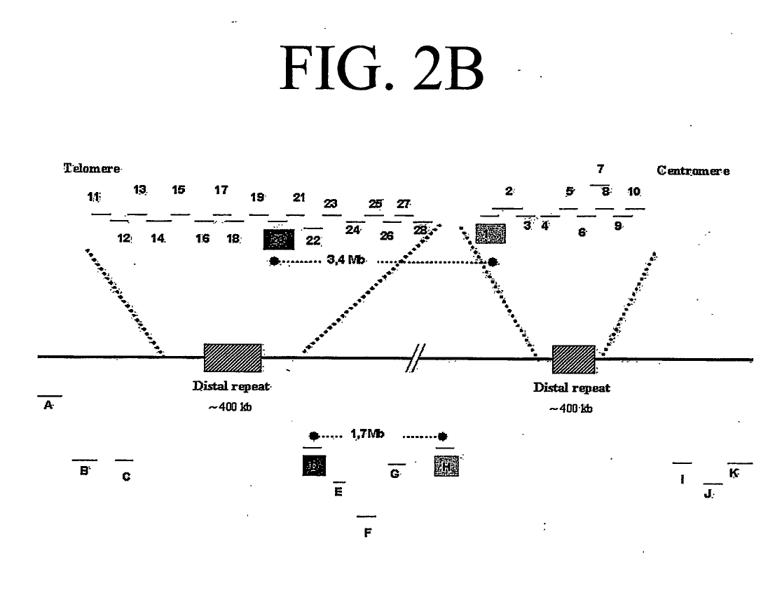

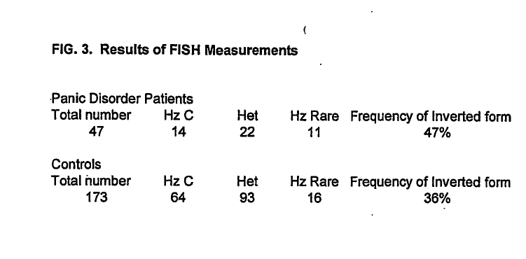

Cell lines were collected from PD patients to investigate the prevalence of DUP25 on chromosome 15q24-26 in Icelandic PD patients. DUP25 has been reported to be associated with anxiety disorders and hypermobility of the joints in a Spanish population (Gratacos, M. et al., 2001. Cell, 106:367-379). DUP25 was not detected in the Icelandic population. Attention then shifted to studying the role of Inv8p23 in PD. FISH data were analyzed (FIGS. 2A and 2B) for the first group of 20 PD patients, and an excess of the less frequent inversion allele was discovered in PD cell lines compared to controls. Subsequent hybridizations confirmed that over 50% of the chromosomes have the inverted allele in PD patients. Subsequent samples and chromosomal spreads were obtained (47 PD patients and 173 controls), and the frequency of the inversion was 47% in PD patients vs. 36%, in controls (two-sided Fisher exact test, p=0.07) (FIG. 3).

While the FISH experiments clearly showed the association of the inverted allele with PD, FISH is not the ideal method to study large sets of patients since it is expensive, time consuming, and requires that cell lines or fresh blood samples are available. Therefore, association of other markers within the region of the inverted segment were searched in order to (1) identify surrogate markers or haplotypes allowing the determination of orientation based on genotypes alone, and (2) to collect genetic data to characterize the inversion with regard to linkage disequilibrium and the evolutionary history of the region, and (3) to look for allelic association to panic disorder at markers in the region.

To identify surrogate markers, DNA from the 173 control individuals with known orientation at 8p23, i.e., samples from individuals that had been studied by the FISH measurements discussed above, was used. Samples were genotyped, and, using microsatellite and SNP markers from the region, results were analyzed using NEMO, a program developed at deCode genetics (Grétarsdóttir, S. et al., 2003, Nat. Genet., 35(2) in press). FIG. 4 summarizes the association for those markers most strongly associated to the 8p23 orientation (R2>0.3). The association of markers with the orientation is strong and extensive throughout the region, even between markers from opposite ends of the inversion separated by a large distance. Recombination is supressed in heterozygotes and the two forms rarely mix by recombination such that each orientation has, over time, developed its own distribution of allelic frequencies at markers in the region, producing extensive linkage disequilibrium (LD) in the region when a random sample of chromosomes is analyzed.

The identification of surrogate markers allows for the increase in sample size for PD and controls, and also for the study additional psychiatric phenotypes.

Use of Surrogate Markers to Determine Inv8p23 Orientation

As an example of how the genotypes of a single marker are used to detect orientation, consider the G allele of SG08S5 (the marker most strongly associated with the orientation) is estimated to have frequency 91.3% in inverted chromosomes, and 9.8% in the common orientation (FIG. 4). Using estimated population frequencies of the two orientation of 36.1% and 63.9%, and with the application of Bayes' rule, one can conclude that a chromosome with the G allele for SG08S5 has 84.1% chance to have the inversion, and a chromosome with the A allele for SG08S5 has 5.2% chance to have the inversion. Any marker correlated with the orientation can be utilized in similar manner.

Use of Multiple Surrogate Markers to Determine Inv8p23 Orientation

Apart from using individual markers separately, using two or more markers jointly as haplotypes can further improve the specificity of predicting PD risk. For example, a haplotype with the A allele for SG08S71 and the G allele for DG00AAHBG has frequency of 43.3% in PD patients versus 29.3% in controls, giving a relative risk of 1.84 compared to other haplotypes, and a two-sided p-value of 1.1×10⁻⁶.

Orientation at 8p23 is Associated with Panic Disorder and Bipolar Disorder

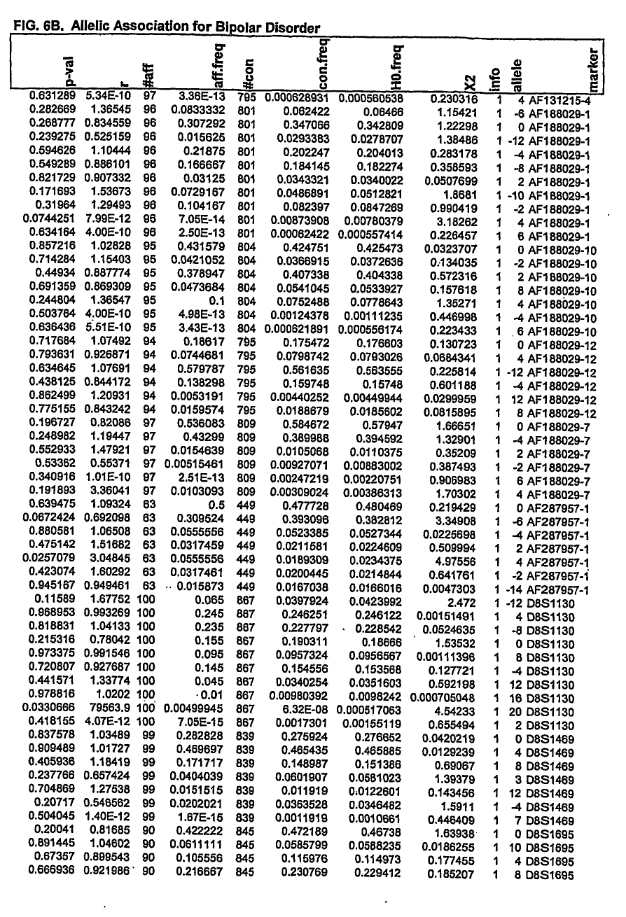

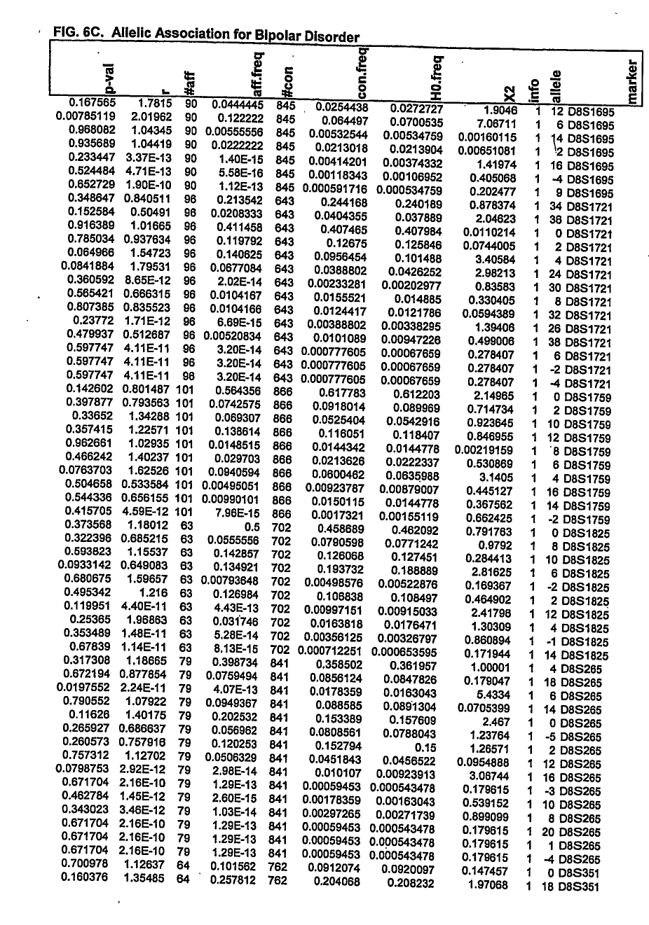

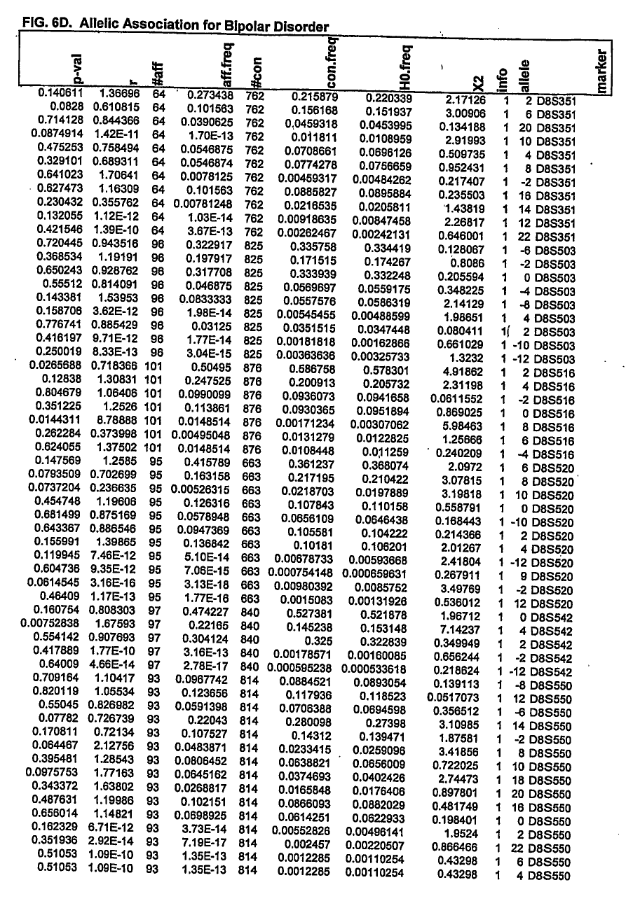

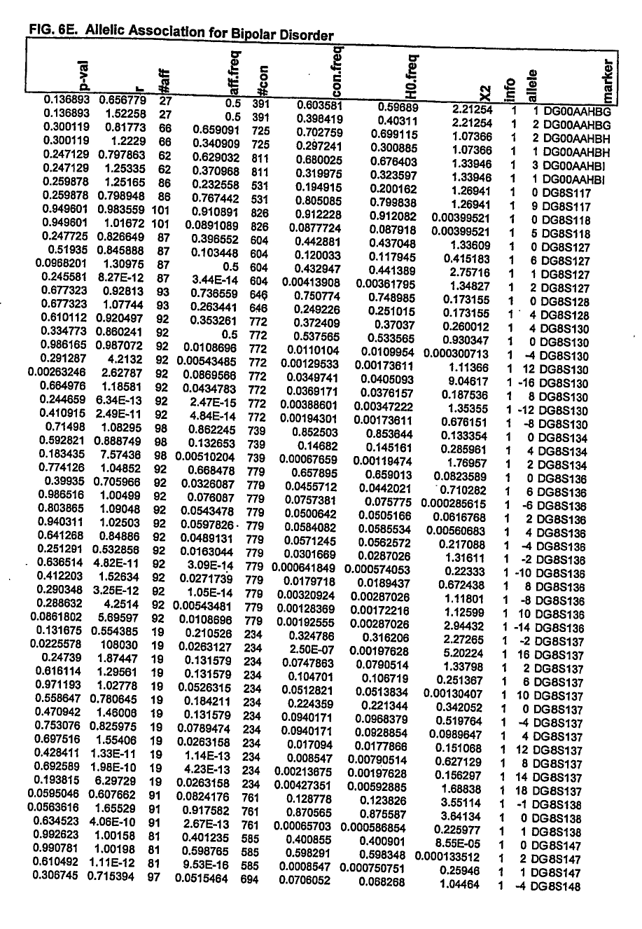

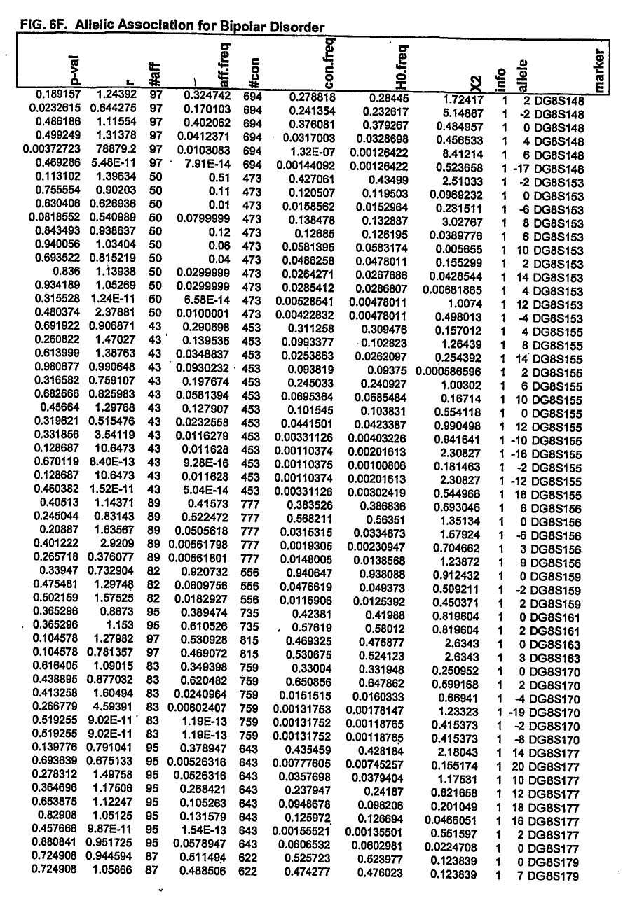

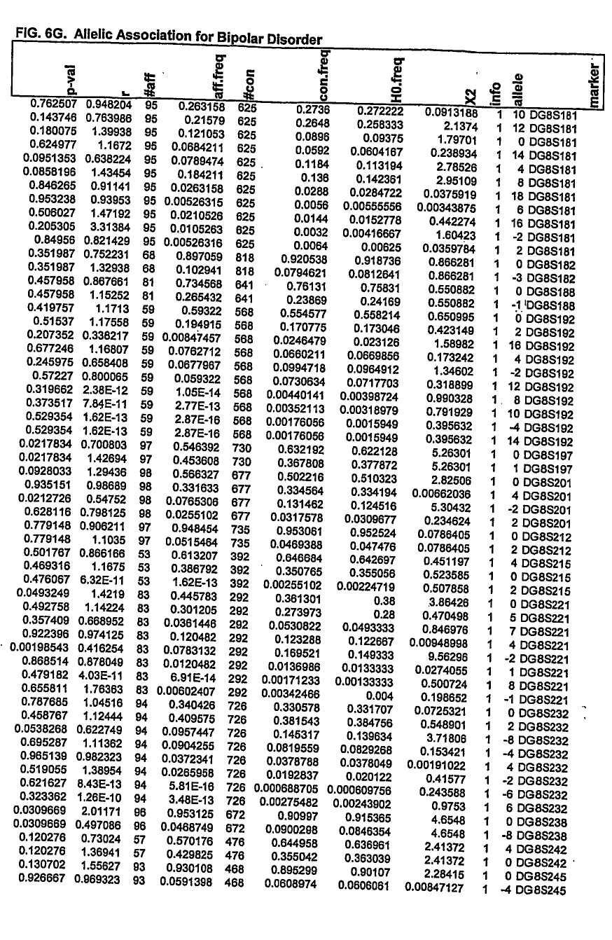

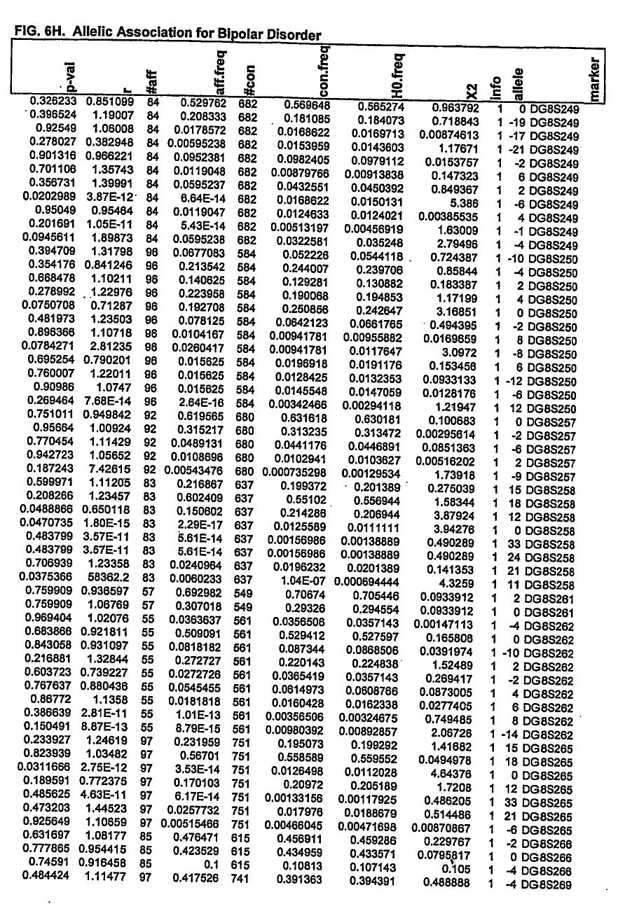

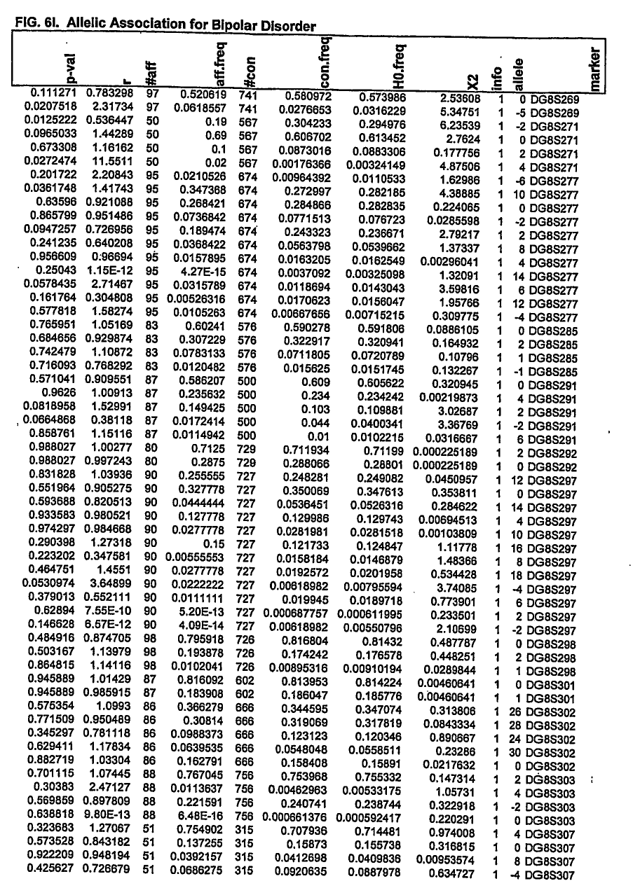

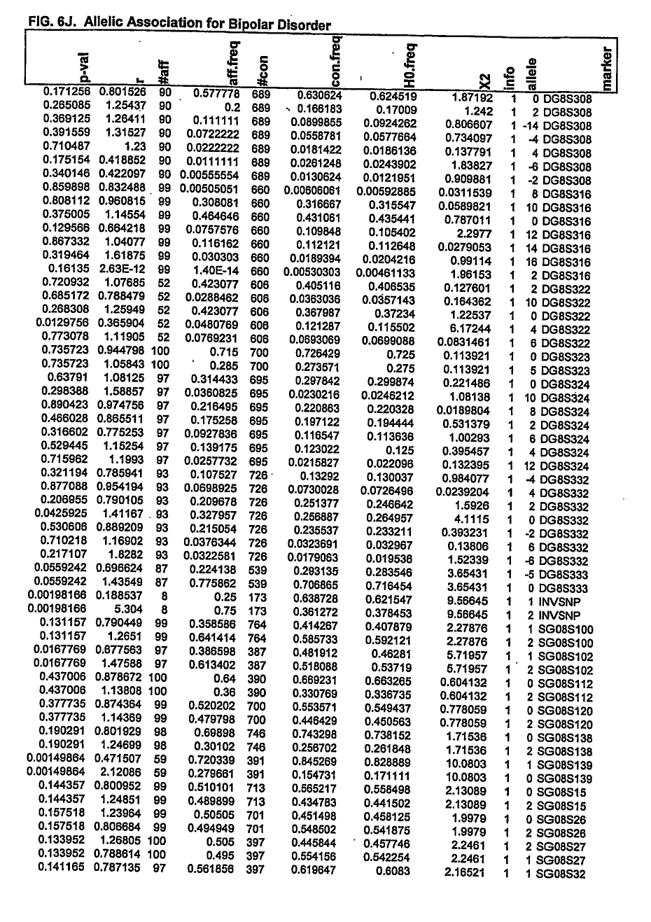

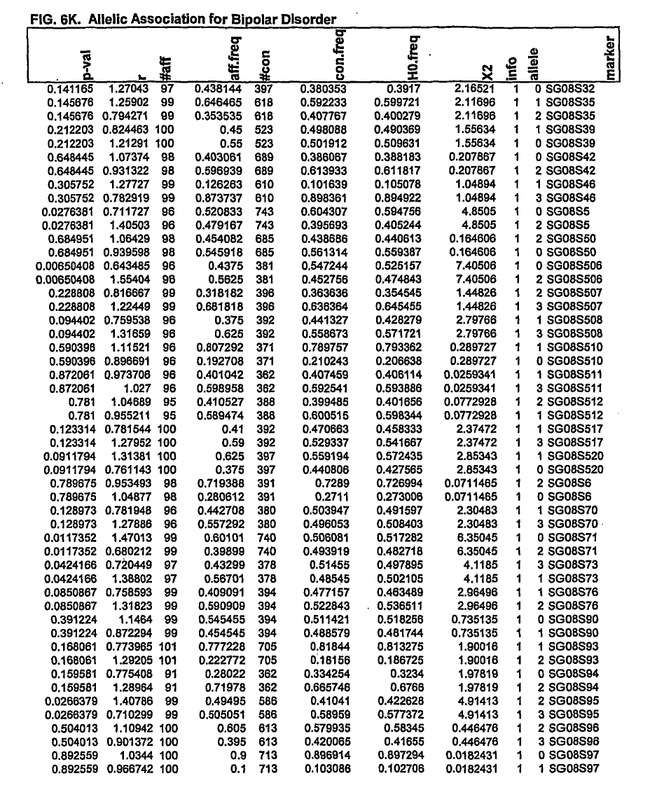

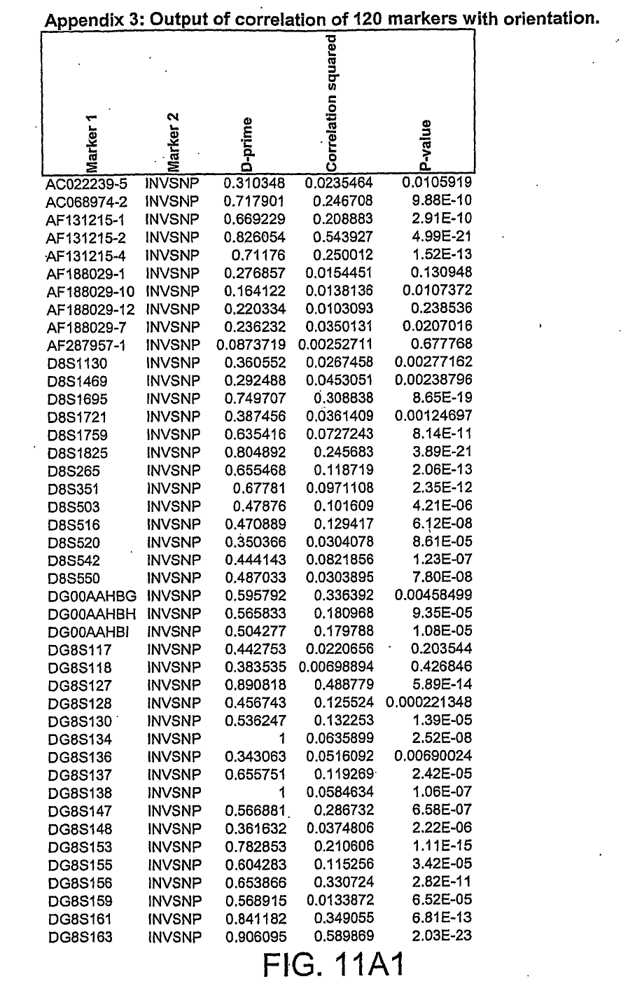

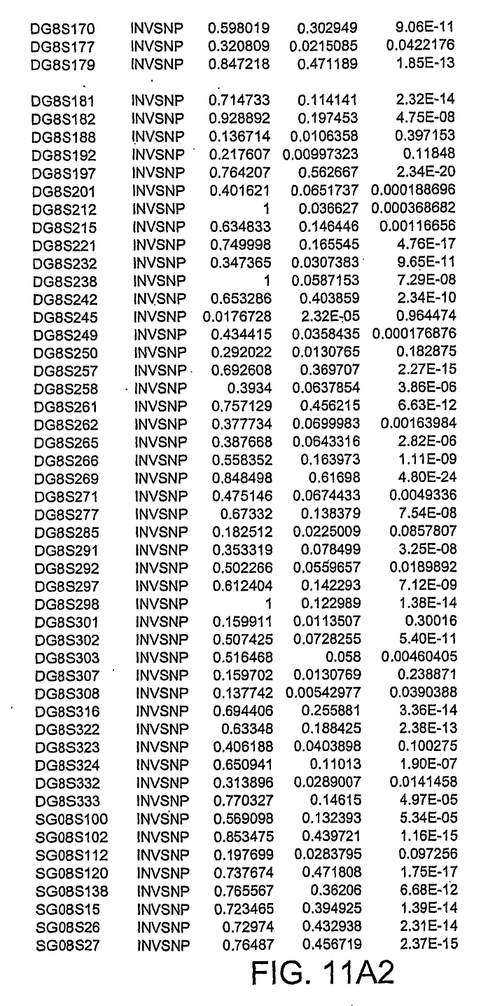

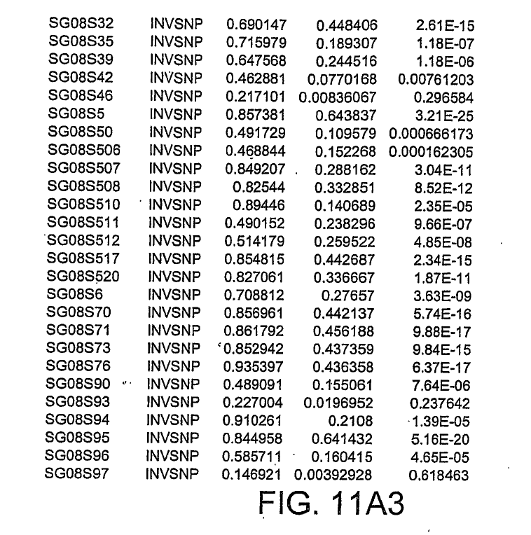

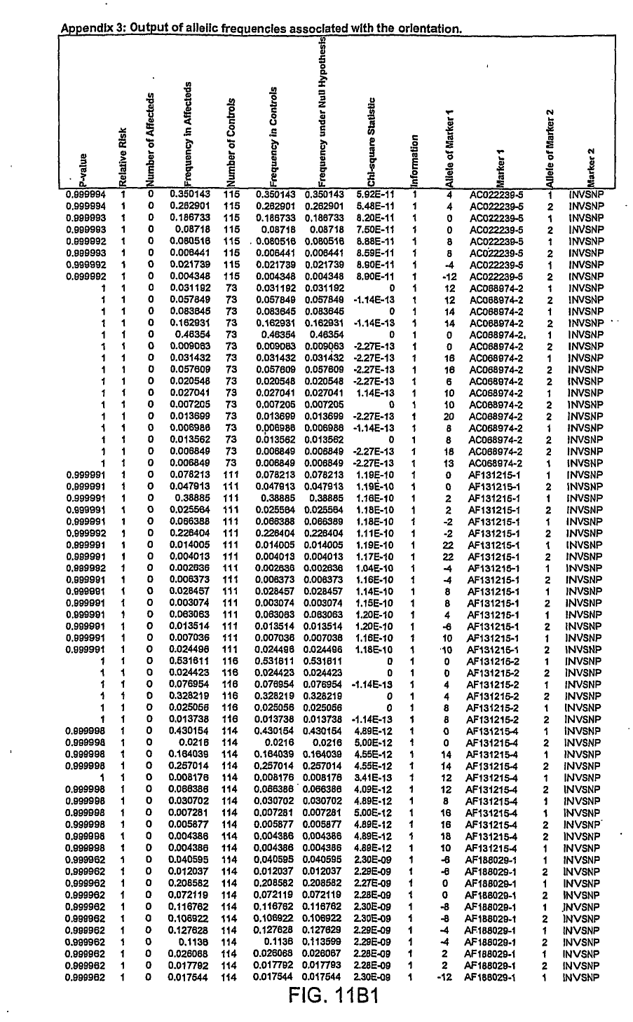

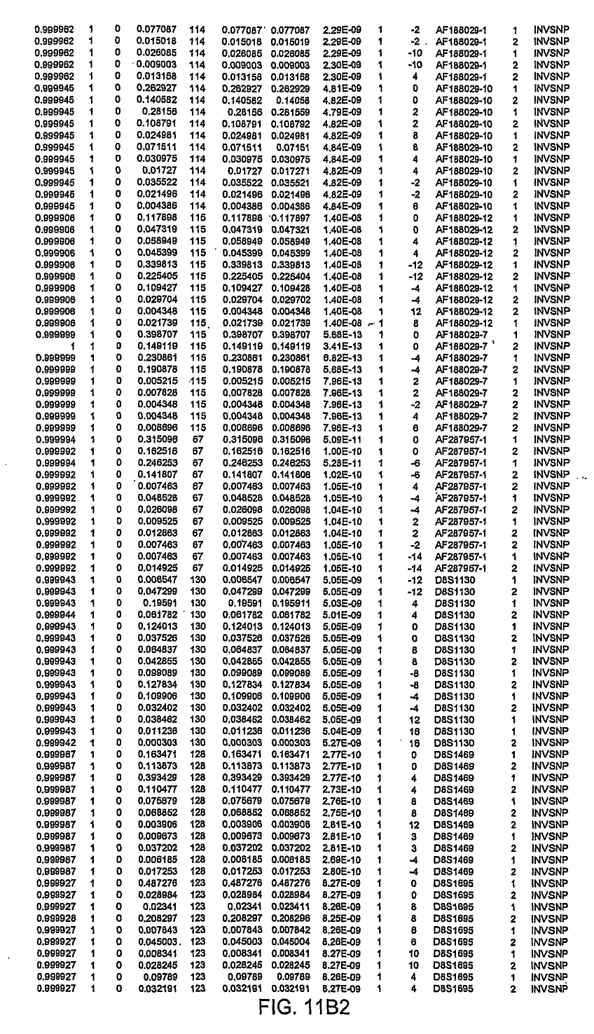

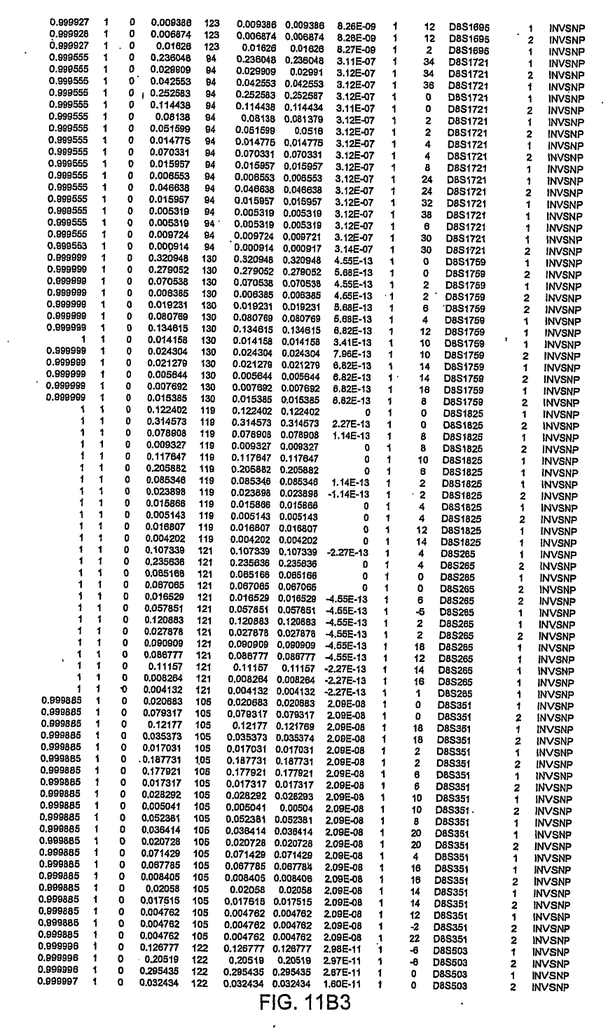

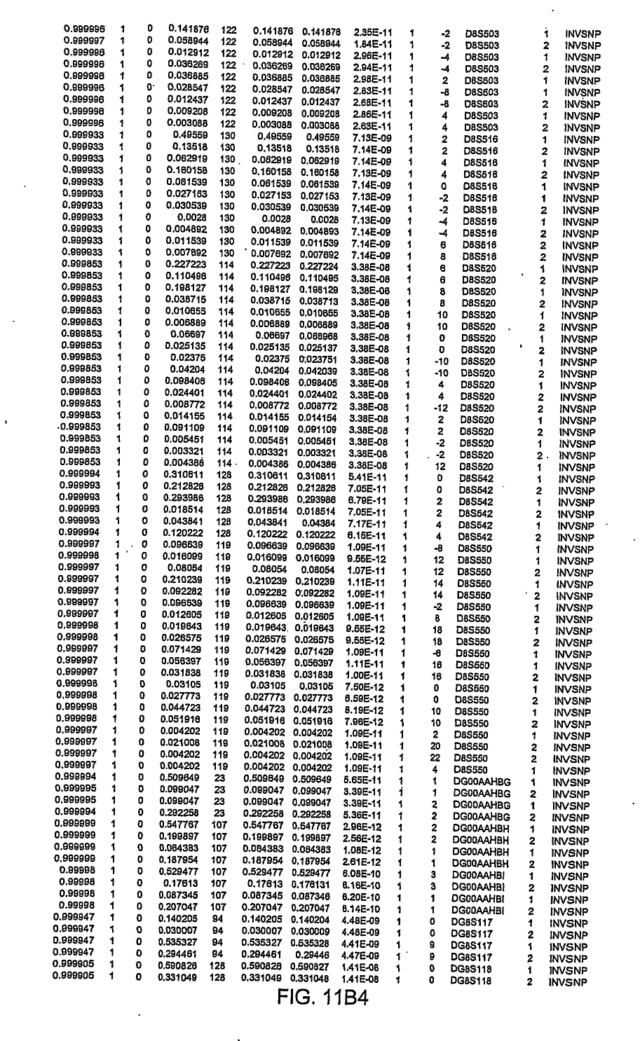

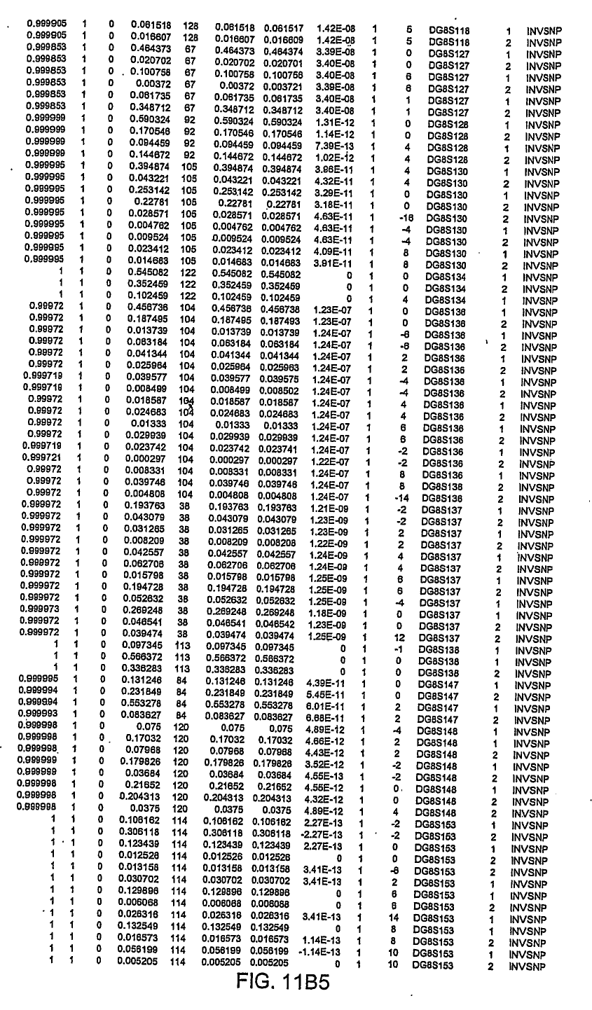

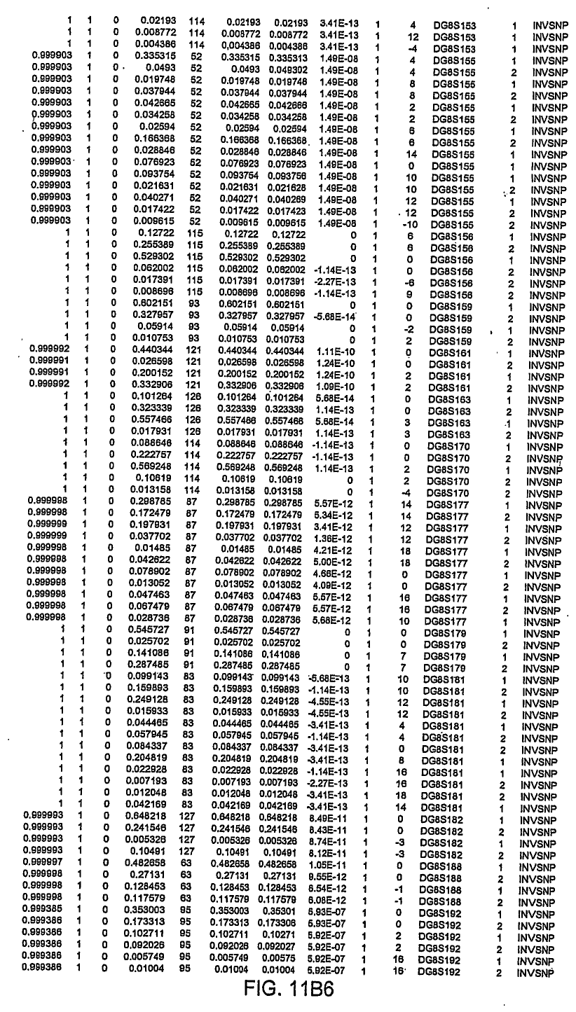

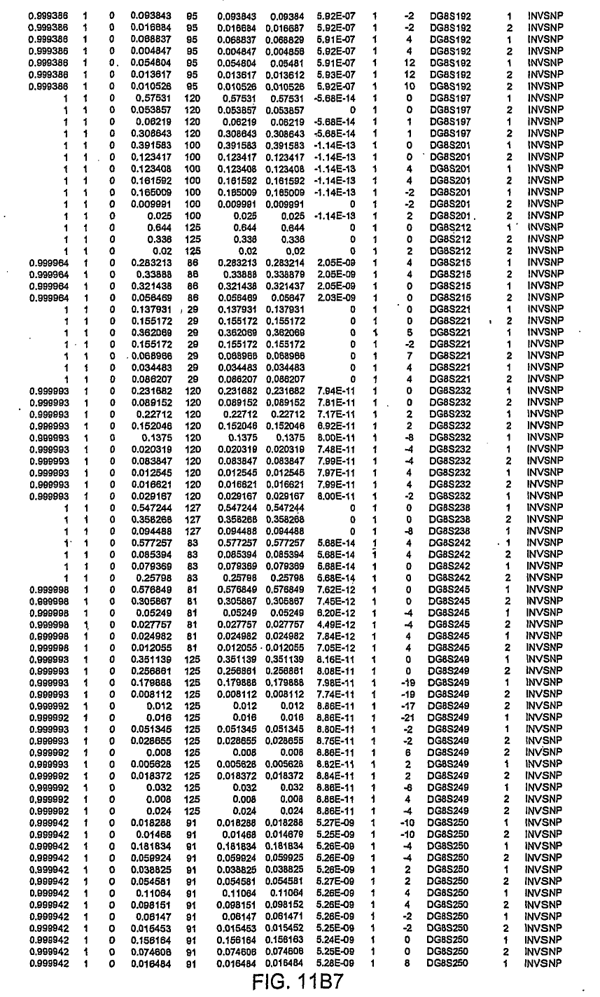

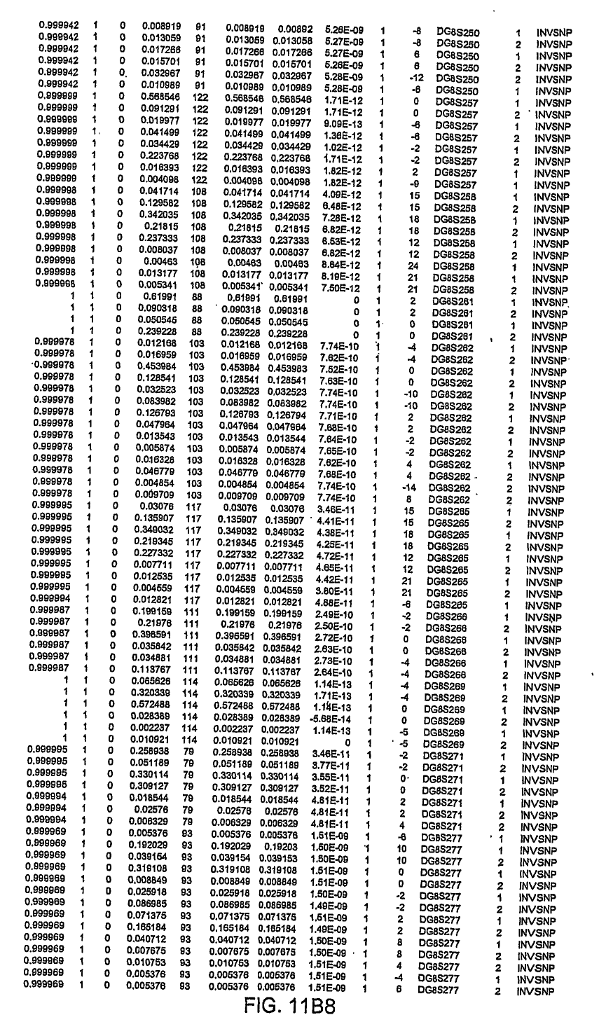

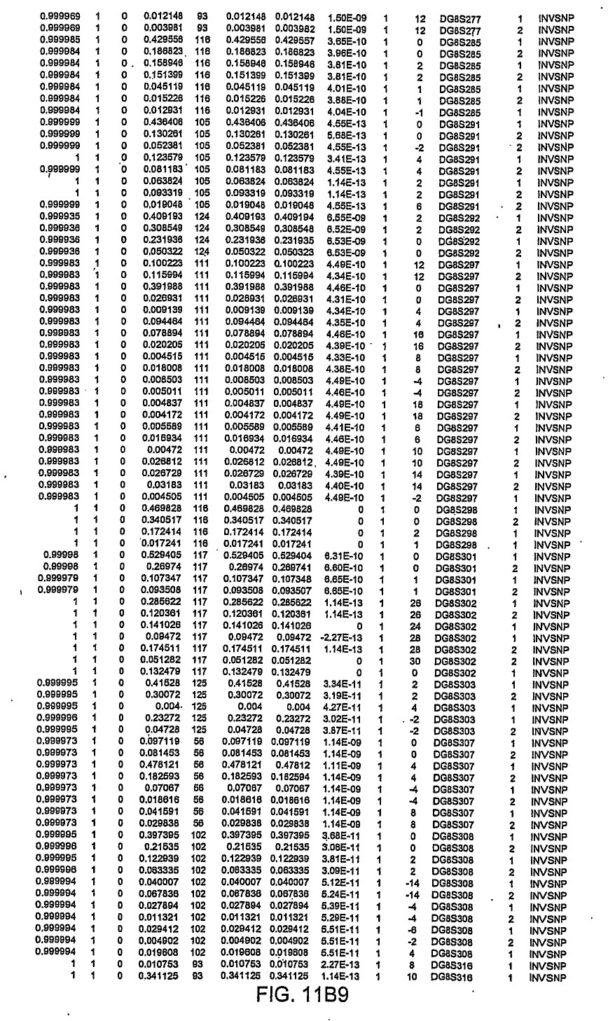

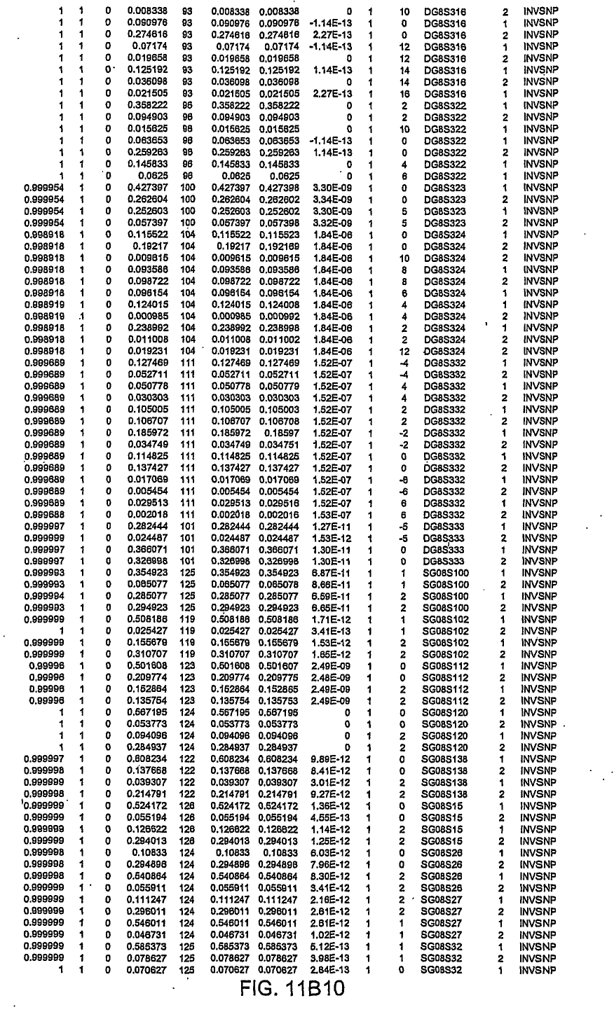

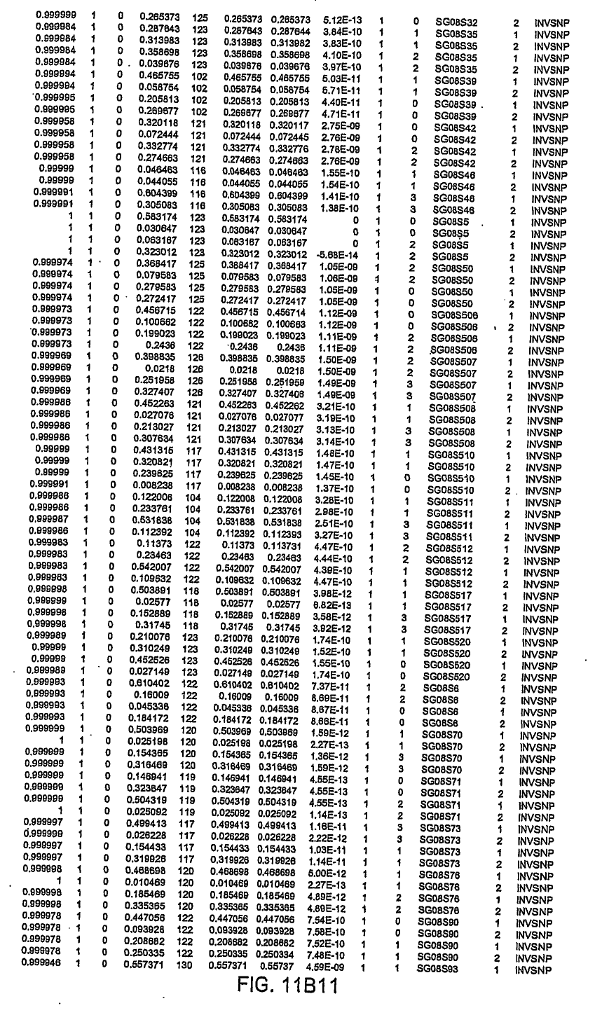

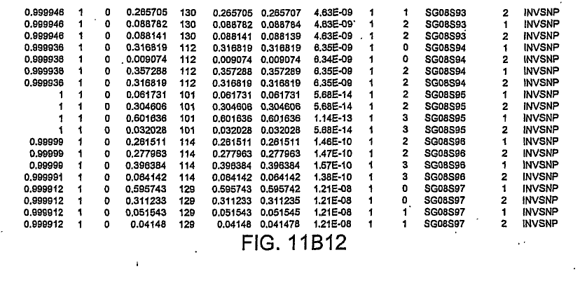

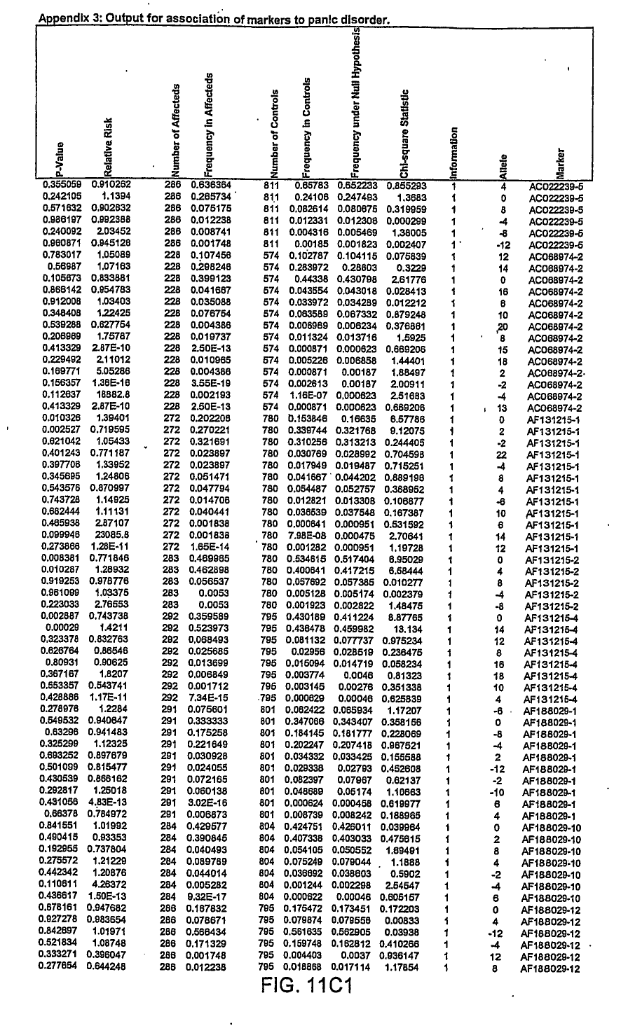

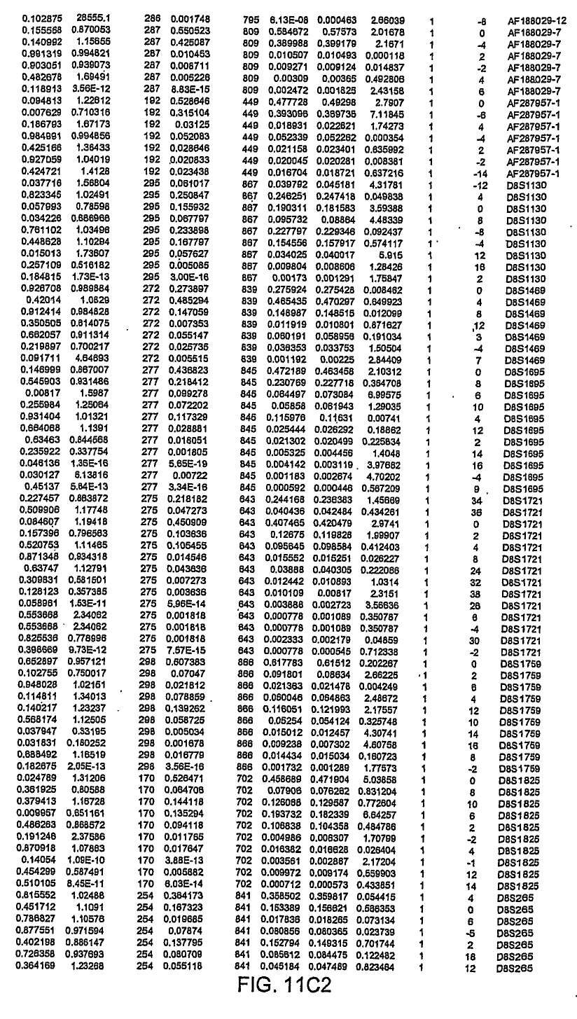

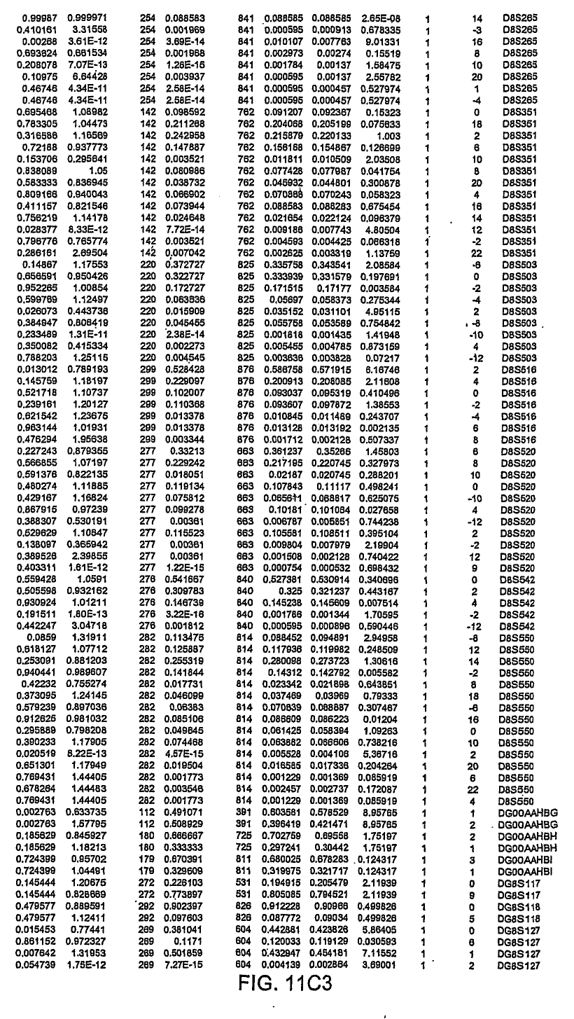

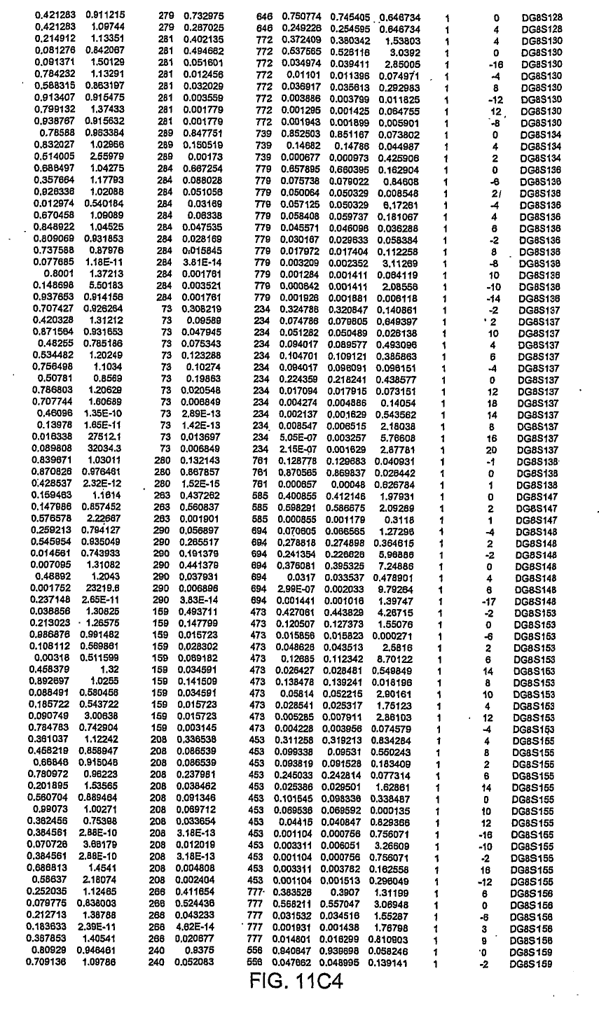

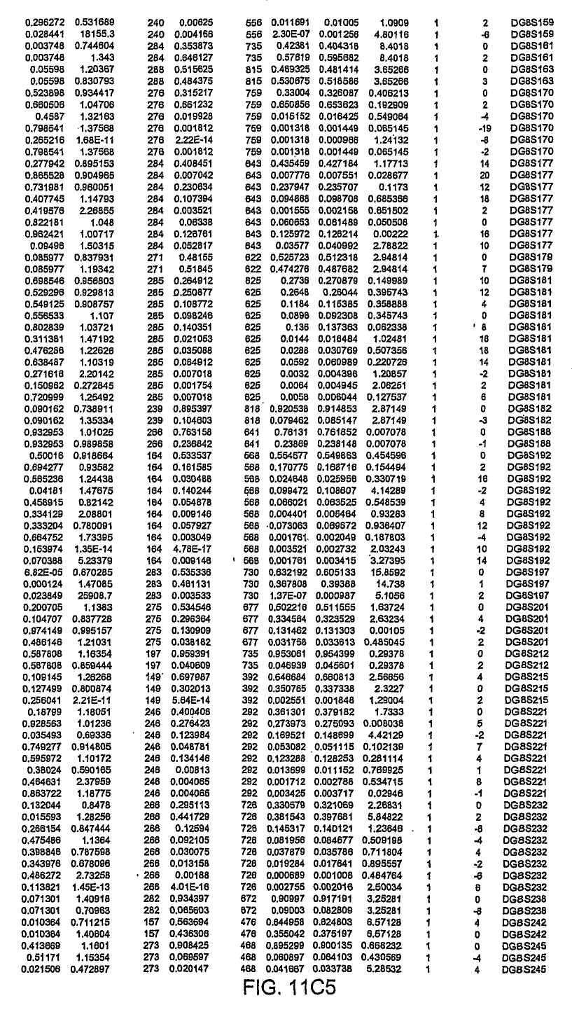

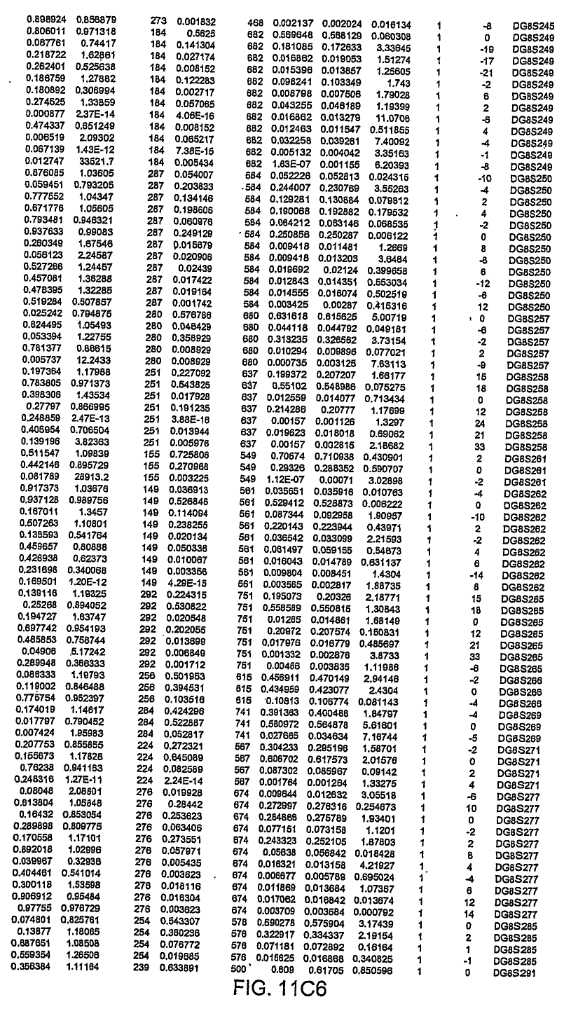

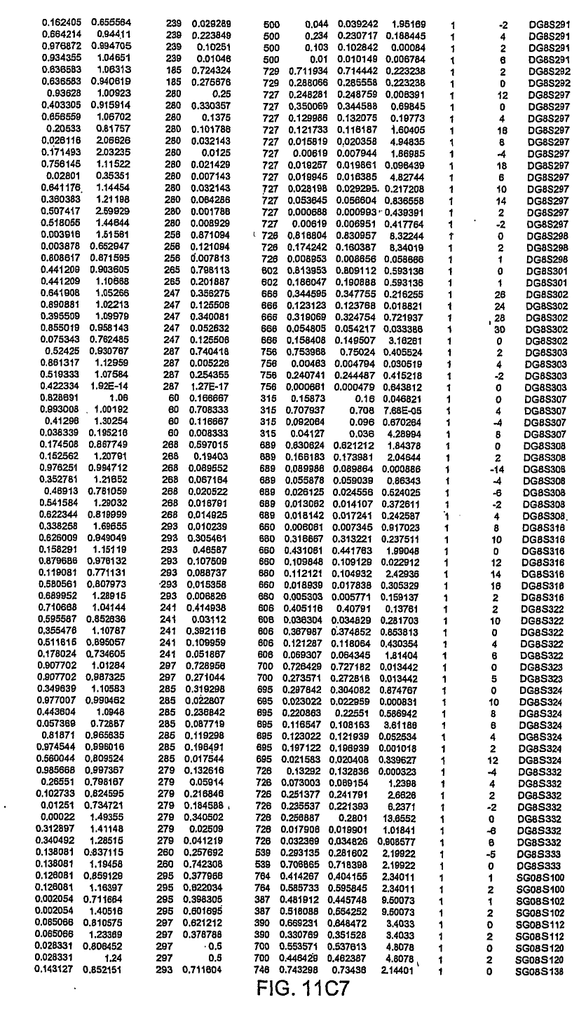

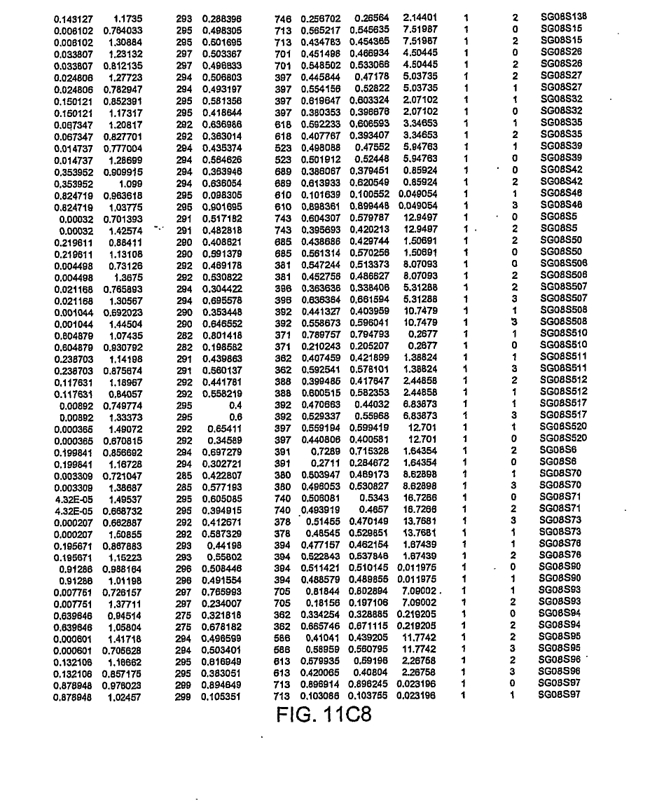

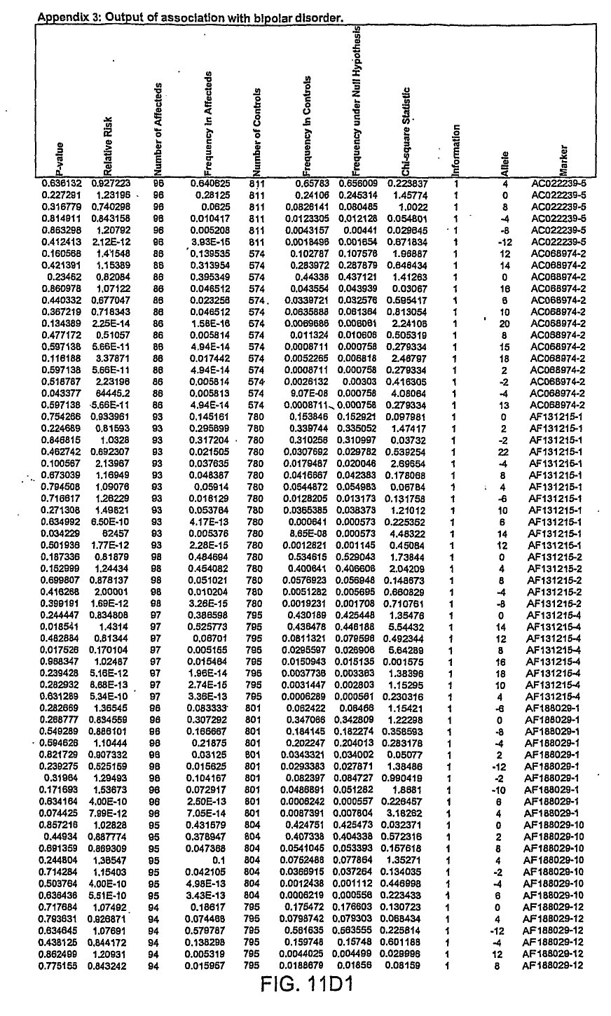

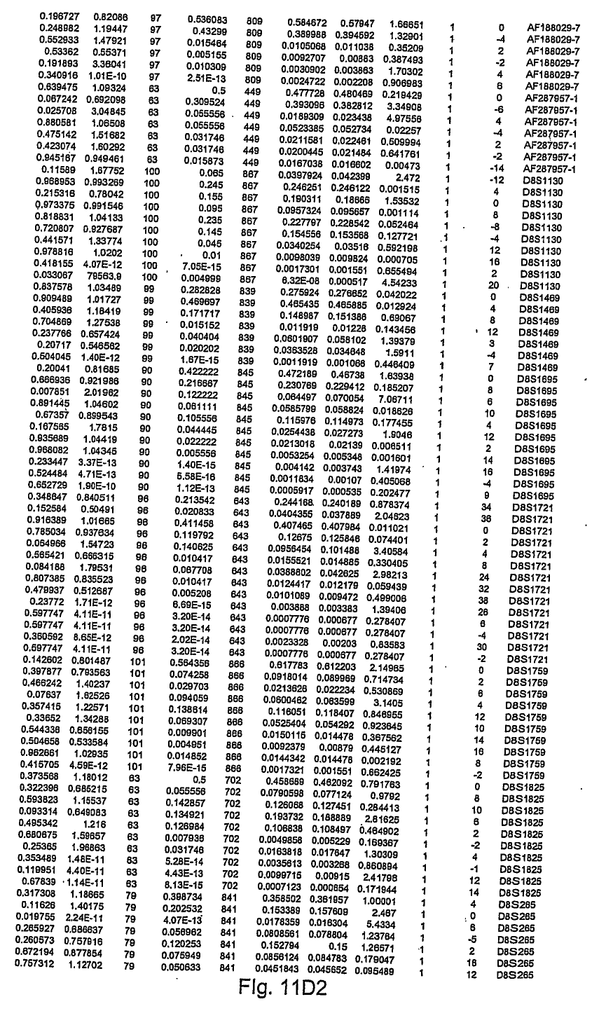

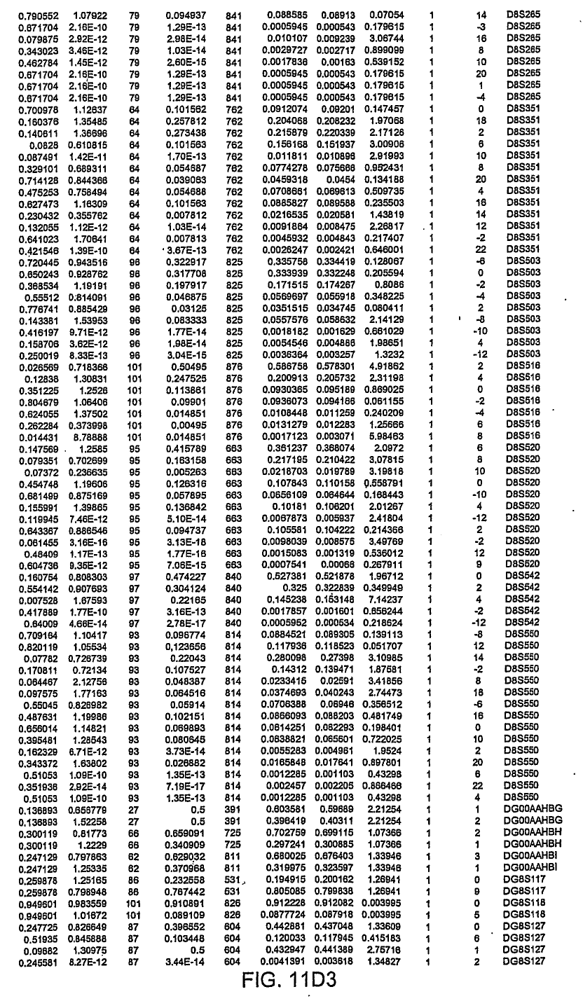

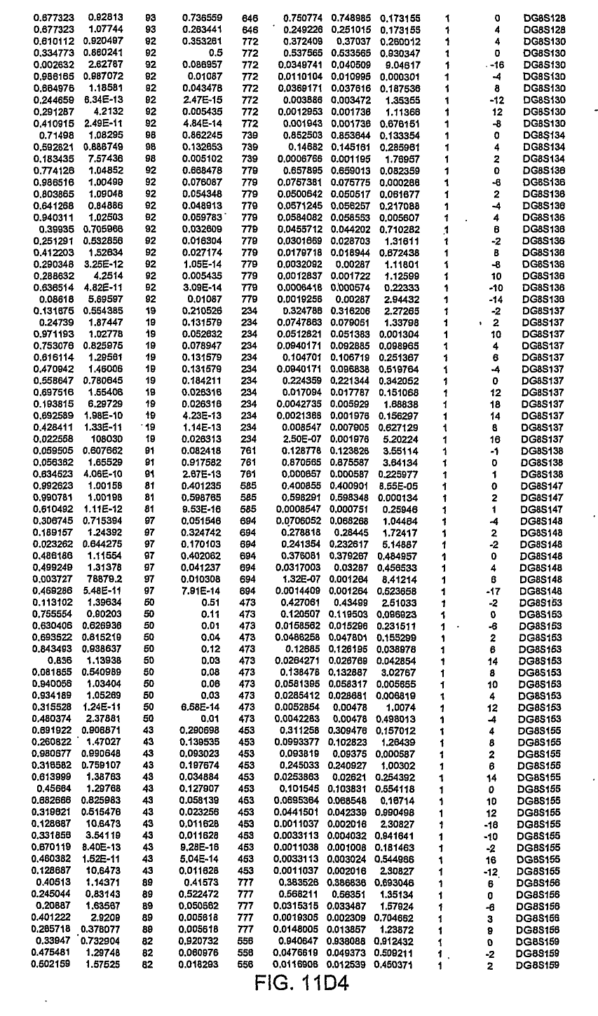

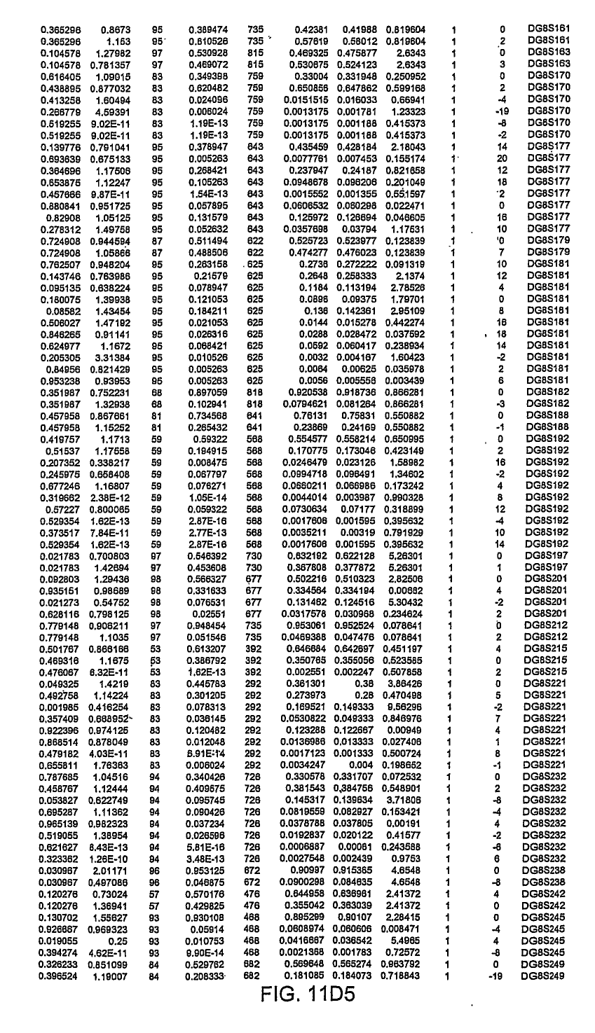

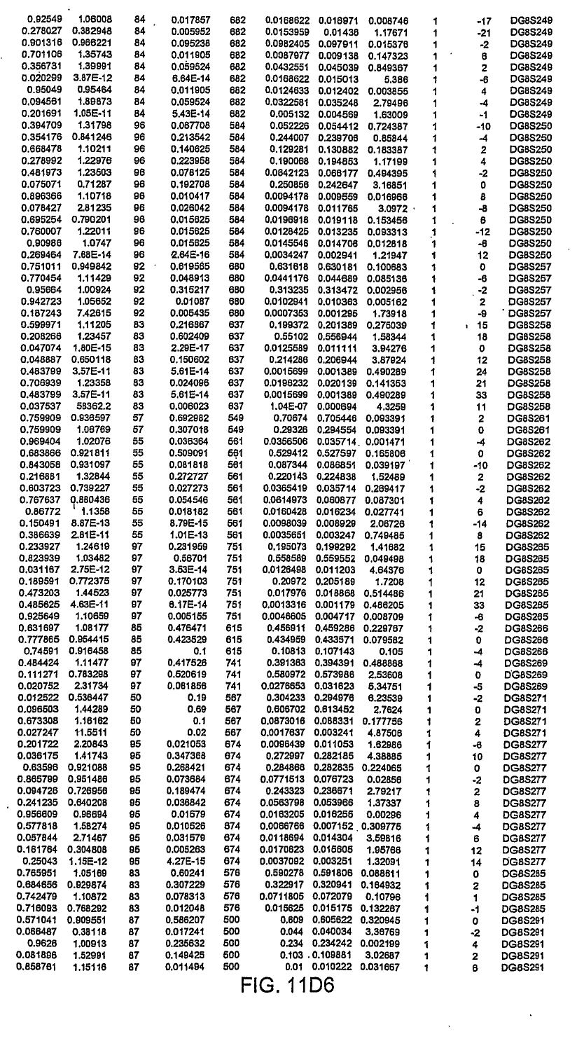

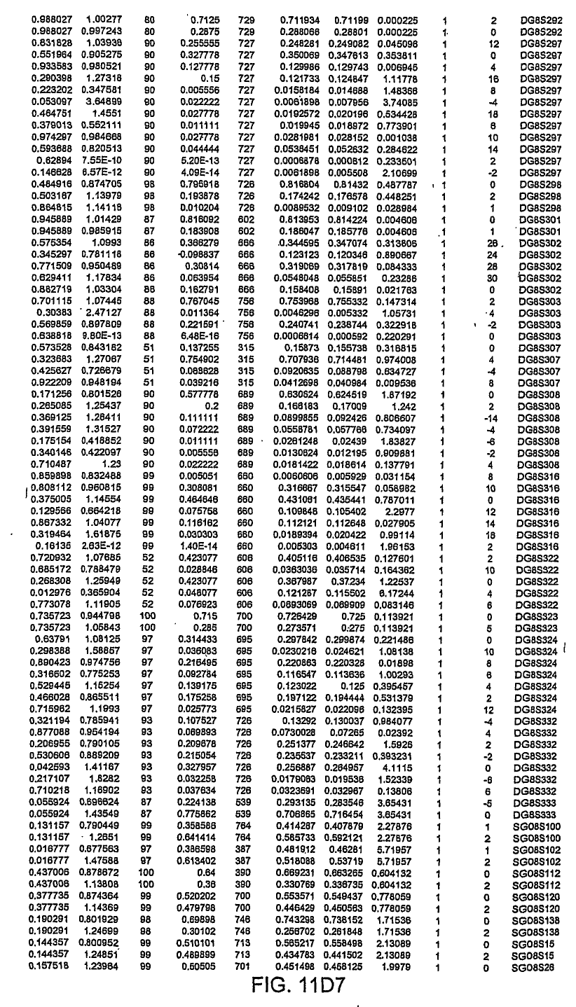

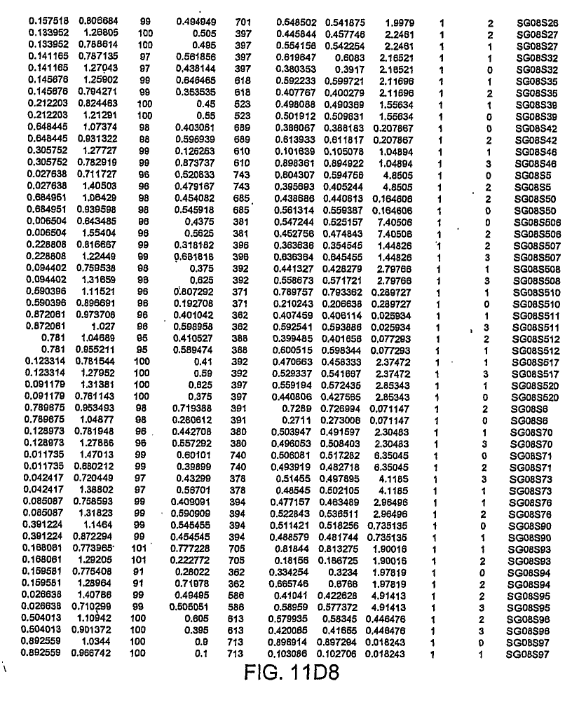

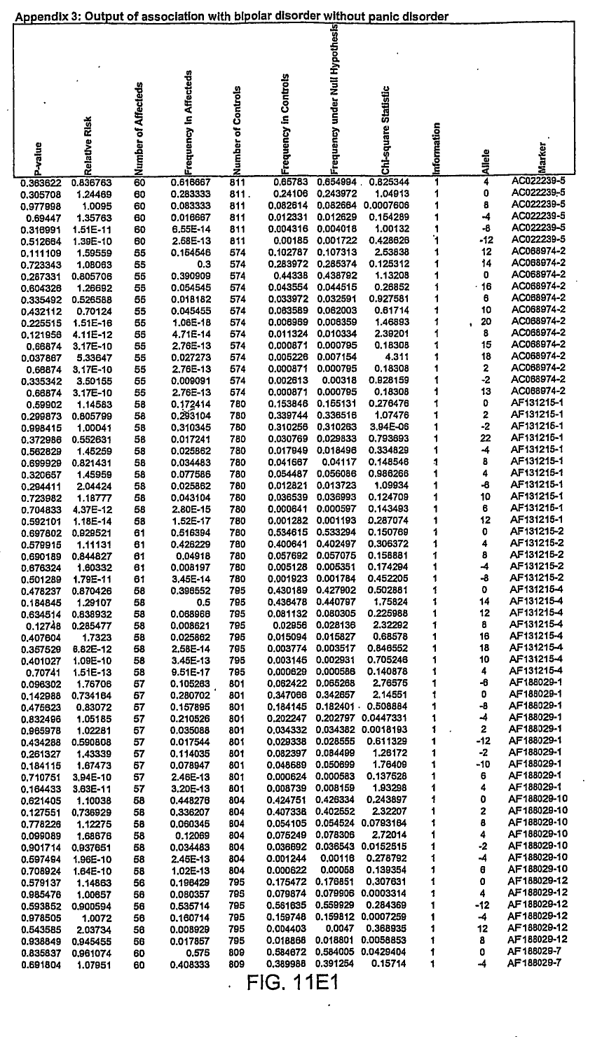

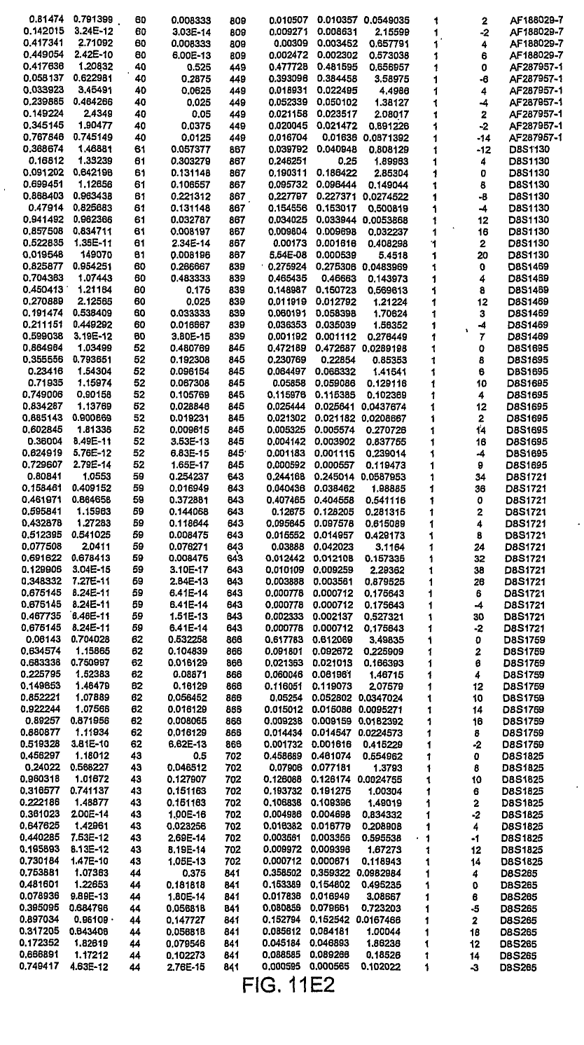

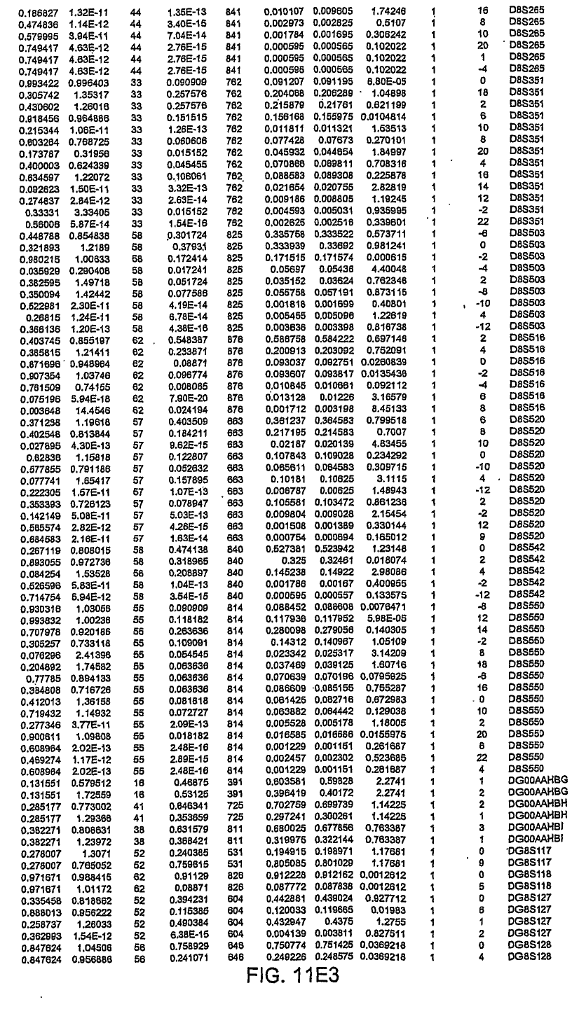

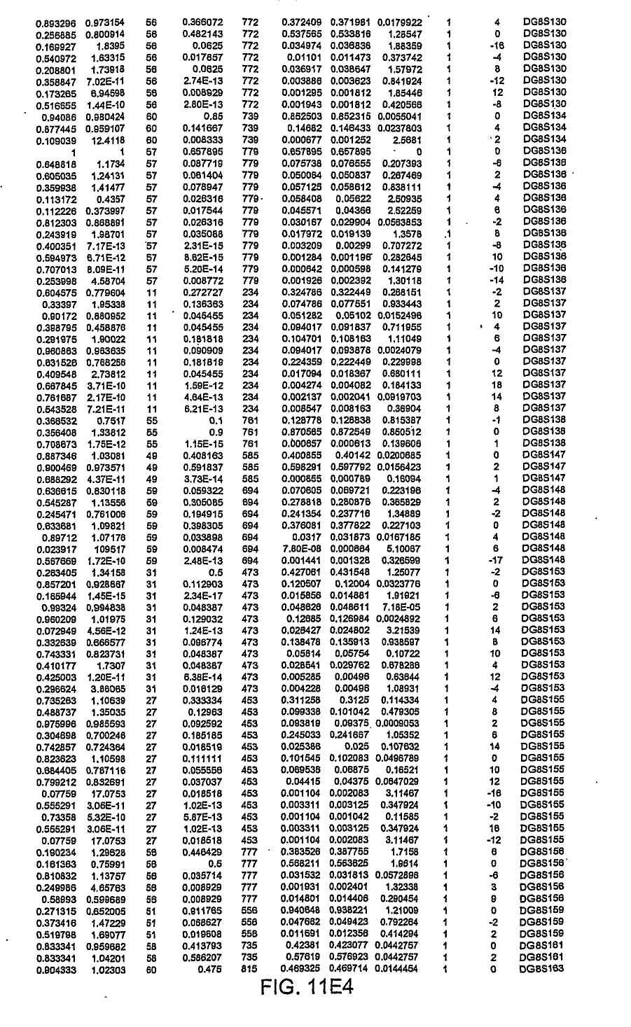

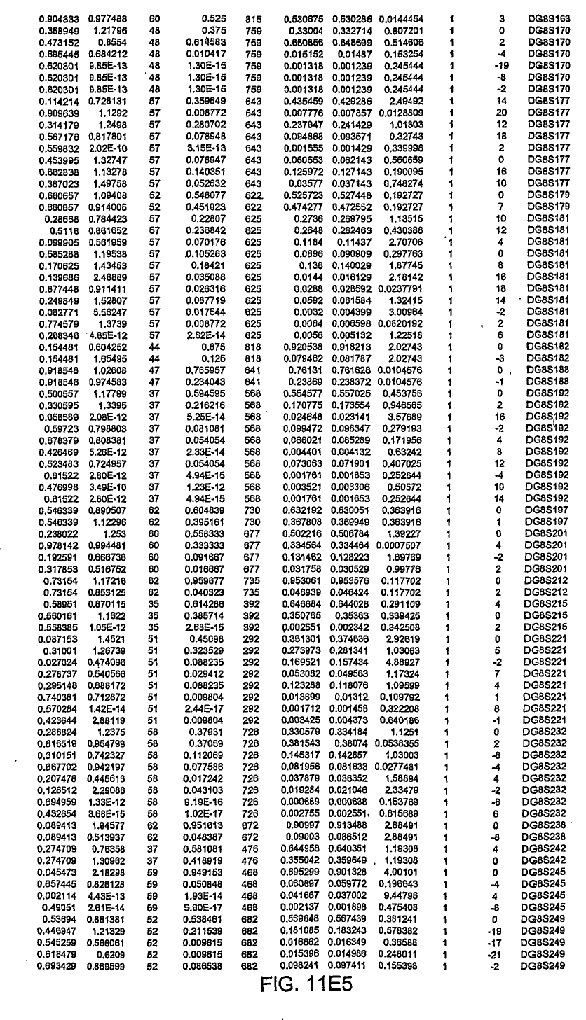

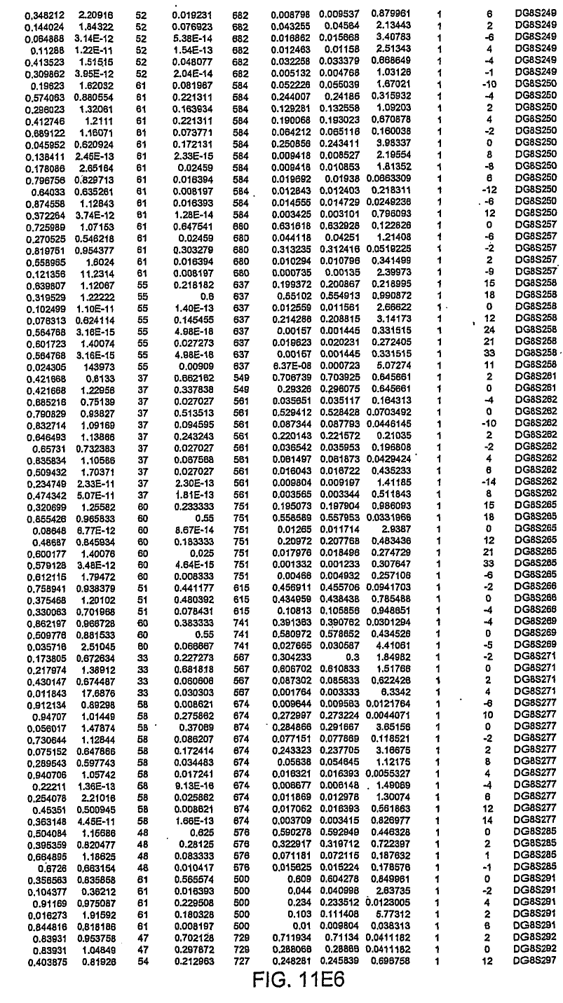

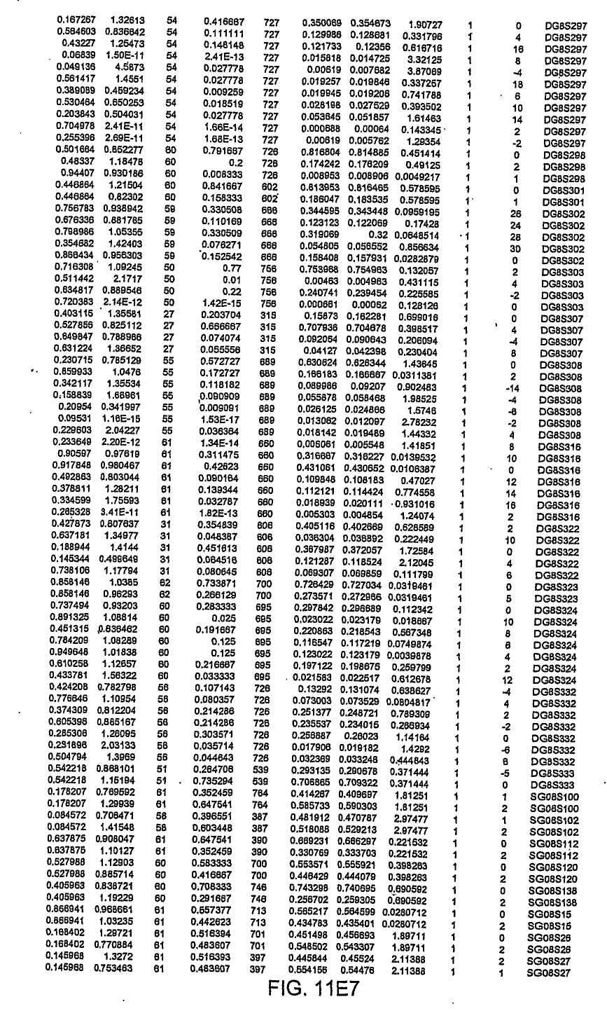

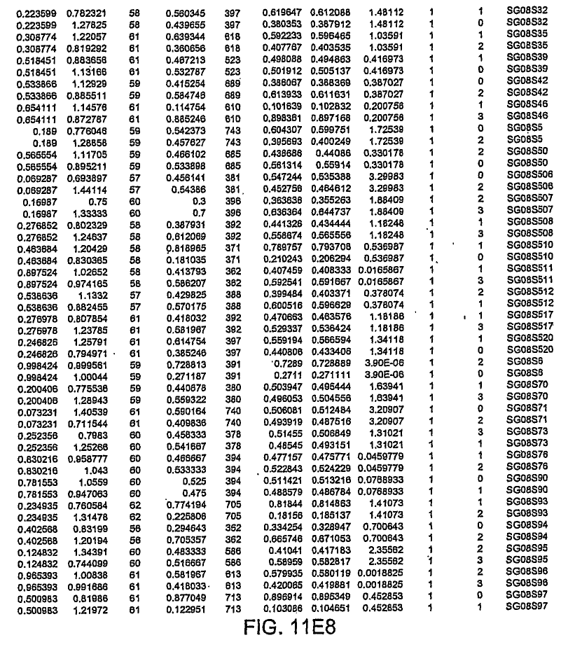

Using the data on the two most strongly correlated markers (SG08S5 and SG08S95), the frequency of the inverted order in 299 panic disorder patients is estimated to be 47% compared 37% in 967 controls (two sided p-value of 0.0002). While the estimates of the frequencies in affected and control individuals are similar to those obtained in the smaller FISH study, the results are statistically more significant due to a large increase in the sample size. This demonstrates that the orientation is a risk factor for panic disorder. Similar results were obtained for bipolar disorder and bipolar disorder without panic disorder (see FIGS. 6A-6K, 7A-7K and 11A1-11A3, 11B1-11B12, 11C1-11C8, 11D1-11D8 and 11E1-11E8).

Allelic Associations to PD and BPD

The allelic association displayed in FIGS. 5A-5D, 6A-6K and 7A-7K is for the association of specific alleles of the markers indicated to panic disorder, bipolar disorder, and bipolar disorder without panic disorder. Each of these markers can be used to diagnose these disorders or to assess risk of developing these disorders. The estimated risks are calculated based on the multiplicative model. For example, a heterozygous carrier of the inversion is estimated to have an estimated 1.52-fold risk compared to that of an individual carrying two copies of the common form, and a homozygous carrier has an estimated 2.31-fold risk (1.52×1.52) compared to an individual homozygous for the common form.

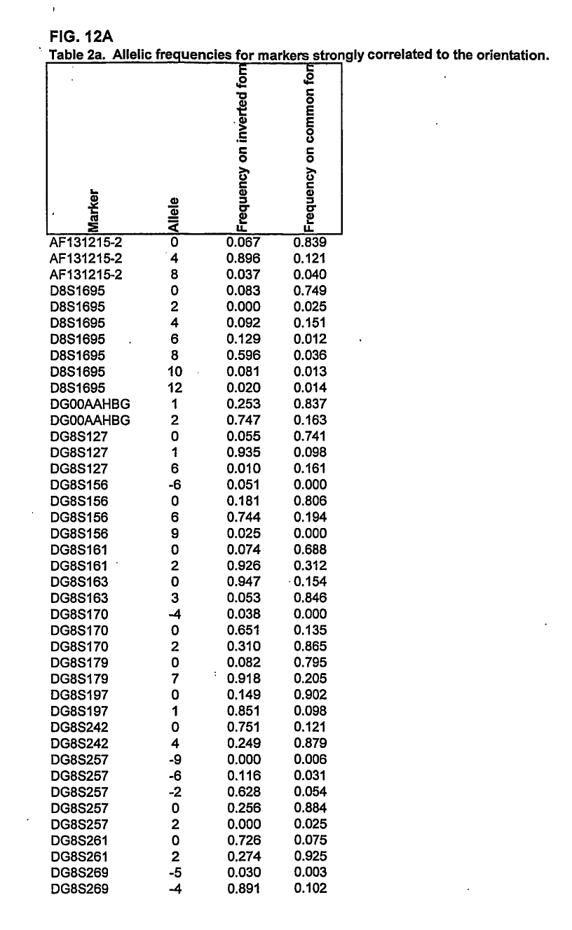

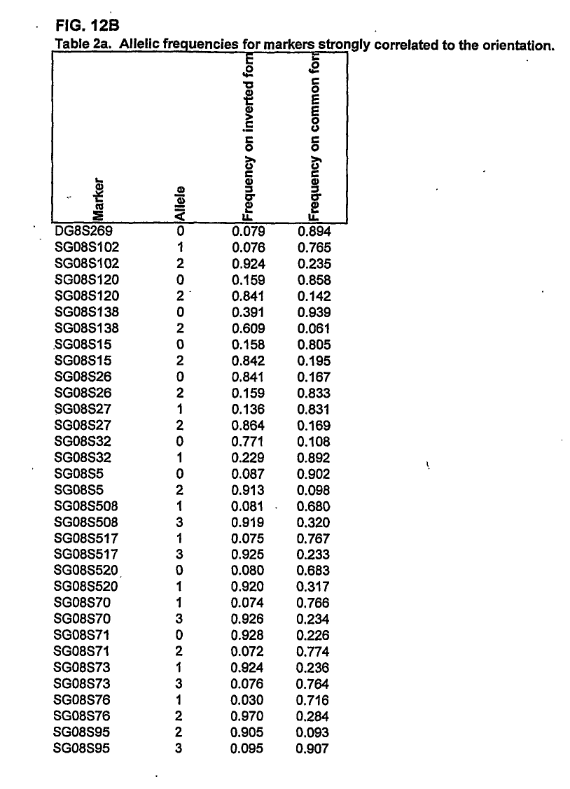

The role of Inv8p23 in individuals diagnosed with psychiatric disorders other than panic disorder was also investigated. Individuals were recruited from the study of the genetics of anxiety disorders. The association with markers within the region show the same general pattern as for panic disorder, but the data is most extensive for panic disorder and bipolar disorder. FIGS. 5A-5D, 6A-6K and 7A-7K list the results of allelic association analysis for panic disorder, bipolar disorder, and bipolar disorder without panic disorder. From the data in FIG. 4, it can be seen that multiple markers in the region show an elevated relative risk. Furthermore, when association is detected, the alleles associated tend to be the same as those associated with the inverted form, but the associations are not as strong as for panic disorder as they are for the inversion itself. Considering all alleles with a relative risk value above 1.0 and prevalence above 5% in the PD cohort (FIGS. 5A-5D; allele frequencies are shown in FIGS. 12A and 12B), it was observed that in nearly all cases the allele associated is either the same allele as is associated with the inverted form of the polymorphism (FIG. 4), or one of multiple alleles associated with the inverted form.

In addition to providing markers useful for detecting susceptibility to anxiety disorders (e.g, PD, OCD, BPD and depression), the markers themselves provide significant insight as to the biological mechanism that causes such disorders. There are several mechanisms that can explain our findings. For example, insights into the biological mechanism can be gleaned from evolutionary history of the inversion allele. It is possible that the inversion occurred in a background containing a mutation that is the true susceptibility variant, or that such a mutation occurred soon after the inversion occurred. In these scenarios the true mutation is enriched on the inverted segment, but the orientation itself is not the actual cause of the effect. A more direct role of the orientation is also possible. Alternatively, the most straightforward explanation is that the inversion polymorphism is associated with the disruption of a gene or genes at the breakpoints. It is also possible that other properties of the genes are affected by the orientation. Thus it is possible that the expression level of a gene or several genes in the region depends on the orientation of the segment. It is also possible that the inversion acts by changing the distance between genes and segments containing regulatory or enhancer elements that are on different sides of the breakpoints, thereby affecting regulation of genes, wherein the misregulation leads to the disorder.

In summary, the association of the rare variant of the inversion polymorphism to several mood disorders with risk ratios of 1.3-1.8 for carriers compared to non-carriers is demonstrated. The 8p23 inversion has strongest association to PD and bipolar disorder.

### Example 2

Other phenotypic effects associated with the inversion allele are also of interest. For example, PD comorbid conditions are of interest. For example, studies have shown that a correlation exists between cholesterol levels and panic disorder (Peter, H. et al., 2002, Can. J. Psychiatry, 47:557-561; Haywood, C. et al., 1989. Am. J. Psychiatry, 149:917-919; Bajwa, W. et al., 1992. Am. J. Psychiatry, 149:376-378; Lacerda, A. et al., 2000. Arq. Neuropsiquiatr., 58(2B):408-411), generally indicating increased cholesterol levels in patients with PD. This is important in light of the fact that mortality due to cardiovascular disease is increased in the group (Fleet, R. and Beitman, B., 1998, J Psychosom Res., 44:71-80). Squalene synthase, the first enzyme dedicated to cholesterol synthesis, is located within the inverted segment. Therefore, a study of the relationship between cholesterol levels and the inversion allele was initiated.

In this context it is interesting, that although panic disorder is classified as a psychiatric condition, many of its symptoms are physical. In particular, 7 of the 13 characteristic symptoms of a panic attack are also symptoms of a cardiovascular disease (Fleet, R. et al., 1998, J Psychosom Res., 44:81-90), and it has been estimated that approximately 25% of patients presenting to the ER for chest pain have PD. Of these patients, 80% are found to have atypical or non-cardiac chest pain (Fleet et al., 1996, Am. J Med., 101:371-380). It is possible that some of the symptoms relating to the function of the heart have to do with the expression levels of the GATA-4 transcription factor, a key element in heart development. In this vein, an altered expression level of GATA-4 might be expected to have widespread effects, since the factor has been shown to regulate the expression of many genes, including genes potentially involved in the etiology of anxiety such as the adenosine Al receptor (Rivkees S. et al., 1999, J. Biol. Chem., 274:14204-14209), and several genes involved in steroidogenesis (Tremblay, J. and Viger R., 2003, J. Steroid Biochem. Mol. Biol., 85:291-298) including one of the key genes, Steroidogenic acute regulatory protein, which is located about 26 Mb centromeric of the inversion on chromosome 8. Several neurosteroids have been shown to be anxiolytic in animal models and potential hypersecretion of neurosteroids in PD patients has been reported (Brambilla, F. et al., 2003, Psychiatry Res., 118:107-116).

There are several other genes located within the inversion that are good candidates for influencing psychiatric conditions within the Inv8p23 genomic region. The idea that the orientation might affect the expression levels of several genes casts PD as a genomic disorder, and suggests that it should perhaps be viewed as a syndrome comprised of signs and symptoms arising from the effects of several genes.

Specifically, the MTMR9 gene is a member of the myotubularin (MTM) family, and forms a complex with MTMR9 and dephosphorylates phosphatidylinositol 3-phosphate and Ins(1,3)P2 in neuronal cells (Mochizuk, Y. and Majerus, P., 2003, Proc. Natl. Acad. Sci. USA, 100:9768-73). MTMR7 is one of the genes flanking the inversion region on the centromeric side. It has been postulated that inositol metabolisim is at the root of bipolar disorder (Atack, J., 1996, Brain Res. Brain Res. Rev., 22:183-90). Cathepsin B and APP secretase have been implicated in brain disorders, for example Alzheimer's disease, and MTSR or methionine peptide sulfoxide reductase is involved in maintaining reduced form of methionine by reducing methionine sulfoxide, and such oxidative processes are important in the central nervous system. In fact, S-adenosyl-L-methionine, has been used as an antidepressant (Mischoulon, D. and Fava, M., 2002, Am. J. Clin. Nutr., 76:1158S-1161S.). Within the duplicated regions at the boundaries the gene for USP17, deubiquinating enzyme is found within a 4.7 kb repeat. These and additional genes in the inverted region, and regions flanking the inversion region are listed in FIG. 9.

### Example 3

The method of high-throughput surrogate FISH genotyping is described. The method first uses FISH to identify the rearrangement status of a small set of individuals used as a training sample. These individuals are then genotyped for genetic variation using standard high-throughput technologies for microsatellite genetic markers, SNPs and INDELs. Markers, either individually or in haplotype combinations, that are highly correlated with the rearrangement are then genotyped on individuals who have no FISH data, and their rearrangement status is predicted. The method described here can be used to determine orientation of genomic rearrangement anywhere in the genome. For rearrangements that are shown to be associated with genetic disorders, this method can be applied as a diagnostic test for the disorder. As described herein, it has been discovered that one form of an inversion polymorphism on chromosome 8p23 is a risk factor for anxiety disorders, depression, and bipolar disorder.

Genetic Study of Anxiety, Depression and Comorbid Conditions

All data, phenotypic information, and DNA samples, have been collected as a part of an extensive study of the genetics of psychiatric disorders. After sending out screening questionnaires to 30,000 Icelanders, over 11,500 responses were received. Analyzing the genealogical relationships among the responders, over 3,600 responders with scores indicative of depression, anxiety or both were identified. During the recruiting of families, additional cases were identified by screening relatives using the same questionnaire. When participants, recruited based on the questionnaire score, donated their blood samples, actual diagnoses were made as participants underwent the Composite International Diagnostic Interview (CIDI) (Wittchen H U, Perkonigg, A (1996) DIA-XSSQ. Swetz und Zeitlinger, Swetz Test Services, Frankfurt; Peters, L. and Andrews, G., 1995, Psychol. Med, 25:1269-1280), which yields diagnoses according to the DSM-IIIR and the ICD-10 systems. Each individual was considered affected by a psychiatric disorder if a diagnosis was made according to one or both systems. The National Bioethics Committee and the Data Protection Commission of Iceland approved the study. All person-identifying data were encrypted by the Data Protection Commission of Iceland using a third-party encryption system developed by deCODE genetics (Gulcher, J. et al., 2000, Eur. J Hum. Genet., 8:739-742).

Fluorescence In-Situ Hybridization Analysis

Metaphase chromosome spreads were prepared after a 24 h harvesting of human Ebstein Barr (EBV) transformed cell lines using standard cytogenetic methods. Cell line cultures were synchronized using bromo-deoxy-Uracil (BrdU, Sigma, St. Louis, Mo.) then the synchronized cultures were treated with a topoisomerase II inhibitor (ICRF154, BIOMOL), as described (Inazawa, J. et al., 1994, Cytogenet. Cell Genet., 65:130-135), in order to get high resolution prometaphase chromosomes. Slides were kept at room temperature (at least for 24 hours) until hybridization.

For hybridization, the slides were pretreated with RNAse A and pepsin, followed by washes in 2×SSC, pH 7.0. Post-fixation of the slides was done with 1% free formaldehyde followed by dehydration in ascending concentrations of ethanol (70%, 90% and 100%) for three minutes each at room temperature. Slides were denatured at 72° C. in 70% formamide/2×SSC pH 7.0 for 3 min, quickly fixed in cold ethanol (−20° C.) in ascending concentrations of ethanol (70%, 90% and 100%).

Probes were generated from BAC clones from the RPCI-11 library. All BAC probes (1 μg of each probe) were labeled by standard nick translation with either biotin 16-dUTP or digoxigenin 11-dUTP (Boehringer Mannheim). 50-60 ng of each probe were dried in a speedvac with 4 μg of cot1-DNA (BiGCO-BRL) and resuspended in a hybridization mix containing 50% deionized formamide, 2×SSC, 10% dextran sulphate pH 7.0. After heat denaturation (75° C. for 5 min), 60 ng of each probe were applied to each slide and sealed with rubber cement. Hybridization was performed overnight in a moist chamber at 37° C. Post hybridization washes were performed in two changes (5 min each) of 0.3×SSC/0.3% Triton X-100 (Merck) (pH 7.0) at 72° C. followed by washes with 4×SSC/0.1% Triton X-100 (for 2 min) and with 4×SSC (for 5 min) at RT. Slides were incubated in blocking solution (Boehringer Mannheim) for 25 min. Detection was performed either with Avidin-FITC (Vector Laboratories), for the probes labeled with biotin, or with anti-digoxigenin-Rhodamine (Roche), for the probes labeled with digoxigenin), for 30-35 min at 37° C. in a humid chamber then washed three times in 4×SSC/1% Tween 20 (Roche). Two subsequent 30-35 min incubation steps were performed with biotinylated anti-Avidin (Vector Laboratories) and avidin-FITC (Vector Laboratories) for biotin detection; and one subsequent 30-35 min incubation with Texas red (Jackson Immuno Laboratories) for the digoxigenin detection. Slides were mounted with an antifade solution with 100 ng/mL of 4′-6 diamino-2-phenylindole (DAPI). Slides were studied under a fluorescent microscope with an automated scanning platform (Axioplan 2-ZEISS) equipped wit the appropriate filter set. Meteafer software from Metasystems was used to search for the metaphases. Images were analyzed using the Isis software from Metasystems. At least 20 metaphases were analyzed for each slide.

Probes Used for Screening

After testing different probes, two BACs from the RPCI-11 collection located inside the inverted fragment were selected to study the inversion status: RP11-10A14 (D) and RP11-177H2 (H).

The two BACs are located 1.7 Mb apart inside the inverted region and do not contain any of the duplicated regions flanking the inversions (REPs containing the Olfactory Receptors). Since there is sequence data, fingerprinting data and FISH data for these two BACs, this BAC combination was selected as the standard combination to search for the inversion.

In some cases where the inversion status is difficult to define using the D and H probe combination, two different BACs were used to confirm the orientation of the fragment: RP11-148021 (1) and RP 1-496N3 (20).

These two BACs are also located inside the inverted fragment but are located ˜3.4 Mb apart (FIG. 2B).

Genotyping Methods

Genotypes were obtained by PCR-based assays, either TAQ-man assay, or FP assay for single-nucleotide polymorphisms, and using fluorescently labeled primers for INDEL polymorphisms and microsatellite markers. Standard techniques for genotyping for the presence of SNPs and/or microsatellite markers can be used, such as fluorescent based techniques (Chen, X. et al., 1999, Genome Res., 9:492), PCR, LCR, Nested PCR and other techniques for nucleic acid amplification.

### Example 4

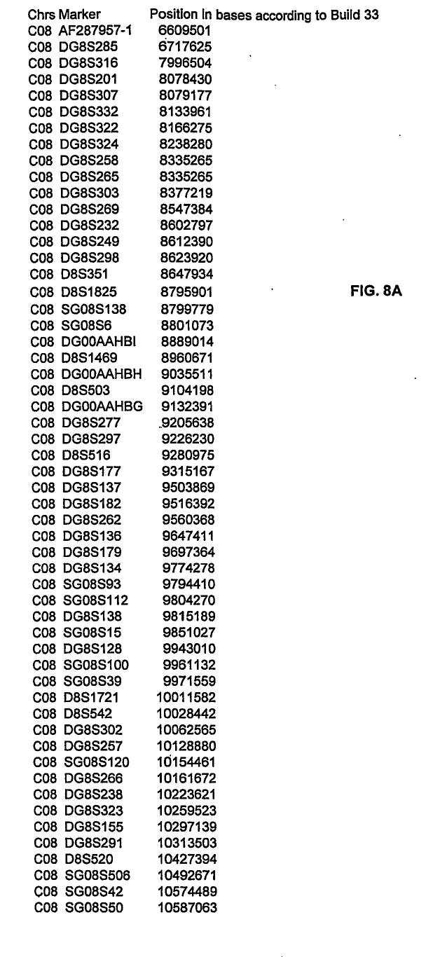

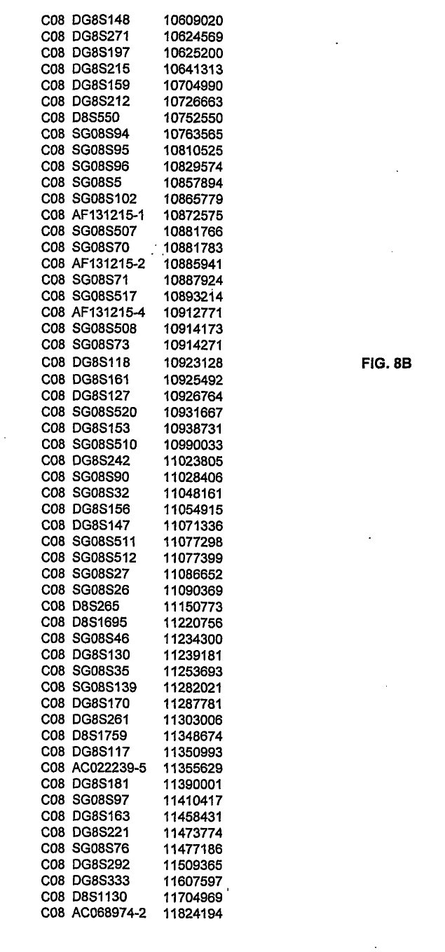

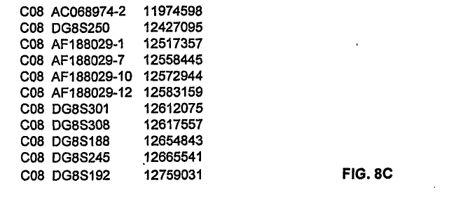

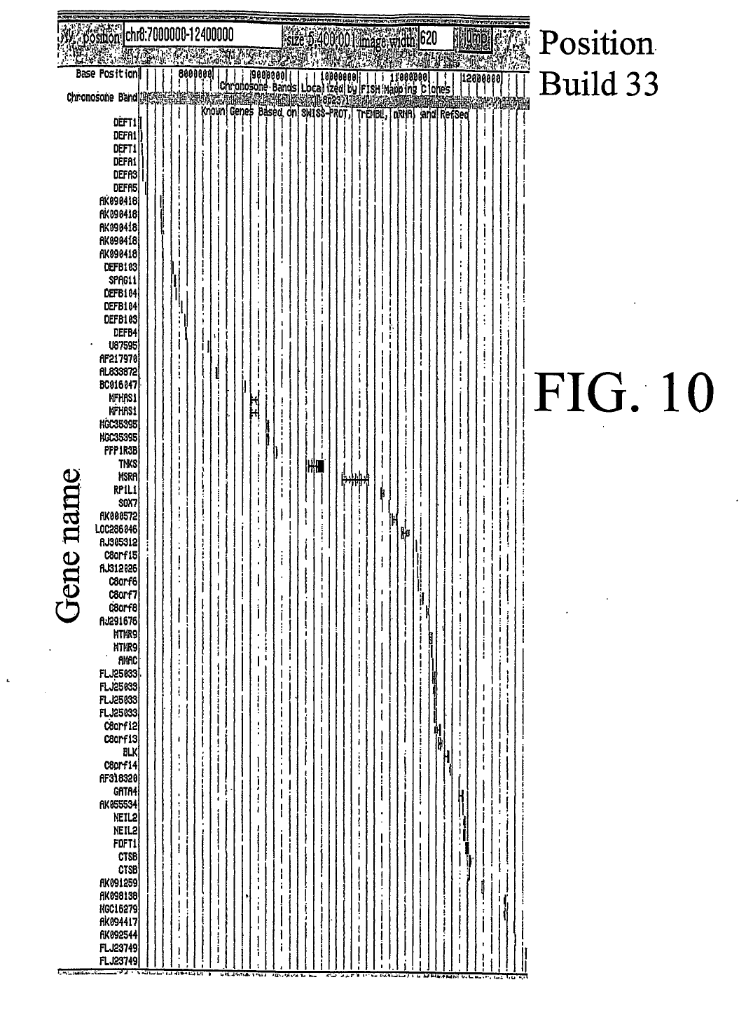

Markers with chromosomal location according to NCBI build 33, their primer sequence and amplimers. The SNPs are with chromosomal location according to NCBI Build 33 and 500 basepair sequence up-and downstream of the IUPAC coded-annotation. Also see FIGS. 8A-C for a list of markers and FIG. 10 for a position map.

For SNPs

IUPAC codes, R=AG, Y=CT, S=GC, K=GT, W=AT, M=AC,

For Microsatellites and INDELs typed by measuring the length of the repeat using capillary electrophoresis, following PCR using labeled primers the allele number is reported as the offset from the smaller of the two alleles of CEPH sample 1347-02 (CEPH genomic repository); thus allele 0 serves as a (CEPH) reference allele.

| | |
|---|---|
| >AF287957-1, chr8, pos 6609501 in NCBI build 33 | |
| Primer pair: | |
| F CTGGCTCTTCCTGCCCTAAT | (SEQ ID NO: 1) |
| R TTTCTGGTGGGCATGTATGT | (SEQ ID NO: 2) |
| length: 197 | |
| Amplimer: | |
| CTGGCTCTTCCTGCCCTAATACCGGCTGCCCGTACGGGACTGCTCACCTCCTGCAG | (SEQ ID NO: 3) |
| GGAGCCGGACGTCTGTGGCGATCTCCCTCCCGCCATGACACCCCCTACCTGTCCTC | |
| CATCATATGGGacacacacacacacacacacacacccctacgcacacccacacCCC | |
| ACATGCACATCATACATGCCCACCAGAAA | |
| >DG8S285 chr8, pos 6717625 in NCBI build 33 | |
| Primer pair: | |
| F: TGGAAGGCCCTCTTTAACAGTA | (SEQ ID NO: 4) |
| R: GCCACCCTAACCCTACCAAG | (SEQ ID NO: 5) |
| length: 159 | |
| Amplimer: | |
| TGGAAGGCCCTCTTTAACAGTAGGTATTTGAAGTGTTATAAAAAAAAAAAAAAGGT | (SEQ ID NO: 6) |
| GAATTTTTCTTTTATTTCTCAGTTTGAAAGAACAGCTTTATTCTTGGTTATTCCTA | |
| ATGTCCACCTAGTCCTCTTTTACTTTTCTTGGTAGGGTTAGGGTGGC | |
| >DG8S316, chr8, pos 7996504 in NCBI build 33 | |
| Primer pair: | |
| F: CACATATTTGTAGGAACTCTCAAAGC | (SEQ ID NO: 7) |
| R: GCATTACACAACCTCTTTACCAG | (SEQ ID NO: 8) |
| length: 189 | |
| Amplimer: | |
| CACATATTTGTAGGAACTCTCAAAGCGTTTTCCAATAAGAATTAAATTGCAAATGA | (SEQ ID NO: 9) |
| CAATTAAGTTTTTAAACCAGTCCCCAAAATCTTAATTTGATTGTAGTTACAAAAGA | |
| ACTAGTTCAAGTTCgtgtgtgtgtgtgtgtgtgtgtgtgtgtgtgtgtgtgtgtCT | |
| GGTAAAGAGGTTGTGTAATGC | |
| >DG8S201, chr8, pos 8078430 in NCBI build 33 | |
| Primer pair: | |
| F: AAACCATTTAACACAGGATAAACTCA | (SEQ ID NO: 10) |
| R: GGGTACACTTCCATCTGACCA | (SEQ ID NO: 11) |
| length: 185 | |
| Amplimer: | |
| AAACCATTTAACACAGGATAAACTCATAGTTACATTAAAAGATAGGAAAATacaca | (SEQ ID NO: 12) |
| cacacacacacacacacacacacacataccacacaaacacacatacatgcacacac | |
| acacacaTTTCGGTTACTAGTTGGTTTCAGTCAAGGATAAAAATTCTTAAATTGGT | |
| CAGATGGAAGTGTACCC | |
| >DG8S307, chr8, pos 8079177 in NCBI build 33 | |
| Primer pair: | |
| F: GACGGATTTCAGAGTCACCAA | (SEQ ID NO: 13) |
| R: TGCAGAAGTCCTCTGTTTGC | (SEQ ID NO: 14) |
| length: 381 | |
| Amplimer: | |
| GACGGATTTCAGAGTCACCAAGGATGGCCAATGATGtggtggttaagagcatgaac | (SEQ ID NO: 15) |
| actggtgcttcacggcctgggttcgggtcctgactcaatgcttactggctgtgtgt | |
| tttggaaaaggcccttaatctctctctgtttcagcttcccatctataaaatgtgga | |
| taatgacaatacatacctcatgcagttattagaaagattcaatgagttattattta | |
| taaactgctcaaaacagcaccatgtacatagaaagtgctcgttaaatggatggatg | |
| gatggatggatggatggatggatggatggatggatgggtgcatggatggatggatg | |
| aatagatcaatggatggatAAACAGGCAAACAGAGGACTTCTGCA | |
| >DG8S332, chr8, pos 8133961 in NCBI build 33 | |
| Primer pair: | |
| F: CCGATGGGTATTTGTTCCAC | (SEQ ID NO: 16) |
| R: GAGGAAAGGACACAGGGACA | (SEQ ID NO: 17) |
| length: 170 | |
| Amplimer: | |
| CCGATGGGTATTTGTTCCACGTTTTCTATTTTAGTCAGTTCTACCTTTAGAGTTCT | (SEQ ID NO: 18) |
| TTacacacacacacacacacacacacacacacaGCATCTCACTTAATTTTATTCAT | |
| CCTTCAAAGTTCATCTTAGGTCATTTCTTCCCCTCCTTTGTCCCTGTGTCCTTTCC | |
| TC | |
| >DG8S322, chr8, pos 8166275 in NCBI build 33 | |
| Primer pair: | |
| F: TTTCTGAAACTCCATAAACTCATCA | (SEQ ID NO: 19) |
| R: GAACTCTACCAAGTTTGTCTTCTGG | (SEQ ID NO: 20) |
| length: 178 | |
| Amplimer: | |
| TTTCTGAAACTCCATAAACTCATCAGATTATTTTTACTTTAAATGCTATAAACCTG | (SEQ ID NO: 21) |
| AAGTATTTCTTTACTTGacacacacacacacacacacacacacacacactcataca | |
| caTTTCATACTTTTGCATCAAAGCTGGTCATAAAATTGGTACCAGAAGACAAACTT | |
| GGTAGAGTTC | |
| >DG8S324, chr8, pos 8238280 in NCBI build 33 | |
| Primer pair: | |
| F: ACATCCTCTTCCAGCAGACA | (SEQ ID NO: 22) |
| R: TGGAAGCTGCTAAGGAGAACA | (SEQ ID NO: 23) |
| length: 373 | |
| Amplimer: | |
| ACATCCTCTTCCAGCAGACACCCACAAAGTACTATTCAGTTTGCACTGTAACAAAT | (SEQ ID NO: 24) |
| GTTATTTCTGGGCCTCAGTGAGATAATGGTAAGTGAATGTAATTCACTCTCATTAA | |
| TATATTAAAATGAGTATGAATTTTAAATTAGAAGGAACAAGTCCATGGTCGAAGAA | |
| TTGAAATTGGATTTATGTGATTTGACTTCGTAGTCATTTATCTACAATACTCATTG | |
| ATACTAATTGCACAGTTTCCTCTTCACATTCCCACTGGGCAGCACgtgtgtgtgtg | |
| tgtgtgtgtgtgtgtgtgtgtgcatgtgtTATGTATTTGAATTAAAAGACACTGAG | |
| AAGTAGCGCCTAAAAATGTTCTCCTTAGCAGCTTCCA | |
| >DG8S258, chr8, pos 8335265 in NCBI build 33, alias name | |
| DG8S265 | |
| Primer pair: | |
| F: TCTTCCGCCCTGTGTCTATC | (SEQ ID NO: 25) |
| R: TCAAGCGGAAGATTTGTCCT | (SEQ ID NO: 26) |
| length: 257 | |
| Amplimer: | |
| TCTTCCGCCCTGTGTCTATCTAGGTCAGgcttctcaaacctcaccatggcagatgc | (SEQ ID NO: 27) |
| atcatttggagaccttgtgaaaatgtagactctgattccctaggtcaagggctgag | |
| attctgcatttctttcaaaatcccaggtgatgctgctgctgctgctgctgctgctg | |
| ctgctgctgctgctgGTCTAGACCACATTTTCAGAAGTAAGGATTTAAACAATCAG | |
| CACCCAGGGAGCTAGGACAAATCTTCCGCTTGA | |
| >DG8S265, chr8, pos 8335265 in NCBI build 33, alias name | |
| DG8S258 | |
| Primer pair: | |
| F: TCTTCCGCCCTGTGTCTATC | (SEQ ID NO: 28) |
| R: TCAAGCGGAAGATTTGTCCT | (SEQ ID NO: 29) |
| length: 257 | |
| Amplimer: | |
| TCTTCCGCCCTGTGTCTATCTAGGTCAGgcttctcaaacctcaccatggcagatgc | (SEQ ID NO: 30) |
| atcatttggagaccttgtgaaaatgtagactctgattccctaggtcaagggctgag | |
| attctgcatttctttcaaaatcccaggtgatgctgctgctgctgctgctgctgctg | |
| ctgctgctgctgctgGTCTAGACCACATTTTCAGAAGTAAGGATTTAAACAATCAG | |
| CACCCAGGGAGCTAGGACAAATCTTCCGCTTGA | |
| >DG8S303, chr8, pos 8377219 in NCBI build 33 | |
| Primer pair: | |
| F: GAAAGAAGCTGCAAACAGCA | (SEQ ID NO: 31) |
| R: GTTGATCCAGAGGTCGGTGT | (SEQ ID NO: 32) |
| length: 366 | |
| Amplimer: | |
| GAAAGAAGCTGCAAACAGCAACCTGGTCTTTGACTGCACAATAATCCTCTAAGGTT | (SEQ ID NO: 33) |
| CAGATCGTCTCAACCAGAGTTAAATTCTAACagagagagagagagagagagagaac | |
| gagagagagagagagagaTTGATCTGGATTCAGGCTTCCTAGATGCAGTCTATCCA | |
| ACTCAGGCAGCAGTGAACGAGGAATACAGGCTCTTTCCCACATGTTTGGAATCCTG | |
| GCCCTGAGCCCTGAGCTGTGCATTCCATTTATCCTCTTTGTGGGCTGAACAGATGA | |
| AATTGCTTTAGCTAAAGGAAGTGGCACGAATTTACTTATTTATTAGATGTGCAGGA | |
| TACATCCATCACACCGACCTCTGGATCAAC | |
| >DG8S269, chr8, pos 8547384 in NCBI build 33 | |
| Primer pair: | |
| F: CCACTTCCAATGCAGACCTT | (SEQ ID NO: 34) |
| R: TGCATGTATATAATGAGTAGGGAGAGA | (SEQ ID NO: 35) |
| length: 412 | |
| Amplimer: | |
| CCACTTCCAATGCAGACCTTGTTCTATAAAGAATATCTAGCACTTTCACATGTTTC | (SEQ ID NO: 36) |
| TGAAGGAAGTGTATTATTTGTAGCCCCTTTTTGGAGAAAAATTattctgcttcaag | |
| gtatttattctacggatatactaacaTgtgtcaaagaatacaatctcgagtcttta | |
| gtgttgtttctggagtaaaatattgaaaataatcaaaatgctcatcaatagaaggc | |
| tggctaaataaagtcggcTtatataatggaatatcacgtggccagtaaaaaagaat | |
| caaacagctctctatatatcaatattttgcagtgtatatattaaacttttaaaaag | |
| catacaaaacactgtttctattctactaccattttgGGGTGGGAGACTTTCTCTCC | |
| CTACTCATTATATACATGCA | |
| >DG8S232, chr8, pos 8602797 in NCBI build 33 | |
| Primer pair: | |
| F: TGCCGGTATAGGTGTGACTG | (SEQ ID NO: 37) |
| R: TGTTTCTTGCTGATTTCTTCCA | (SEQ ID NO: 38) |
| length: 293 | |
| Amplimer: | |
| TGCCGGTATAGGTGTGACTGAACAATACATCCATTGGTAGACTACTATGCTATATT | (SEQ ID NO: 39) |
| TGTAGGATATACTATAACATTCTacacacacacacacacacacacacacacacaca | |
| cacacacacaTAATAATCTTCTATAACAGGGTTCTAACTGTTCatatggaggcatc | |
| tcaaaaatatattttgaagtgatcaaatgcgaggtgcagaacaaggagtacagcat | |
| gATCTCATTCCTGTTAAAATATATGCAAATACATGCTTTATTTTCCCTGGAAGAAA | |
| TCAGCAAGAAACA | |
| >DG8S249, chr8, pos 8612390 in NCBI build 33 | |
| Primer pair: | |
| F: TCACCTCTTCACGGACAAAG | (SEQ ID NO: 40) |
| R: TCTTAAGTCCATCTCTGCACAAG | (SEQ ID NO: 41) |
| length: 309 | |
| Amplimer: | |
| TCACCTCTTCACGGACAAAGGGGAATAACCTCAGAGTATGACATAAAATATCCACT | (SEQ ID NO: 42) |
| AAATAAAAAATACTggttgggtatggtggctcacgcctctaatcccaacattttgg | |
| gaggctgagtggggaggaccatttgaggccaggagatcaagaccagcttgggcaac | |
| ataaaaaggccctatctctatttcacaaacacacacacacacacacacacacacac | |
| acacacacacacaaaaagaaaaaaaaaaTTAAAGAAAAAATACTTTAGGAAATTCT | |
| AAACTACTTGTGCAGAGATGGACTTAAGA | |
| >DG8S298, chr8, pos 8623920 in NCBI build 33 | |
| Primer pair: | |
| F: TTCAGATGGCTCAGGGTAGC | (SEQ ID NO: 43) |
| R: AGAAGCTGCAGGATGGAGAA | (SEQ ID NO: 44) |
| length: 265 | |
| Amplimer: | |
| TTCAGATGGCTCAGGGTAGCCCCACCCACACTCCCTCCCAGAGACAGTCAATTTTA | (SEQ ID NO: 45) |
| CAACAAATATTCTGAGttatctaggctgaccctttttttcccccacagaggaggaa | |
| atgggctcaaagtaagtgacttctcaatcagccatcaaagtagagtagaggcagga | |
| ctGCTAACTCCCCGTGTGGAATGTATTCCCCTGTGATCATCACCTGTACTCACACT | |
| GTTCTTGAGCCAGACCCCAAATTCTCCATCCTGCAGCTTCT | |
| >D8S351, chr8, pos 8647934 in NCBI build 33 | |
| Primer pair: | |
| F: AGCCAGAAATTGAGGAAGTG | (SEQ ID NO: 46) |
| R: CTGCAAGCTCTTTCAGTTGA | (SEQ ID NO: 47) |
| length: 109 | |
| Amplimer: | |
| AGCCAGAAATTGAGGAAGTGCTCAAACACACACACACACACACACACACACACACA | (SEQ ID NO: 48) |
| CACACAAAGGAGTATGTCATAGGTACAGAGAAGTCAACTGAAAGAGCTTGCAG | |
| >D8S1825, chr8, pos 8795901 in NCBI build 33 | |
| Primer pair: | |
| F: GACGGATTTCAGAGTCACCAA | (SEQ ID NO: 49) |
| R: TGCAGAAGTCCTCTGTTTGC | (SEQ ID NO: 50) |
| length: 381 | |
| Amplimer: | |
| GACGGATTTCAGAGTCACCAAGGATGGCCAATGATGtggtggttaagagcatgaac | (SEQ ID NO: 51) |
| actggtgcttcacggcctgggttcgggtcctgactcaatgcttactggctgtgtgt | |
| tttggaaaaggcccttaatctctctctgtttcagcttcccatctataaaatgtgga | |
| taatgacaatacatacctcatgcagttattagaaagattcaatgagttattattta | |
| taaactgctcaaaacagcaccatgtacatagaaagtgctcgttaaatggatggatg | |
| gatggatggatggatggatggatggatggatggatgggtgcatggatggatggatg | |
| aatagatcaatggatggatAAACAGGCAAACAGAGGACTTCTGCA | |
| >SG08S138, chr8, pos 8799779 in NCBI build 33, alias | |
| name, rs920974 | |
| CTCAAAAACCAAAGGTGTGATGAAGGTGCTACAGTTTGAACTCTTTAAAGGAAGGC | (SEQ ID NO: 52) |
| ATCGGCCATATAGAGTGAGCCACAGGGGAGGACTTCTCCCGTTTCCCTGTAGAATG | |
| GGTTACCAAGTTAAAGGAGTCAATTATCCCGTCCTATCTGGAGAAAGCATTCCTCA | |
| GATGAATAAACTGGAAACGGAAAACTGGAGAAGGTGTTTTTATTTCTTTTCGTAAT | |
| TAGGACATCATTTACAAGACTTATATTTCTTGGATGTTCCCCAAATTTTTCACATA | |
| GAGCTGGCATTACTAGAAACTTAAATACTTGTTGCTTTTAATTATATTGAATTCCA | |
| CCGTGGGAGCTTAAAGGCTAGGCATTTTGTGATGGGTGTGCATTCTACTCCCAAAT | |
| GTAATAACTAGAATAGAAATTCCAGAAAAGGAAAAGTATTTATCAAACACTGAAGC | |
| TGCTTTGAGAAATGGCTTTGTCAAGTTAACTGGTTATCATTAGATTTATTAC | |
| [R] | |
| GTGGTTAGGAAAAACTGACCTCGTAGATGTCTGTCTATAACAATGCAATCATCTGC | |
| TTAGAATAATGCCCCGCGTTAGACAGCTGTAAACACAAGAACTTTCCCTTGCGAGT | |
| TCAATAATCTTAGCAACAGTTCTCTTTCCAAACAGGCCAAGAAAGATATGTTGCTT | |
| TGGGAAACTGGAAATCAACAGACCAAAACAGCCAGAAGAAATGGGTGGAGAGAAGA | |
| TAGAGCCCGTTCACTCTGCAGTCTCCGCAGGGGTACAGAGTGATGGCAGCCATGGG | |
| TGCCCTTGTAAGTCTCTGTCCCAGCTCCCAACCCTGCCACCTGGGGCCACCACCAT | |
| GATTCCCTGCCCGGCCCTGCACACATGGGCTGCAAAAATGCTGAGGAAAAAGGAGA | |
| TTTCAAACTAATTCATCCCCAAGTTACAAACGTGGTTCATGGAGCTTTAGtaaaaa | |
| ttatttttaaatttttaCTTTGATCCACAGACATGCGACTTGAACCAGATTC | |
| >SG08S6, chr8, pos 8801073 in NCBI build 33, alias | |
| name, rs2028806 | |
| tgcattccagcctgggtgacagagcaagaccctgtcACACACGTACACACACGCAA | (SEQ ID NO: 53) |
| AAATGACAGAGAGGCAGAATTCTCCTAAGTGGAAATGAAATACAGAATACCATGAT | |
| TTAGTTTTCCTGTAGTTCTTTCCCTAACGTTTGACAATAGCTTTCCTTTTGGGTGA | |
| TCAGTGTCCTTTGGTTTTACCTCATAGCCCTGTGAGGTTGCCGTGTTGAGTCTTGT | |
| TTTCATACCACATTGACGGTCCTTTCTAGTGGCCTGAAGGTTTTTGTTATTAtttt | |
| gaaaagctttattgatatataattcacataccatacagttcactcatttgaagtgg | |
| acatttcaatatttggaagcctattcacagcatatgcgcaaccattaccacagcca | |
| attttaggataatttTTTCTTTCTGTTTTTTACTGTggggttttgcagtgaaaacc | |
| agaaaacctgctagacaaattccaaaagagctgtaacacGCGatttcagaac | |
| [R] | |
| tttaatcacctcaagaagaaacctgaaggatccttccgtcgccgcctctatctctg | |
| tcccctccagccctcagaaacaactaatctatgttctttctttaAAAAAAAAAAAT | |
| CTTTGAAGCCTTCATAAATCAGCCCTTTGATTTAAATCTCCATCTCACTCCGCCAC | |
| TATTTTTGATCAATTCTTCACCAGAGCTTCATCTTGACATGTGCTCTGCCACAGTG | |
| CTAAGGAACAGAGTGACCCCCCACCCCACTCCCGACAGAAGCAGCCCCAGAGAGAG | |
| AAGCAGAGGGTCAGGGTCAGGGTCAGCACCGAGTGTGCTCGGGTGAACTGCAAGTC | |
| TTGACTTAGTCTTGAGGACCTCCTCAGTCTTGCACCCCTTCCTTCAGCAACACCTG | |
| CCGGGATGCGTCTTTCGGCCTCCTCTGAAATACAAAAACATTTTGTGGTCTAGCTG | |
| CTCACTGTATTTTCACTCTGTGGTTTTCTTTAATTTACACCCCTCTTCTACT | |
| >DG00AAHBI, chr8, pos 8889014 in NCBI build 33, alias | |
| name, rs330938 | |
| TACACATGAAAGTTGACTTGGCTGAATATAAAATGCTTTTAGATGCTTCTCCATTG | (SEQ ID NO: 54) |
| TTTTCTGACTGTAGTAGTACAAAGAGGTCAGAAGTCAGTCTGGTATTTGTTCTTCC | |
| ATCAACAACTTGTTTGGGATTGGGGGTGGTATTTCCTGTGTGGATAACTTGCAGca | |
| cttcctcttcttcttttttttttttggtctttgtaactaaaaaatgtggtcaatat | |
| gtgtctaggtgtgggtgttttaaaattgattttacctggaatttgtgagcccagtc | |
| aatctatatactccagtctttttccagcctgaaaatgttttcttcaataaagtcat | |
| tatcacttAtttctgttgttctggtttcttgattagtaatactgttaagtcttaaa | |
| ctgaattcccattgtttatatttatcagaatctatcacttttcttagttaactatt | |
| tattttcacttatcatgtctaactctatgctcttttcctgtaaaagacctct | |
| [Y] | |
| aaggttcacctccaaatcaacgtttccattttctacactgtcaattttgcttcttt | |
| ccacctccatgagggattttaattcttggattgcatttttttttgacatccattct | |
| tatcgcatctctctttgtatcttgtcttcctaacttttcatcttatctctgtgtgt | |
| ggttttctgtaattcatagaccatgtcttcctgcaatccaagatgtttttaaaatt | |
| ttcttttgtttcctgTAGTAAAACTATTTCACGGGGAAATTTGGCAAACTGGTGAT | |
| GCCCTTGGAATAGTCACCATACACTTGATAGTTTACAAATGTGTCAGCATGTAAAT | |
| TTGTGTTTCATTTTCATATACCCCAACATCTTATAATGGAGGGAAAGGCAAGTCTT | |
| TGTTTTCCAAGGTCTTGGCTCTTTTAGCCGCAAAGTGGTGCTAACAGCTCCTTCAT | |
| GTTCCAGGAGCCTCTGGAGAAACTGCTTCCATAAAGTGTTTGGGAATTCTGG | |
| >D8S1469, chr8, pos 8960671 in NCBI build 33 | |
| Primer pair: | |
| F: GCTTTAGAAGGCGGAGGTAG | (SEQ ID NO: 55) |
| R: GAGGGGGTTAAAGGTGTCAT | (SEQ ID NO: 56) |
| length: 221 | |
| Amplimer: | |
| GCTTTAGAAGGCGGAGGTAGTAGGTAGGTAGATAGGTAGATGATAGATAGATAGAT | (SEQ ID NO: 57) |
| AGATAGATAGATAGATAGATAGATAGATACAGATATACAGATAGAGTTGTATACAT | |
| NAAATATATATTATGNAAATATATACATAAGAAGGATGACATTAACAGGCATTTTC | |
| TAGTAAATTAAGAGTTAGCCAGGAAATGTAACCATGACACCTTTAACCCCCTC | |
| >DG00AAHBH, chr8, pos 9035511 in NCBI build 33, alias | |
| name, rs330062 | |
| GAAGAACAGAGGCGACTCACAGTTTCCGTGATAATGATAAGCTGCAGACGACTATT | (SEQ ID NO: 58) |
| TAGAGCATCCCAACATTTATTTCAAAGTAAAGACAGTAGAAAACAACTGGACTGCA | |
| AGATGGGAGTCTTGGTCactcactgtgtgatattaacagagtcactcgacctcctt | |
| ggactcagtttcttcttgtctaaaatggggctgttgtcctcactcagctctaaagg | |
| ctcctcTTAAAGCAAAAGTGATGGTTCTTGGAATTTCTTTTATTTCTCCAGTGAGA | |
| ATCACTTCAATCTTCAGGCAAGATACCTGCCTGTCTCCTGCCCCTCTCTCCCATTC | |
| TGTCCCGGATATTGTGAAGCTACTTCTTCAGTTTCATGAACCTGGATTTTGGCCAA | |
| ACCCTTGATCATTCATCTTAGAAGCTAGATTTCCTTTTCGAAGCCACAACTCTGGG | |
| AAAGGTCTTCACAGCCAGTTCCTGATGTTGCTGAGCTGATCTTGTCCATTCT | |
| [S] | |
| AGTCAAGGTAGGATGACAGCTCCCCGTGAGAAAAAAAAATAGGTGTTGCATAAGAG | |
| AACATCTTGGCTATTTATGAAAGATTTTCTATGCTTCTGTTTTAAGTTTGTTTTTC | |
| AATTACAAAAGGGACTCATTCTTTTGTATAAAATTTGGAAAGCTAAGTTAAGTTTA | |
| GAGAAGAGGGTAAAATCATTCTTAATCCCATAATTCTACCATGGAGAAATTTTGTT | |
| AGTATTTTGGTGTATTCTCAATTTCCTCtgcagttttttacattgttgaaatcatg | |
| ctatttatactatttcatcctttcttcccactgaaaattgtatgataagcatttcc | |
| tcatgtcactgaagtcactgataagtaatattttaatagcaccataatattttatt | |
| ttgtgggttttgtcctaaggttgaacagataggttgtttctagttttattttttta | |
| aaaatattattagcaatgctgagatgaacatttgtgtgtatatatctctgga | |
| >D8S503, chr8, pos 9104198 in NCBI build 33 | |
| Primer pair: | |
| F: GACCATGATTAAGCAAAACAAA | (SEQ ID NO: 59) |
| R: TCGCTCAGAAACAAACCAA | (SEQ ID NO: 60) |
| length: 222 | |
| Amplimer: | |
| GACCATGATTAAGCAAAACAAATAACACAAANCAAAAATCTTCCTATTTCCCAGAG | (SEQ ID NO: 61) |
| TCCTGGGTTTATCACAAATGCTATTAAGGTTACGAGTTTTGTCCTTTGATAAAAGA | |
| NGANCCACGTTTGGAAATTGTCATTACCCTTTATTTTTCAACACACACACACACAC | |
| ACACACACACACACACACACACACACACACACTCCTACATTGGTTTGTTTCTGA | |
| >DG00AAHBG, chr8, pos 9132391 in NCBI build 33, alias | |
| name, rs898137 | |
| CAAGGAATTGCTACAGCACATGCTGTTGGGGTGCCTGGTGTGGGGCTCCTAGAGGG | (SEQ ID NO: 62) |
| CTCCTTTAAGCCTGCCTCTCCCTCTCTGGTAGTTGTAACTAGAAAGGGTATTCAGG | |
| AAAAAACACAAATTTCTCTCTAGGTCTTCTCAGCCTCCTTACCAGGCAGCAAGAGC | |
| TGAGAGAACTTGGAGTAGAATATTCTAAACCTTGCTCCTGTATCTGCTTTCTTGCC | |
| TTAAGAGAAAAATCTTTTCCCCCAGATTCTGCTGTCTTTACACTCATTCTCATCTT | |
| ACCGATCTCTTTAAAATTTCAGTCATTCTCGGAGACcatagggcagaacgcaaaga | |
| acataacataggagtcaaatggagccgaacacttcagtcactcacgtgatggctgt | |
| gtgtccttgggtaagttctgtagcttctctgagccccaacttccttatAACATCAT | |
| TGAAGTCCTAACAGCTGTGAGAATGACACATGATGCCTGCAAATTTCATAAA | |
| [W] | |
| CAGTGCTTGGTGGTTAGTAGTTGGTTTTGAAAAGGTTATGCTAAAATTCCAGGGTG | |
| ATACTTTTCTAGGTAGTCCCTTTTTGCAGGTAGCTTTCAGAGGTAAAACCTCAGAC | |
| CCCAACACGGTCCACCTCTGCAtttttttttttttttttttgacatggagtctcgc | |
| tctgtgcccaggctggagtgcagtggcgtgatgtcggctcactgcaagctccgcat | |
| cccgggttcacgccattctcctgcctcagcctcccgagtagctgggactagaggct | |
| caggacaccacgctcggctaattttttgtattttttagtagagaccgggtttcacc | |
| gtgttagccaggatggtctcgatcttctaacctcgtgatccgtccgcctcggcctc | |
| cctaagtgctgggattacaggcgtgagccaccgcgcccggcctttttgtttgcttg | |
| ttttttgagatggtttcttggtctgttgcccagactctagtgcagtggcacg | |
| >DG8S277, chr8, pos 9205638 in NCBI build 33 | |
| Primer pair: | |
| F: GTCCTCTGGGTGTTTGCAGT | (SEQ ID NO: 63) |
| R: CAGGCTCTGCTCTCCTTAGC | (SEQ ID NO: 64) |
| length: 259 | |
| Amplimer: | |
| GTCCTCTGGGTGTTTGCAGTGCTGAGTGCATTGGGGTTtgtgtgtgtgtgtgtgtg | (SEQ ID NO: 65) |
| tgtgtgtgtgtgtgtgagagagagagagacagagagagggagagagGAGCACAGTA | |
| GCTTGTGCAAAGACCTCCTTTGCTATAGAAGCCTGATTCCAAACCTGTCTTCTTTC | |
| CCAGAAGTAATTACAATACACATTGCTGCTTCTCTTCAATGTGCCTGTGTTCTGGA | |
| AGCTGTGTGTCTCCAGCTAAGGAGAGCAGAGCCTG | |
| >DG8S297, chr8, pos 9226230 in NCBI build 33 | |
| Primer pair: | |
| F: CAAATCAATATACCACTTCAGGACT | (SEQ ID NO: 66) |
| R: GCAGTAGGCACATGGCAAAT | (SEQ ID NO: 67) |
| length: 168 | |
| Amplimer: | |
| CAAATCAATATACCACTTCAGGACTGGgtgtgtgtgtgtgtgtgtgtgtgtgtgtg | (SEQ ID NO: 68) |
| tgtTTcttctcttccctcccctcctccccttcctcctcctccttctTTAGACAAGT | |
| ACTATGTTTTAAGATTTAGGTATATAATTCTACTTAATTTGCCATGTGCCTACTGC | |
| >D8S516, chr8, pos 9280975 in NCBI build 33 | |
| Primer pair: | |
| F: GAGAATGCTTGACCCCAAAAAATC | (SEQ ID NO: 69) |
| R: CCTAAGAGAGTGCTATGTGCTCCC | (SEQ ID NO: 70) |
| length: 162 | |
| Amplimer: | |
| GAGAATGCTTGACCCCAAAAAATCAAGATCAAAGATCAGCCTGGGCAACAAAGTGA | (SEQ ID NO: 71) |
| GACCCTGTCTACACACACACACACACACACACACACACACACACACAGACACACAC | |
| AAAGTATACCCAAGTACTACAAAAATGGGAGCACATAGCACTCTCTTAGG | |
| >DG8S177, chr8, pos 9315167 in NCBI build 33 | |
| Primer pair: | |
| F: CCCAGATAAGATCTTGGTTCAG | (SEQ ID NO: 72) |
| R: ACCACGGTGACCCTCAATTA | (SEQ ID NO: 73) |
| length: 253 | |
| Amplimer: | |
| CCCAGATAAGATCTTGGTTCAGAAAAAAATGTTAAAACAGCCAGtattatagaatt | (SEQ ID NO: 74) |
| tatatttaaattataatatagtctatataatttatatCTAAAACgtgtgtgtgtgt | |
| gtgtgtgtgtgtgtgtatgAAGTTAGGTGGTAAATAATCCAATTGACTTGTTAAGT | |
| TTTGGGCTAATAATATGCAGAGTTATCAGCAATAGGGAAGACTGAAGACTTTGCTC | |
| CTCTTAGAGTAATTGAGGGTCACCGTGGT | |
| >DG8S137, chr8, pos 9503869 in NCBI build 33 | |
| Primer pair: | |
| F: CTTCAGATTGGAAAGTCAGGAGA | (SEQ ID NO: 75) |
| R: AAAGCTCTCAGCAAGGACTTTA | (SEQ ID NO: 76) |
| length: 240 | |
| Amplimer: | |
| CTTCAGATTGGAAAGTCAGGAGAGATTTTCAATCTTCGTTTCTTCCCACTAAATGT | (SEQ ID NO: 77) |
| ACTAAAATAGAAACTGTTGTTGTTTTTAACTAAAATCAGAGCAGACTGGAATTACG | |
| GAAAAGAATATTATGAATGGTTCtatatatatatatatatatatatatatatatat | |
| atatatatatatgtaGACAGAACTTAACATTTATGTTTTTTTGTTATTTTTAAAGT | |
| CCTTGCTGAGAGCTTT | |
| >DG8S182, chr8, pos 9516392 in NCBI build 33 | |
| Primer pair: | |
| F: GATCTTGGCTGGCAGAAGAA | (SEQ ID NO: 78) |
| R: GCTCCGAGAAGAACATATGGA | (SEQ ID NO: 79) |
| length: 289 | |
| Amplimer: | |
| GATCTTGGCTGGCAGAAGAATAGAATCAAGAAAATTTTCTCAAAGGAAGAAGAGAA | (SEQ ID NO: 80) |
| TTGCACTGAAGCTTTGGGAATAAAAAGAAGTTAGCCACGCAAAGATAGAGTCTTCC | |
| AGGTGAAGGAAAGGCATATACAAAGGAATGGCAGTAAGAAAGAACAAATCATGTTC | |
| AAGAAGCTGGAAGGAGTTGGCCGTGGCTGAGCGTTGGGTGAGATGACAGTGGAGAG | |
| GTGAAGAGGCCGACAGNGGGGGCAGGGCCAGAAGCAGAGAGGGTTCCATATGTTCT | |
| TCTCGGAGC | |
| >DGBS262, chr8, pos 9560368 in NCBI build 33 | |
| Primer pair: | |
| F: TGCATATGTCTGGCCTGTCT | (SEQ ID NO: 81) |
| R: TTTCTTCCTGGCTTTCCTTG | (SEQ ID NO: 82 |
| length: 350 | |
| Amplimer: | |
| TGCATATGTCTGGCCTGTCTCCTGGCACCTCTGCTTTCTCTTCATGAAGCACCCAG | (SEQ ID NO: 83) |
| GTAACCCATTATCCAGAGCTCTTACTAATTCTGTTCAGTGTTTGTTTCTTGCTGCT | |
| GGGGCAGGAGGTGGAGAACAAAGGGAATGAGGGAACATTGAGAAATTTCTCTTCAT | |
| TGTGACCAGCTAGGGCAAATTGTCCTTGGTCTTCTAACCCAGCAGCAAGTATTCAT | |
| TGCGAAAacacacacacacacacacacacacacacacacacacacacacacacgca | |
| TGCCATTTATGCAAAACACATTAGTGAGGGTATTTTTCCTCTTTAAGCACCAAGGA | |
| AAGCCAGGAAGAAA | |
| >DG8S136, chr8, pos 9647411 in NCBI build 33 | |
| Primer pair: | |
| F: GCACTCACAGCTTTGCAAGTA | (SEQ ID NO: 84) |
| R: TCCCTGAGTGGAGAATCTGG | (SEQ ID NO: 85) |
| length: 138 | |
| Amplimer: | |
| GCACTCACAGCTTTGCAAGTATTGCTGCTCAGTGAAAATGTAAGTGCCATACATGT | (SEQ ID NO: 86) |
| GTACCATcacacacacacacacacacacacacacacacacacacacacacCCCCTT | |
| CTAGACCCAGATTCTCCACTCAGGGA | |
| >DG8S179, chr8, pos 9697364 in NCBI build 33 | |
| Primer pair: | |
| F: AGGATCAGCATGGAATTTGG | (SEQ ID NO: 87) |
| R: CCCATCCGTAAATGTTGC | (SEQ ID NO: 88) |
| length: 383 | |
| Amplimer: | |
| AGGATCAGCATGGAATTTGGCCAAAACAGATATAAGTCAGATTTAGGTCTCAAGCA | (SEQ ID NO: 89) |
| TTGAGGCCTGATGCAGCAtttatttatttatttagagacagggtctctgtcgcaag | |
| actggagtgcactgctgcaacctcagttcactgcaatctcagccttccgggctcaa | |
| gctattctcccacctcagcctcctgaatagcaggggctacaggtatgcaccaccac | |
| acccggctaattttttgtagttttagtagaggcagagttttgccacattgcccagg | |
| ctggtcttgaactcctgagctcNcacttgcctcagcctcccaaagtgctgggatta | |
| caggtatgagccactgtacctggccTGATGCAACATTTACGGATGGG | |
| >DG8S134, chr8, pos 9774278 in NCBI build 33 | |
| Primer pair: | |
| F: TCCTGAGTCCAGGCTATTTCA | (SEQ ID NO: 90) |
| R: GCCTCCAGAGTACATGGACAG | (SEQ ID NO: 91) |
| length: 303 | |
| Amplimer: | |
| TCCTGAGTCCAGGCTATTTCATAAGTGAATTATGAAACTATTAtttttttctgaat | (SEQ ID NO: 92) |
| tgaaaaataaatgattataaaagaaaaaattaagaaaaaagtgaaagttatctata | |
| tttctaccatcagagacaactgctgttaacagcctggatatattctttcaggcttt | |
| ttctatTCTCTTTTacacacacacacacacacacacacGTGTGTGCATGCACACTT | |
| AATAAGACCTAAAATAACTGCATTTTGTTAAAGTTACATGTTGAAGGAAAAAAGTC | |
| TACTGTCCATGTACTCTGGAGGC | |
| >SG08S93, chr8, pos 9794410 in NCBI build 33, alias | |
| name, rs2898232 | |
| CTAGATAACTTAAAAAATGTTTTTTTTCTTCAGGCTTATGCTCATACTAACAAGCT | (SEQ ID NO: 93) |
| CTGTCGAATTATTTCAATGTGCGGAATAAAAGGCAAGAATTATTTTCTGGTGCAGT | |
| TTAGACCTTGGATGAGTAGGGTTATGCAGCTGTTTGCTGCAGTAGTTTTGGGGAGA | |
| CACACACCTGACTTAAGCTATGTGAATTTGGATATGAAGTTCCAAGTGTAAGATAT | |
| GAACCAAAGGATTTCTCTTAACGTAACGATGGAACTCAAGCCTGAACTATTTTTGT | |
| TCATTAACAACCTGGCAGTTATTTTTTCAGAATAAGGAGATTTATGAAAGAGCTGA | |
| AGTCTGGGCTTCATTTTGCGTGTACATTTGCTTCCGCTGTTGCCGGATGGTTGGTA | |
| AAGGAAATTGATAGAGTTTTTAAAGTGAGGACTGTATTGTTTACTTTATGTGTTGT | |
| TTTAAAGTAGGAAGGAACACAGTCGCCCTGCTATCAGCCTCTGGTTTCTTGT | |
| [S] | |
| CCAGTGGCGCTAAGAGTCAACTCTTCTGCCTGACAGTGCCTGCTCCTACCGTGCCT | |
| GTGCTGAGATAGCTCCTCCTGGCTTCAGGGCCTTTATGGCTGAAACTTCAattata | |
| tatataaaatatataaaataattattaatataacttaatataataatatataataA | |
| Cttttttgagacagagtcttgctctgtcggccaggctggagtgcagtggcatgatc | |
| tcggctcactgcaccctccgtctcccggattcaagcgattctccatacctcagcct | |
| cttgagtagctgggattacaggcgcccagcagggtttccccatgttggccaggctg | |
| gtcttgaactcctgacctcaggagatccacctgccttggcctcccagagtgctggg | |
| attacaggcgtgagcccctctgcccggccAACTTTGTATTTTTGCTCAAAGTTTGA | |
| TCTGTACATTTTGAATCATTTTTATCCTTTTTCCAATTTCCCAACTAACCAA | |
| >SG08S112, chr8, pos 9804270 in NCBI build 33, alias | |
| name, rs3735823 | |
| GTTACATGATGACCATTAGTTAAATGAACTAAAGAATGATTGAGCTTATATTCTGT | (SEQ ID NO: 94) |
| AGTATCGTATTTGGAAGTTGTGTGTTCAATAAAACTCTTTTAGTATAATTCAGGCC | |
| AATAGGTATTAATATTAATGAATGTCAGTAAATGGAAGCTATGTTTTTACCTTCTA | |
| GCACAAACATCTTTAGAAATTTTATTACGACTGTGTATGTGTGTCCAGTGGCTGAC | |
| TTTCCAAGCAGTTATTAGAGGAGATCTGAGTTTTTAGCTTCTGCATTATGATTCAT | |
| GTTGAATATTTATGGAAGAGAAGTGTTTCTACAAATATGTAAAAATATTGGTGAGT | |
| GAAAGAAATGGCTCCCAGTATGACAGAAGAAAATATCCTAAAGAGATCCACAGTTA | |
| TCTGCAGTTTCCCCAAGGTTGTGTTTACATAAAAAAGACATTGTTTTATGTTCTAG | |
| CATCAAGAGATGATTTTACGATATAACAAGTTCCACAAAGAACTCTCGTAAG | |
| [R] | |
| TGGTTCTCAGTCCCGGCATAACTGCTACGGAGATCACAGAGCAATATTATTCTCTG | |
| GATTTATTGGGTTTGCTGCATTCTGTTAGCATCATTCATATTTTTCTCCCATGGGT | |
| ACCACTTTCCTCTCTTTTCCTAATACCAAGATATGGAGACTCATTTATGCCGTGGA | |
| GTGTGATGCTGGGAAATGAATGCTTGCTTATTACCTCTCTCCACAGGACCTTTCAT | |
| GACCATACGTCGATGTCTGCCGCCTCAGTATAAATAGGCACATTCagaaatgtgtt | |
| ctctagtgaagggcatgttggcttggtggaaagcacagggacttcacgtctggact | |
| gcgagtcagagctgtgcgtcatgtgcttactggctgtgtgaccttggataaatttg | |
| cctcagttttctcatttgtaaaacagacagtcgctatttctgggaatagatgagat | |
| aataaggaaagaacctagaatggtacctggcTCCTGCCAGTTGCACAGAATG | |
| >DG8S138, chr8, pos 9815189 in NCBI build 33 | |
| Primer pair: | |
| F: TGGCGGTTGTTATTAATACGTG | (SEQ ID NO: 95) |
| R: TCCATTCTCATTCTCATTCTCA | (SEQ ID NO: 96) |
| length: 299 | |
| Amplimer: | |
| TGGCGGTTGTTATTAATACGTGATTTCACTTTTCATTTATTTCATTTTTATGTCCA | (SEQ ID NO: 97) |
| TTGTGgcttctaacctcatatttcacacatagcaggtactcagtaaatacttaata | |
| aatcaatgaatGCAAGTAATGACTATGTATATACTagtggagaaggaaggagggga | |
| gggaaaggagaggagaggcgaagagaggTGGGCCAGGCAGAggagagaagagaggg | |
| agggagagggagagagagagggagagggagagggagagggagagagagGAGAATGA | |
| GAATGAGAATGAGAATGGA | |
| >SG08S15, chr8, pos 9851027 in NCBI build 33, alias | |
| name, rs2062331 | |
| TTGTAGGACTTTTAGAAAACATGGGGTTGTGCCTTTGGCCACACGCATGCTTGTGG | (SEQ ID NO: 98) |
| ATCTACAAGAACAGCGGTCCTGTAACTCTTCAGGGAAGGGGCACCACATATCTGTC | |
| CTGTCACCATGGCAAAGCTGGAAGGGTCTGCAGAGCTACCCAGCATGCTGCTGGTG | |
| TTGTTGTAACCAAGCAGAGGGCAAGATTCTCGCCATGAGAATTGATGTACATGTCT | |
| AGCATGTGAAGCATCCTAAGGGCTGAGGTGGGTTCCTGAAACCTGTGGAGGAAAAT | |
| GCTCAGTGCAAGAAGCCAAAGAAAAAGGCACCAGGCTCAGCGGGAGCACCCGCCTG | |
| GAGAAGCATACTTTGTGAGGATCAGCAGAAAGGAGCTGAGTGTGGAAGCTGTCCCC | |
| AAGTCATGGCACAAAAGTATTCAAAAGAAAGGATTTCTGGATTGTTTTTTAAAAAA | |
| CAAAACTGTGATGTAAATGATGAATTGTGCTCTGTGGTCTGATTAGGAATGT | |
| [R] | |
| AGTGGATCCAGAGTACAGTGGGGCTGAGGCAGTGGAAGTATTTTTTTGTGtttttt | |
| tttttaacttttaggtcagggatacgtgtgcatgtttgtttaatgggtaaacttgt | |
| gtcacgggggttcgttgtacagattattttgtcacccggataccaagcctagcacc | |
| ccaatagttattttttctgctcttgtccttcctcctgccctctacactcaaggagg | |
| ccccagtgtcttttgttcccatctttgtgtccatgtgttcacatcatttagctccc | |
| acttctaagtaaaaacatgaggtatttggtttcctgttcctgtgttagtttgctaa | |
| ggataatatccgccagctccatccatgttgctgcgaaagacatgatgtcgttcttt | |
| tttatggtggcatagtactccatggtgtatatgtaccacattttctttttacattc | |
| tgtcattgggcattaggttgattctacatctttgctattgtgaatagtgctg | |
| >DG8S128, chr8, pos 9943010 in NCBI build 33 | |
| Primer pair: | |
| F: TCAAAGGGAAGTGTCTTGGTG | (SEQ ID NO: 99) |
| R: CCCTCCAGAGTTCACAGAATG | (SEQ ID NO: 100) |
| length: 137 | |
| Amplimer: | |
| TCAAAGGGAAGTGTCTTGGTGTCTCACTGGCACATATCCAGCATGATGTTGGTAAA | (SEQ ID NO: 101) |
| TAACCGAGTCCCGGTGTGGCGTATTTCTCCCTGAATCTTGACTGANAAACTACTGA | |
| AGCCCATTCTGTGAACTCTGGAGGG | |
| >SG08S100, chr8, pos 9961132 in NCBI build 33, alias | |
| name, rs2975734 | |
| GTGATACTGATGACAGTGGTCTGAAAACTGGCCTTTGGAAGTCATAGACACAATGA | (SEQ ID NO: 102) |
| ATTTACCTGTCACCACCACCACCTCCCCTAGGAACTTCTGAAGGACATCTACATTC | |
| CGTAGAAATAAAGTTTTAAATTGAAGGAAAAAAATATTCAAACTTACATCATGACT | |
| TAAGCACCTAAGAGACTTAAAGAACATATCAAAATTACAACTGTGTCACTGAATCA | |
| AATTTACATTTTTGACACAATCATTACAAAATCATTACTTGGTAAGAATTTTCCAA | |
| TAGTCCTACTGGATTGTTTTTATTTAGAATTACCTTAAGATTCCTGCATTTCTACT | |
| CACAATTTTAATCTGTCATTACTCATGAATATCTGTGTCTATGAGATTTTTTATTA | |
| TGAGATTTTAGTTTCCCTTAAGATTTGGGTTCTCATATGAAATCTTCAGGAAGAAC | |
| CTTAAAGAAAGTTCAAATTTTCATAAAGCCCTTTTCCAAACACATTGACACT | |
| [S] | |
| CAAATTTTGACCTGACTGGTAAAGATCTGTGATTGTGATTGTTCAAATGTGATTCT | |
| CTAAAAATACCTAAGAGGCCGACCACTACATCTTCCGCACTCATGAAAGGCAGTTT | |
| TCCAGATCTGACATGTCCTATGGGTTCACTACATAAATTGGCTAGGGCAAGTTCTA | |
| CTAACTAGTACACTCCATTCTCTTGCTAACTAGCACACTCCTGTTAACTAGAATGC | |
| CCCACTCTCCACCTCTGCCTACTAAGGGTACCACTGAATAACAAACCCTCCAACAA | |
| CAGATGGGGTAGGAAGAGCAGTCTGTCTTGTCAGAGTGGAAACCAACAGGGAGGCT | |
| GGGCTCCCATTAGAACATGTGCAGTTACCGCATGTTCCTTCAGTGTCTTATCCAAA | |
| TGCTCCCTCTCTTCCAGCTCTTTCCCCTGCTTTTAGACTTCACTCAGAACACAGCC | |
| ACGTACACAACAATTTCCAGGGCAGCCTCCACCCCTGGGATCCTAGAAAGTT | |
| >SG08S39, chr8, pos 9971559 in NCBI build 33, alias | |
| name, rs2272597 | |
| tgtttgcctaataacagtgcattgaaatatatgtttgttttgtgtggtttttttgc | (SEQ ID NO: 103) |
| atcagttttgttttataacaaaagGCTAAaaataagtatttaaagaaaatagtgca | |
| tactatattttatttgctgatattcataatgatcaccagattattgaaatttatga | |
| gtaattttgctataaataagcctgttttctttgtttaaACACACACACACACATTT | |
| TCACACTCACACCTTCAAAGCCACATAATAGAATGTTTAGCTTAAACCTGCAGCCG | |
| CTAGTTGAAATGTTGCTTCATGGAGTTTTATCCTCCTAACAACCTGTGTCCTAAGT | |
| CACATTCCTCTCCAGAAATGTGGACATTGACCATATTCCAGTCCCTGAGACGCTGT | |
| TTCAGCCACACGTGGCACCCCAGACCCTTGCCCACCTGCATCCTGGTCATTCATCC | |
| TCCTCCTCATGGGGTCATTTCTTGATCCCTATTAAGCATTAAAAGGGGATTA | |
| [M] | |
| ATATCTCTCTACTTGCAGCTAATGTTTTGCTTGGTTTGGCCAAGAACATTTTAAGT | |
| TTTAAAAACCTGGGGCTATTGGAGTGGGACCATGGGCAAAGGTCAGGACAGGCTAG | |
| CTACTAAAATGGCCTGCCACGGACCTTGTACGTGAAGGTTGAAGGATTCTGGTGCT | |
| CTCTGGTGCCATCGCTGTTAGTCGTTGTGCAGCACAGAAATATTTTATTCAACAAA | |
| CTCTGCAGACTCCTGAACTTTAGGGGTGGGCTGCCTTCTGCCTGGTGCTCTGCACA | |
| GATCCTGGAGCTCTCGTGGTCATTTATGTGCAGTGAAGCTGCTCCACTCACCTACA | |
| GCTTGTCCTTTTCCAGAGAATCCCTATCATCCTCCCCTCATCCCAAGGAATGCAAC | |
| AAAGGAAAATTAATAGTGAATGCTTTTGCCGGAGACCTGTGGATACTTAATTTTTA | |
| TAGATACTCAATAAATATTTATTTATATTCACTAGCAGCAAGCAATTCACTT | |
| >D8S1721, chr8, pos 10011582 in NCBI build 33 | |
| Primer pair: | |
| F: GACTTTCCTAAAAGCCCAGC | (SEQ ID NO: 104) |
| R: GCATCTTGCATGGTGTATTG | (SEQ ID NO: 105) |
| length: 170 | |
| Amplimer: | |
| GACTTTCCTAAAAGCCCAGCCAGTTCAGATGATAGGTGCAGACACATCATATTGCA | (SEQ ID NO: 106) |
| TATATTCACATTACACACACACACACACACACACACACACACACTCTCACCCTTCT | |
| CTTTGCTGGGGAAAGGTTTGTTGCAGAAGTTACCATTCCAATACACCATGCAAGAT | |
| GC | |
| >D8S542, chr8, pos 10028442 in NCBI build 33 | |
| Primer pair: | |
| F: AATCACCTANACTACTGCCA | (SEQ ID NO: 107) |
| R: ATCTGATGGGGAGTTATGTATTC | (SEQ ID NO: 108) |
| length: 241 | |
| Amplimer: | |
| AATCACCTATACTACTGCCACATAAGCACTATCAATAAATTTTATCAATCTCTTCC | (SEQ ID NO: 109) |
| TGGGTGCCTACCAGATGTGTGCATGCACGCGTGcacacacacacacacacacacac | |
| acaAATTTCTTCCACTGCATTCAttacagcatgcttttctctcttaccactatatt | |
| gggaatacttccccatgtcactaaaacttttagaaaacaccatttataatgaatac | |
| ataactccccatcagat | |
| >DG8S302, chr8, pos 10062565 in NCBI build 33 | |
| Primer pair: | |
| F: GCCATTCGTGTGGTCTGATA | (SEQ ID NO: 110) |
| R: AAATGTTTCTGCTGCCATCC | (SEQ ID NO: 111) |
| length: 268 | |
| Amplimer: | |
| GCCATTCGTGTGGTCTGATAACAGCAGCAGCATTAAGTTCCCGTCCATTGGCTGCA | (SEQ ID NO: 112) |
| AGCAGGGAGGAAAAAAGGCCCCAGCGCCTACTGCCTGCTTTCCTGCCTGCGTTAAT | |
| ATCATCTCTTATCTTACCAACTAACATATAGGGgtgtgtgtgtgtgtgtgtattta | |
| tgtgtgtgtgtgtgtgtgtgtgtgtgtCTGGGTATATATACACACACATTTATATT | |
| CGTTAATTTCCGTGGAAAAGAAAGGGATGGCAGCAGAAACATTT | |
| >DG8S257, chr8, pos 10128880 in NCBI build 33 | |
| Primer pair: | |
| F: CCATGGCCTATGACCTATTCA | (SEQ ID NO: 113) |
| R: TCTCCTCCCAGCAGTCACAT | (SEQ ID NO: 114) |
| length: 147 | |
| Amplimer: | |
| CCATGGCCTATGACCTATTCAGGCTCtgtgtgtgtgtgtgtgtgtgtgtagtgtgt | (SEQ ID NO: 115) |
| agtgtgtAGGGAAAGATACACGGTGGATGAATGAGAGCTGGGGCTGGGGATATCAA | |
| GCCTATTGACTCCCCATGTGACTGCTGGGAGGAGA | |
| >SG08S120, chr8, pos 10154461 in NCBI build 33, alias | |
| name, rs3750310 | |
| CCTGGGCCTGCAGGTGGCTGCGAAGGGAGGAGGAGGAGGGGAGGTGGGCAGTGGCG | (SEQ ID NO: 116) |
| CTGGCCTCCCTGCGTGGACCCACTTCCTCCCACGCTGTGCTCAGAGAATCTTCTGG | |
| AGACCGCAGCTGTGCCTGGGAGGCCATCCTTGTGCCTAGGAGGACAGGGAAGAGGG | |
| TGGATCTCAGACACAGGCAGGCTGGGAGGTCTGCACAGGTGTGGCCATAGAACATG | |
| GACGCCTCCAGTACGCAGGCACAGGCAGCTCAGGGCCGGGAGCGAGGCCCGTCTCA | |
| GCAGGCGGTGTCAGCCGCGGAGTGGGTAGGTCCTCTGAGGACGATCACACCTGTGG | |
| GCAAGAGCACACCCGGGCTCTGGGCCAAGTAAGCCTGTGAATCCCACTGGCGTTGT | |
| GAACCCGGAGCCCTTGGGATCCGATTTTTTATTTGCTATTTGGATACAGCTGTAAG | |
| AGATGACAGATTATTTTACATCCCTCAGTTCTCCGAACTTGCCTTGGACCAG | |
| [R] | |
| AATGTCAGGCCCTCACCGTGCCTTTTTCTCTTCTCCAAACTCTCTGGTGCTGCCTG | |
| GAGCAGATGGCACCCCCCACAGACGTCGTCCTTATTGTTGTCACCAGAATATTCCA | |
| TTTCCACAGCCACCTGGCATCCCAAAGCCTTCCTTCAGTGGGCAGCCTCTTCACAG | |
| GCAAATGCTAGCGATGGTTCAAGTCACACGGCCAGCACATACTCCATTTCCAAGGA | |
| GGTCATTGCTAACTCTAAATCTACCCCTGTTAGTTAGCCAACCCCACGTGCTCATT | |
| CTTAGAGAGGTTCTGTTCCCTGAAAACAGTCTGGAGCCAAATGCTGTGTGAGCTGG | |
| GGCCCGGTCATGGAAACAGAAAACTTCCATTCCGTCAAGCTGGATGGATTCTACAG | |
| AAGGAATTCGGTGTTTACAGAATCGTTAGCAGGGCTGTTCGCGTGAAGGTCAGGGA | |
| AAAGCACCCCAAGATTTCAGGATACCAAGAAGTTACTGAAATTGCCAAAAGT | |
| >DG8S266, chr8, pos 10161672 in NCBI build 33 | |
| Primer pair: | |
| F: GTGCTTTGCTGACATCTGGA | (SEQ ID NO: 117) |
| R: GGACAGGGTGGACTCACAAA | (SEQ ID NO: 118) |
| length: 412 | |
| Amplimer: | |
| GTGCTTTGCTGACATCTGGAAATTCcacagaggctggtggagcgatcagctggagt | (SEQ ID NO: 119) |
| gaagtgagacagacctgagggaaaatgctagctctgcctcttatagattgagtgac | |
| cctgcagaagtcacatgatcattctgaggctcagtttctttgtgtgtaaaacagcg | |
| ataatcatacccatgttgcaggacttggggaagattaaatACTATGCATACACACA | |
| CATATATAtgtgtgtgtgtgtgtgtgtatatgtatgtatgtatgtatatactttgt | |
| acagagcctgagatacagtaagtgttctctacatggtagatattattattGTCTTC | |
| TTGTAAAGGAGAGAAGGGGATTATTTGCTGAGAACTTTAAAAAAATCTCATTCGCT | |
| TTTGTGAGTCCACCCTGTCC | |
| >DG8S238, chr8, pos 10223621 in NCBI build 33 | |
| Primer pair: | |
| F: TTCCAGTGCCTGTTTCACAA | (SEQ ID NO: 120) |
| R: CTGGGAGGTCCTTTCTTGGT | (SEQ ID NO: 121) |
| length: 141 | |
| Amplimer: | |
| TTCCAGTGCCTGTTTCACAAAGTATCtgaatgaatgaatgaatgaatgaGCAGCTG | (SEQ ID NO: 122) |
| AATGTCTTTCTTTTTTATGGGGCCACATATGATTGTCTCCTTTGTAGCTATGCCAG | |
| GTAGACATAACCAAGAAAGGACCTCCCAG | |
| >DG8S323, chr8, pos 10259523 in NCBI build 33 | |
| Primer pair: | |
| F: TTGTGGGCTGTGTAGAGTGC | (SEQ ID NO: 123) |
| R: GCTGTGCCCAGAAACCTAAA | (SEQ ID NO: 124) |
| length: 250 | |
| Amplimer: | |
| TTGTGGGCTGTGTAGAGTGCTCTAAACCCAGCTCGGCCTTTGCTGTATTAGACAGA | (SEQ ID NO: 125) |
| AGCACCTCATTCATATCCCTGGGGCCCCTGATGGTGCAGTGGTCTGGCTGTGGTCT | |
| GCACACCAGCTAttctgttttgttttgttttgttttgttttTTCCTACCTTTTTCC | |
| AATCCTCACACCTTCTGATCAACAGCCCCAGTAGGGTTTAAAGGTCCTAGAGCTAC | |
| ATGGGATTTAGGTTTCTGGGCACAGC | |
| >DG8S155, chr8, pos 10297139 in NCBI build 33 | |
| Primer pair: | |
| F: TTGCATGGAGATGAACAACC | (SEQ ID NO: 126) |
| R: TCCACTCAGAGAAAGCAAGGA | (SEQ ID NO: 127) |
| length: 396 | |
| Amplimer: | |
| TTGCATGGAGATGAACAACCAGGTTTGTGGCCACATCTTGCCgtgtgtgtgtgtgt | (SEQ ID NO: 128) |
| gtgtgtgtgtgtgtgtgtgtgtgtgtgtAttgagacagggtcttgctcttttgctc | |
| aggctggagtacaggcgggtgatcatagctcacttgcagcctcaaactcctgggct | |
| caagcaatcctcccacctcagcctcctgagtagctgggtctacaggtgcagagcac | |
| cgcgcgtacctaattcttttaactttattttttgtagagacaggttctccccatgt | |
| tgcccaggctggtctcaaactcctgggcacaagtgatccgcctgcctcagcctctc | |
| aaagtgctgggatttcaggcaagagccaccgggcctggTTCCTTGCTTTCTCTGAG | |
| TGGA | |
| >DG8S291, chr8, pos 10313503 in NCBI build 33 | |
| Primer pair: | |
| F: TGCTGAATGTCAGGGTTTGA | (SEQ ID NO: 129) |
| R: CCACCCTAGCAGGTCTCTGT | (SEQ ID NO: 130) |
| length: 361 | |
| Amplimer: | |
| TGCTGAATGTCAGGGTTTGACTGTTTCCATAACAGGAAGCTGCTCACTGTCTCACT | (SEQ ID NO: 131) |
| GTATTAAGGAACTCTGGTCTACACAATAGAGTTCCAACAAAACCCTAAACACTCCA | |
| TTTGCTGGGGGAACCTCATTGAATCCAGCTCTCATTGTTTCTTTTATAGGCTGAAT | |
| CCTGTATTTACAGTGAGAGGGgtgtgtgtggctgtgtgtgcacgtgtgtgtgtgtg | |
| tgtgtgtgtgtgtTCGCGCATGCACATGTGGGTTTAACAAGATATGAAGCCTGGCT | |
| TGTCACCTTCCAAGTTCTCCACTTGAACTTGAGCATAGATCAGGGTGCCATGATTC | |
| CCCAGACAGAGACCTGCTAGGGTGG | |
| >D8S520, chr8, pos 10427394 in NCBI build 33 | |
| Primer pair: | |
| F: CTGAAGAGCAAATGGCCCT | (SEQ ID NO: 132) |
| R: TAAGATCACATGGCCCCCT | (SEQ ID NO: 133) |
| length: 189 | |
| Amplimer: | |
| AGCTGAAGAGCAAATGGCCCTGGGAAGTATTCCTTTAGGGTTACACACACACCACA | (SEQ ID NO: 134) |
| CACACACACACACACACACACACACACACACACACACGAAAATCTCTAAAGAGCAA | |
| TGAGCATAGCAGCCTGGATGGTGCTCATCCAAGGATAAGTCTCCAGACAAATAGCA | |
| CANCAGGGGGCCATGTGATCTTAGTTCACGAAGACATTCAATAAAGACCCAACAAA | |
| ACCCACGCAACAGTCTATGTCTCTGGCCCCCTGCAGGGACCTTGCTCTAGCACACG | |
| GAGCAGGGTGGGGCATGGCCACAGTGGCCCCTACTGCCCTGCACTTCCCACAGCT | |
| >SG08S506, chr8, pos 10492671 in NCBI build 33 | |
| TTCCATGCATTCCACTTCTTTCTGGATCTCTGGTTTCACAGGCAAGATGGGACAGG | (SEQ ID NO: 135) |
| CAGAGAGAACCTGGGCATGTGCCCTCTGTGGAGAAAGTGACTTCAGAAACCGCTGA | |
| GGCTCTATTAGCCTGGGATTCTAAACTCGGGGGGACATGAAAAACTCAAGAGACGA | |
| GTCATCAGGCTCTATATTCATAAGACTCTTCTCTGTGTGTGTGTGTGTCTCTTTTC | |
| AAACAAATAGCACTGCGCAGCATCCTTAGAGACTACAGCCAAATGTCCTTCATGTA | |
| TTTTCTCTACATTTCAAGAATCTCGGGACCATGCTTCCTATCTAATGTGTGACCTT | |
| GAGAGTTAAAATCAAGGGGAAAAGGTCACCGAATTGGGGGCAAGTTTGAGTTCCCG | |
| TCACCAGCCACAATCTCTATATCAAATGGAGGACAACACACCACCTGGGCCTCAGC | |
| CAGGTTTGCCTGAAGCAGGGCCAGGCAGCCTCAAGGCCTCCATGGTAGGCTG | |
| [R] | |
| GGACATGGGGACGTGGGGAAAGGGGGTGCAGGGAAACTGGGAACTAGGAGGGGAGC | |
| GTGAGAAAGAGGGAATAAATGCGTACGCGGATGAAGAGGAACAGCAGGAGGAGATG | |
| AAGGCGGCGCACAGGGCAGAACGGCAGACACAGGGCTGGGAAGGTGGCAGGGCCGG | |
| ACTCCAGAACCTCAGCTGAGCGTTTTCTTCTCCTGTGTCCCAGGGATGGTGTGAAG | |
| TGTCTACAGGCATCCGAGTGAACCCAAAGGGAGAGTTTGGCTGGCACACGGGGAGA | |
| CGGGCCAAGGCGCGGCGGGCGAGGGCGGCACAAGCATGGCGCTGCGACACCACTGC | |
| TGGGAGCAGGGCTGAAAGGTGTCTTTTGCTGTAAGGACTTTCATAAGGCAGTCCCA | |
| ATCCAAAGACTGGCTTTAATTTCACGGCCTTAGCCTCTCAGTTTCTTAAGCCTTCT | |
| GAGGACCTCCTGATCATGACAATTAAGTCACTATTTACAGCCATGTGACAGA | |
| >SG08S42, chr8, pos 10574489 in NCBI build 33, alias | |
| name, rs2278335 | |
| atgtggatgatctaccactataggtgtaatctttaacatcatcttattccttctta | (SEQ ID NO: 136) |
| aagtaagttatccgcttgtaaactgcttatttctttggggcattgtccccataaac | |
| tttttataaagcatcagtgatttcaccattccacccaagcttcaccataaatttgg | |
| tgtttgttcttgcttcaattttagcagaattcatgttgttctgaaagggggctctt | |
| tcaaattgatgtcttagtgcctcaaactagatcatgttctaacatgttataacaag | |
| ttattacaagtgtattttggtgcaaaaaaattgaaatccatgcataatatgacctt | |
| tccatgaagttttggaagacctctcCTATGCTTATGCATACACTCCCCAAACGTAT | |
| CAATCCAGTTGCTATTGCCCAAGGAACAGAAGGCTCATCACTCCATGGAGGGTTTT | |
| TCCTGCAGCCCCTACCTAAGACCTTCTCACTTTCTCTGACAGTCCTATCATC | |
| [R] | |
| TGTCGTAAAAGGCCTGCCCACTTAGTCCAACACACTGGAAATGGATGATTGACAAC | |
| ATGTTTATTTACCCATCCCCTGGGGGAAAGTCTCAGATTTTGTGAGGTTGTTGCCC | |
| CTGCAATGTGCTTTAAACTCAGCTTTCTGTTGCTTGTGTCTCTGGGTCAGAAGAAT | |
| TTGTCAGTGATAATGTTTTTGTTAAAGTCCTATGCCCAGTTAATGCCAACTCAGCG | |
| CTCTCATCCCCTAGGGCTCCTGTAATCATTTTTCTTGCCTTCTCTTACAGTTTCTG | |
| TATGTTATAGAAGTTCAAAGAAGACAAACTCTAGCCAAGAGCAGTGTGAAGAAAAG | |
| AAGACGCTATATTAATCACAGTCCAGGGATGCCTTCTGGCTTCCTGGCAGCAATTC | |
| CGGCCTGAGATTCCTTCTCTGTGCATACTTCCTGTCAACATTGTGTGATGTCAAGC | |
| TGTGGCCGTCACAAAAGTACTGTGAACACCTGTAAATCCCAACTATCAAAAA | |
| >SG08S50, chr8, pos 10587063 in NCBI build 33, alias | |
| name, rs2292369 | |
| TTGTTTTGATCCTAAGAAAAATGGGTGTCATTTTATCCAGGAATCTAAGaattata | (SEQ ID NO: 137) |
| ataataaattaataaaGTGAATGTGATAATCAAACTGTGAGGATACGAACAACATA | |
| AGATTTAATGATCGTTGTCAAAACCAGTCCGTAGGGCTGTGGAACTTTATCGTACA | |
| ATTCGACTTTGATATGTGTTTAAATATATTTTCTAAGTTATCCACAACCCAAAACA | |
| GGACCccttagaggtaatctagaggaatccctcacgttacagacagagccactggt | |
| taagggtctagagtcacacagggagttactgcagaatcactactggaaccctgtgc | |
| tcttTCTGCAGGGATTCGGATATTTTGGTTGGATTTGCATTCTTACGTCAATGTAT | |
| GTTCTCCAACTCTGCTCTTACATATTGAAAGGCAGGCAGCTATTTTTAAACACCCT | |
| GCCTATTAGCCTTCGGAACATAATAATAATGGCAAGCACCCTTTATTGCTTC | |
| [R] | |
| CCGAGCTGCAGACACCCTTCTAGGGTGTGAACAGAGCTCAGTAAAGATAGCAGCCT | |
| CAGGTCTGTGTGTTGCTTTGAGCCACGAGCTGGTCTGCAGGCAGCAGCCATGGGCC | |
| GTGCCTGTGTTGGTATGTTTAAGAACATTGGCGAATACAGGAATTACATGGACTAG | |
| GTTTAGAAAACAAACAGTAACGTACAAAAAGGAAGGTTTGATATGGACTGCAAGGA | |
| CATAAAGCAGGTGCACATGCGTGCACTACCAGAATAGCTACACGGTGGGAAGGAAT | |
| TCCAGAACCACGTGAGAAAGAGTTGTTAGGACAATGCAGTCGTGAAATACCATGTT | |
| TCCAACCCTATCACTCtattttaaaatagataataattataatttttattaatatC | |
| AAACAAATTAGCTTTGGGACCTATGGCCCTAACTTAGGGGTCACGGCTGCAGTCCC | |
| CTTTCTTGCAGACCTGGCAGGCTGCGCAGATAACTGCCCCCAGCGTTGGCCA | |
| >DG8S148, chr8, pos 10609020 in NCBI build 33 | |
| Primer pair: | |
| F: CCAGACATTTCACACACTGGA | (SEQ ID NO: 138) |
| R: TTTGCCAGAACTAGCGGTGT | (SEQ ID NO: 139) |
| length: 140 | |
| Amplimer: | |
| CCAGACATTTCACACACTGGAACATATATACAGTacacacacacacacacacacac | (SEQ ID NO: 140) |
| acacacacacacacaTGCTAGCATGAAACATCTGAAGTACACAGCCATCCTTTGAA | |
| AGGACCCCACACCGCTAGTTCTGGCAAA | |
| >DG8S271, chr8, pos 10624569 in NCBI build 33 | |
| Primer pair: | |
| F: AAATCGCAGCTACACACAGC | (SEQ ID NO: 141) |
| R: TTTCTGCAGGTGTTGCAAGT | (SEQ ID NO: 142) |
| length: 259 | |
| Amplimer: | |
| AAATCGCAGCTACACACAGCAAAGACTAACAGTATTTACTTAAAAATATTGTGTGT | (SEQ ID NO: 143) |
| GTTtatatatatatatatatatatatatacttattatatatCTTTTTTGTGATTTT | |
| TTTTCTTTTCCTTTTTTTTTGTGCCCAAGTAGAGATACGATGCGATTGAAACGATG | |
| CCCTAGAACAGAAATATTCTTTAAAGGAACAATACTTTGaaaaataaaaaaaaatt | |
| taaatCGTTGAACATACTTGCAACACCTGCAGAAA | |
| >DG8S197, chr8, pos 10625200 in NCBI build 33 | |
| Primer pair: | |
| F: GGTGAAAGACAGAAGCACCA | (SEQ ID NO: 144) |
| R: TGGTGGGAAGCCTTAAATTG | (SEQ ID NO: 145) |
| length: 185 | |
| Amplimer: | |
| GGTGAAAGACAGAAGCACCAAACAGTCTTTGAAATGGGTCAGTTATTACAATTTTG | (SEQ ID NO: 146) |
| ACTTTTtatatatatgtatatatatatatatatatatTCTAGTTTTCCTCTTTGTG | |
| TTATTTTTTTTTTTAAAAAAGCACAAATGAAAAATGAAGAATTCTTTCCAGATCAA | |
| TTTAAGGCTTCCCACCA | |
| >DG8S215, chr8, pos 10641313 in NCBI build 33 | |
| Primer pair: | |
| F: ATAAAGAGGGTGTGTATGTGTGC | (SEQ ID NO: 147) |
| R: CTCATCTTCTCTCTACAGATGTACTCG | (SEQ ID NO: 148) |
| length: 210 | |
| Amplimer: | |
| ATAAAGAGGGTGTGTATGTGTGCATATATATAGAGAGAGAGGCGAGTATATATACA | (SEQ ID NO: 149) |
| TATATATATATAGAGAGAGAAAGAGATAGGGTGTGTGTATAGATAGAGAGAAAGAG | |
| GGTGTGTGTGTTTATATATAAAGAGAGGGCGAGTATATCTATATGTAGAGAGTGTA | |
| TATATCTATAGAGGGCGAGTACATCTGTAGAGAGAAGATGAG | |
| >DG8S159, chr8, pos 10704990 in NCBI build 33 | |
| Primer pair: | |
| F: GCAGGACAGGACCTGAGAAC | (SEQ ID NO: 150) |
| R: CCACATCGCTATTGGAGGAT | (SEQ ID NO: 151) |
| length: 399 | |
| Amplimer: | |
| GCAGGACAGGACCTGAGAACCAGATACGCCTGCAGGTGCCTGTCCCTCTGCGCCCC | (SEQ ID NO: 152) |
| CCGGGTGGTGTTAGGGCTCCCTGTGCACGGAGGCCTGCAAtcatttggacaacaca | |
| tggttaccaggtgtctgctatgtgccaaacgatggtcacaggagggtgagaaagac | |
| agtctccacgttcaagagtacaaagtccgtgatccaggaagacaATGAGGCAGCCA | |
| CTGTGTCTCATTTCTGGATGAATGGATGTCACAAAGCCATGGAAGTGGTTCAGTGG | |
| CTTCCATATCACTAGGCTACCTCGCCTGtctctctctctctctctctctctctgtc | |
| tctctctctctctcAGAGCAGGCTACCTAGGATTTTACTTGCAATCCTCCAATAGC | |
| GATGTGG | |
| >DG8S212, chr8, pos 10726663 in NCBI build 33 | |
| Primer pair: | |
| F: TCTAAGATTCGCCAGCTTCC | (SEQ ID NO: 153) |
| R: ATTCTAGGGCTTGCAGGTCA | (SEQ ID NO: 154) |
| length: 278 | |
| Amplimer: | |
| TCTAAGATTCGCCAGCTTCCCCCGCCAGAGAGCGTCCAGCACTCACTTCTAAGATC | (SEQ ID NO: 155) |
| ACCCCTTCTCCCACTGAGACAGCTAGCCTTGCACAAGGCATTCCCAAGCAAGCTCC | |
| CCAACAATATAAGGAGAAGAAAGAGAAGGAGTGGCTacacacacacacacacatac | |
| acacacacacacacCTCTTAGTTGTCATTTTGAACCTAATTGTTTTAACACCAGCT | |
| GTCACATCTGCAGAATTCTCTTCTCTGGTACTAGTGACCTGCAAGCCCTAGAAT | |
| >D8S550, chr8, pos 10752550 in NCBI build 33 | |
| Primer pair: | |
| F: CCCAAAGTCATGAAATGAGA | (SEQ ID NO: 156) |
| R: ACAACATACCTGTTAGGAGGTG | (SEQ ID NO: 157) |
| length: 103 | |
| Amplimer: | |
| AGCTCCATTTCACTAATAAGGAGACAGATGTGGAGGTTGGGGAGTTGGTC | (SEQ ID NO: 158) |
| CCAGGTCACCCAACTGGGGAGGGCAGAGGTTGGGGAGGGACAGGAGTCAA | |
| TAACCCAAAGTCATGAAATGAGAAAGGAAGTAAACACTTGGNTGGAGANT | |
| CACACACACACACACACACACACACACACACACACACCTCCTAACAGGTA | |
| TGTTGTCTGCAACAAGGCAAAAATAATTCATTAATATCTCATTTAAACTT | |
| GAGGGCGAGGGAATTCCTGAACCACCTCTCTGGAGCAAATAATGGAAATT | |
| GGAAATTGATTGTCATTTACCTTTGAGGAAGACTTCGGGATGTGCCATGT | |
| CTTTGGTATAGGGCTGCGTGGTGTTGTGACGCATGT | |
| >SG08S94, chr8, pos 10763565 in NCBI build 33, alias | |
| name, rs2898254 | |
| gattggcttttactctatgggcaacagagagccatggcaggctttccaggaaggga | (SEQ ID NO: 159) |
| gtgacatgcaccttagacaggtcagcctgacagcagcttaaaactagatggaatgg | |
| gagacaaCTTTGTCCCTAAGCTCAGTCCCCTAAAGATACCAGCACATGACTGTCAG | |
| GCCCCTGCTGGGACAGCTGCCCCTCCCTAGGCCTGTCCATTCTCTTACCTCCCTCC | |
| TGCCTCTGATGGGGAAGGGGTGATGGGTTGGAAGTGGGTGTGTGCAACATTTACCA | |
| TGGCCAGGTCTGCTCTGTGCTCTGTCCCCACCCAGCACACCCATCTCCATCCATAC | |
| CGGCCAGCCTTGCCTGTTCCCTCACAGTGATGCATAAGCTGGGCTTCTCCTGCGGT | |
| GTGATACTAATGTACTAGCCAAACCCTGAGAGGCCACATATGGTGGGTGAGGGATG | |
| TGGGACTGCCAGACTGCCAGCCAGTGCCCTGAAGACTCTGCATTTCATATGC | |
| [R] | |
| TACAcatttagtagtagtgtgaccctgggccagttactgattctttctgagcttcg | |
| gtttcctcatctgtaaaatggggatgatgatacttaccttaaagggctgccatgag | |
| gtcgaaagacaaactatgacaaacagccagtctcgtgcccagcccagcgtgggtgc | |
| aagctatctggtggctGCTCGgatgatgatgatgacgatgacaacgatgacgTAGC | |
| ACCCCATTTCCAGCTCACACCATCGGGATCACCGCCAGCATCAGCAGCATCATCAA | |
| GCCATCTTCCTGCGTTGTGGCAGCTTGGGCCCCCACTGGCCATGCAGGAGCCAGGA | |
| GATCAAATCATGAATGGGGCTCTTTGCACTTCAGGCAAAGTGCAACTCCAGGAAAG | |
| AGAGAAGATTAAGGCCAAATCTCTGCACCCAAACAGGATCCAAGAAGTGGGGTAAT | |
| CTGGGACTCATCACATCTACATAAAGGGAGGAGGAAGCCCCAGGGTGGCCTG | |
| >SG08S95, chr8, pos 10810525 in NCBI build 33, alias | |
| name, rs2898260 | |
| TCACACATCAAAATGTGGACATTTAATTCATTTTAATCGAGAAATTAAATGCATCT | (SEQ ID NO: 160) |
| GCCTTGCTTCCTCTCCTGGGGCTCTTCCATCTCAGGAAATTCCCACACCAGCAGGT | |
| CTGGACAAGTCCTCGGCAGTAACTTCACTCAGCCTGAATTCTTCTTCCTTTCCCCA | |
| CGGCTCTGACTCCAAGTTCTGATCATCAAGTTGAAAGGGAAACTTACAACCAAAGG | |
| AGATGTAAACAAGAATAGTCTCTGTCAGTTCAGTGGAGAGAGAGAGAGAAGCTTTA | |
| ATGGGCACTAGTCAGTCAGAGGCTTATTCTGCAAGTGTTCATTAGGAATCAGTGGA | |
| ATTTCCACTGTTTCCCTGGTGTCACTTGGGCTGCTGCCTCTTGGCCTGTGTCAAAG | |
| ACAACAAAGGAAAATGGTCCTTGCCCCTCGAGGTGGGACTGGATGCCAACCAGCCC | |
| GACAGGCAGTGGGTGGTTCACGGTTCTGTTCCCACTGGAGGATGCTCTTGTC | |
| [K] | |
| GCCTACCCTCTCGCCTGAGACCTGGAAGGAAGTGCATGCCCAAGGGTGCCAGTTGG | |
| AGGGGAGCTAGCAGTCAGACCAGGCTGGTGTAGGCTTTGCAGACAGAGACTCACCT | |
| CCTTCCACTGCCAGAAGATGCTGCCGTCGGGTGAGGAGCTGTGACCTGGGCAGAGG | |
| AAATTCAAGGAGCCAATTTCTGCTCTGTACATAGAAAAGGTGGTCCTCTCCTGTTT | |
| GTTCGGGGGGCATCTCTGAAGCCCAGCTCCACTCTTTACCATCTTGCTAAGAACCA | |
| GGAGTCTGGAACATCTCCCAAAGTCTACGTGGGGCTCAATATCATGTGCAATCACT | |
| TTGCACCCCGTTACGAATGTGGGAGCAAGAGTTGGTCAATTTTGGAAGGGCTTGGT | |
| TAAGACAGCTGGTAAACCTCAGCTGAGATAATATCTCTATTCTCCTCTCCAAAGAG | |
| GTTGGCAGCTTCACCGGGCAAACAGTGCCCAGAGAGGCCTGCATAAGCCACA | |
| >SG08S96, chr8, pos 10829574 in NCBI build 33, alias | |
| name, rs2898261 | |
| CACGGATAGAAGGCCACCACTGAGCAACTGTAAGTGTGCAAGTCCAATCAGACCAC | (SEQ ID NO: 161) |
| TTCCAGAAGGTGCTTTCCCCTACAACTAAGACAGCATTCACACTTAACCCTTGTAG | |
| CAACTTCCTACACTGAGAAACACAACAGAATTTTGCTGTATGATTCTCATCTTCTC | |
| AGAAAAATGTGTTGTCTCTTTGATCTGCCTAATTAGGCTAATTGAACTAGGAATCA | |
| AAGCAGTTTCTGGGGAGGAAGGTAGGAAGTTCTGTTTTTAGTTTGGCTATGATTTG | |
| TCCCAATCATTTTATGCTACAAAAGCTTTTGTTGGCGTTGGCCTCCGAGTCAGTGC | |
| TTTGAAAGGTGGCCGCAAATGTGATTTATGGGAAGGTGCTGCCGGGGGCATGCACT | |
| TTATGGGCAGGTGGTGCCGGAGGAAGTGGTTAGGAGACAGTTTCCTCACCCATCTC | |
| CTGGAGAGACCTCCATCTCCCTTACCCACCCTGCAGTGGTACCACGCACATC | |
| [K] | |
| GACGAAAGAGGCTGTCGCTAA.AACGCTTTGAAAAGCATACACACGTGCACACACAC | |
| AATGCTCACGGGTAGTATTTGCAGTACAGAATTCTAGTACTGTGCACCTCAGCTAC | |
| AGACATCCCAATTTTTGAAAGTGTCCATAATTTATAGCAAGAGATATTTGGGTAAG | |
| TGCAGAAATTATACACGAGAGTCATTGAAACTGAGTTTATAAGAGTCAAAAATTGG | |
| AAAGAACCTGAATAACAAGAATTGTAAACTGCTGGACTTCCAGCAAGAGGGAGCTG | |
| GTTATATTCATGCAGAGCGGCCTTGAAAAAGATGCCGTGATTGGATAACGTACACT | |
| GTACACGGCTGAGAACAAAGGAATCTGAAATGACAATGAATGGAGTATTAGCAGCA | |
| GTGACCTAGTGAATTTTGTTCTGTTCATTTTTGTGCACTCTCTAAAATTATTTACA | |
| AATTATGTCATTTTTTATGATAAAAAGTTGTCTGAATTTTGGAAAAACAAGG | |
| >SG08S5, chr8, pos 10857894 in NCBI build 33, alias | |
| name, rs2001329 | |
| TTTATTGTGAACTTGCAGAAAATGGAAAGGATTATGCTTTAAAGACAGTTGGCTTG | (SEQ ID NO: 162) |
| GCTGGATAGAAAAGATCCCTCTGTCCTGTTTCCCTGTCCTCCTTCCCACATCGATT | |
| TAAAAAATTAGATGCAAATGCAAAATCCTTAAATTATAGATTTATGATAAATTTAA | |
| ATTCTGGTAGAATCAAGGTTTTATAACATTTAAAGTGTCTGACACTAAGTGTATAT | |
| AATCTTTTAAGAAACGTCTTCTTAACAGCGCATGGTATTCTGTGACTGTTCGTGTA | |
| CCATGAATATTCTTATTGGGTTCTAGAGTTAGTTACTGACTCTTGAAGATGGGCAT | |
| CTAATGGTCCTCCTGTGGAAGTGGAGAGCAGCTCTCCACTGTTTGATAACATTTAA | |
| AGCCAAGGGTGAACCACTCAAGAAACATTTGGTGGTTATAATATTTTTTTGTTGTT | |
| GTTAAGTACCATCAATAAAACTGAAAAATCTCTTAAGTACCTGACTCCTGCA | |
| [R] | |
| TGATACAACTGCAGTGATAAAACTTTTAGCTTTTTACATCAGGGGTATTAGGTATT | |
| TTCTCACAGAAATAGCCTTttgaggtgaaattcacataacatacaattaaccattg | |
| taaaatgaacaattcagtggcgtgtaagagtatgtttacaatgttgagcaaccatc | |
| acctctgtctagttgcaaaatgttttcatcactccaaaagaaactcctttattcat | |
| CATAGCCCAAAGTtggaagtattttcttgattgggctcttgattacatggatgcat | |
| ctgagtcattgaattgaagcctaagatgtgcttaatttcactgtgtgtaagtttca | |
| cctcagttAACAAGAGAGAACAGAACAAACCAAAAATCTTAATTCTTTTGAAaaaa | |
| agactttctggctgctttattaaagaagccaggggaacaaggttaaaaggaaatca | |
| gttagcagtgaccaaggcaagagatgatggtggcttggctgaagatggtgac | |
| >SG08S102, chr8, pos 10865779 in NCBI build 33, alias | |
| name, rs3021495 | |
| ggatggcatctgaatcctggatttcccagacctcagaaccagaaggaatacatttc | (SEQ ID NO: 163) |
| cattgtttaagccacccaggcaatgatatttctgttataaaagcccaaactaagat | |
| aCCCACACAGAGAACACCTACACACAGTGTGGTTACAGGTTGCATCATTTCTTTTT | |
| CTTTTTCAATATTTGCATATTCTCTAAATTTTCTACAATGACCCACCACATGAATT | |
| CTTTTAAAAGAAAAAAATGGTAAATATGAAATAGAATAGTAGTGTTGACCCTTAAG | |
| AGGAAAAAGATGGTAGAAGACACTATGTTGCTTACAGTAGACTACAAATGTGCGTG | |
| AAATTTGTAAATAAAAGATGAATACTTATAAATGTCACCACCTCCCTCTCTGATGT | |
| TTCTGAAACCAGAGCATATGTGGTTAACCTTGCTCTAGCTCCAGTCCATCCATCCA | |
| TCATCATGCTAAAACATACAGCTGTAGGCAGTGGAGAAGAGCTGTATGTGGT | |
| [S] | |
| AGGAAAGCGGGAGACAGGAATTCCAGAAATGTCTACTAAAGCAGTGCTTTAAGTTT | |
| TAATTTATTCAAGAAACCAATACATATCAGAGCATAAGTGAGAAAAAGAAAACAAT | |
| TATAAAAAATACAAAGGAGTCCAGGATAATAGAAATCTTTCTTCATTCACATATTC | |
| TAGCTAGAATAGTGAGAAGAAATTCTCCCTCAAACGTGGACAGTCCCTTACATCTT | |
| CAGCCGACACGGAAGTCTTATCTGAGAATAGAATCTCTGCTACACTAACCTAGGAG | |
| ACGGCCAGGCAACTGCTGCGGTATACCCATCACCCCAGTGTTCTGGAAGAAAAAGA | |
| CAGCAGGGAGAAGTTCTCTTTAGAACCAGCTCTTCTACACCAAATGAACTCAGGAG | |
| ACAATGAATGGAAACACCATGCCATGGTGTGAGCAATGCAATGTGGAGCACAAGCA | |
| GCGGAGAGTCTGCTGAAGAAGCTACTCCCCTGAAATAGGAAAGAAGAAAACC | |
| >AF131215-1, chr8, start pos 10872575 in NCBI build 33 | |
| Primer pair: | |
| F: GCCAGCCAGACTGGATTAAG | (SEQ ID NO: 164) |
| R: AGCCGAGAAGACCTGTGAAG | (SEQ ID NO: 165) |
| length: 257 | |
| Amplimer: | |
| gccagccagactggattaagaccccccgtcaatgacctcgtttaaccctagttacc | (SEQ ID NO: 166) |
| tctttcaaggtccaaacatagtcatactgggggtcagggcttcacatatgaatttg | |
| ctgagggggcttgagggatgcacaattcagtccataaACGCTGTATATATTTATTT | |
| GATGTAGTTTTGTTTTAAATAAAAAgtgtgtgtgtgtgtgtgtgtgtgtgtgtgtg | |
| tATCTAAAGTAGGCTTCACAGGTCTTCTCGgct | |
| >SG08S507, chr8, pos 10881766 in NCBI build 33 | |
| GCCAAGAAATGACATGTTGATCCTCAACTAGCTTGTGGACAGAGTGTTTCTTTTCT | (SEQ ID NO: 167 |
| GGTCATTCCTTTCAGCCACTGATATAAACAAATATAATTATCCAATCAAAATTCTG | |
| AATGATGAGAAGTTTCCTATGCAGTCCTAAGCATACTGGTTTTACTTTCCATAGTT | |
| CAGCAAAAATATTACTGGATTACTGGGGCTTTAAAATGGCCCAAGCTGTAGCCCAC | |
| AGATCTGCACTAGCTCACAGAATGCCACGGTTTGGTTTGTTTCTGACTATGATCAC | |
| AGAGTAATACTAACAAAATCTTGCTATTTGAAGGAATTATTAATTTTTGAATTACA | |
| ATTAGAATACAATTAGATTATTCCACATTACCCAGTGAATTATTATTATAGGTGCC | |
| AACATTCACAGTTTAATCCAATGAAGAAACTGAGCCTATATAAAAATAACCACCAC | |
| CAAAGCAGAAGAAAAGCTACGTGAAGAACTGAACTCAATCTTAATGGTTCCT | |
| [K] | |
| CAGATAACTACTCCCAATTGACCCAAATAAACCAATTTACTGGGTCAAGAGAGAGC | |
| ATGAAGGAACTAAGGACTCTGTTAGAAGTGAGGAAATATGGAATTACTCGTGCATG | |
| TAGCATGTATAACATACAGAACAAGCATTTCTGAAAATGTGAGCAGTATCAATAGG | |
| TTGGATAACTTTAGCCCCAAAAACTCTACTACTACTGCTTTTTGGAAATAATTAAA | |
| AATATCTCAATACAGTTTATAAACTTTGATAAAGTCAATATAAAAGTAATAACATC | |
| ATATAAACCGGTCTTTTGCTCATTTGAACTCCTGACATGGGGATTATAAGCCATAA | |
| CAGATTTCTTTTTTCAAATATCTGAAATACAAGGAATAATTTTCTTTAAATGAGTT | |
| GCAATATACCAACCAGTATTGGGCTGGTTTCTGTGATTTCCTCTTAATTGGTGGTA | |
| GCAGCAGTAATCCTCTAATTCTTAGGATGGACAACTGACTTTTGAATATCTC | |
| >SG08S70, chr8, pos 10881783 in NCBI build 33, alias | |
| name, rs2409716 | |
| GATCCTCAACTAGCTTGTGGACAGAGTGTTTCTTTTCTGGTCATTCCTTTCAGCCA | (SEQ ID NO: 168) |
| CTGATATAAACAAATATAATTATCCAATCAAAATTCTGAATGATGAGAAGTTTCCT | |
| ATGCAGTCCTAAGCATACTGGTTTTACTTTCCATAGTTCAGCAAAAATATTACTGG | |
| ATTACTGGGGCTTTAAAATGGCCCAAGCTGTAGCCCACAGATCTGCACTAGCTCAC | |
| AGAATGCCACGGTTTGGTTTGTTTCTGACTATGATCACAGAGTAATACTAACAAAA | |
| TCTTGCTATTTGAAGGAATTATTAATTTTTGAATTACAATTAGAATACAATTAGAT | |
| TATTCCACATTACCCAGTGAATTATTATTATAGGTGCCAACATTCACAGTTTAATC | |
| CAATGAAGAAACTGAGCCTATATAAAAATAACCACCACCAAAGCAGAAGAAAAGCT | |
| ACGTGAAGAACTGAACTCAATCTTAATGGTTCCTTCAGATAACTACTCCCAA | |
| [Y] | |
| TGACCCAAATAAACCAATTTACTGGGTCAAGAGAGAGCATGAAGGAACTAAGGACT | |
| CTGTTAGAAGTGAGGAAATATGGAATTACTCGTGCATGTAGCATGTATAACATACA | |
| GAACAAGCATTTCTGAAAATGTGAGCAGTATCAATAGGTTGGATAACTTTAGCCCC | |
| AAAAACTCTACTACTACTGCTTTTTGGAAATAATTAAAAATATCTCAATACAGTTT | |
| ATAAACTTTGATAAAGTCAATATAAAAGTAATAACATCATATAAACCGGTCTTTTG | |
| CTCATTTGAACTCCTGACATGGGGATTATAAGCCATAACAGATTTCTTTTTTCAAA | |
| TATCTGAAATACAAGGAATAATTTTCTTTAAATGAGTTGCAATATACCAACCAGTA | |
| TTGGGCTGGTTTCTGTGATTTCCTCTTAATTGGTGGTAGCAGCAGTAATCCTCTAA | |
| TTCTTAGGATGGACAACTGACTTTTGAATatctcagtaatgagatctccatt | |
| >AF131215-2, chr8, pos 10885941 in NCBI build 33 | |
| Primer pair: | |
| F: GGAAGCTGATGAGGTGTATATGG | (SEQ ID NO: 169) |
| R: GAGTCTGAGGTGGGAGCATC | (SEQ ID NO: 170) |
| length: 242 | |
| Amplimer: | |
| Ggaagctgatgaggtgtatatggatactctgtgctatctttaagcttttctgtaaa | (SEQ ID NO: 171) |
| cataaaaaacctaaaattattttaaaataaaaGgtatgtatgtatgtatgtatgta | |
| tgtatgtatgtatgtatgAtttttagagatgcagtctctctctgttgcccaggctg | |
| gtgtgcagtggcgtgatcatagctcactgcagcctcgaattcctggacccaaggga | |
| tgctcccacctcagactc | |
| >SG08S71, chr8, pos 10887924 in NCBI build 33, alias | |
| name, rs2409719 | |
| CATACTGCATACAAGCCAAGAACATAAAATGAACCTCTCAGTCTTACCCTTCCTGC | (SEQ ID NO: 172) |
| AACTGAGGACCCGCTTGCCGGCACTcagtaggacacgtgattaaaagtgtggcttg | |
| tgaggccaaactgcatggttctgaaacctggttctaccatttacaagctgtatgac | |
| attaggcaaattacttaccttctttaagccacagtttcctccttgagacaggtgga | |
| cattaacagtactagctcatgaatttagttggccgtttcaatgagttaatacaCAT | |
| CAGCTGTTACTAACATCCACCATATATTCCCAGAGGGGTACCCAATTCTTTGGGGT | |
| CTCAATGACCCTTGTCCTTCACCCTCTAGAAAGCATGTCATCAGAGAATAACAAAC | |
| ATTATCTTCAACTTACTTGATCCACTGCTGCATATAATTTAAGTAAGTCATTCTCA | |
| AAACTTACTTTACTAATAACATAGTCTATACAACCCCCAAGTAATGACCACA | |
| [R] | |
| TGCAGTCTGTTACGACAGCTATGGCAAATACTGACCTAGATCGCGAGAGAAAAGAA | |
| CAGCTGCTGTCCTCACAGCTGCCCCGCCTCActttctgctaacagacgctgcttct | |
| gtatggccatcagcttgccatgtgctttcaggcaggctggacccatccccattccc | |
| tacatcagcagcatcagcttcaatcaggaacttgtgaaaaacacaaattgtcagtc | |
| cccaatccaaactagagcagaaactcttcaggtggggcctggcaatctgtgttttg | |
| ataagtcctccaagtcattctgatgcagaccagtctgaaaactactgACCAAGAAC | |
| CACTGAACTAATAATGGCAACTGCGTATCTCTAAgtttagaaatggggtatacaac | |
| aattctagccaaggaggggcaacttctagaaattttgcttactcttaaaaatgaac | |
| acaaagaaggtaccttatctcttctggcctttagaatgttgttgattagaga | |
| >SG08S517, chr8, POS 10893214 in NCBI build 33 | |
| CTTCAGCTTCAATTCAGGTAGAGCAGTGAGGTTTGAAAGTGCCTCAAGCAGAGCCC | (SEQ ID NO: 173) |
| ACAGTTCTCTGATCCTTTACAATATCACACTCTGTAATTGTGTGGCATAGCAGCCA | |
| TGCTAGGAACGAGGTCAATTACTTAGGTACTCGCTAGACTTTTTCCTTTTCTCCAC | |
| CCCTGGGGTCCAGGCTCTTTTCCCAGCACTTACTCAGGGCTGTCATTAGCCCTTTC | |
| TCCTCAGTTTCATCGCCCCTGCATTTACGTTATTCTAAGTCTTCTCCCCTATGGGT | |
| TCCTGTGGGGAAAATAAAAGATCCGAAAGGGAAAAAAGCAGAAAAGAATGAAATAA | |
| AGTGAAAATTCAAGAGGTTCTTGTTTTAAGTCCCTATCTTAAAAGATATATGGCTT | |
| TGTCACTTTCAAAAGCATTACATTATAAGGTATGTGGCCAAAACACAATCAATAAA | |
| CAAACACACGCAGACAGATACAACTAAATACACACAAACATACATGCCACAA | |
| [Y] | |
| AGAGAGGGTCTTTGATTCTTAGGATCCCCCTTTTCTTTTCCATCCATTAATTCCTA | |
| ACTACACTGTTCTTCTCTAACCATGTAACTATTTCTCAATATCCATTTGTCACATG | |
| TAAAATATTCTCAAGACCACTCCTAGCCTTGTATACCTGAGACCTGTCTCCCATAC | |
| CAACACCATCACTTAATTAAGAAACAATGGCACTAAAGCTTTGCTTACAAATCTGT | |
| GAAACAAAGGTCATCCCACCTGCCTACCTTCCCACTTCACCTTACTAATAGGAGGT | |
| TTAAAGGAGATATGTGCTTAAGTACACCAAAGAACCAGAGGTACCAACAGGGTTAA | |
| GATACGCCTTGAATCCAAGAAAATCCCCTGAAGCAGCATGTCAATACTGAGTAACA | |
| CAACCATTCCCTAGGCTATCACCTTTTTTTTTTTTTTTTTTTTTTTTTTTGAGACA | |
| GAGTGTCGCTTTGTCACCCAGGCTGGAGTGCAATGGCACGATCTTGGCTCAC | |
| >AF131215-4, chr8, pos 10912771 in NCBI build 33 | |
| Primer pair: | |
| F: AGCCACACAGGTCACAGATTT | (SEQ ID NO: 174) |
| R: TTCTGACATTCTTAATGGGCTTT | (SEQ ID NO: 175) |
| length: 248 | |
| Amplimer: | |
| AGCCACACAGGTCACAGATTTTGGCTTTTTAAGAAGAAACAAGAGCCCTCATGCAG | (SEQ ID NO: 176) |
| ACCCCTGGTACAGTCTCAACTGGTGGAGATACTATGTAAAGGAGCTTTTAAATTAT | |
| TAAATAGCCTCtaaataaatacatattttatatatatatatacacacatacacaca | |
| cacacacacacacacacacacacacTTATATTACATTTATTAGTAACCTAATTTTT | |
| AAAAGCCCATTAAGAATGTCAGAA | |
| >SG08S508, chr8, pos 10914173 in NCBI build 33 | |
| CCTGTGATGGGATGGCACCCTGTCCAGGACTGGTGCCCGCCGTGTGCCCTGAGCTC | (SEQ ID NO: 177) |
| CTAGGATAGGCTCTGACCACCTGTGACCAGGTGGAATAAGTGGGTAAGAATTATCT | |
| CACGTTGCATTAATCTTTCTTAAATATAGGTATGGCTCACATTTATTTCAACGTTT | |
| AATGCTAGAAGTGTTTTGGTCTTTACTTAAAAGTTTGGTGGTGTTTTTGTGAACAG | |
| AAATATGCCACAGAAACTTAATCTTGTTTGTATCAATTAGCCTATGGGAAAACTGG | |
| TTTCCATATACGTAGTTTCACTTCAAATTGCAGTTTCTAAGAACTCACTGATGACA | |
| GTGAAGATTTACTGTATGGGGTTTTAGAGTAAATTTCTAAATGTACGTACAATTTT | |
| TCACATTTTTTAAATATCTATTTGGTGATCTATATATTCAACAGATGAGAATCAGT | |
| AGTCACTTTTAGGGATAGTTTCCTGGGAGATGGCACCCAATAAAGTCTCCAA | |
| [Y] | |
| GATGGGACATGATTTTGAAAGAGTACATTAGCTGTGCTCACAAACCAAGATCCAAT | |
| CTTTCCTCAACCAGATGAACTTTTCCTTAAGACCTGAAACACTGATGAGTCTTGGG | |
| CACATGGCTACAATACTTTTCATTGAGTCCCTGAAGGCCATTTTTACCTCAATGAA | |
| ATATCATCTAAAGAAAAATTATTTAAAACTCCAGTTGTATAATTTCAAGATAGTTT | |
| AGTGTATTTAGTATGACTCACTCTTCATTAAACTTCACAACTATTTTTAAAAGCTA | |
| ATTTAAATAGTTACCTGTTTGAGCTGATCGATGGAAACAGGGCTTGGGCTATTTCT | |
| GTACCACCCTCAGACTAAGAATGCTTTTTATATTTTTCGAGGGGACTGTGCATCAG | |
| AGGCCTTCTGTGGCTACACATCTTAAAATACTTCTTTACAGAAAAAGCTTGCCAAG | |
| TCCCGAATCAAAACAGAAATCAAAGTTTTAAAGGGAAATCGTCTCTTGTACT | |
| >SG08S73, chr8, pos 10914271 in NCBI build 33, alias | |
| name, rs2409727 | |
| ggtaagaattatctcacgttgcattaatctttcttaaatataggtatggctcacat | (SEQ ID NO: 178) |
| ttatttcaacgtttaatgctagaagtgttttggtctttacttaaaagtttggtggt | |
| gtttttgtgaacagaaatatgccacagaaacttaatcttgtttgtatcaattagcc | |
| tatgggaaaactggtttccatatacgtagtttcacttcaaattgcagtttctaaga | |
| actcactgatgacagtgaagatttactgTATGGGGTTTTAGAGTAAATTTCTAAAT | |
| GTACGTACAATTTTTCACATTTTTTAAATATCTATTTGGTGATCTATATATTCAAC | |
| AGATGAGAATCAGTAGTCACTTTTAGGGATAGTTTCCTGGGAGATGGCACCCAATA | |
| AAGTCTCCAATGATGGGACATGATTTTGAAAGAGTACATTAGCTGTGCTCACAAAC | |
| CAAGATCCAATCTTTCCTCAACCAGATGAACTTTTCCTTAAGACCTGAAACA | |
| [Y] | |
| TGATGAGTCTTGGGCACATGGCTACAATACTTTTCATTGAGTCCCTGAAGGCCATT | |
| TTTACCTCAATGAAATATCATCTAAAGAAAAATTATTTAAAACTCCAGTTGTATAA | |
| TTTCAAGATAGTTTAGTGTATTTAGTATGACTCACTCTTCATTAAACTTCACAACT | |
| ATTTTTAAAAGCTAATTTAAATAGTTACCTGTTTGAGCTGATCGATGGAAACAGGG | |
| CTTgggctatttctgtaccaccctcagactaagaatgctttttatatttttcgagg | |
| gGACTGtgcatcagaggccttctgtggctacacatcttaaaatacttctttacaga | |
| aaaagcttgccaagtcccgAATCAAAACAGAAATCAAAGTTTTAAAGGGAAATCGT | |
| CTCTTGTACTCTGCAATCAATAGCATTTTTTTTTATACATACACACACATAGACAC | |
| ATTCATGCCCCCCCATCCCCATCCCACTTTAATCTGGAAGGTACCTGATCTA | |
| >DG8S118, chr8, pos 10923128 in NCBI build 33 | |
| Primer pair: | |
| F: TGCAGACAGCACGTTGTAAA | (SEQ ID NO: 179) |
| R: AGGCTGGTGCTCCTGAAAT | (SEQ ID NO: 180) |
| length: 263 | |
| Amplimer: | |
| AATCTTTCCATCCCACAGAATCTTTCCAACATTACAGAATCTATCCANTTGCATAA | (SEQ ID NO: 181) |
| GCCTGACTAGGCAATTGACCTTATGAATAAGTCTATAGTATCAAATGATGTTGAAG | |
| ACAG | |
| >DG8S161, chr8, pos 10925492 in NCBI build 33 | |
| Primer pair: | |
| F: CAGCCCAGCAACATTCACT | (SEQ ID NO: 182) |
| R: GTGGTAGAGGGTTGCCTTCA | (SEQ ID NO: 183) |
| length: 174 | |
| Amplimer: | |
| CAGCCCAGCAACATTCACTGCAGATTTTGTAGAGAGCTGCATATCCAAATTCCACC | (SEQ ID NO: 184) |
| AGTCTCAAATCAGAAAACAACGCTAAAACAGAGCTGTAGACCGCTCAACTGGATGG | |
| TGCCATTATAAAATGCAAAATGCCTTTTCCTTTTTACTCTCCTGAAGGCAACCCTC | |
| TACCAC | |
| >DG8S127, chr8, pos 10926764 in NCBI build 33 | |
| Primer pair: | |
| F: GCAAACAACATGGCTAGCAG | (SEQ ID NO: 185) |
| R: TGTTTCTTGGCAAAGTGGAA | (SEQ ID NO: 186) |
| length: 403 | |
| Amplimer: | |
| GCAAACAACATGGCTAGCAGGTATTAAAACAGCAGACCATGTTCCTGCAGTATTTC | (SEQ ID NO: 187) |
| AAGCAAAACCATCTAACTGGGaaaaaaaatttttttaataaaatCCTTCCTCAGTA | |
| AATACTGCTTTTGAAGTATAGCTATGTTAGAAGAAATAACTTACTAAAATTAGCAT | |
| GTCTTTTAATAAGTTAACTTTAGGAAATATTTAGAGATATATTCTAATCTTGAAAA | |
| AAGATGTAAAAAAAAAACTAGACAGTAAAGTCACAGGCACTTTATATCAATGCAGA | |
| GGAAAGTTAAGATCAGAAAAAAAAAAAATACTACCCTACATACAACTACAAAAGCT | |
| AAATTGACATTTTAAATGTACTTTTCAGTTTGCCCTAAAATCTGGACTTCCACTTT | |
| GCCAAGAAACA | |
| >SG08S520, chr8, pos 10931667 in NCBI build 33 | |
| TTTGAAGCCAGATAGCCAAAATAGGGCAAGCTACATGGTTACAGTTGTTCCTGATC | (SEQ ID NO: 188) |
| AGATGAAATGAACATTTTACAGTTAAAAAAAAGAATGAGGGGGAAAAAAATCCCTG | |
| AATTTTCTCATTGACTTCCCTAGATTTTTGAACTCATTTTTGTGATTCTGTCTACT | |
| TCTCCATTCACTAAAGTCTTCTAATAATGCCAATAACTGTCTTTAGAATGTTAAGA | |
| GTACAAATTAGGTAATATTTATATGGCTGGAGGTTCTATGGCAGAAAGGTGCGTTT | |
| GACAACTTCAATAGTTACTTTGATACTATTGAATACTATGGCACCTATGAGTTTTG | |
| GGAGTGGCAGGGTAGATGGGGATACTACATTTTAGGACACAGCTTTTCATGAGTAT | |
| ATATGCCAGTGTGAAATCTCTGAAGACTTTAGAAAAATTACTAATAGTGAATTTTT | |
| ACTCCCATACATTGGGAAGAGGGGAGTGATTCCAAAATCAACTTTTAGAAAC | |
| [M] | |
| AGCCATATAACTGTATCCATGTATTTCATGCTATGATTTAAGCCTCATACTCCCTA | |
| TGGTATGTAAAACTCATACTCATATGTAAGCCTCATACTCCCTATGGTAGTAAAAC | |
| TTAAGGCCAGCAGGTAAAGATTATTTCTGCATATAGATGGGATTCTGTTTCTTTGC | |
| TGAATTTGAATGAATAACACCTTACATGGCATAAATATAGAGTAGGATTGCCCAGG | |
| TATGAACCCCAATTTCACTAAAATAGTAACATGAATAATGTGAGCAAGATTACCTC | |
| TTCAAATCTCAGTTTTCACCTTGATATAATAGAAATAACAACAGTGACTTTTCTGA | |
| AAAGTTGCTGGGCAGAGTAAAGGTGGTAATCCTTTCAAGGATCTCAATATGATACC | |
| TGATAGGCAGCTAAGCACTAGAGAGTAACTGCTATTATTATTACTGTTGTTATTAT | |
| TATGTTTGCATAATACTGACATGTTTCTACTTAAATTCTATCGCTGAGTGTA | |
| >DG8S153, chr8, pos 10938731 in NCBI build 33 | |
| Primer pair: | |
| F: AAAGTTGCATAGCTTCCTCAGTTT | (SEQ ID NO: 189) |
| R: TTAAACCACTGGCTTTCCTG | (SEQ ID NO: 190) |
| length: 176 | |
| Amplimer: | |
| AAAGTTGCATAGCTTCCTCAGTTTTAATGTTTGAAATGTCTTTTTCTTAATGGCAG | (SEQ ID NO: 191) |
| GAATACTGGGCTTAGAAGTtgtattagttagggctcttccgagaaacagaaTgaga | |
| gagagagagagagagagagagagagagagagagaCCTATCACTGCAGGAAAGCCAG | |
| TGGTTTAA | |
| >SG08S510, chr8, pos 10990033 in NCBI build 33 | |
| TATTTGAGAAAGGGTGTTGTTGGATCAGTCGGACTTCCTGTCCTGATTGCAGTAGT | (SEQ ID NO: 192) |
| GGGTGGGGTGAATTTCCTTCTAGCAGCGTGGAAAAGGGGCATGGGAATCAATGCAG | |
| GTGGAACAGTGGTTCCTGATGTGACGTAGGCAACCATTGGACATTGGGCTTTTTTA | |
| CATCCTCAGATTCAAGAGCCTCTTGAAAATGTCTCATTTTGATCATATCGAGTTCT | |
| GTCTGTGAAAGCGATGGCAAGTCTGGGTTAACTAGTGAACTAGTCTAGTCGAGTTA | |
| GCTTAAGACTCTTTCTTATAATGCATGGACATGTAAAAATCAGGAATTTCTTGGTG | |
| AAAAAATTTGTTTCCTTAGAACCAGAACAACCCATAATGCAAACGCATAAAAAAGA | |
| TTTGCAAATTGATGTCCTCAGTCTCTCTAGATACATTTCAGGTGTTCAAGATCCAC | |
| GTATAGCTAGTGGTGACCATATTGACATCATGGAAATACCTACTGGGCCGTG | |
| [M] | |
| TGGTTTACACCATACTCTCTGAAACACCGCTTAGGCATTTACCCCATGATTCTGTG | |
| TATGACTGCTTTTAGTAGCTGCTGCTGCTATTTGCTACCACGAAGGCCGCCTCCTC | |
| CTCCCGTGGTCGGTAGGTAAGTTTAGGTTCTTGATCTCACCACACAAAAGAATTTG | |
| AGAGTGACTCCAAAGGAAGAGTAGCCAAAGAAGCTTATTGTAAAGCGAAAGTACCC | |
| TCTGAGAGGCTGAGTGGGCTGCTTAAAGGGAGAGACAGCAACTAGTGCCTTCAGAG | |
| GAATTCCTTTTGCGGGAATTGTTCGTATATATTCATAAAATACTGGTGAGGTCAAG | |
| TACGTAAAGACAGACCTGCGGTTGACACATGCGCTCAGCATCTGCATGCTGTAACA | |
| TGCAATGCATGTATCATTAGCATATAAAATCTCCGCCTAGGGGTGTGTTTTTTTAC | |
| TATTAAAATGAAGAAAAGGTTACTATGAGCTAAACCTTGAGCCTAGCTGCAC | |
| >DG8S242, chr8, pos 11023805 in NCBI build 33 | |
| Primer pair: | |
| F: CTGGAATGGAGGAATGCTTG | (SEQ ID NO: 193) |
| R: TCCACAAAGCCATTGGAAA | (SEQ ID NO: 194) |
| length: 304 | |
| Amplimer: | |
| CTGGAATGGAGGAATGCTTGAATATAGCCAGTTCCATTGAGGTAAGTATTTTGGAA | (SEQ ID NO: 195) |
| GCAAAATCTAATGAAACATAATTTTATATTATGACTCAGTGTAGCTCTTCCATTTC | |
| TTCATTAGATAATTTAGTCATGTTCTCTGACTCAAATACTGAAGACTGATAGGAAA | |
| AGCCTCACCCTGGTTCATCGTCATATGAGTGTAATGGAACTTTCTTGACTTCCAGC | |
| AGTGTCTGGTGTTACTCACGTTATATGAGTAGCTCAATTCCATGAGTTGCTTGGAA | |
| TTCCATTTCCAATGGCTTTGTGGA | |
| >SG08S90, chr8, pos 11028406 in NCBI build 33, alias | |
| name, rs2736387 | |
| tgccagacactgttctaagccttttacacacattatctctcttaatgcttcaacaa | (SEQ ID NO: 196) |
| cactatgaagtaggtatgttatttcccccattttacagttgaggaaactgaggcat | |
| agagtggttacatgactttcctactgcactgctaggatttggaatttcagtccggc | |
| attctcattcccatctgactgtagacctctaggctgtaTCATCCTTTTTTACAGTT | |
| ACTAACCCACCCTGATTTCAAATAATTTACATAAGTTTATTTAGGTAATAACTGGA | |
| TTTTGAGCCAAGACCTTACTGACTAGCCAAAAACTGATCCCCAGAAATACTTAGAC | |
| CTTTTTATTAAGCTTTATTAATTGATGTCGAGGTGTTATTTCATTTTTCTTCTAGA | |
| ATTGGTATGCACATTTTTTTGTTCTTTTTTTCATCCCGTCAACACTTTTGAGTGTG | |
| TGTTATGTGGCAGATGCCTTTGTTAGATACTAGAAGCAAAGAAATCAGCTTC | |
| [M] | |
| GTAAGACTAAAATTGTATCTGGTGATAAACACAATGTTAGAGAAACATTCTGGGTG | |
| CCCATCATTATTAGACCATGTTTGCTTAATACTAATTTGTCAGTTAGAATATGTTT | |
| CCAGTTGTGGATGTTTCTTTTTTGTCTTTCTTTTCTTTTTGCCCCCAGGCATTGTC | |
| TACTCTGGACTCCATCACTCTGATGTACCCTTTCTTTTACCGTCCTATGTTTGAAG | |
| TGATAGAAGATGGCTGGCATTCCTTCCTTCCTGAGCAAGAATTTGAACTCTATTCT | |
| TCAGCTGTGAGTTAACTTTTGAGAACTGTGGATTATGAGAAGTAACCCAATACCTT | |
| ATTTGACTTGTGAAAATGATCACTTCTTTTGAAGAGTAATAAGGTGAAGTTGACTT | |
| ATCCATTCCTAATcttaatatatttaaaaggattgaagccatgcagagtatgatct | |
| ctgatcacaaaggaattagattaataatcagtaatactaagatatctaggaa | |
| >SG08S32, chr8, pos 11048161 in NCBI build 33, alias | |
| name, rs2251473 | |
| AATTAGAAAGTGGTTATCAAACAATGTAAATAATGAAGACCCTGGGGGTCTTTCCA | (SEQ ID NO: 197) |
| GACATTCATATTTGTAAGCTATCCTGGTTGTTTCTGCACAACAAGCCCTTTCTTAA | |
| AGAAACTAGAAAAATAAATAGGACATAAATGTCAAAAAGTGTATAATTTTTATGTT | |
| TATATTATAGGCTTCTCAGAAACAAAAAGGTTAGAAAGTTTTTTTATGCTTAGCTA | |
| TTTTTAATTAAAATAGAATCCCAAATATAACAAAGGACTTTTGTGTACAGTAATGT | |
| TCTCTGGGTTAAGGTTTAACACCAAACCTGATGTGACCAGATTCTGTTTTTATCCT | |
| CCTGCCAGCTTCTTGGAAGCCTGTAAAATACTCTTTGTTTTGTTGTTGTTGAGAGT | |
| TCTAATGCCGATTGAGCTTTTTGACAAATCTATTGATTTTTCAACACTTTGTTTCT | |
| CTACCAAAAGTCTTGTATTCTATCTTCTTTCATACTGAGAAGAAATTGTCCT | |
| [M] | |
| GTAAGAGGAGCACTCAATAATGGTTGTTATAAATTAATTACTTTAATGGCAGTGTT | |
| CTTTCTTGATCAGATGTAAGTTGAAGCTACAGCAGAAGACGATGTCTTTGTGGTCC | |
| TGGGTTAATCAGCCCAGTGAGCTGAGTAAATTCACCAATCCCCTCTTTGAAGCCAA | |
| CAACCTTGTCATCTGGCCTTCAGTTGCTCCGCAGAGTCTTCCACTGTGGGAAGGTA | |
| AACCACGCATCCTTTGCAAACTTCTTAACGGTCAGGTGTGCATGCGGCTGCCTGTG | |
| AGTGTGTGCTGTTGGTGATGTATGAAGATGGTGAGCTGGACGTGGCCCTCAGACCT | |
| GTGTGAATTGTCATTCTCAGTGTGGGCATGTTTTTCTCTTTCAAATCAGTTATCTA | |
| GCCACACTTTTTTTTTTTTTCAGTTACCATTGAGAAATTAACAGTGTTTCTTTACA | |
| TTGCTGTTTATGTTGGATATTTTTCTAGATAAGAAAGTACCTTACTCTTTGC | |
| >DG8S156, chr8, pos 11054915 in NCBI build 33 | |
| Primer pair: | |
| F: GGACCAGAAATGGGCAATAG | (SEQ ID NO: 198) |
| R: CTCTTCAGTTCTGAGGGTTGC | (SEQ ID NO: 199) |
| length: 153 | |
| Amplimer: | |
| GGACCAGAAATGGGCAATAGTTACAATAGTTGATCCTCTGTTCTGGAAGCTTTGAA | (SEQ ID NO: 200) |
| ATTTATCAGAGAATGAAGTCATTCAGTACATCTGATAAAGTTttgttgttgttgtt | |
| gttgttgttgttTTAATTGGGCAACCCTCAGAACTGAAGAG | |
| >DG8S147, chr8, pos 11071336 in NCBI build 33 | |
| Primer pair: | |
| F: AACGGAGAAAGAGGGTGTCC | (SEQ ID NO: 201) |
| R: CCCTTCCAGTTGCAGGAGTA | (SEQ ID NO: 202) |
| length: 382 | |
| Amplimer: | |
| AACGGAGAAAGAGGGTGTCCATAGCCTACAGAACTTTCTCTCAGAACTTCTAGGTc | (SEQ ID NO: 203) |
| agtgctgttctttgggaatctaatatgagccacatatataatttaaaaatttctat | |
| taatcacacaagagtaaaaaaaacaggtgaaatgaattgtaaNtgttttatttaac | |
| ttaccttactaaaaatattttccatttaacatacaatatgaaattcattaacggat | |
| agtcacatttttaaacgccatatcttcaaaatctggtgtttgacagcacatttcag | |
| ttcaaactagctacgttgcaaggatttaatagccctatgtggctagtgactattgt | |
| atggaacaTTATCGTTCTAGACCCTCTACTCCTGCAACTGGAAGGG | |
| >SG08S511, chr8, pos 11077298 in NCBI build 33 | |
| TCTAGCTTTCAGATCATCCCCACGTAAAGTTCAGACTTTACCAGCCCAGAGAGTTT | (SEQ ID NO: 204) |
| AAAAAAAAAAAAAGAGAGAGAGAGAAAGCGAATGTGGATTGAGCCTTTACACTGAC | |
| CGCGCAGTTTGCACAGTGCTTTTCATAGATTGACTGCTTTTATTAAACGCTCTCAA | |
| CAGTCTATTAGGATGGCATGGTGATTGCCCCCTTTCTGAGGACGCGGAAACTTGAG | |
| ATTTGGCGAGGCAAGAAGCCAGGCGCACACAGCTAGGCGGGCCGCGGGCCGCGACC | |
| CCCTGGCTGGTCCGTGCTCTCCCCCTGGGGAGGGGTGCAGGCTGCCAGGAAAGGTG | |
| CCCCCTGCGTGGCCCTGGGGGTGTTTCTTCCTCTTTGTCTCTTCTTAGGCATCTGA | |
| TCTCATCTCTTAAGTGGGAAGAGTCGGGGTGGTGGAAGTAGAGGGTATGGGACACG | |
| GTGGACCTACCTCACTTGGTAGTTAGTAACTGCCTCACCTTGGGCGGGTCAG | |
| [Y] | |
| GGATTCTGAACAATGGGGAAAAGGTCCCAGCTTCAGGGTTGCTGTGAGGGTTTAAG | |
| AAGAGTTCAGGAAAGCAGATGCTTCACCAACGCTCCGTAGTTACCAGGCGCCTGAT | |
| TTTTCCTTGGATCATTACTATTAAGAGGATGCATTGGTGATGATGATGATGTAATG | |
| AGTCAGAGGTTTTAAAGCCCAGACTGCCTTGAAAATGCGTCTGGTAAACCTTCTTG | |
| CTCCTTAAAGCAGAATAAGATTGGAGTGGGGGAACGCAGTGAAAATGAAGGTGGGC | |
| ATGGACATATAAGTATTAAGTTAGAAGTGGGGAGGGGGCAGGGGGCATTGGCGCCA | |
| GGAAGTTGTAAACTGGGCAATTATCACCCAGTCCAGAGCAGGGAAGGCCCGTTGTG | |
| AGGGGCTAGGCATGAAGGTACCAGCAGCGTACATGCTCCTGCAGACCCCTGAGGCT | |
| GGAAGGAAGGAGCGGGCAGTGGGAGAGTAATAGGTTTAAGCACGTTTGCAAG | |
| >SG08S512, chr8, pos 11077399 in NCBI build 33 | |
| TTTACACTGACCGCGCAGTTTGCACAGTGCTTTTCATAGATTGACTGCTTTTATTA | (SEQ ID NO: 205) |
| AACGCTCTCAACAGTCTATTAGGATGGCATGGTGATTGCCCCCTTTCTGAGGACGC | |
| GGAAACTTGAGATTTGGCGAGGCAAGAAGCCAGGCGCACACAGCTAGGCGGGCCGC | |
| GGGCCGCGACCCCCTGGCTGGTCCGTGCTCTCCCCCTGGGGAGGGGTGCAGGCTGC | |
| CAGGAAAGGTGCCCCCTGCGTGGCCCTGGGGGTGTTTCTTCCTCTTTGTCTCTTCT | |
| TAGGCATCTGATCTCATCTCTTAAGTGGGAAGAGTCGGGGTGGTGGAAGTAGAGGG | |
| TATGGGACACGGTGGACCTACCTCACTTGGTAGTTAGTAACTGCCTCACCTTGGGC | |
| GGGTCAGTGGATTCTGAACAATGGGGAAAAGGTCCCAGCTTCAGGGTTGCTGTGAG | |
| GGTTTAAGAAGAGTTCAGGAAAGCAGATGCTTCACCAACGCTCCGTAGTTAC | |
| [S] | |
| AGGCGCCTGATTTTTCCTTGGATCATTACTATTAAGAGGATGCATTGGTGATGATG | |
| ATGATGTAATGAGTCAGAGGTTTTAAAGCCCAGACTGCCTTGAAAATGCGTCTGGT | |
| AAACCTTCTTGCTCCTTAAAGCAGAATAAGATTGGAGTGGGGGAACGCAGTGAAAA | |
| TGAAGGTGGGCATGGACATATAAGTATTAAGTTAGAAGTGGGGAGGGGGCAGGGGG | |
| CATTGGCGCCAGGAAGTTGTAAACTGGGCAATTATCACCCAGTCCAGAGCAGGGAA | |
| GGCCCGTTGTGAGGGGCTAGGCATGAAGGTACCAGCAGCGTACATGCTCCTGCAGA | |
| CCCCTGAGGCTGGAAGGAAGGAGCGGGCAGTGGGAGAGTAATAGGTTTAAGCACGT | |
| TTGCAAGTGGAGGCGGAGAGAGGACAAGGGCTGGGGGGGTTGGAGTTTGCTGGGTC | |
| TCTGGGGGCAATATTGATCTATGTTAGGCGAGTTTTCTCACTCTTCAGATAC | |
| >SG08S27, chr8, pos 11086652 in NCBI build 33, alias | |
| name, rs2249804 | |
| TGGTTTCTCCCTGCCTCTTTTCCCTTTCATATCCCAGTCCACTTCTAATGGAGGAT | (SEQ ID NO: 206) |
| GGGATTCTGCCTCATGTCACCAGAGGTGGATATGAATCTGTTCATACTGGTTTTGA | |
| ATGATTTTGTCACCCATAGCAGATAAGCTTCAAAGTTCATGAAAATAATGAAGGCC | |
| AAGATTGAGTTCCTGCCCCAAGAAATTCCAGACCTGTGTCTGGCTTTCATGAGATT | |
| TTTCTCTTCTAATGCCCTTGCTTCTCCTCTTTCTCGGAACCACTCCATGCTGGTAA | |
| GTGTTGTCTCTGAAACGAATGTTACCTGTATTGGTCTCTGTCCTAGCATGGGGGAG | |
| ATCATTGCATTTCTAAGCGCTGCACCACGTTCCTGGGAAGATTGGAAGTAAGCAGC | |
| AGTTATATCAGTGCAACCTAGGACTTACGTAGTTAGCTAAGACTGAAAACTAGTCT | |
| CACTCAGTTATTACATTCTGGGAATAATTGAACTGTTTAGATTTGCATTAAA | |
| [S] | |
| CTTCACTTTTTTTTCTTCTTCATCTAGGGGCTCTTGGCCAGCTGGGAGTGGGGCTT | |
| GCTAATCTTTTGAGGTAAGAGCCCTAAAAACTTGAAATTTAAAATCTGAGTTGTTA | |
| AGTATATGGAGCTCATTGGGATGCCTTTTAAACTTCTTTTCTCTCTCCTCTTGCTC | |
| CTTACCATTGTTAAGatatatctaaataactgctatatatagctatagatatagat | |
| atatagagatatagatatagatacagattttttttttttgagttggagcctcagtc | |
| tgtcacccaggctgtagtgcagtggtgcaatcccggctcactataacctccacctc | |
| ctgggttcaagtgattctcctgcctcagcctcccgagtagctgggactacaggcac | |
| ataccaccacgcctggctaattttttttatttttggtagagatgggctctcgctat | |
| gttgcccaggctggtcttctaactcctggcctcaagtgatctgcccgcctca | |
| >SG08S26, chr8, pos 11090369 in NCBI build 33, alias | |
| name, rs2246606 | |
| AAAGGTCCATTTAGTTCACAACCCTTTTCACGTTCGTGGTTTCAATTTATGTTCCT | (SEQ ID NO: 207) |
| TGCAGGTCCATTCATTTATTCTGATATCTTGGATTACAAGAATCTTCGGGAGATCG | |
| TGGTAAACAACCGCATCACCTGGCTGTTTCATTACAGCGCTTTGCTCAGCGCCTTT | |
| GGAGAAGCAAATGTTTCCCTGGCGAGAGCAGTGAATATAACTGGTAAGCATCTGGC | |
| TCTGGCTGGATGTGATTTATTTGCCAGTTTTTCTAGTTCTTTAAGAAGAGATGTTT | |
| TCAGATTCTGATAGTGTCTGTTCATTTCAGGCCTGCATAACATCCTGGATGTCGCT | |
| GCGGAACACAATCTGCAATTGTTTGTGCCTAGCACGATTGGGGCTTTTGGACCCAC | |
| CTCTCCCCGGAACCCAACCCCCGATCTCTGTATTCAGAGACCCAGGACCATCTATG | |
| GGGTGTCCAAGGTCCACGCGGAGCTCATGGGAGAAGTAAGCATCACTCAGCT | |
| [R] | |
| GATTGCTGAATGTGCCCTGGCTGTCACGATTTGCTGTTTGCTTTCTCATTCGTTTT | |
| GCCTCCAAGGCCTGGTGATTCATCCCTGGAGGAACTTTACCTCTTCTTGGATCCCA | |
| GCCCCAGAGTCGCTTACTTAACTCACTGGGTTTGCCATGTAGCAGGTGTCTCCAGC | |
| TCCTGAAACCTCCTCAGCCATATGGGAACACTCAGCACTTCCTGGGTGCCCCGTGC | |
| CCAGCCCCGATCTCTTCATTTGCTGCTTGTCTTGTACTCCACCATTCTTTCTGGCT | |
| CCTAGTATTGGTAGCCATTGGTAGTAACTCTAAAACCTCAAACATCTTGGGtttgt | |
| tttgtttgtttgtttgttttatgagacagaatcttgctctgtcacccaggctggag | |
| tgtggtggcgtgatctcagctcatagcagcctccgcctcctgggttcaagggatcc | |
| tcatgcctcagcctccgaagtagctgggattataggcacgtgccaccacacc | |
| >D8S265, chr8, pos 11150773 in NCBI build 33 | |
| Primer pair: | |
| F: ACCTCTTTCCAGATAAGCCC | (SEQ ID NO: 208) |
| R: CCAATGGTTTCGGTTACTGT | (SEQ ID NO: 209) |
| length: 213 | |
| Amplimer: | |
| ACCTCTTTCCAGATAAGCCCTTGAGGTCTCGGGCTGACCTACACACACACACACAC | (SEQ ID NO: 210) |
| ACACACACACCCCCCCCCACACACACACACGACAGAGAACATGCCATAAACATCCT | |
| TGAACCCATGCAGGAAAGCCCATCCCATATTCTGAAAAAATGCCAAATTAGGTTTT | |
| TCTTTCTTTTTGGAAATCAGTCATTACAGTAACCGAAACCATTGG | |
| >D8S1695, chr8, pos 11220756 in NCBI build 33 | |
| Primer pair: | |
| F: AACCCAGCATCCTACAAAG | (SEQ ID NO: 211) |
| R: CATCTGGAACCCATGAG | (SEQ ID NO: 212) |
| length: 273 | |
| Amplimer: | |
| AACCCAGCATCCTACAAAGAAAATACATGGTCTGTCTACCCAAGGTTAGAGTGGGA | (SEQ ID NO: 213) |
| GGGGATGTGAGAGTTTGCAGGGAGGTGTGCTGGCCCTTATGTGATCTGTGATAAGA | |
| CATCACCTTTATGCCCACCCCAACAGACAGAGGTTGGAAAATAACAATACCAGaca | |
| cacacacacacacacacacacacacacacacacacacacacacacGATTCCAGCAG | |
| CCACTCAGAAAGAAAACAAGGAAATGACTTTGCTCATGGGTTCCAGATG | |
| >SG08S46, chr8, pos 11234300 in NCBI build 33, alias | |
| name, rs2280804 | |
| AGTATCATCCTTCACAAAGTTCTTTCTATTCTTTCTACTGTACAAAGTTTTCTGTT | (SEQ ID NO: 214) |
| GTCAAATAGCAAGAGATCTCTGTTTTCTACTTGGAATGGGCCTGGAGAAGGGAGAC | |
| AGCACCCGCTCCCTCCACCCCTTGTCCCTGAGCACAGCATGGTGACCTGCCAAGCC | |
| AGAGGGTGACCTGGACACTCATAACTCAATGCAGGGCCAACTGTAGCCTCTGGCCG | |
| GTGTCCCTGAGTGAGGGCAAAGTTGTAATAACACTTGTTCTCTCCTTTCTCCAATT | |
| TGCTCCCAAGCTCCATTGCTTTCGTTCAGGCCCTCCCCCTTCTAGACTGGGCAGTT | |
| CCGCATCCTTGGAGCTCATTTCTCTGTCTTCAGAATCTGATGCTCCAATTCATCCC | |
| ATGTGTGGCTGCCAAGGTCTTTCTAAAACTCAAATGTGGCCCTATCACCGCACAGG | |
| GTAAAGCCACCATAAACTCCTCTGTGTTTGAGAACAAGGGCCAAGTCTCCCA | |
| [Y] | |
| TGAGGCCTCCAGGGAGTGGACAGTCTGGGTCTCCTTtcttctccaagcacgctggg | |
| cccatctgtcctgtccctgaggactccctggcacacatgacacttcagagcttttg | |
| ccaactccactccctgcctgaaatgcccatctccttcagagagcttctatgtatcc | |
| ttggaggtccagtcctaatgtccctgcctccgataagacctctccccatcttccTC | |
| TCGCCCTGCTCCTGTCCCCGCCAGGCATGACAAATCTCTTCCCACAGTGGGCCCAA | |
| CAGGGAGGCAGATGGTAGAACAGGTTTTGGGCCAGGTGCCAGGTGCACGTGGCTCT | |
| TCATCCTGGTTCCCCACCGCACACCTGGAGAGCTGAGTGCTTTTCCTGAGGTCACG | |
| CAGAAGGTTACCAGCCTGGCTCTGGAGCTGTCTCTTTGCCACATCGTGGGGTGTCT | |
| TTAAGGTGACCTTGAATGTGCTTGAAGCTGTTTTATGTCCTATTTGCAGACC | |
| >DG8S130, chr8, pos 11239181 in NCBI build 33 | |
| Primer pair: | |
| F: CTGGGAATCCGAGATTGAAA | (SEQ ID NO: 215) |
| R: GGCCATAATCAAGGCAGAAT | (SEQ ID NO: 216) |
| length: 288 | |
| Amplimer: | |
| CTGGGAATCCGAGATTGAAATGAAAGAAATCGAAAGATCTTTGCCTACATACAGAG | (SEQ ID NO: 217) |
| GTCCAGTAATGGGATAGGGAATATATTATCCccgggatagcgccactgtactccag | |
| ccaggatgacagagactccatctcaaataaataaataaataaataaataaataaat | |
| aaataaataaatacataaataaaGTGCCTCTTTGTTAAGGCAGTTGCTTCTATTTC | |
| TACTTTTTTAACCAAAGCTAATTGCTAATGTGTTAAAGTACGAGATTCTGCCTTGA | |
| TTATGGCC | |
| >SG08S35, chr8, pos 11253693 in NCBI build 33, alias | |
| name, rs2252797 | |
| aagatatgaggaaagagaaagggcatgagcaaaggacatttttgcagcatgtttat | (SEQ ID NO: 218) |
| gatcttgagaaaatggaaacagctggggtgtgcggcagaagaagtggggaaaatga | |
| caacggttcattaaacctcacgatcagatgctgacagcccctcacaggttgctgca | |
| gacaaaacagggaacgacaggaaaaagatgaccgtgatacgctctgctaaaagcag | |
| gtcgcaaaacaggatgtagataatgatcccattttgcttttttacaaaaaaaaaaa | |
| aaggccatggaaaattacatatcacgaatgttcagagtggctgtctctggatgatg | |
| gcattggagttaattttatctttcactctattttctgaatttcctatatCAAAAGC | |
| AAATTGATGGTGTGAAGGGGAAAGCATATTTAATGTGATTCCTAAAAGGCTCAGCC | |
| CTCCCTGCATGGATTGAGCACTGAAAGAAGAGGGTTCTGTCACCTCTTTCGT | |
| [S] | |
| CTGACCCTTGCCTTTTCTAATGTTGCTCAGAGGCACACAGACGTATTTGCTTTAAG | |
| TAATTGCTTGTCTGTTTTTAATATCACATTTTGAAAAGGTATTTAGACAACATGAG | |
| TTTATTACTTTCTGTTTAACCCAAATCCTTCAGAGGTACTTAAAGCAAAATGTAAA | |
| GTCCTCTTATCCCTTTGTGaatttcagtccccagaagtctcactgttagtagtttg | |
| atttttaccaaaaatgtccaggtattttcttttcatctgcaaatgtgttaatagac | |
| tcctttttttaaatttcacacaagcaggattatatcatacaaaacattctgcaatt | |
| tactcttttcatgtaacaataatgtatcctgggtatttttctttgccagttcagat | |
| ctcttttatccttttACTAATTTATTTACCTATCTATTCATTTGCTTAACTTGATT | |
| TTATTATTATACAAGTTATCCATGAATATTGTTTTCAAAAATTTAAACAGTC | |
| >SG08S139, chr8, pos 11282021 in NCBI build 33, alias | |
| name, rs936550 | |
| atacacatgcaaacacatacacatgtccacgcatgcacatatacacacacacgcac | (SEQ ID NO: 219) |
| acatatacaTGTGCACATATGCACAGATGCAATGAACACGTGTGCAacacatgtac | |
| acaccttacacgtacatatgcacacacacacacaACTCCAAAGCAAGACCCCTCTG | |
| CTTCTCCGAGCCACAGCAGTGAATGCAAGACAGGGATGGAAGCAGGGGAGTGAGTT | |
| CTACCCTTCGTGGCCTCCGGGGTGTCCTTGAGCCTCTCAAGCCTCAGTTTACTGGT | |
| GTCTATGTGAGGATAGACTAGTTTCACAGCTCAAAGGCAGGCGGTCCTTCAGTGCT | |
| GAGAAATCTTCATCTCAGAGCCAGGCCCTGCCTGCCCAGGGCAGTCCAGACATACC | |
| Acagaggcaggggatccaggttttgtgaaactgaagctgataggatctgaggtcgt | |
| ctttacaaaggacaccaaattgtcagaagccatcagggacggggcctcagag | |
| [M] | |
| agccaggcaagtgaggggtctaaagcaccagcttGGGAAGCGTCACTGCGTGGAGA | |
| GCGGGCTCCTGGGCTCATCGCCCGAGGCACCCGACACAAGTGCAGCCTACAAAATG | |
| GAGAGAAAAGCCCTTGATGAATGAACTCCCTAAGGCCAGGCTCGGGTTCCTTAGAG | |
| ACTGGGGGCACAGCTGCACCCGGGCAGGGTCGGGGAGACAGTTTGCAGCCTCTGGG | |
| CTGAGGCTGGGGTGGGGGTGTGGAGGGGCTGTGGCAACAGCATGGCGTACGCCTCT | |
| GGGTGTCCTTTTGCAAGTAGGTGATGAGAGAGGCACATTGGCTGAGGGAAACTGGA | |
| GGATGGAAGGGGGTTGAGGCAGGGGAACTGACAGGAGAGGAAAGAGCCTTAAGTCA | |
| AACAGGACCGCGGAAAACCAAGCGTCCACAACGAGAACGAGGGGTCCGTGCCTGAC | |
| CCCTGGCGGGGAGGCGTGGTACTGCTCGAGGTAGGCGCGGACTCGGGGAACC | |
| >DG8S170, chr8, pos 11287781 in NCBI build 33 | |
| Primer pair: | |
| F: GCAGCCTCTAACCACATGCT | (SEQ ID NO: 220) |
| R: CTTTGCATGGCTTCCTATGG | (SEQ ID NO: 221) |
| length: 380 | |
| Amplimer: | |
| GCAGCCTCTAACCACATGCTGACCATGCCAATGGCTCTCTAAGcacacatgtacac | (SEQ ID NO: 222) |
| acacacactctcacacacataaaaacacagactcacacacacacggacaaacacaa | |
| acacatacacagactcacacagacacgcaaactcacacacagacagacacacacac | |
| agacacacagactcacacacacaaactcacacagacacacaaatacacaGACTCAG | |
| ACTCAAacacaaactcacacaaacacatttacacaaactcacaaactcacacacac | |
| aaacacacacacaaacacgcaaacttacacacacatgagcagacacacacCCGGCC | |
| CTTCTGGGCTCTTCTTTTCTTACTCCATAGGAAGCCATGCAAAG | |
| >DG8S261, chr8, pos 11303006 in NCBI build 33 | |
| Primer pair: | |
| F: GAATGGGCACATCCATAGGT | (SEQ ID NO: 223) |
| R: CGCCCTTCCTTATCCCTCT | (SEQ ID NO: 224) |
| length: 257 | |
| Amplimer: | |
| GAATGGGCACATCCATAGGTTCTGATTTTGACACATGGCCAAGACTATCAAGTGAG | (SEQ ID NO: 225) |
| GGGAAAGGGTGCAGAAAAACACATACATGCAGCATGATGTacacacacacacacac | |
| acacacaATTTTATGTTCATCACACACATGCATATTTGTGTAAACATGCAGCAAAG | |
| GGATCCCAGTGATACCAACCAAAGAGAGCCCCGTGACCTCCGAGGAGGGAGCGGCT | |
| GGGGCTGTCAGCGCAGAGGGATAAGGAAGGGCG | |
| >D8S1759, chr8, pos 11348674 in NCBI build 33 | |
| Primer pair: | |
| F: GAGACTGACAATCTCCTCGTCTTAT | (SEQ ID NO: 226) |
| R: CTATTGCCTAGCTTAGCACATTTGA | (SEQ ID NO: 227) |
| length: 125 | |
| Amplimer: | |
| GAGACTGACAATCTCCTCGTCTTATCCACGTTCTCACTCCAAATTCATTAAGTTAA | (SEQ ID NO: 228) |
| ATACACACACACACACACACACACACACACACACTAAGACAGTTTCAAATGTGCTA | |
| AGCTAGGCAATAG | |
| >DG8S117, chr8, pos 11350993 in NCBI build 33 | |
| Primer pair: | |
| F: CCTAAGCATTTCTTGGCTTCC | (SEQ ID NO: 229) |
| R: CAGTGAGAGCACCCTACTTTGA | (SEQ ID NO: 230) |
| length: 153 | |
| Amplimer: | |
| CCTAAGCATTTCTTGGCTTCCCCCAGGTGCCCTGTTTTTGAATTAACCTGAGATTA | (SEQ ID NO: 231) |
| TGGCAGACCACAAGGGCTGCATCACACCAAGTTCTCCCCAAGATTTGCCATATTTC | |
| CTCTACCACCAGGTGGGGTTCAAAGTAGGGTGCTCTCACTG | |
| >AC022239-5, chr8, pos 11355629 in NCBI build 33 | |
| Primer pair: | |
| F: TCCACAGCAGGGTTCAATAA | (SEQ ID NO: 232) |
| R: CCCACTCATCCATCTATCCA | (SEQ ID NO: 233) |
| length: 275 | |
| Amplimer: | |
| tccacagcagggttcaataagtgattgctgCTCATTACCTAGCTATACAGGTAGAT | (SEQ ID NO: 234) |
| Atggatggatggatggatggatggaaggatggatgatggatggatggaaggataga | |
| tagatggttggataggtggattgatagatgatggatggatggatggatggatggat | |
| aaatggataaatggatggatggatggatggatatctggatggatggataaatggat | |
| ggatggatggatggaTGAATAGATTATtagatggatagatggatgagtggg | |
| >DG8S181, chr8, pos 11390001 in NCBI build 33 | |
| Primer pair: | |
| F: GGCTCGCTCCAGCTTTATCT | (SEQ ID NO: 235) |
| R: GGGTGATGCATAGCAGACG | (SEQ ID NO: 236) |
| length: 268 | |
| Amplimer: | |
| GGCTCGCTCCAGCTTTATCTGCCTCTTAGGTGTGACCAAATTGTCgtgtgtgcgtg | (SEQ ID NO: 237) |
| tgtgtgtgtgtgtgtgtgtgtgtgtgtgtgtgtgtTGGCTCCAAAGGTTTATTCAC | |
| GAATAGATCCCAAAGAAATGTCACAGAGAAATAGTGACTTGAAGTCCAAAGAGGAA | |
| AAAAAGGGAGGCCGCAGGCACATGATGGATCTGTGCAATAGTCATACGTAAGCCGC | |
| CGTGATGTCCACACCACGGAGACCCCGTCTGCTATGCATCACCC | |
| >SG08S97, chr8, pos 11410417 in NCBI build 33, alias | |
| name, rs2898291 | |
| AAAAACTCCTGGCAGACCCTTCCGGGATCACGCGTGGCTCAACTCGGGGGCCGTAG | (SEQ ID NO: 238) |
| CTACGATCCCCGCGCAGACGCCGGAATccggggcccggtccccgcgcggggtgcgg | |
| cgctcgcggggggggggggggggATGGGGTCGGTCCCTCTCGGGAACGGCTGCTGT | |
| TGTTTCTTTAGATACTGAATATAATTTCTCCCTCCTCCACCCCACTCGCTGTTCTT | |
| AACAATTTTATTTATTGGTTTACTATTGTCTTGTGAACGTTTCTTGTCTCCTCCTT | |
| GCCTTTTTTCATCCCCTTTCTCTCTTCATTTCTCTCTTTTTCCTTAATTCTGTTGC | |
| AAAGTTTCCTTTTCTTGCTTAATCAAAATTCTCCCCGCTTACTTTGTTCTTTGCCC | |
| ACAGCATTCGTTCTTCTTTTCTCCTTGCCTGCCTGTCTTCTTTCCCGCTGTTCTTG | |
| GCCGTGGGCAGACCCGGCTGATGTAAGGACTGCAGCTTTTCCCTGGCATACT | |
| [M] | |
| TGCGCCTTCAGATGTGGTCTGCGTCTGCCTGGGTCTCTTCCCACCTCAATCTGAGA | |
| TCCTTGCCCCTCACAATAAATTCGTTTTTATTCATTCTGATGTTTGTCTACAGAAG | |
| TTACTCGATAAAGATGTTTTGTTTCATGAATCAAAAGGCTTCTTGTCTGTGAATTA | |
| TTTTAATTTCTGGATATTAAACTGCACAGTAGCTATTTTATTTGCCTTTAATAAAT | |
| TTCTTAGGTTTTTACCTCTAACTAATGGCACATTTTAAATAATTTTCCAAGCACTA | |
| GGTGGTGTCTGACAAGATTGATTCACTCAAAAACGATGCAGAATTTCTTAAATGTA | |
| GAATCTTTTAAAACGGTGTCGGATGGCTTCTCCTGCTACATCGTTTATTTGTAGCT | |
| TCCACTAACTCTAAAGATTGAACAGGAAACTGATATGGTAGAAATAGATAACTTTG | |
| CCTTGTTCACTAGCTAAGATTTTATTTGCTTTCTGTTAGATCACAGTAGTGC | |
| >DG8S163, chr8, pos 11458431 in NCBI build 33 | |
| Primer pair: | |
| F: AATTCCTGGATATTCCTACCACTT | (SEQ ID NO: 239) |
| R: GATCCTTACTCCAGCCCACA | (SEQ ID NO: 240) |
| length: 359 | |
| AATTCCTGGATATTCCTACCACTTACTAtttgttgtcgttgtttctattgtttttg | (SEQ ID NO: 241) |
| agagaaggtcttgctccattgcccaggctggagtgcagtggcgtgatcatggctca | |
| ctgcagtctttacctccagggttcaaggaatcctcacacctcagcctcctgagtag | |
| ctggaattactaccatgcccagctaacgtctatattttttggaggtagggttttgc | |
| catgttgcccaggctggtcttgaactcatgagctcaagtgatactcctgcctcagc | |
| ctcccaatgtgctgggattacaggcataagccatcgtgcctggccTCAGTGAGTGG | |
| TTTTGTGGGCTGGAGTAAGGATC | |
| >DG8S221, chr8, pos 11473774 in NCBI build 33 | |
| Primer pair: | |
| F: AGATCACGCTCCAGGGATT | (SEQ ID NO: 242) |
| R: TCCCACACTACACTGATGTAAAGAA | (SEQ ID NO: 243) |
| length: 390 | |
| Amplimer: | |
| AGATCACGCTCCAGGGATTCCTGCGTCCTTTAATAAGATTCTGGGGTGGGCACAGT | (SEQ ID NO: 244) |
| TCTGGGGTggacatggtggctcacgcccataatcccagaactttggaaggctgagg | |
| tgggaggatcgcttgagcttaggagttcaagaccagtctgtacaacacagtgagag | |
| cttgtctctcccaaaaaaaaaaaaaaaaaaaaaaaaattagcaaggcatggcagca | |
| tgcacctgtagtcccagatacttgggaggctgaggtgggaggattgcttgagccta | |
| ggaggttgaggctgcagtgagccgagatcgcagcactgtactccagcctgggggac | |
| agagtgagaccctgtctcacaaaaaGTTTTTCTTTACATCAGTGTAGTGTGGGA | |
| >5G08S76, chr8, pos 11477186 in NCBI build 33, alias | |
| name, rs2409814 | |
| gggaggcagaggttgcagtgagctgagatcgcaccattgcactctagcctgggcaa | (SEQ ID NO: 245) |
| caagagtgaaactccgtctcaaaaagagaaaagaaGTCTCACAAAGGgctgggcac | |
| agtggctcatgcatgtagtctcagcactttgggaggctgaggctggagtatcgctt | |
| gagcccaggggttcaaggctggactgagttatgactgcaccactgtactccagcct | |
| gggtgacagagtgaccctgtctctaataaaaagaataaaataaATACAGTCTTACA | |
| AAGGATACAATAGAACCAAATGCTCAAAACATTAGTGACAATCTGGATTTTCTTTA | |
| TATATTTTGGCACTAATTTTCCTAAGGTAAATATTTATTATATCTTTATGCAAAAG | |
| GAAAAGTAATCTTACTAACTTTGAAAGGGAAAAAGAGAGAGCAAGGTTTGCGTGGA | |
| CCTCAGTGTGAGGTGAGAGGCCTAGGGCTGGAGGCTCTGAATGTGATACCTG | |
| [S] | |
| ACTGAAATCCAGGTGTCCCGCCTCCCAGCCCAGGACGTGGGTGATCACTGCAACTT | |
| TTTCCTCTTCTCGTGCTCAGGGGAACTCTCAGTGTCTGGGATTAGGGAGCAGGGGC | |
| TGAAGTCAGAGTGAGGAAGAGCAAGAGCAGCCCGAGGTGGTCTTCTCTTTCCAAGG | |
| AAAGGGCATTGTTTCTGTGCGCTCTAGATTCTCAGATGTGAGAGCTGGGCATAAAC | |
| AAAGAATTAATCCTCTGTGTCTTTTCTTGTCTGTTCCCCCCAACTCAGTAGATATG | |
| TTTGACGACTTCTCAGAAGGCAGAGAGTGTGTCAACTGTGGGGCTATGTCCACCCC | |
| GCTCTGGAGGCGAGATGGGACGGGTCACTATCTGTGCAACGCCTGCGGCCTCTACC | |
| ACAAGATGAACGGCATCAACCGGCCGCTCATCAAGCCTCAGCGCCGGCTGGTAAGC | |
| ACGTGCCTCGCAGCCTCCTCTGGGCACCTGGCTGCGGAGCTCTCGCCTTGGT | |
| >DG8S292, chr8, pos 11509365 in NCBI build 33 | |
| Primer pair: | |
| F: TTCTGGCCTTAGGAAAGTGC | (SEQ ID NO: 246) |
| R: CCAGACCACAGAAGCTACTCC | (SEQ ID NO: 247) |
| length: 424 | |
| Amplimer: | |
| TTCTGGCCTTAGGAAAGTGCTAGCTGAGCTGAAATCTCATGAATGttaggtcgttt | (SEQ ID NO: 248) |
| gtgtacttcttatcaatgtaatgaagcttttgcacagaaagtctgtttgtttttgt | |
| gacatgtgttgccagtattgtttcaagtctgtcctctgtcctttgattgtgcttat | |
| gatgtctcttggcatttgggattttaaatttttatatcatcaacggtgggtatttt | |
| tcttggttgcttgtaggtttccccttttgctaaaaaaaggccccttctgcccccag | |
| agaaagtcacatgccttctattttctgaagttttataacttGTAAAAATGTTTAGA | |
| AGTGTAGTCTTTATTTGTGTGGCCTGACGTAGGTACCATAGGATGCTATGGGCTGT | |
| AAAAATAACTCGGAGTAGCTTCTGTGGTCTGG | |
| >DG8S333, chr8, pos 11607597 in NCBI build 33 | |
| Primer pair: | |
| F: GCATGTGAAATTGGACTTGTACTC | (SEQ ID NO: 249) |
| R: CACTGCAAGCCTAGAGAAGGA | (SEQ ID NO: 250) |
| length: 292 | |
| Amplimer: | |
| GCATGTGAAATTGGACTTGTACTCCAGAGATATCCATGTTTGTATTCATGTAAAAA | (SEQ ID NO: 251) |
| TAATGTCCTTCTTaattatctgggggtggtggtgtgtgcctttagtgccagctact | |
| tggaaggctgaggcaggagaatcacttggaccaaggaggcagaggttgcagtgagc | |
| tgagatcgcgccattgcactccagcctgggtgacagagagagactctgtcccaaaa | |
| aataaaataaaataaaaataaatacataaaataaaataaaataaaaGTCCTTCTCT | |
| AGGCTTGCAGTG | |
| >D8S1130, chr8, pos 11704969 in NCBI build 33 | |
| Primer pair: | |
| F: GAAGATTTGGCTCTGTTGGA | (SEQ ID NO: 252) |
| R: TGTCTTACTGCTATAGCTTTCATAA | (SEQ ID NO: 253) |
| length: 145 | |
| Amplimer: | |
| Gaagatttggctctgttggagacagactcatagatagatagatagatagatagata | (SEQ ID NO: 254) |
| gatagatagatagatagatagatagatgatagatagatcttatttaaaagtttatt | |
| aacttattatgaagctatagcagtaagaca | |
| >AC068974-2, chr8, pos 11824194 in NCBI build 33 | |
| Primer pair: | |
| F: TGGGAGATTTCAGCCTTTCA | (SEQ ID NO: 255) |
| R: TCAAAGACCAGTGCCAGAGA | (SEQ ID NO: 256) |
| length: 352 | |
| Amplimer: | |
| Tgggagatttcagcctttcaaaaaaatataatgtcttgtactatggattttcctgg | (SEQ ID NO: 257) |
| agtgaaagagaagaaaatctcttttggctcatctctttttactcctacacacacac | |
| acacacacacacacacacacacacacacactctatatgatagattataacagatgt | |
| atctttcaaaagtagaactgaaatttagacctaaaagataatatactttaattgtt | |
| agagaggatatttttcctgttgaagggaacaatattcctatgtgtttaatacacaa | |
| atatatctgtgccAGTACTTGTTACCCCCTGAGACTTCACACACTACTTATATCTC | |
| TGGCACTGGTCTTTGA | |
| >AC068974-2, chr8, POS 11974598 in NCBI build 33 | |
| Primer pair: | |
| F: TGGGAGATTTCAGCCTTTCA | (SEQ ID NO: 258) |
| R: TCAAAGACCAGTGCCAGAGA | (SEQ ID NO: 259) |
| length: 352 | |
| Amplimer: | |
| Tgggagatttcagcctttcaaaaaaatataatgtcttgtactatggattttcctgg | (SEQ ID NO: 260) |
| agtgaaagagaagaaaatctcttttggctcatctctttttactcctacacacacac | |
| acacacacacacacacacacacacacacactctatatgatagattataacagatgt | |
| atctttcaaaagtagaactgaaatttagacctaaaagataatatactttaattgtt | |
| agagaggatatttttcctgttgaagggaacaatattcctatgtgtttaatacacaa | |
| atatatctgtgccAGTACTTGTTACCCCCTGAGACTTCACACACTACTTATATCTC | |
| TGGCACTGGTCTTTGA | |
| >DG8S250, chr8, pos 12427095 in NCBI build 33 | |
| Primer pair: | |
| F: TCCATCCCAACTCAAGATCC | (SEQ ID NO: 261) |
| R: AGCCTGGTCTCTACCATAAGC | (SEQ ID NO: 262) |
| length: 405 | |
| Amplimer: | |
| TCCATCCCAACTCAAGATCCCAggtaacaataatacctgcttcttgatataaggat | (SEQ ID NO: 263) |
| tcaacaattttttaaagcgctgagaccatgcctgttacatagtaggcacttaacac | |
| acgctgattatttacatctaaatcttcacaaccaccctaagaagtacatgttatta | |
| ttcccatcttacaatagagaaaataagctcagattaattaattttcttgggtctta | |
| cagcaagtaagtgatggtactggtatctgtacttatattgaatggtttgactgtaa | |
| aattcttcttttctctatatcaaatagtcccACGAGGAAtgtgtgtgtgtgtgtgt | |
| gtgtgtgtgtgtgtgtgtgtgtgtATTTTAAATGAGAACCAAGCAAAAGCTTATGG | |
| TAGAGACCAGGCT | |
| >AF188029-1, chr8, pos 12517357 in NCBI build 33 | |
| Primer pair: | |
| F: TCCTTGCAAATGTCTCTTTCTTC | (SEQ ID NO: 264) |
| R: ATGGGAAGGAATTTGGGACT | (SEQ ID NO: 265) |
| length: 171 | |
| Amplimer: | |
| TCCTTGCAAATGTCTCTTTCTTCCCCCTGGTACCATACCCCTGTATCTCTTAAGAC | (SEQ ID NO: 266) |
| AacacacacacacacacacacacacacacacacaTTCTCTCCCTCTCTCACTCCCT | |
| ACTTTTTTCCTTCCCACTGAGAGATTCAAACCTTCAAAAAGTCCCAAATTCCTTCC | |
| CAT | |
| >AF188029-7, chr8, pos 12558445 in NCBI build 33 | |
| Primer pair: | |
| F: CACCATTCTGTCGGCTGTAA | (SEQ ID NO: 267) |
| R: AAAGGGCTTGGTAACTCCTC | (SEQ ID NO: 268) |
| length: 180 | |
| Amplimer: | |
| Caccattctgtcggctgtaaaagcacggcaccagcatctgctcggcttcttgtgag | (SEQ ID NO: 269) |
| gcctcaggaagcttttactcatggttgaaggtgaatgcagagcaggtatatcacat | |
| ggtgagagggggagtgagagagagagagagagagagagagagagagaggaggagtt | |
| accaagcccttt | |
| >AF188029-10, chr8, pos 12572944 in NCBI build 33 | |
| Primer pair: | |
| F: CACGACCACACCAGCCTAAT | (SEQ ID NO: 270) |
| R: AAAGGCAGGCAGGCACAG | (SEQ ID NO: 271) |
| length: 195 | |
| Amplimer: cacgaccacaccagcctaattttgtgtgtacgtgtgtgtgtgtgtgt | (SEQ ID NO: 272) |
| gtgtgtgtgtgtgtgtgttttggtagaggcagagtttcactatgttgcccaggctg | |
| gtcttgaactcctgggctcaagtgatctgccccacctcggcctcccgaagtgctgg | |
| gattacaggtgtgagcctctgtgcctgcctgccttt | |
| >AF188029-12, chr8, pos 12583159 in NCBI build 33 | |
| Primer pair: | |
| F: GAATGGAAGCAAGGATGAGC | (SEQ ID NO: 273) |
| R: GACGCTGGTCTATTTCAGGTG | (SEQ ID NO: 274) |
| length: 304 | |
| Amplimer: | |
| GAATGGAAGCAAGGATGAGCTGCTGCATTTCTGTAGCTGGCATTCAGCTCAAGAAT | (SEQ ID NO: 275) |
| ACGTAAAACCAGACTCGTGGttttttctttctttctttctttctttctttTTGAAT | |
| GTGAGGCCTTTACAGAAAAAGAAAATGTCAGTCTGATTATCCAGGGCATGAGGATA | |
| AAGAGAAGCCCAAACAAAGGTTTCCCCCACTCCACCCCACCCAATATACTGTGGCA | |
| CTAGAAAACGATTCCAGAATCAGAAACTATATGCTGACGTCCATTAGCCCTCTTAG | |
| TAGCACCTGAAATAGACCAGCGTC | |
| >DG8S301, chr8, pos 12612075 in NCBI build 33 | |
| Primer pair: | |
| F: CAATCAAGCCTGTGTCGAGT | (SEQ ID NO: 276) |
| R: AGGAAGGCATTTGAATGAGC | (SEQ ID NO: 277) |
| length: 169 | |
| Amplimer: | |
| CAATCAAGCCTGTGTCGAGTTAAGAATTAAATGggaggttgcagtgagccaatatc | (SEQ ID NO: 278) |
| atgccactgcactccaggctgggcgacaggataagactccatctcaaaataaaaaa | |
| aataaaaaaataaaGGTTTGTATTTCTTTTTTCTTAAGCTCATTCAAATGCCTTCCT | |
| >DG8S308, chr8, pos 12617557 in NCBI build 33 | |
| Primer pair: | |
| F: GGATGGCCTTTGGTAACTGA | (SEQ ID NO: 279) |
| R: GGAAATGAACATGATAACATCTGG | (SEQ ID NO: 280) |
| length: 175 | |
| Amplimer: | |
| GGATGGCCTTTGGTAACTGATCTCATGACCAATATTAAGCTGTGAGCTCTCTTTTC | (SEQ ID NO: 281) |
| CGAATTTTTACATTATCCTCTTACAACCACCTCCCTCAacacacacacacacacac | |
| acacacacacacacacacacTCTCTCTCACACTCCCCACCCAGATGTTATCATGTT | |
| CATTTCC | |
| >DG8S188, chr8, pos 12654843 in NCBI build 33 | |
| Primer pair: | |
| F: CCATTTACGCTTTGGTCTGC | (SEQ ID NO: 282) |
| R: CCCTTTGTCAAGTGCTTTCA | (SEQ ID NO: 283) |
| length: 102 | |
| Amplimer: | |
| CCATTTACGCTTTGGTCTGCAGAGACTATTAATTATTTGGTTGTTTTTGTTTTCAT | (SEQ ID NO: 284) |
| GTTTGAATAAGCACAGATTCTGGCATTGAAAGCACTTGACAAAGGG | |
| >DG8S245, chr8, pos 12665541 in NCBI build 33 | |
| Primer pair: | |
| F: TTCCGAGGTAAGCCTTTGTG | (SEQ ID NO: 285) |
| R: ACCCTCTTTCAGAGCCAGGT | (SEQ ID NO: 286) |
| length: 307 | |
| Amplimer: | |
| TTCCGAGGTAAGCCTTTGTGGCCCCTGACCCTAATACAGAAGAGACACTAATTTAT | (SEQ ID NO: 287) |
| TTTCCTGCTCTGTGGTCCCAGAGTTATGTGAATTTCCTTTTGAAATTCATCATGCA | |
| tatttatttatttatttatttatttatttatttaAGCATATttctctatcagagta | |
| tacctgtcaccatggcagggatttgtctgcctctttctctttcactgaagtaccca | |
| cagtacccggcatagtgctggcgctgttcagggtgcccggtaaacttgtgtgaatg | |
| aatTTTTACCTGGCTCTGAAAGAGGGT | |
| >DG8S192, chr8, pos 12759031 in NCBI build 33 | |
| Primer pair: | |
| F: AATCGCTGCTACAGGGACAC | (SEQ ID NO: 288) |
| R: AACTGCATAAATATTTGACGTGGA | (SEQ ID NO: 289) |
| length: 113 | |
| Amplimer: | |
| AATCGCTGCTACAGGGACACACATATCTCTCTATCcatacacacacacacacacac | (SEQ ID NO: 290) |
| acacacacacacacGTGTACGTATTTCTAGTATTCCACGTCAAATATTTATGCAGTT | |
| >DG8S132, chr8, pos 11305452 in NCBI build 33 | |
| Primer pair: | |
| F: GTCCAGGCTCACCTGAAGTC | (SEQ ID NO: 291) |
| R: CGGAGGGAGCTAGGAACAG | (SEQ ID NO: 292) |
| length: 138 | |
| Amplimer: | |
| GTCCAGGCTCACCTGAAGTCTGAGATTTTGGGAGCTTTGGAGAATTCTGG | (SEQ ID NO: 293) |
| ATAAAATCCCTTACTGGACTTAGCAGGAATCTCCGATCTGTGGAGAAGT | |
| CTCCTCNAGAGACTGAGCATCTGTTCCTAGCTCCCTCCG | |

### Example 5

Characterization of the Effect of Inv8p on SNRI or SSRI Response

Drugs Included:

Effexor® (Venlafaxine)—SNRI (see below)
Tingus®, Serol®, Fontex®, Prozac® (fluoxetine)
Cipralex® (escitalopram)
Cipramil®, Oropram® (Citalopram)

Depressive Illness is among the most common and destructive of illnesses prevalent in the United States today and according to WHO statistics; major depression is the leading cause of disability worldwide (Murray C., Lopez A., eds. Summary: The global burden of disease: a comprehensive assessment of mortality and disability from diseases, injuries, and risk factors in 1990 and projected to 2020. Harvard University Press, 1996. Cambridge, Mass.). Depressive disorders affect an estimated 9.5 percent of adult Americans ages 18 and over in a given year, or about 18.8 million people in 1998. An estimated 35-40 million Americans living today will suffer from major depressive illness during their lives. For each person directly suffering, three or four times that number of their relatives, employees, associates, and friends will also be adversely affected. Of those 35-40 million afflicted, a substantial percentage will commit suicide if not treated with appropriate medication.

Standard criteria for depression include an abnormal sense of sadness and despair, disordered eating and weight control, diminished sexual interest and abnormal sleeping patterns. Furthermore, depression can be classified as exogenous or endogenous, major or minor, and unipolar or dipolar depending on its time course, severity, and cyclicity (if present). In addition to major depression, many people suffer from manic-depressive illness (bipolar disorder; BPD) that is characterized by radical mood swings from severe depression to exaggerated, inappropriate elation. Evidence from twin studies suggests that many depressive illnesses demonstrate a genetic disposition although a precise etiology remains undefined. However, all major theories of depression address neurophysiological mechanisms as part of the cause of depressive illness.

At the synapse or junction between nerve cells, neurotransmitters such as serotonin are released producing either excitatory or inhibitory input to the nerve cell's neighbor. The activity of neurotransmitters and neurotransmission is modulated through a variety of mechanisms including the synthesis and release of neurotransmitter(s), the catalytic breakdown of a neurotransmitter following its release, the reuptake of the neurotransmitter by the nerve cell that released it or by its surrounding cells, and the diffusion of the neurotransmitter out of the synapse. Most antidepressant medications decrease the uptake of a neurotransmitter (e.g., fluoxetine or Prozac® and the tricyclic antidepressants), decrease the catalytic breakdown of neurotransmitters (monoamine oxidase inhibitors; MAO inhibitors), and/or regulate the synthesis of neurotransmitter. Nearly all standard allopathic pharmacological treatments influence catecholaminergic and serotonergic neurotransmission suggesting that serotonin levels are important in giving an emotional timbre to thoughts and perception.

The selective serotonin reuptake inhibitor (SSRI) category of antidepressants is one of the newest and most widely used class of antidepressants in the United States. The SSRI class includes block-buster drugs such as Zoloft® (Sertraline), Prozac® (fluoxetin) and Paxil® (paroxetine). They have been found to be the most effective class of antidepressants in use. They are also widely used for treating anxiety disorders.

The serotonergic system seems to be the most heavily implicated system based on the clinical success of SSRIs, as well as findings in clinically depressed patients of decreased levels of serotonin metabolites in cerebrospinal fluid, decreased plasma tryptophan levels (the amino acid precursor to serotonin), and abnormalities in serotonin transport in platelets. Thus, production of serotonin (or 5-hydroxytryptamine) appears to be central for maintaining a positive affect as well as regulating some drives such as satiety, sexual interest, and the sleep-wake cycle. These findings are subsumed under the biogenic amine hypothesis of depression, which implicates a deficiency in the regulation of serotonin and possibly norpinephrine as the biochemical etiology for most clinical unipolar depression.

To study of the effect of Inv8p and marker associations to drug response DNA from selected individuals under recruitment for another study of the effects of gene expression on drug response were studied with markers from the inversion region.

Subject and Patient Criteria

1. Inclusion Criteria

The criteria the subject had to meet to enter the study, included the following:
a. Anxiety disorders or depression diagnosed by a CIDI interview as part of the recruitment to a genetic study of anxiety and depression, the diagnoses are based on ICD-10 and the DSM-III-R systems.
b. Patients with mild, moderate or severe disease who are taking antidepressants, serotonin-norepinephrine reuptake inhibitor (“SNRI”) or SSRI, were recruited to participate as blood donors for the study. The Drug-Response Phenotypes were determined in an interview resulting in the rating of the effect of the drug in question on the symptoms of anxiety and/or depression (1=very good, 2=rather good, 3=rather small, 4=very small, 5=none, 6=negative).
C. Age 12-70 years
e. Both males and females
f. White Caucasian (Icelandic patient population)
g. Regular use of the drug for more than 8 weeks.
h. Response to SSRI and SNRI: Patients are categorized as very good responders (1, above), responders (1,2 above) or non-responders (3,4,5,6 above). SSRI response is defined by any of the following:
i) improved control of depression/anxiety symptoms and/or fewer episodes of exacerbation/attacks when taking the drug
ii) improved quality of life/well being as judged by the patient response to a standard questionnaire, on SSRI therapy
iii) SSRI non-responders experience little or no improvement in the above measures
   2. Exclusion Criteria for Expression Study
   A precise list of criteria that excluded subjects from entering the study included:
a. Therapies that could interfere with evaluation of efficacy or the incidence of adverse effects, including:
Other investigational drugs
Concurrent medication
b. Diseases or conditions that could interfere with the evaluation of efficacy or the incidence of adverse effects, including:
Pregnancy or lactation
Hypersensitivity or serious adverse experiences to anti-depression drugs in the past
c. Sensitivity to the study drug or its components
d. If compliance to medication was questionable

Summary of Study Design

Patients with anxiety or depression diagnosed by the above criteria (see inclusion criteria) who are taking SNRI or SSRI drugs were invited to participate in the study. One hundred and fifty patients diagnosed with anxiety disorders and depression were selected to study the effect of the inversion, approximately 60% of whom are responders and one-third very good responders, as judged by the clinical criteria described above. Patients were allowed to use sleeping pills if taking them on a regular basis. The dose of SNRI/SSRI was kept stable for at least 4 weeks prior to blood donation for a study of gene expression. A single physician, who was blinded to the expression array studies and genotyping results, phenotyped all patients. All patients participating signed an informed consent authorizing his/her participation in the study.

Treatment

a. Treatment Plan

All patients recruited had been treated with SNRI/SSRI drugs for minimum of 8 weeks and were examined by the study psychiatrist.

b. Diet/Activity/Other

No diet restrictions were implemented.

Safety Measurements

Patients were already taking SSRI as recommended by their clinicians. All blood examined were encrypted (×3) by a third party (Icelandic Data Protection Committee governed by the Icelandic Government) to allow for complete protection of patients privacy.

Markers for Responsiveness

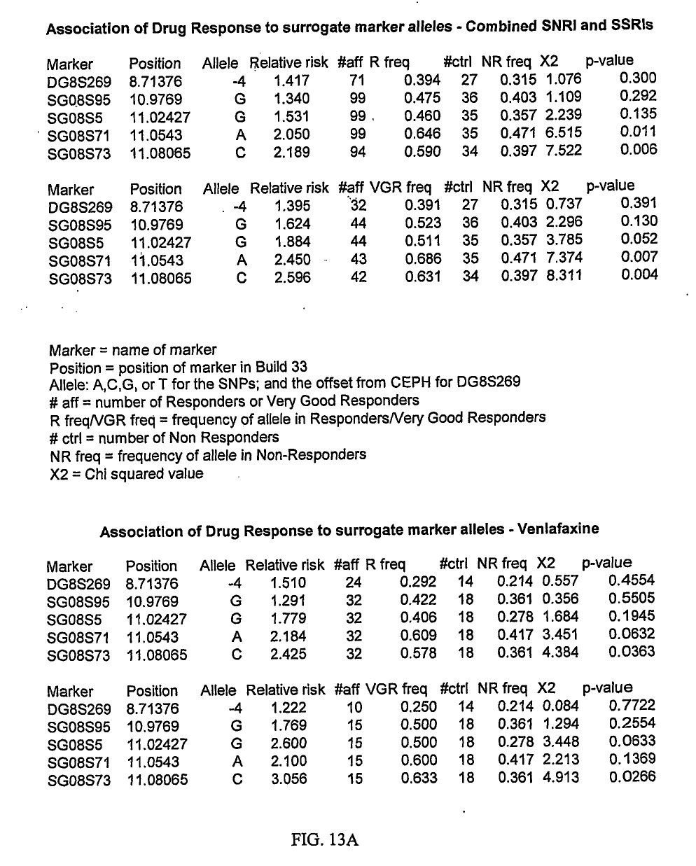

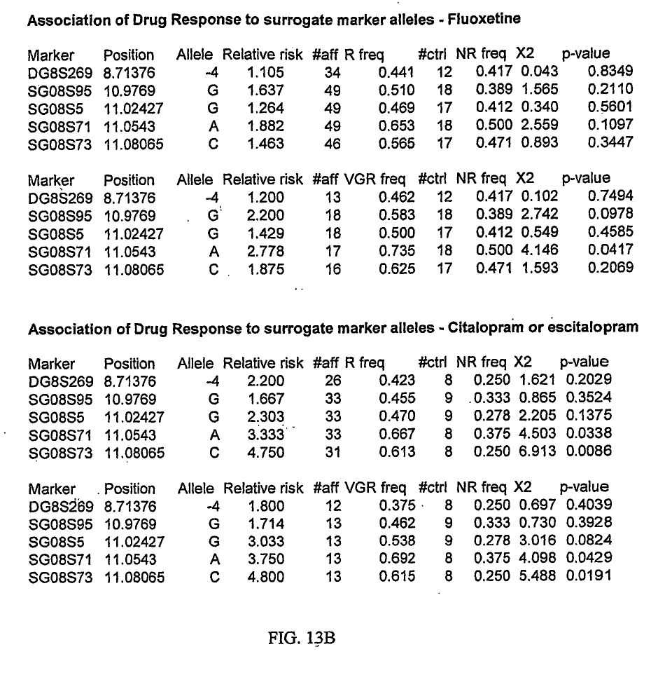

Markers were identified that were associated with responsiveness and non-responsiveness to drugs (see FIGS. 13A and 13B). The trends in association shown in FIGS. 13A and 13B clearly indicate that the marker alleles are clearly associated with responsiveness, since there is a trend on the association that parallels responsiveness (e.g., going from non-responders to responders to very good responders).

### Example 6

Yet another marker was examined that was found to be in LD with Inv8p23, which has two alleles 0, 7. The association of this marker, DG8S132 was studied in a group of Icelandic populations and controls and found it to be in LD with SG08S5 (r²=0.437). As DG8S132 is in LD with a marker that is in LD with the Inv8p23 genomic region, DG8S 132 is likely to correlate with the inversion. When tested by genotyping 58 Hz rare and 128 Hz common individuals, the frequency of the 0 allele is 93% in the rare form of the inversion and 6% on the common form of the inversion. This marker is thus an excellent surrogate marker for the inversion, and other markers can be identified in a similar manner.

While this invention has been particularly shown and described with references to preferred embodiments thereof, it will be understood by those skilled in the art that various changes in form and details may be made therein without departing from the scope of the invention encompassed by the appended claims.

What is claimed is:

1-55. (canceled)

56. A method of diagnosing a psychiatric disorder or a comorbid disorder in an individual comprising detecting the orientation of the Inv8p23 genomic region, wherein the orientation of the Inv8p23 genomic region is indicative of a psychiatric disorder.

57. The method of claim 56, wherein the psychiatric disorder is an anxiety disorder.

58. The method of claim 57, wherein the anxiety disorder is panic disorder or bipolar disorder.

59. The method of claim 58, wherein the inverted orientation of the Inv8p23 genomic region is indicative of panic disorder.

60. The method of claim 56, wherein the comorbid disorder is selected from the group consisting of: depression, bipolar disorder, obsessive-compulsive disorder, histrionic personality disorder, family denial and dysfunction, hypercholesterolemia and substance abuse.

61. The method of claim 60, wherein the comorbid disorder is selected from the group consisting of: depression, bipolar disorder and hypercholesterolemia.

62. The method of claim 56, wherein the orientation of Inv8p23 is determined by detecting one or more markers at one or more polymorphic sites, wherein the one or more polymorphic sites are in linkage disequilibrium with Inv8p23, and wherein a particular allele at the one or more polymorphic sites is indicative of a particular orientation of Inv8p23.

63. The method of claim 62, wherein the one or more markers are selected from the group consisting of: SG08S5, SG08S95, DG8S269, DG8S163, DG8S197, AF131215-2, DG8S127, SG08S120, DG8S179, SG08S27, DG8S261, SG08S71, SG08S32, SG08S517, SG08S70, SG08S102, SG08S73, SG08S76, SG08S26, DG8S242, SG08S15, DG8S257, SG08S138, DG8S161, SG08S520, DG00AAHBG, SG08S508, DG8S156, D8S1695 and DG8S170.

64. The method of claim 63, wherein the one or more markers comprise the A allele for SG08S71 and the G allele for DG00AAHBG.

65. The method of claim 62, wherein the inverted allele of Inv8p23 is detected by detecting a haplotype comprising one or more genetic markers.

66. The method of claim 65, wherein one or more genetic markers of the haplotype are selected from the group consisting of: SG08S5, SG08S95, DG8S269, DG8S163, DG8S197, AF131215-2, DG8S127, SG08S120, DG8S179, SG08S27, DG8S261, SG08S71, SG08S32, SG08S517, SG08S70, SG08S102, SG08S73, SG08S76, SG08S26, DG8S242, SG08S15, DG8S257, SG08S138, DG8S161, SG08S520, DG00AAHBG, SG08S508, DG8S156, D8S1695 and DG8S170.

67. The method of claim 66, wherein the haplotype comprises the A allele for SG08S71 and the G allele for DG00AAHBG.

68. The method of claim 62, wherein the one or more surrogate markers comprise a marker in linkage disequilibrium with one or more markers selected from the group consisting of: SG08S5, SG08S95, DG8S269, DG8S163, DG8S197, AF131215-2, DG8S127, SG08S120, DG8S179, SG08S27, DG8S261, SG08S71, SG08S32, SG08S517, SG08S70, SG08S102, SG08S73, SG08S76, SG08S26, DG8S242, SG08S15, DG8S257, SG08S138, DG8S161, SG08S520, DG00AAHBG, SG08S508, DG8S156, D8S1695 and DG8S170.

69. The method of claim 68, wherein the one or more surrogate markers comprise DG8S132.

70. A kit for diagnosing a psychiatric disorder or a comorbid disorder comprising at least one agent useful for detecting the orientation of the Inv8p23 genomic region, wherein the orientation of the Inv8p23 genomic region is indicative of the psychiatric disorder.

71. The kit of claim 70, wherein the orientation of Inv8p23 is determined by detecting one or more markers at one or more polymorphic sites, wherein one or more markers is selected from the group consisting of the markers listed in FIGS. 6A-6K.

72. The kit of claim 70, wherein bipolar disorder occurs without panic disorder.

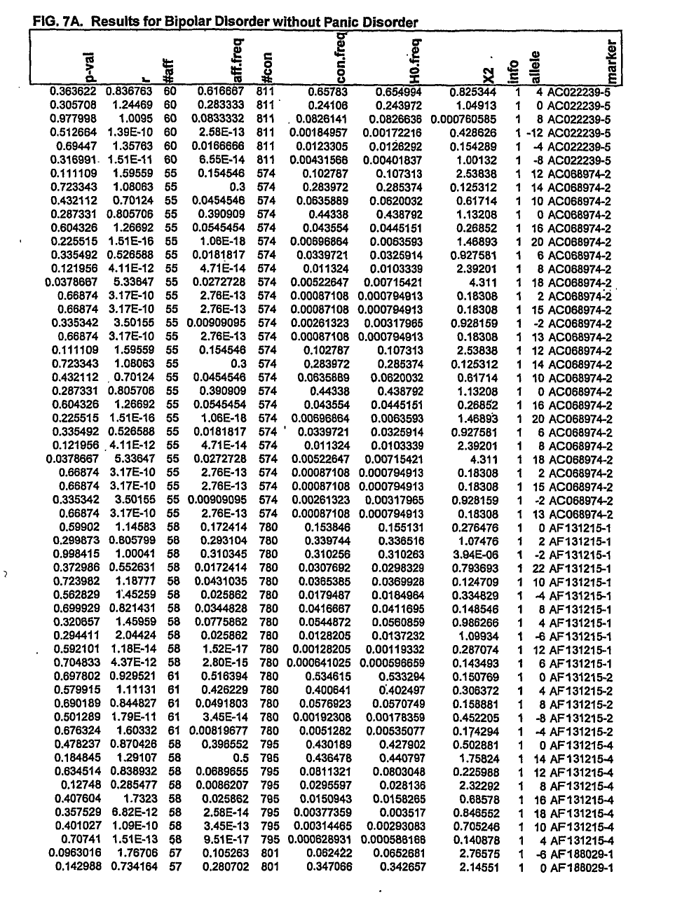

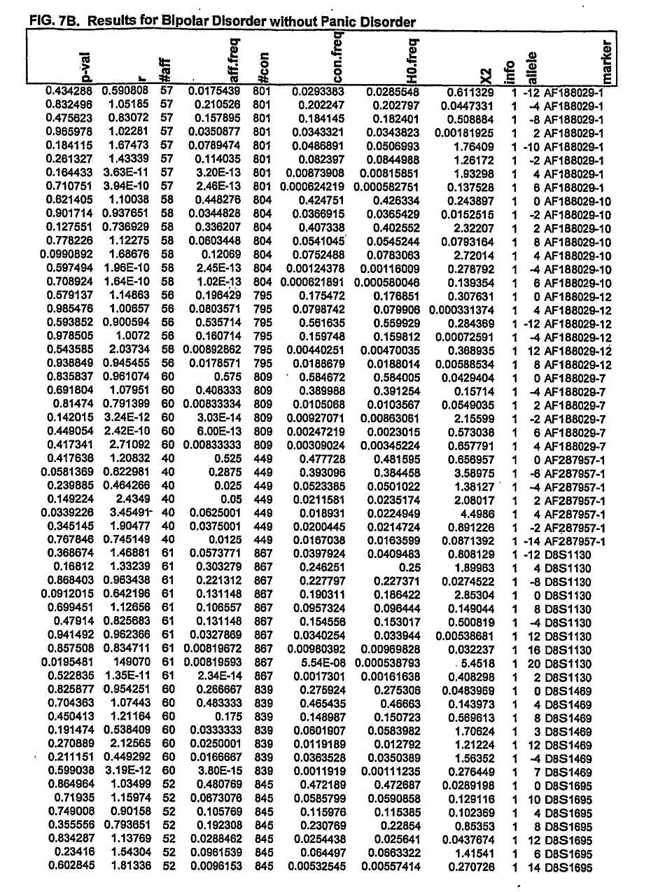

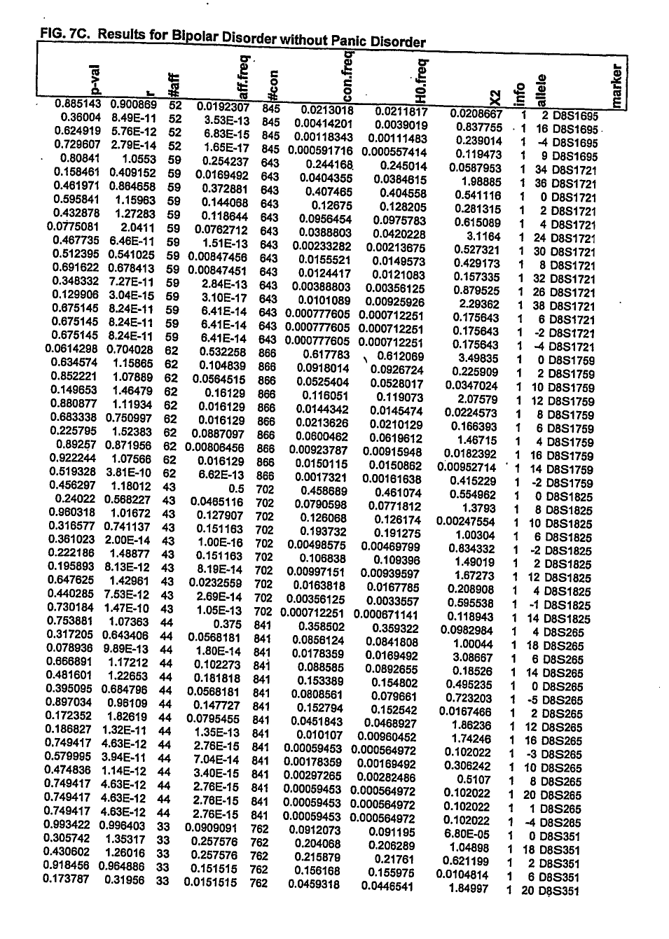

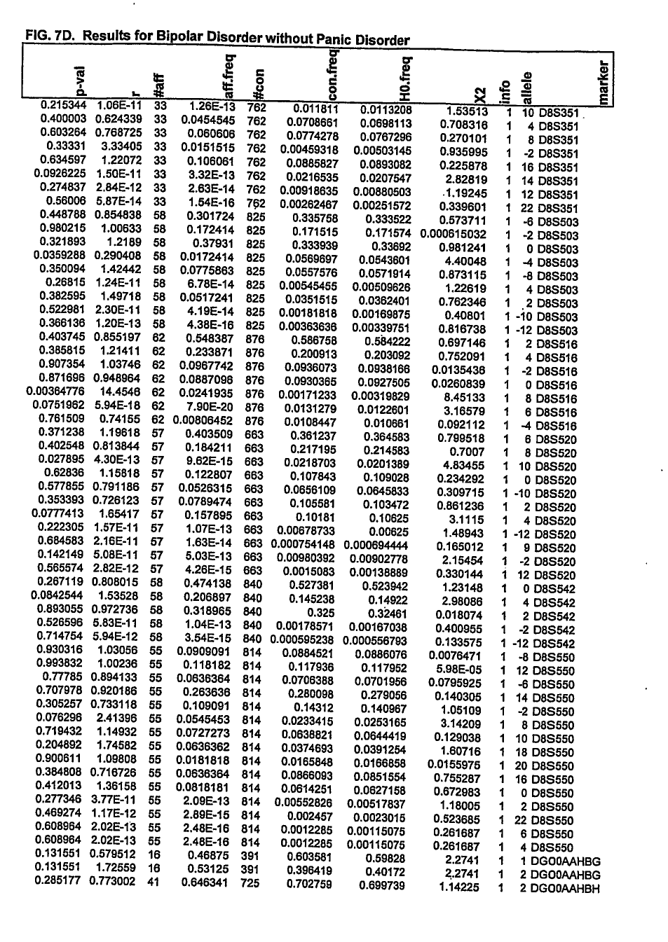

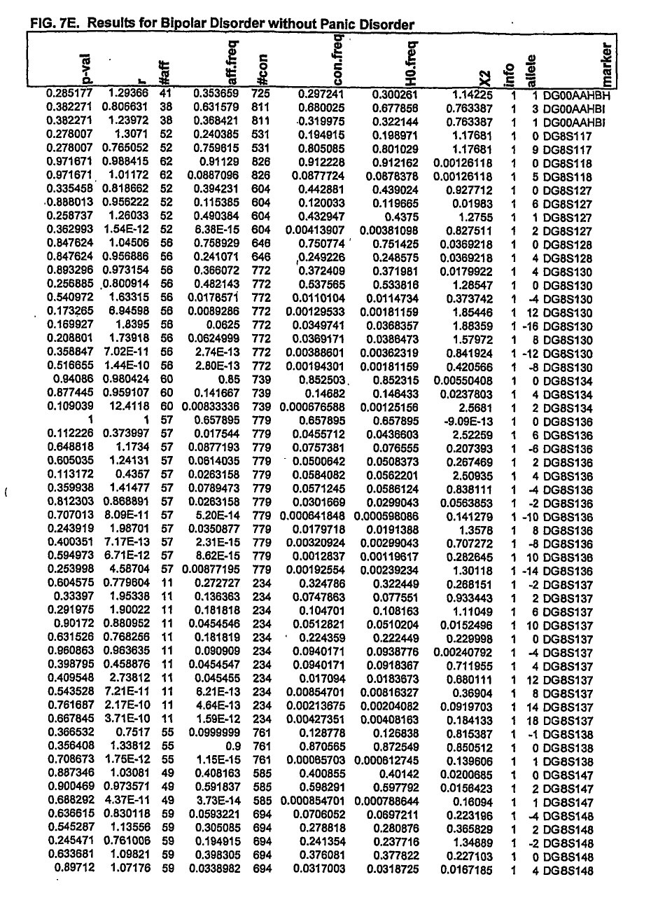

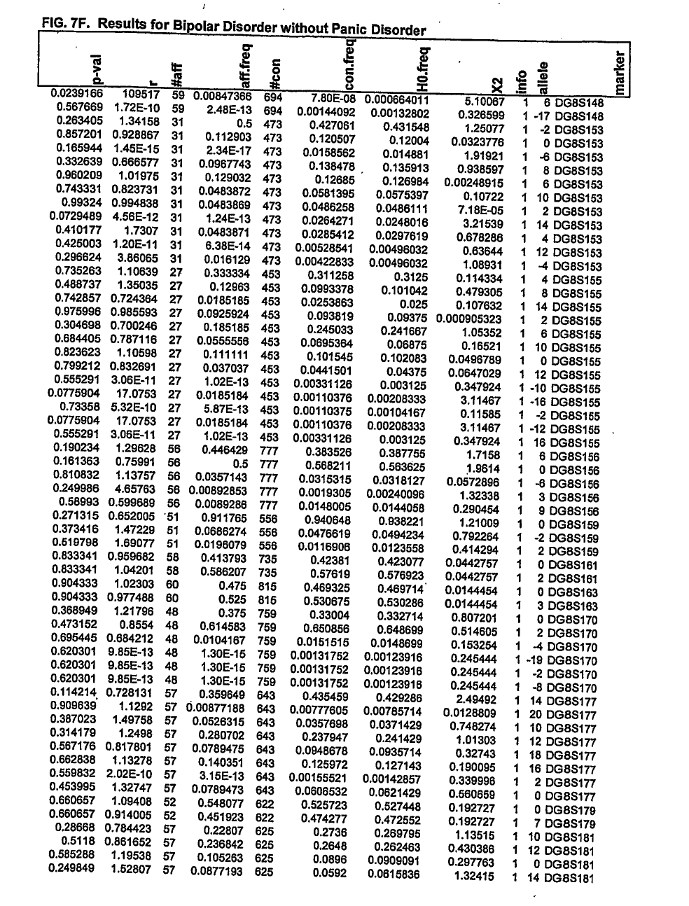

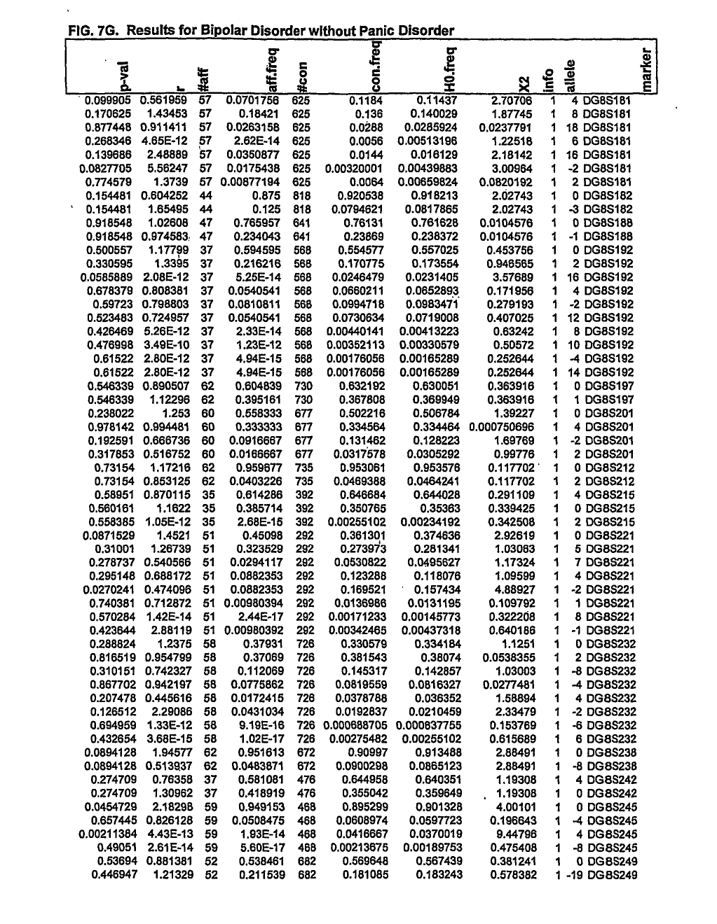

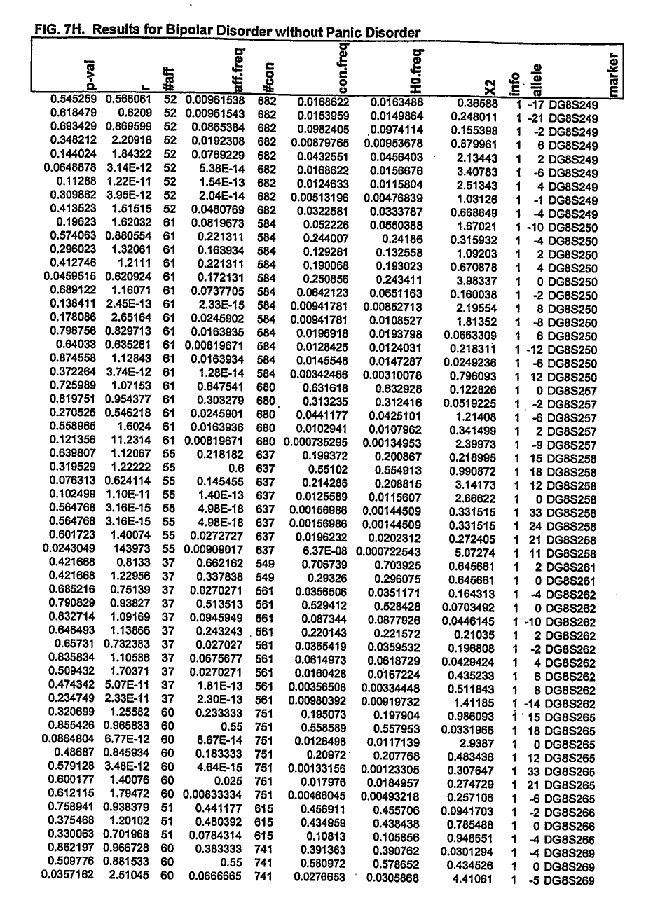

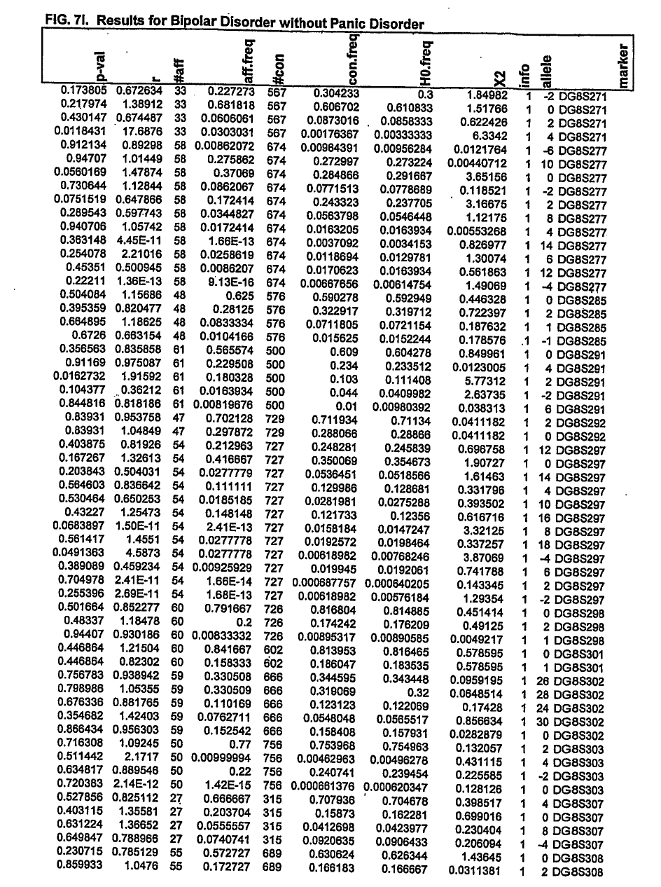

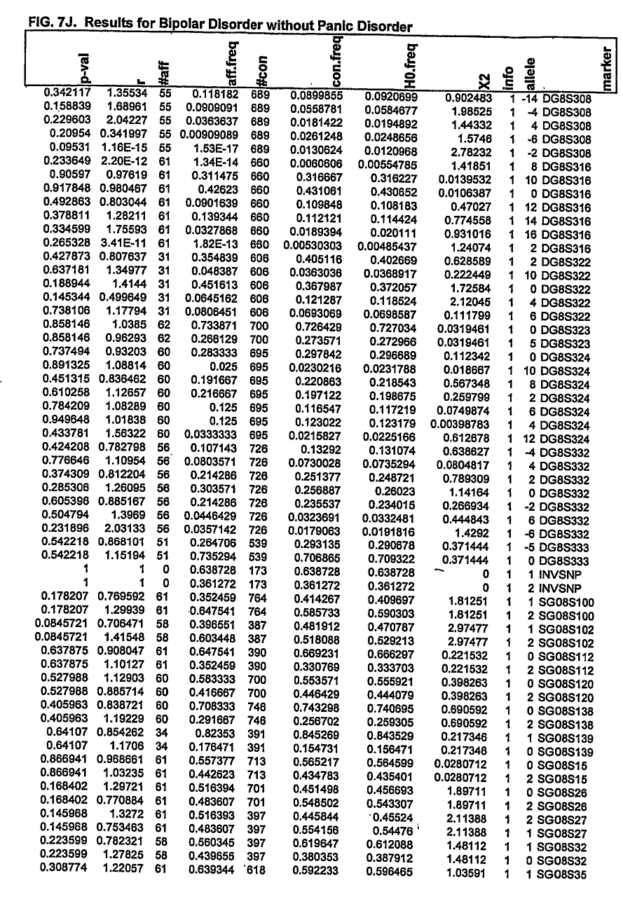

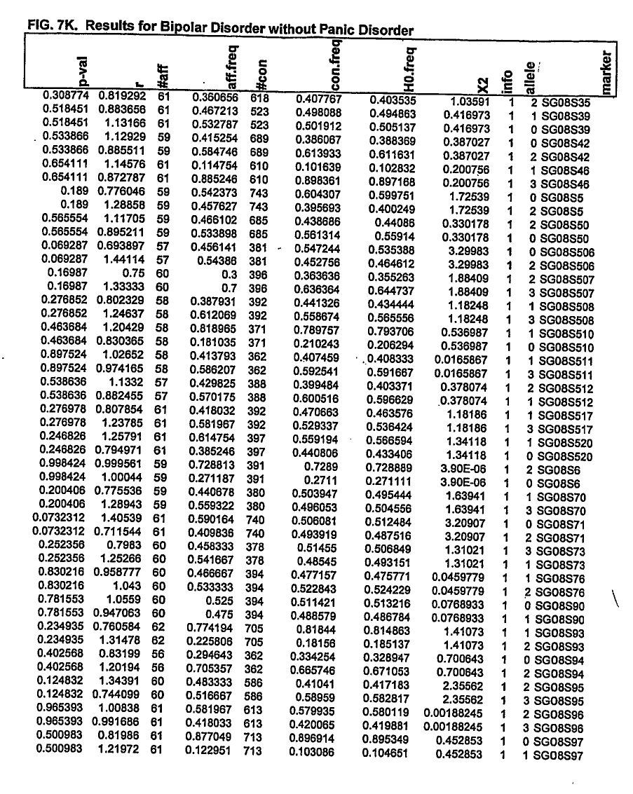

73. The kit of claim 72, wherein one or more markers are selected from the group consisting of the markers listed in FIGS. 7A-7K.

74. A method for predicting the efficacy of a drug for treating a psychiatric disorder or a comorbid disorder in a human patient, comprising determining the orientation of the Inv8p23 genomic region, wherein the orientation of the Inv8p23 genomic region is indicative of responsiveness or non-responsiveness to the drug in the human patient.

75. The method of claim 74, wherein the drug is selected from the group consisting of: amine reuptake inhibitors, selective serotonin reuptake inhibitors, selective norepinephrine reuptake inhibitors, combined serotonin-norepinephrine reuptake inhibitors, combined dopamine-norepinephrine reuptake inhibitors, monoamine oxidase inhibitors, reversible/selective inhibitors of monoamine oxidase-A; 5-HT 2A receptor antagonists, combined 5-HT 2A antagonists with serotonin reuptake inhibition, tricyclic drugs, and combined 5-HT 2A, 5-HT 2C and alpha-2 antagonism.

76. The method of claim 75, wherein the drug is a selective serotonin reuptake inhibitor.

77. The method of claim 74, wherein the drug is selected from the group consisting of: venlafaxine, sertraline, paroxat, fluoxetine, escitalopram and citalopram.

78. The method of claim 74, wherein the psychiatric disorder is anxiety disorder or depression.

79. The method of claim 78, wherein the anxiety disorder is panic disorder or bipolar disorder.

80. The method of claim 74, wherein the orientation of Inv8p23 is determined by detecting one or more markers at one or more polymorphic sites wherein the one or more polymorphic sites are in linkage disequilibrium with the Inv8p23 genomic region and wherein the one or more markers are indicative of the orientation of the Inv8p23 genomic region.

81. The method of claim 80, wherein the one or more markers are selected from the group consisting of: DG8S269, SG08S95, SG08S5, SG08S71 and SG08S73.

82. The method of claim 80, wherein the drug is selected from the group consisting of: venlafaxine, fluoxetine and Citalopram.
